SEQUENCE LISTING (209 sequences)

SEQ ID NO: 1 - protein, 832 aa, Synthetic construct
RGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 2 - protein, 31 aa, Synthetic construct
RGMLPLFEPKGRVLLVDGHHLAYRTFHALKG

SEQ ID NO: 3 - protein, 25 aa, Synthetic construct
LTTSRGEPVQAVYGFAKSLLKALKE

SEQ ID NO: 4 - protein, 11 aa, Synthetic construct
DGDAVIVVFDA

SEQ ID NO: 5 - protein, 16 aa, Synthetic construct
KAPSFRHEAYGGYKAG

SEQ ID NO: 6 - protein, 185 aa, Synthetic construct
RAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERL

SEQ ID NO: 7 - protein, 6 aa, Synthetic construct
RAFLER

SEQ ID NO: 8 - protein, 110 aa, Synthetic construct
LEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNT

SEQ ID NO: 9 - protein, 16 aa, Synthetic construct
TPEGVARRYGGEWTEE

SEQ ID NO: 10 - protein, 78 aa, Synthetic construct
AGERAALSERLFANLWGRLEGEERLLWLYREVERPLSAVLAHMEATGVRLDVAYLRALSLEVAEEIARLEAEVFRLAG

SEQ ID NO: 11 - protein, 6 aa, Synthetic construct
HPFNLN

SEQ ID NO: 12 - protein, 348 aa, Synthetic construct
SRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 13 - protein, 234 aa, Synthetic construct
RGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAH

SEQ ID NO: 14 - protein, 478 aa, Synthetic construct
RGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAG

SEQ ID NO: 15 - protein, 76 aa, Synthetic construct
GSWYQPKGGTEMFCHPRTGKPLPKYPRIKIPKVGGIFKKPKNKAQREGREPCELDTREYVAGAPYTPVEHVVFNPS

SEQ ID NO: 16 - protein, 108 aa, Synthetic construct
SDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 17 - protein, 108 aa, Synthetic construct
SDKIIHLTDDSFDTDVLKADGAILVDFWAEWCGPSKMIAPILDEIADEYQGKLTVAKLNIDQNPGTAPKYGIRGIPTLLLFKNGEVAATKVGALSKGQLKEFLDANLA

SEQ ID NO: 18 - protein, 67 aa, Synthetic construct
DTDMGLLRSGKLPGKRFGSHALEAWGYRLGEMKGEYKDDFKRMLEEQGEEYVDGMEWWNFNEEMMDY

SEQ ID NO: 19 - protein, 31 aa, Synthetic construct
AWGYRLGEMKGEYKDDFKRMLEEQGEEYVDG

SEQ ID NO: 20 - protein, 25 aa, Synthetic construct
GEMKGEYKDDFKRMLEEQGEEYVDG

SEQ ID NO: 21 - protein, 18 aa, Synthetic construct
EYKDDFKRMLEEQGEEYV

SEQ ID NO: 22 - protein, 1328 aa, Synthetic construct
XXXXXXXXXXSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKXXXXXXXXXX

SEQ ID NO: 23 - protein, 1071 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGGGGSGGGSGSSGGSGSSGGSSGGGGSGGGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 24 - protein, 1118 aa, Synthetic construct
XXXXXXXXXXRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAXXXXXXXXXX

SEQ ID NO: 25 - protein, 1051 aa, Synthetic construct
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGDGGSSGSGGGSSGGSGSSGGGGSGGGSGSGSSGSGSSGSSGSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 26 - protein, 1335 aa, Synthetic construct
XXXXXXXXXXSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAXXXXXXXXXXXXXXXXXXXX

SEQ ID NO: 27 - protein, 1219 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGDGGSSGSGGGSSGGSGSSGGGGSGGGSGSGSSGSGSSGSSGSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 28 - protein, 920 aa, Synthetic construct
XXXXXXXXXXRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKXXXXXXXXXX

SEQ ID NO: 29 - protein, 911 aa, Synthetic construct
MKHHHHHHMRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 30 - protein, 911 aa, Synthetic construct; identical to SEQ ID NO: 29

SEQ ID NO: 31 - protein, 668 aa, Synthetic construct
MDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 32 - protein, 903 aa, Synthetic construct
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFSHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 33 - protein, 903 aa, Synthetic construct
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPSELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 34 - protein, 903 aa, Synthetic construct
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFSHPRTGKPLPKYPRIKIPKVGGIFKKPKNKAQREGREPSELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 35 - protein, 1071 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 36 - protein, 1071 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGGGGSGGGSGSSGGSGSSGGSSGGGGSGGGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKTPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 37 - protein, 1066 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 38 - protein, 1061 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 39 - protein, 1056 aa, Synthetic construct
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGDGGSSGSGGGSSGGSGSSGGGGSGGGSGSGSSGSGSSGGSSGGSSGSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 40 - protein, 1051 aa, Synthetic construct; identical to SEQ ID NO: 25

SEQ ID NO: 41 - protein, 1046 aa, Synthetic construct
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGDGGSSGSGGGSSGGSGSSGGGGSGGGSGSGSSGSSGSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 42 - protein, 1219 aa, Synthetic construct; identical to SEQ ID NO: 27

SEQ ID NO: 43 - protein, 1185 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGSGSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLASSGGGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 44 - protein, 1299 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGGGSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGSGSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLASSGGGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 45 - protein, 1122 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGGGGSGGGSGSSGGSGSSGGSSGGGGSGGGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTDTDMGLLRSGKLPGKRFGSHALEAWGYRLGEMKGEYKDDFKRMLEEQGEEYVDGMEWWNFNEEMMDYAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 46 - protein, 888 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGGGGSGGGSGSSGGSGSSGGSSGGGGSGGGSGSSGGSGSSGSGSSGSSGGGGSGGSSMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTDTDMGLLRSGKLPGKRFGSHALEAWGYRLGEMKGEYKDDFKRMLEEQGEEYVDGMEWWNFNEEMMDYAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 47 - protein, 1086 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGGGGSGGGSGSSGGSGSSGGSSGGGGSGGGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAAWGYRLGEMKGEYKDDFKRMLEEQGEEYVDGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 48 - protein, 1090 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGGGGSGGGSGSSGGSGSSGGSSGGGGSGGGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLGEMKGEYKDDFKRMLEEQGEEYVDGLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 49 - protein, 1064 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGGGGSGGGSGSSGGSGSSGGSSGGGGSGGGSGSSGGSGSSGSGSSGSSGGGGSGGSSRGMLPLFEPKGRVLLVDGHHLAYRTFHALKGEYKDDFKRMLEEQGEEYVDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 50 - protein, 903 aa, Synthetic construct
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 51 - protein, 109 aa, Synthetic construct
MKKIKTVKTDEELEKILEESNKYTIVLFGAEWCGPCKMFLQENGETLEKISKEKGFEVLLVNIDQNPGTAPKYGIRGIPTVVVFKNGKKIATKVGALSKGQLLEFADAV

SEQ ID NO: 52 - protein, 109 aa, Synthetic construct
MVKKVTEEELEKLIEEAKKKGEKIMVIFSAEWCGPCKMLLKELEEIEDELKALGIKEVLELNIDQNPGTAPKYGIRGIPTIMFIDANGIVYTKVGALSKGQILELAKAV

SEQ ID NO: 53 - protein, 108 aa, Synthetic construct
MIKEVNGEELDKIIKEESPKRKIIIDFGAEWCGPCKMLKAELEKIAKELEEKYGYDIYLLNIDQNPGTAPKYGIRGIPTLIILTPSGKKLTKVGALSKGQILQLVKAA

SEQ ID NO: 54 - protein, 1101 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSGSSGGSGSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 55 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSGSGSSGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 56 - protein, 1093 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 57 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 58 - protein, 1011 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLARGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 59 - protein, 1016 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLASGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 60 - protein, 1021 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLASGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 61 - protein, 1026 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 62 - protein, 1031 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 63 - protein, 1036 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 64 - protein, 1041 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGSSGGSSGGGGSGSGSGSSGGSGSSGSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 65 - protein, 1046 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGSSGGSSGGGGSGSGSGSSGGSGSSGSSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 66 - protein, 1051 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGSSGGSSGGGGSGSGSGSSGGSGSSGSGSSGSSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 67 - protein, 1084 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGGSGSSGGSSGGGGSGSGSGSSGGSGSSGSGSSGSSGGGGSGGSSGSSGGSGSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 68 - protein, 1111 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSGSSSGGSSSGGSSGGSGSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 69 - protein, 1121 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGGSSGSGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 70 - protein, 1141 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGGSSSSGGSSGGGGSSGSGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 71 - protein, 1161 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGGSSSSGGSSGGGGSGSGSGSSGGSGSSGSGSSGSSGSGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 72 - protein, 1181 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGGSSSSGGSSGGGGSGSGSGSSGGSGSSGSGSSGSSGGGGSGGSSGSSGGSGSGSSGSGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 73 - protein, 1211 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGGSSSSGGSSGGGGSGSGSGSSGGSGSSGSGSSGSSGGGGSGGSSGSSGGSGSSSGSSGSSGGGSGGGSGSSGGSGSSGGSSGGSSGSGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 74 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGAEAAAKEAAAKEAAAKEAAAKALEAEAAAKEAAAKEAAAKEAAAKASGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 75 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGAEAAAKEAAAKEAAAKEAAAKALEAEAAAKEAAAKEAAAKEAAAKASGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAARLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 76 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLASLEPTTEDLYFQSDNDSGSSGSSGSSGGGSGGGSGSSGGSGSSGGSSGGGGSGSGSGSSGGSGSSGSLEPTTEDLYFQSDNDRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 77 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSTAAGAATAAGSTGAAGAATAAGGSAGGTGSGSATGSSGASGTGTAGGTGAGSGTGSGAAGAATAAGTSGAAGAATAATSGRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 78 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSGGCASSIDYKRISRMPSKIMDAVIDTLNICKLANCESGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 79 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSGGCASSIDYKRISRMPAVLADAVIDTLNICKLANCESGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 80 - protein, 1066 aa, Synthetic construct
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGDGGSSGGSSGGSGSSGSGGGSSGGSGSSGGGGSGGGSGSGSSGSGSSGGSSGGSSGSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 81 - protein, 1061 aa, Synthetic construct
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGDGGSSGSGSSGSGGGSSGGSGSSGGGGSGGGSGSGSSGSGSSGGSSGGSSGSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 82 - protein, 1056 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 83 - protein, 1041 aa, Synthetic construct
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGDGGSSGSGGGSSGGSGSSGGGGSGGSSGSSGSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 84 - protein, 1036 aa, Synthetic construct
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGDGGSSGSGGGSSGGSGSSGGSSGSSGSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 85 - protein, 1070 aa, Synthetic construct
MSEHILQVSDDSFETDVLKAEAPVLVDFWAEWCGPCKMIAPILDDVAAEYAGKVTVAKVNIDQNPNTPPKFGIRGIPTLLLFKNGQVAATKVGALSKTQLKQFLDSNIGSSGSGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 86 - protein, 1092 aa, Synthetic construct
MSEHILQVSDDSFETDVLKAEAPVLVDFWAEWCGPCKMIAPILDDVAAEYAGKVTVAKVNIDQNPNTPPKFGIRGIPTLLLFKNGQVAATKVGALSKTQLKQFLDSNIGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGTWYQPKGGTELFLHPRTGKPLGKYPRVKYPKQGGIYKKPKNKAQREGREPCDLDTRDYVEGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 87 - protein, 1092 aa, Synthetic construct
MSEHILQVSDDSFETDVLKAEAPVLVDFWAEWCGPCKMIAPILDDVAAEYAGKVTVAKVNIDQNPNTPPKFGIRGIPTLLLFKNGQVAATKVGALSKTQLKQFLDSNIGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYAPKGGKEFFRHPRTGKDLPKYPRVVYPKVGGIFKKPKNKAQRLGLEPCERDTRDTMEGAPFTPITYVEFNPGSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 88 - protein, 1081 aa, Synthetic construct
MSEHILQVSDDSFETDVLKAEAPVLVDFWAEWCGPCKMIAPILDDVAAEYAGKVTVAKVNIDQNPNTPPKFGIRGIPTLLLFKNGQVAATKVGALSKTQLKQFLDSNIGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGSSWYRPKGGKAFFRHPVTGKDLTNYPRVIYPKAGEIYTKGGKLAKTLYCKDRPFTPIEYTVFNPGSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 89 - protein, 1070 aa, Synthetic construct
MSDSIVHVTDDSFERDVLQSSEPVLVDYWADWCGPCKMIAPVLDEIATEYAGRIRVAKLNIDENPNTPPRYGIRGIPTLMLFKDGEVEATKVGAVSKSQLTAFIDSNLGSSGSGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 90 - protein, 1075 aa, Synthetic construct
MSDSIVHVTDDSFERDVLQSSEPVLVDYWADWCGPCKMIAPVLDEIATEYAGRIRVAKLNIDENPNTPPRYGIRGIPTLMLFKDGEVEATKVGAVSKSQLTAFIDSNLGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGTWYQPKGGTELFLHPRTGKPLGKYPRVKYPKQGGIYKKPKNKAQREGREPCDLDTRDYVEGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 91 - protein, 1075 aa, Synthetic construct
MSDSIVHVTDDSFERDVLQSSEPVLVDYWADWCGPCKMIAPVLDEIATEYAGRIRVAKLNIDENPNTPPRYGIRGIPTLMLFKDGEVEATKVGAVSKSQLTAFIDSNLGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYAPKGGKEFFRHPRTGKDLPKYPRVVYPKVGGIFKKPKNKAQRLGLEPCERDTRDTMEGAPFTPITYVEFNPGSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 92 - protein, 1081 aa, Synthetic construct
MSDSIVHVTDDSFERDVLQSSEPVLVDYWADWCGPCKMIAPVLDEIATEYAGRIRVAKLNIDENPNTPPRYGIRGIPTLMLFKDGEVEATKVGAVSKSQLTAFIDSNLGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGSSWYRPKGGKAFFRHPVTGKDLTNYPRVIYPKAGEIYTKGGKLAKTLYCKDRPFTPIEYTVFNPGSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 93 - protein, 108 aa, Synthetic construct
MSDSIVHVTDDSFERDVLQSSEPVLVDYWADWCGPCKMIAPVLDEIATEYAGRIRVAKLNIDENPNTPPRYGIRGIPTLMLFKDGEVEATKVGAVSKSQLTAFIDSNL

SEQ ID NO: 94 - protein, 108 aa, Synthetic construct
MSEHILQVSDDSFETDVLKAEAPVLVDFWAEWCGPCKMIAPILDDVAAEYAGKVTVAKVNIDQNPNTPPKFGIRGIPTLLLFKNGQVAATKVGALSKTQLKQFLDSNI

SEQ ID NO: 95 - protein, 1071 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGGGGSGGGSGSSGGSGSSGGSSGGGGSGGGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYAPKGGKEFFRHPRTGKDLPKYPRVVYPKVGGIFKKPKNKAQRLGLEPCERDTRDTMEGAPFTPITYVEFNPGSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 96 - protein, 1060 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGGGGSGGGSGSSGGSGSSGGSSGGGGSGGGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGSSWYRPKGGKAFFRHPVTGKDLTNYPRVIYPKAGEIYTKGGKLAKTLYCKDRPFTPIEYTVFNPGSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 97 - protein, 1071 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGGGGSGGGSGSSGGSGSSGGSSGGGGSGGGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGTWYQPKGGTELFLHPRTGKPLGKYPRVKYPKQGGIYKKPKNKAQREGREPCDLDTRDYVEGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 98 - protein, 903 aa, Synthetic construct
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYAPKGGKEFFRHPRTGKDLPKYPRVVYPKVGGIFKKPKNKAQRLGLEPCERDTRDTMEGAPFTPITYVEFNPGSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 99 - protein, 892 aa, Synthetic construct
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGSSWYRPKGGKAFFRHPVTGKDLTNYPRVIYPKAGEIYTKGGKLAKTLYCKDRPFTPIEYTVFNPGSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 100 - protein, 903 aa, Synthetic construct
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGTWYQPKGGTELFLHPRTGKPLGKYPRVKYPKQGGIYKKPKNKAQREGREPCDLDTRDYVEGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 101 - protein, 76 aa, Synthetic construct
GSWYAPKGGKEFFRHPRTGKDLPKYPRVVYPKVGGIFKKPKNKAQRLGLEPCERDTRDTMEGAPFTPITYVEFNPG

SEQ ID NO: 102 - protein, 65 aa, Synthetic construct
SSWYRPKGGKAFFRHPVTGKDLTNYPRVIYPKAGEIYTKGGKLAKTLYCKDRPFTPIEYTVFNPG

SEQ ID NO: 103 - protein, 76 aa, Synthetic construct
GTWYQPKGGTELFLHPRTGKPLGKYPRVKYPKQGGIYKKPKNKAQREGREPCDLDTRDYVEGAPYTPVEHVVFNPS

SEQ ID NO: 104 - protein, 610 aa, Synthetic construct
MKELQLYEEAEPTGYEIVKDHKTFEDLIEKLKEVPSFALALETSSLDPFNCEIVGISVSFKPKTAYYIPLHHRNAQNLDETLVLSKLKEILEDPSSKIVGQNLKYAYKVLMVKGISPVYPHFDTMIAAYLLEPNEKKFNLEDLSLKFLGYKMTSYQELMSFSSPLFGFSFADVPVDKAANYSCEDADITYRLYKILSMKLHEAELENVFYRIEMPLVNVLARMELNGVYVDTEFLKKLSEEYGKKLEELAEKIYQIAGEPFNINSPKQVSKILFEKLGIKPRGKTTKTGAYSTRIEVLEEIANEHEIVPLILEYRKIQKLKSTYIDTLPKLVNPKTGRIHASFHQTGTATGRLSSSDPNLQNLPTKSEEGKEIRKAIVPQDPDWWIVSADYSQIELRILAHLSGDENLVKAFEEGIDVHTLTASRIYNVKPEEVNEEMRRVGKMVNYSIIYGVTPYGLSVRLGIPVKEAEKMIISYFTLYPKVRSYIQQVVAEAKEKGYVRTLFGRKRDIPQLMARDKNTQSEGERIAINTPIQGTAADIIKLAMIDIDEELRKRNMKSRMIIQVHDELVFEVPDEEKEELVDLVKNKMTNVVKLSVPLEVDISIGKSWS

SEQ ID NO: 105 - protein, 680 aa, Synthetic construct
MKELQLYEEAEPTGYEIVKDHKTFEDLIEKLKEVPSFALALETSSLDPFNCEIVGISVSFKPKTAYYIPLHHRNAQNLDETLVLSKLKEILEDPSSKIVGQNLKYAYKVLMVKGISPVYPHFDTMIAAYLLEPNEKKFNLEDLSLKFLGYKMTSYQELMSFSSPLFGFSFADVPVDKAANYSCEDADITYRLYKILSMKLHEAELENVFYRIEMPLVNVLARMELNGVYVDTEFLKKLSEEYGKKLEELAEKIYQIAGGSWYQPKGGTEMFCHPRTGKPLPKYPRIKIPKVGGIFKKPKNKAQREGREPCELDTREYVAGAPYTPVEHVVFNPSSPKQVSKILFEKLGIKPRGKTTKTGAYSTRIEVLEEIANEHEIVPLILEYRKIQKLKSTYIDTLPKLVNPKTGRIHASFHQTGTATGRLSSSDPNLQNLPTKSEEGKEIRKAIVPQDPDWWIVSADYSQIELRILAHLSGDENLVKAFEEGIDVHTLTASRIYNVKPEEVNEEMRRVGKMVNYSIIYGVTPYGLSVRLGIPVKEAEKMIISYFTLYPKVRSYIQQVVAEAKEKGYVRTLFGRKRDIPQLMARDKNTQSEGERIAINTPIQGTAADIIKLAMIDIDEELRKRNMKSRMIIQVHDELVFEVPDEEKEELVDLVKNKMTNVVKLSVPLEVDISIGKSWS

SEQ ID NO: 106 - protein, 1071 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKAAEEAPWPPPEGAFLGFSFSRPEPMWAELLALAGAWEGRLHRAQDPLRGLRDLKGVRGILAKDLAVLALREGLDLFPEDDPMLLAYLLDPSNTTPEGVARRYGGEWTEDAGERALLAERLFQTLKERLKGEERLLWLYEEVEKPLSRVLARMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 107 - protein, 109 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLA

SEQ ID NO: 108 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAARLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 109 - protein, 1071 aa, Synthetic construct
MSDKIIHLTDDSFDTDVLKADGAILVDFWAEWCGPCKMIAPILDEIADEYQGKLTIAKLNIDQNPGTAPKYGIRGIPTLLLFKNGEVVATKVGALSKGQLKEFLDANLAGSSGGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 110 - protein, 1093 aa, Synthetic construct
MMDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 111 - protein, 1093 aa, Synthetic construct
MSDKIIHLTDDSFDTDVLKADGAILVEFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 112 - protein, 1093 aa, Synthetic construct
MMDKIIHLTDDSFDTDVLKADGAILVEFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 113 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDKRVYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 114 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDNREYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 115 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDNRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 116 - protein, 1093 aa, Synthetic construct
MMDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSGSGSSGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 117 - protein, 1093 aa, Synthetic construct
MMDKIIHLTDDSFDTDVLKADGAILVDFWAPWCGPCKMIAPILDEIAEEYQGKLTVAKLNIDQNPGTAPKYGIRGIPTLLLFKNGEVAATKVGALSKGQLKEFLDANLAGSSGSGSGSSGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 118 - protein, 1093 aa, Synthetic construct
MEDKIIHLTDDSFDTDVLKADGAILVDFWAPWCGPCKMIAPILDEIAEEYQGKLTVAKLNIDQNPGTAPKYGIRGIPTLLLFKNGEVAATKVGALSKGQLKEFLDANLAGSSGSGSGSSGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 119 - protein, 1093 aa, Synthetic construct; identical to SEQ ID NO: 116

SEQ ID NO: 120 - protein, 1093 aa, Synthetic construct
MMDKIIHLTDDSFDTDVLKADGAILVDFWAPWCGPCKMIAPILDEIAEEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 121 - protein, 1093 aa, Synthetic construct
MEDKIIHLTDDSFDTDVLKADGAILVDFWAPWCGPCKMIAPILDEIAEEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 122 - protein, 1093 aa, Synthetic construct
MSDKIIHLTDDSFDTDVLKADGAILVEFWAPWCGPCKMIAPILDEIADEYQGKLTVAKLNIDQNPGTAPKYGIRGIPTLLLFKNGEVAATKVGALSKGQLKEFLDANLAGSSGSGSGSSGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 123 - protein, 1093 aa, Synthetic construct
MSDKIIHLTDDSFDTDVLKADGAILVEFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 124 - protein, 1093 aa, Synthetic construct
MEDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSGSGSSGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 125 - protein, 1093 aa, Synthetic construct
MEDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 126 - protein, 1093 aa, Synthetic construct
MSDKIIHLTDDSFDTDVLKADGAILVDFWAPWCGPCKMIAPILDEIAEEYQGKLTVAKLNIDQNPGTAPKYGIRGIPTLLLFKNGEVAATKVGALSKGQLKEFLDANLAGSSGSGSGSSGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 127 - protein, 1093 aa, Synthetic construct
MSDKIIHLTDDSFDTDVLKADGAILVDFWAPWCGPCKMIAPILDEIAEEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 128 - protein, 1093 aa, Synthetic construct
MSDKIIHLTDDSFDTDVLKADGAILVDFWAPWCGPCKMIAPILDEIADEYQGKLTVAKLNIDQNQGTAPKYGIRGIPTLLLFKNGEVAATKVGALSKGQLKEFLDANLAGSSGSGSGSSGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 129 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRTYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 130 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRSYVAGAPYTPVEHVVFNPRSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 131 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRSYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 132 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRAYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 133 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDNRSYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 134 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRTYVAGAPYTPVEHVVFNPRSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 135 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRAYVAGAPYTPVEHVVFNPRSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 136 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDNRSYVAGAPYTPVEHVVFNPRSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 137 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDNRAYVAGAPYTPVEHVVFNPRSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 138 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDNRAYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 139 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDNRTYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 140 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDNRTYVAGAPYTPVEHVVFNPRSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 141 - protein, 1093 aa, Synthetic construct; identical to SEQ ID NO: 115

SEQ ID NO: 142 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDNRVYVAGAPYTPVEHVVFNPRSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 143 - protein, 1093 aa, Synthetic construct
MSDKIIHLTDDSFDTDVLKADGAILVDFWAPWCGPCKMIAPILDEIAEEYQGKLTVAKLNIDQNQGTAPKYGIRGIPTLLLFKNGEVAATKVGALSKGQLKEFLDANLAGSSGSGSGSSGGGSGGGSGSSSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 144 - protein, 1093 aa, Synthetic construct
MMDKIIHLTDDSFDTDVLKADGAILVDFWAPWCGPCKMIAPILDEIAEEYQGKLTVAKLNIDQNQGTAPKYGIRGIPTLLLFKNGEVAATKVGALSKGQLKEFLDANLAGSSGSGSGSSGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 145 - protein, 1093 aa, Synthetic construct
MSDKIIHLTDDSFDTDVLKADGAILVEFWAPWCGPCKMIAPILDEIAEEYQGKLTVAKLNIDQNQGTAPKYGIRGIPTLLLFKNGEVAATKVGALSKGQLKEFLDANLAGSSGSGSGSSGGGSGGGSGSSSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 146 - protein, 1093 aa, Synthetic construct
MMDKIIHLTDDSFDTDVLKADGAILVEFWAPWCGPCKMIAPILDEIAEEYQGKLTVAKLNIDQNQGTAPKYGIRGIPTLLLFKNGEVAATKVGALSKGQLKEFLDANLAGSSGSGSGSSGGGSGGGSGSSSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 147 - protein, 1093 aa, Synthetic construct
MEDKIIHLTDDSFDTDVLKADGAILVDFWAPWCGPCKMIAPILDEIAEEYQGKLTVAKLNIDQNQGTAPKYGIRGIPTLLLFKNGEVAATKVGALSKGQLKEFLDANLAGSSGSGSGSSGGGSGGGSGSSSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 148 - protein, 1093 aa, Synthetic construct
MEDKIIHLTDDSFDTDVLKADGAILVEFWAPWCGPCKMIAPILDEIAEEYQGKLTVAKLNIDQNQGTAPKYGIRGIPTLLLFKNGEVAATKVGALSKGQLKEFLDANLAGSSGSGSGSSGGGSGGGSGSSSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 149 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 150 - protein, 1093 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 151 - protein, 1071 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 152 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSGSGSSGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 153 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPKSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 154 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 155 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRKYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 156 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRLYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 157 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGKEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 158 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 159 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVKHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 160 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDKREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 161 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPKIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 162 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPRSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 163 - protein, 1093 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFIDANLAGSSGSGSGSSGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 164 - protein, 1084 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFIDANLAGSSGSGSGSSGGSGGGSGSSGGSGSSGGSSGGGGSGSGSGSSGGSGSSGSGSSGSSGGGGSGGSSGSSGGSGSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 165 - protein, 1084 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSGSGSSGGSGGGSGSSGGSGSSGGSSGGGGSGSGSGSSGGSGSSGSGSSGSSGGGGSGGSSGSSGGSGSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 166 - protein, 1112 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLGEMKGEYKDDFKRMLEEQGEEYVDGLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 167 - protein, 1071 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGGGGSGGGSGSSGGSGSSGGSSGGGGSGGGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 168 - protein, 1088 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGSGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 169 - protein, 1101 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSGSSGGSGSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 170 - protein, 1071 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 171 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDKREYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 172 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDKRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAFRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 173 - protein, 1070 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGSWYQPKGGTEMFCHPRTGKPLPKYPRIKIPKVGGIFKKPKNKAQREGREPCELDTREYVAGAPYTPVEHVVFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 174 - protein, 1071 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 175 - protein, 1070 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 176 - protein, 1093 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 177 - protein, 1071 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSNPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRAAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 178 - protein, 1071 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSNPNLQNIPVRTPLGQRIRRAFIAEEGWLLVALDYSQIELRVLAHLSGDENLIRAFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 179 - protein, 1071 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRAFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRAAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 180 - protein, 1071 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSNPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAARLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 181 - protein, 1071 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAARLSQELAIPYEEAQAFIERYFQSFPKVRAWIEKTLEEGRRRGYVETLFGRRRYVPDLEARVKSVRAAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 182 - protein, 1071 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRAFQEGRDIHTETASWMFGVPREAVDPLMRRAAKTINFGVLYGMSAARLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 183 - protein, 1071 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSNPNLQNIPVRTPLGQRIRRAFIAEEGWLLVALDYSQIELRVLAHLSGDENLIRAFQEGRDIHTETASWMFGVPREAVDPLMRRAAKTINFGVLYGMSAARLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 184 - protein, 1071 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSNPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAARLSQELAIPYEEAQAFIERYFQSFPKVRAWIEKTLEEGRRRGYVETLFGRRRYVPDLEARVKSVRAAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 185 - protein, 1071 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSNPNLQNIPVRTPLGQRIRRAFIAEEGWLLVALDYSQIELRVLAHLSGDENLIRAFQEGRDIHTETASWMFGVPREAVDPLMRRAAKTINFGVLYGMSAARLSQELAIPYEEAQAFIERYFQSFPKVRAWIEKTLEEGRRRGYVETLFGRRRYVPDLEARVKSVRAAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 186 - protein, 1071 aa, Synthetic construct
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSSGGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTREYVAGAPYTPVEHVVFNPSSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRAFQEGRDIHTETASWMFGVPREAVDPLMRRAAKTINFGVLYGMSAARLSQELAIPYEEAQAFIERYFQSFPKVRAWIEKTLEEGRRRGYVETLFGRRRYVPDLEARVKSVRAAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 187 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGAEAAAKEAAAKEAAAKEAAAKALEAEAAAKEAAAKEAAAKEAAAKASGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRAFQEGRDIHTETASWMFGVPREAVDPLMRRAAKTINFGVLYGMSAARLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 188 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGAEAAAKEAAAKEAAAKEAAAKALEAEAAAKEAAAKEAAAKEAAAKASGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSNPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAARLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 189 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGAEAAAKEAAAKEAAAKEAAAKALEAEAAAKEAAAKEAAAKEAAAKASGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAARLSQELAIPYEEAQAFIERYFQSFPKVRAWIEKTLEEGRRRGYVETLFGRRRYVPDLEARVKSVRAAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 190 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGAEAAAKEAAAKEAAAKEAAAKALEAEAAAKEAAAKEAAAKEAAAKASGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSNPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAARLSQELAIPYEEAQAFIERYFQSFPKVRAWIEKTLEEGRRRGYVETLFGRRRYVPDLEARVKSVRAAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 191 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGAEAAAKEAAAKEAAAKEAAAKALEAEAAAKEAAAKEAAAKEAAAKASGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSNPNLQNIPVRTPLGQRIRRAFIAEEGWLLVALDYSQIELRVLAHLSGDENLIRAFQEGRDIHTETASWMFGVPREAVDPLMRRAAKTINFGVLYGMSAARLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 192 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGAEAAAKEAAAKEAAAKEAAAKALEAEAAAKEAAAKEAAAKEAAAKASGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRAFQEGRDIHTETASWMFGVPREAVDPLMRRAAKTINFGVLYGMSAARLSQELAIPYEEAQAFIERYFQSFPKVRAWIEKTLEEGRRRGYVETLFGRRRYVPDLEARVKSVRAAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 193 - protein, 1093 aa, Synthetic construct
MSDKIIHLTDDSFDTDVLKADGAILVDFWAPWCGPCKMIAPILDEIADEYQGKLTVAKLNIDQNPGTAPKYGIRGIPTLLLFKNGEVAATKVGALSKGQLKEFLDANLAGSSGSSGSSGGGSGGGSGAEAAAKEAAAKEAAAKEAAAKALEAEAAAKEAAAKEAAAKEAAAKASGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFCHPRTGKPLPKYPRIKIPKVGGIFKKPKNKAQREGREPCELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSNPNLQNIPVRTPLGQRIRRAFIAEEGWLLVALDYSQIELRVLAHLSGDENLIRAFQEGRDIHTETASWMFGVPREAVDPLMRRAAKTINFGVLYGMSAARLSQELAIPYEEAQAFIERYFQSFPKVRAWIEKTLEEGRRRGYVETLFGRRRYVPDLEARVKSVRAAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 194 - protein, 952 aa, Synthetic construct
MARLFLFDGTALAYRAYYALDRSLSTSTGIPTNAVYGVARMLVKFIKEHIIPEKDYAAVAFDKKAATFRHKLLEAYKAQRPKTPDLLVQQLPYIKRLIEALGFKVLELEGYEADDIIATLAVKGCTFFDEIFIITGDKDMLQLVNEKIKVWRIVKGISDLELYDSKKVKERYGVEPHQIPDLLALTGDEIDNIPGVTGIGEKTAVQLLGKYRNLEDILEHARELPQRVRKALLRDREVAILSKKLATLVTNAPVEVDWEEMKYRGYDKRKLLPILKELEFASIMKELQLYEEAEPTGYEIVKDHKTFEDLIEKLKEVPSFALDLETSSLDPFNCEIVGISVSFKPKTAYYIPLHHRNAQNLDETLVLSKLKEILEDPSSKIVGQNLKYDYKVLMVKGISPVYPHFDTMIAAYLLEPNEKKFNLEDLSLKFLGYKMTSYQELMSFSSPLFGFSFADVPVDKAANYSCEDADITYRLYKILSMKLHEAELENVFYRIEMPLVNVLARMELNGVYVDTEFLKKLSEEYGKKLEELAEKIYQIAGSSWYRPKGGKAFFRHPVTGKDLTNYPRVIYPKAGEIYTKGGKLAKTLYCKDRPFTPIEYTVFNPGSPKQVSKILFEKLGIKPRGKTTKTGEYSTRIEVLEEIANEHEIVPLILEYRKIQKLKSTYIDTLPKLVNPKTGRIHASFHQTGTATGRLSSSDPNLQNLPTKSEEGKEIRKAIVPQDPDWWIVSADYSQIELRILAHLSGDENLVKAFEEGIDVHTLTASRIYNVKPEEVNEEMRRVGKMVNFSIIYGVTPYGLSVRLGIPVKEAEKMIISYFTLYPKVRSYIQQVVAEAKEKGYVRTLFGRKRDIPQLMARDKNTQSEGERIAINTPIQGTAADIIKLAMIDIDEELRKRNMKSRMIIQVHDELVFEVPDEEKEELVDLVKNKMTNVVKLSVPLEVDISIGKSWS

SEQ ID NO: 195 - protein, 963 aa, Synthetic construct
MARLFLFDGTALAYRAYYALDRSLSTSTGIPTNAVYGVARMLVKFIKEHIIPEKDYAAVAFDKKAATFRHKLLEAYKAQRPKTPDLLVQQLPYIKRLIEALGFKVLELEGYEADDIIATLAVKGCTFFDEIFIITGDKDMLQLVNEKIKVWRIVKGISDLELYDSKKVKERYGVEPHQIPDLLALTGDEIDNIPGVTGIGEKTAVQLLGKYRNLEDILEHARELPQRVRKALLRDREVAILSKKLATLVTNAPVEVDWEEMKYRGYDKRKLLPILKELEFASIMKELQLYEEAEPTGYEIVKDHKTFEDLIEKLKEVPSFALDLETSSLDPFNCEIVGISVSFKPKTAYYIPLHHRNAQNLDETLVLSKLKEILEDPSSKIVGQNLKYDYKVLMVKGISPVYPHFDTMIAAYLLEPNEKKFNLEDLSLKFLGYKMTSYQELMSFSSPLFGFSFADVPVDKAANYSCEDADITYRLYKILSMKLHEAELENVFYRIEMPLVNVLARMELNGVYVDTEFLKKLSEEYGKKLEELAEKIYQIAGGTWYQPKGGTELFLHPRTGKPLGKYPRVKYPKQGGIYKKPKNKAQREGREPCDLDTRDYVEGAPYTPVEHVVFNPSSPKQVSKILFEKLGIKPRGKTTKTGEYSTRIEVLEEIANEHEIVPLILEYRKIQKLKSTYIDTLPKLVNPKTGRIHASFHQTGTATGRLSSSDPNLQNLPTKSEEGKEIRKAIVPQDPDWWIVSADYSQIELRILAHLSGDENLVKAFEEGIDVHTLTASRIYNVKPEEVNEEMRRVGKMVNFSIIYGVTPYGLSVRLGIPVKEAEKMIISYFTLYPKVRSYIQQVVAEAKEKGYVRTLFGRKRDIPQLMARDKNTQSEGERIAINTPIQGTAADIIKLAMIDIDEELRKRNMKSRMIIQVHDELVFEVPDEEKEELVDLVKNKMTNVVKLSVPLEVDISIGKSWS

SEQ ID NO: 196 - protein, 963 aa, Synthetic construct
MARLFLFDGTALAYRAYYALDRSLSTSTGIPTNAVYGVARMLVKFIKEHIIPEKDYAAVAFDKKAATFRHKLLEAYKAQRPKTPDLLVQQLPYIKRLIEALGFKVLELEGYEADDIIATLAVKGCTFFDEIFIITGDKDMLQLVNEKIKVWRIVKGISDLELYDSKKVKERYGVEPHQIPDLLALTGDEIDNIPGVTGIGEKTAVQLLGKYRNLEDILEHARELPQRVRKALLRDREVAILSKKLATLVTNAPVEVDWEEMKYRGYDKRKLLPILKELEFASIMKELQLYEEAEPTGYEIVKDHKTFEDLIEKLKEVPSFALDLETSSLDPFNCEIVGISVSFKPKTAYYIPLHHRNAQNLDETLVLSKLKEILEDPSSKIVGQNLKYDYKVLMVKGISPVYPHFDTMIAAYLLEPNEKKFNLEDLSLKFLGYKMTSYQELMSFSSPLFGFSFADVPVDKAANYSCEDADITYRLYKILSMKLHEAELENVFYRIEMPLVNVLARMELNGVYVDTEFLKKLSEEYGKKLEELAEKIYQIAGGSWYAPKGGKEFFRHPRTGKDLPKYPRVVYPKVGGIFKKPKNKAQRLGLEPCERDTRDTMEGAPFTPITYVEFNPGSPKQVSKILFEKLGIKPRGKTTKTGEYSTRIEVLEEIANEHEIVPLILEYRKIQKLKSTYIDTLPKLVNPKTGRIHASFHQTGTATGRLSSSDPNLQNLPTKSEEGKEIRKAIVPQDPDWWIVSADYSQIELRILAHLSGDENLVKAFEEGIDVHTLTASRIYNVKPEEVNEEMRRVGKMVNFSIIYGVTPYGLSVRLGIPVKEAEKMIISYFTLYPKVRSYIQQVVAEAKEKGYVRTLFGRKRDIPQLMARDKNTQSEGERIAINTPIQGTAADIIKLAMIDIDEELRKRNMKSRMIIQVHDELVFEVPDEEKEELVDLVKNKMTNVVKLSVPLEVDISIGKSWS

SEQ ID NO: 197 - protein, 680 aa, Synthetic construct
MKELQLYEEAEPTGYEIVKDHKTFEDLIEKLKEVPSFALALETSSLDPFNCEIVGISVSFKPKTAYYIPLHHRNAQNLDETLVLSKLKEILEDPSSKIVGQNLKYAYKVLMVKGISPVYPHFDTMIAAYLLEPNEKKFNLEDLSLKFLGYKMTSYQELMSFSSPLFGFSFADVPVDKAANYSCEDADITYRLYKILSMKLHEAELENVFYRIEMPLVNVLARMELNGVYVDTEFLKKLSEEYGKKLEELAEKIYQIAGGTWYQPKGGTELFLHPRTGKPLGKYPRVKYPKQGGIYKKPKNKAQREGREPCDLDTRDYVEGAPYTPVEHVVFNPSSPKQVSKILFEKLGIKPRGKTTKTGAYSTRIEVLEEIANEHEIVPLILEYRKIQKLKSTYIDTLPKLVNPKTGRIHASFHQTGTATGRLSSSDPNLQNLPTKSEEGKEIRKAIVPQDPDWWIVSADYSQIELRILAHLSGDENLVKAFEEGIDVHTLTASRIYNVKPEEVNEEMRRVGKMVNYSIIYGVTPYGLSVRLGIPVKEAEKMIISYFTLYPKVRSYIQQVVAEAKEKGYVRTLFGRKRDIPQLMARDKNTQSEGERIAINTPIQGTAADIIKLAMIDIDEELRKRNMKSRMIIQVHDELVFEVPDEEKEELVDLVKNKMTNVVKLSVPLEVDISIGKSWS

SEQ ID NO: 198 - protein, 680 aa, Synthetic construct
MKELQLYEEAEPTGYEIVKDHKTFEDLIEKLKEVPSFALALETSSLDPFNCEIVGISVSFKPKTAYYIPLHHRNAQNLDETLVLSKLKEILEDPSSKIVGQNLKYAYKVLMVKGISPVYPHFDTMIAAYLLEPNEKKFNLEDLSLKFLGYKMTSYQELMSFSSPLFGFSFADVPVDKAANYSCEDADITYRLYKILSMKLHEAELENVFYRIEMPLVNVLARMELNGVYVDTEFLKKLSEEYGKKLEELAEKIYQIAGGSWYAPKGGKEFFRHPRTGKDLPKYPRVVYPKVGGIFKKPKNKAQRLGLEPCERDTRDTMEGAPFTPITYVEFNPGSPKQVSKILFEKLGIKPRGKTTKTGAYSTRIEVLEEIANEHEIVPLILEYRKIQKLKSTYIDTLPKLVNPKTGRIHASFHQTGTATGRLSSSDPNLQNLPTKSEEGKEIRKAIVPQDPDWWIVSADYSQIELRILAHLSGDENLVKAFEEGIDVHTLTASRIYNVKPEEVNEEMRRVGKMVNYSIIYGVTPYGLSVRLGIPVKEAEKMIISYFTLYPKVRSYIQQVVAEAKEKGYVRTLFGRKRDIPQLMARDKNTQSEGERIAINTPIQGTAADIIKLAMIDIDEELRKRNMKSRMIIQVHDELVFEVPDEEKEELVDLVKNKMTNVVKLSVPLEVDISIGKSWS

SEQ ID NO: 199 - protein, 76 aa, Synthetic construct
  VARIANT 66: note = X is threonine, valine, or cysteine
GSWYQPKGGTEMFLHPRTGKPLPKYPRIKIPKVGGIFKKPKNKAQREGREPVELDTREYVAGAPYXPVEHVVFNPS

SEQ ID NO: 200 - protein, 108 aa, Synthetic construct
  VARIANT 32: note = X is threonine, valine, or cysteine
  VARIANT 35: note = X is not cysteine
SDKIIHLTDDSFDTDVLKADGAILVDFWAEWXGPXKMIAPILDEIADEYQGKLTVAKLNIDQNPGTAPKYGIRGIPTLLLFKNGEVAATKVGALSKGQLKEFLDANLA

SEQ ID NO: 201 - protein, 1093 aa, Synthetic construct
MSDKIIHLTDDSFDTDVLKADGAILVDFWAPWCGPSKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFLHPRTGKPLPKYPRIKIPKVGGIFKKPKNKAQREGREPVELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAARLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 202 - protein, 1093 aa, Synthetic construct
MSDKIIHLTDDSFDTDVLKADGAILVDFWAPWAGPSKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFLHPRTGKPLPKYPRIKIPKVGGIFKKPKNKAQREGREPVELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAARLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 203 - protein, 1093 aa, Synthetic construct
MSDKIIHLTDDSFDTDVLKADGAILVDFWAPWAGPSKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFLHPRTGKPLPKYPRIKIPKVGGIFKKPKNKAQREGREPVELDTRVYVAGAPYVPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAARLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 204 - protein, 1093 aa, Synthetic construct
MSDKIIHLTDDSFDTDVLKADGAILVDFWAPWCGPSKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFLHPRTGKPLPKYPRIKIPKVGGIFKKPKNKAQREGREPVELDTRVYVAGAPYCPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAARLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 205 - protein, 1093 aa, Synthetic construct
MSDKIIHLTDDSFDTDVLKADGAILVDFWAPWDGPSKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFLHPRTGKPLPKYPRIKIPKVGGIFKKPKNKAQREGREPVELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAARLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 206 - protein, 1093 aa, Synthetic construct
MSDKIIHLTDDSFDTDVLKADGAILVDFWAPWGGPSKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFLHPRTGKPLPKYPRIKIPKVGGIFKKPKNKAQREGREPVELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAARLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 207 - protein, 1093 aa, Synthetic construct
MSDKIIHLTDDSFDTDVLKADGAILVDFWAPWPGPSKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFLHPRTGKPLPKYPRIKIPKVGGIFKKPKNKAQREGREPVELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAARLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 208 - protein, 1093 aa, Synthetic construct
MSDKIIHLTDDSFDTDVLKADGAILVDFWAPWSGPSKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFLHPRTGKPLPKYPRIKIPKVGGIFKKPKNKAQREGREPVELDTRVYVAGAPYNPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAARLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

SEQ ID NO: 209 - protein, 1093 aa, Synthetic construct
MSDKIIHLTDDSFDTDVLKADGAILVDFWAPWSGPSKMIAPILDEIADEYQGKLTVAKLNIDQNPGTAPKYGIRGIPTLLLFKNGEVAATKVGALSKGQLKEFLDANLAGSSGSSGSSGGGSGGGSGSSGSSGSSGGGSGGGSGSSGGSGSSGGSSGGGGSGSGSGSSGGSGSSGSGSSGSSGGGGSGGSSRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGGSWYQPKGGTEMFLHPRTGKPLPKYPRIKIPKVGGIFKKPKNKAQREGREPVELDTRVYVAGAPYTPVEHVVFNPNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAARLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKGD

(19) United States
(12) Patent Application Publication
(10) Pub. No.: US 2026/0062740 A1
(43) Pub. Date: Mar. 5, 2026

(54) REDUCING-AGENT-FREE DNA POLYMERASE WITH REDUCED ARTIFACT FORMATION

(71) Applicant: Promega Corporation, Madison, WI (US)

(72) Inventors: David MOKRY, Madison, WI (US); Nicholas A. COURTNEY, Madison, WI (US); Robert S. MCLAREN, Madison, WI (US); Kimberly SCHNELL, Madsion, WI (US); Kristopher ZIMMERMAAN, Madison, WI (US)

(21) Appl. No.: 19/319,451

(22) Filed: Sep. 4, 2025

Related U.S. Application Data
(60) Provisional application No. 63/690,674, filed on Sep. 4, 2024.

(51) Int. Cl. C12Q 1/6848; C07K 14/47; C12N 9/12

(52) U.S. Cl. CPC C12Q 1/6848; C07K 14/47; C12N 9/1276; C07K 2319/00

(57) ABSTRACT

Provided herein are compositions and systems comprising a DNA polymerase domain, a thioredoxin binding domain (TBD), and thioredoxin (TRX) engineered to synthesize DNA with reduced stutter artifacts in the absence of a reducing agent. Kits comprising the DNA polymerase/TBD/TRX compositions and systems herein and methods of use thereof in the absence of a reducing agent are also within the scope herein.

### CROSS-REFERENCE

This application claims the benefit of U.S. Provisional Patent Application No. 63/690,674, Sep. 4, 2024; which is incorporated by reference herein in its entirety.

### SEQUENCE LISTING

The text of the computer readable sequence listing filed herewith, titled “PRMG_43544_202_SequenceListing.xml”, created Sep. 5, 2025, having a file size of 375,041 bytes, is hereby incorporated by reference in its entirety.

### FIELD

Provided herein are compositions and systems comprising a DNA polymerase domain, a thioredoxin binding domain (TBD), and thioredoxin (TRX) engineered to synthesize DNA with reduced stutter artifacts in the absence of a reducing agent. Kits comprising the DNA polymerase/TBD/TRX compositions and systems herein and methods of use thereof in the absence of a reducing agent are also within the scope herein.

### BACKGROUND

Microsatellites, or short tandem repeats (STRs), consist of tandemly repeated DNA sequence motifs of 1 to 8 nucleotides in length. They are widely dispersed and abundant in the eukaryotic genome and are often highly polymorphic due to variation in the number of repeat units.

Forensic Short Tandem Repeat (STR) profiling relies upon accurately determining the number of repeated DNA sequences at a given genome locus, with each repeat unit typically consisting of 3 to 6 base pairs. Traditional polymerase chain reaction (PCR) methods result in a population of amplicons that include products with incorrect insertions or deletions of the repeated sequence in a phenomenon known as strand slippage or “stutter.” These stutter products can complicate the analysis of STR profiles and can potentially mask trace DNA contributions in STR profiles derived from more than one individual.

Microsatellite instability (MSI) is an established biomarker that often signals susceptibility to cancer development and can be found in a broad range of solid tumors. MSI provides genetic evidence of an impaired DNA mismatch repair mechanism, which is known to be one of the most frequently mutated sets of genes in cancer. MSI can also be predictive of Lynch syndrome. MSI results in the addition or deletion of nucleotides during DNA replication, which are then inherited by daughter cells. Mononucleotide repeats are particularly sensitive to these types of MSI-induced errors. While these anomalous insertions or deletions can be detected by PCR-based assays, stutter artifacts-which are particularly problematic when amplifying mononucleotide repeat sequences-significantly impair the sensitivity of such testing.

Stutter signals differ from the PCR product representing the genomic allele by multiples of repeat unit size. For dinucleotide repeat loci, the prevalent stutter signal is generally two bases shorter than the genomic allele signal, with additional side-products that are 4 and 6 bases shorter. The multiple signal pattern observed for each allele especially complicates interpretation when two alleles from an individual are close in size (e.g., medical and genetic mapping applications) or when DNA samples contain mixtures from two or more individuals (e.g., forensic applications). Such confusion is maximal for mononucleotide microsatellite genotyping, when both genomic and stutter fragments experience one-nucleotide spacing.

Reduced stutter polymerases have been described in U.S. patent application Ser. No. 18/595,339; incorporated by reference in its entirety. Certain reduced stutter polymerases require the presence of a reducing agent to function properly. This requirement has several drawbacks, including the need to eliminate the use of bovine serum albumin (BSA) from reaction conditions. BSA is commonly used as a blocking agent against PCR inhibitors. In the presence of a reducing agent, the 17 disulfide bonds in BSA are reduced, which results in the destabilized BSA to precipitate out of solution. As a consequence, the blocking efficiency of the BSA is reduced, and the sample must be centrifuged to remove the precipitate prior to analysis. Therefore, there is a need for a reduced stutter polymerase that does not require a reducing agent to achieve suitable activity.

### SUMMARY

Provided herein are compositions and systems comprising a DNA polymerase domain, a thioredoxin binding domain (TBD), and thioredoxin (TRX) engineered to synthesize DNA with reduced stutter artifacts in the absence of a reducing agent. (e.g., DTT, TCEP, etc.) Kits comprising the DNA polymerase/TBD/TRX compositions and systems herein and methods of use thereof in the absence of a reducing agent (e.g., DTT, TCEP, etc.) are also within the scope herein. The DNA polymerase/TBD/TRX compositions and systems herein are engineered to reduce stutter and to produce fewer stutter artifacts. Kits comprising the DNA polymerase/TBD/TRX compositions and systems herein and methods of use thereof are also within the scope herein. In particular embodiments, provided herein (1) chimeras of a DNA polymerase, a thioredoxin binding domain, and thioredoxin; (2) chimeras of a DNA polymerase and a thioredoxin binding domain in the presence of TRX at a TRX:TBD ratio of 0.1 to 2000 (e.g., 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700. 800, 900, 1000, 1500, 2000, or ranges therebetween (e.g., 0.1 to 800, 0.6 to 600, etc.)); and/or (3) chimeras of a thermostable DNA polymerase and thioredoxin in the presence of a thioredoxin binding domain.

In some embodiments, the DNA polymerases and DNA polymerase systems herein are provided in the absence of a reducing agent, for example, in the absence of one or more (e.g., 1, 2, 3 . . . all) of tris (2-carboxyethyl) phosphine (TCEP), dithiothreitol (DTT), mercaptoethanol, cysteine, thioglycerol, thioglycolic acid, cysteamine, glutathione, N-acetylcysteine (NAC), β-mercaptoethanol, sodium sulfite, thiourea, and dimethylsulfoxide (DMSO).

In some embodiments, provided herein are DNA polymerase systems comprising: (a) a DNA polymerase domain; (b) a thioredoxin binding domain (TBD), wherein the TBD has non-cysteine amino acids at positions corresponding to positions 14 and 52 of SEQ ID NO: 15; and (c) a thioredoxin (TRX) domain, wherein the TRX domain has a non-cysteine amino acid at a position corresponding to position 35 of SEQ ID NO: 16. In some embodiments, the amino acid at the position corresponding to position 14 of SEQ ID NO: 15 is a leucine. In some embodiments, the amino acid at the position corresponding to position 52 of SEQ ID NO: 15 is a valine. In some embodiments, the amino acid at the position corresponding to position 35 of SEQ ID NO: 16 is a serine. In some embodiments, the TBD has a substitution at a position corresponding to position 66 of SEQ ID NO: 15. In some embodiments, the amino acid at a position corresponding to position 66 of SEQ ID NO: 15 is threonine, valine, or cysteine. In some embodiments, the TBD has a substitution at a position corresponding to position 32 of SEQ ID NO: 16. In some embodiments, the amino acid at a position corresponding to position 32 of SEQ ID NO: 16 is alanine, cysteine, aspartic acid, glycine, proline, or serine. In some embodiments, the DNA polymerase system is capable of synthesizing a DNA product from deoxynucleotide triphosphates in the presence of a DNA template and under appropriate reaction conditions. In some embodiments, the DNA polymerase system exhibits reduced stutter proclivity compared to a DNA polymerase comprising the DNA polymerase domain in the absence of the TBD and/or TRX. In some embodiments, the DNA polymerase system exhibits at least 10% (e.g., 10%, 15%, 20%, 25%, 30%, 35%, 40%, 45%, 50%, 60%, 70%, 80% 90%, 95%, 99%) reduced stutter proclivity compared to a DNA polymerase comprising the DNA polymerase domain in the absence of the TBD and/or TRX. In some embodiments, the DNA polymerase system exhibits at least 10% (e.g., 10%, 15%, 20%, 25%, 30%, 35%, 40%, 45%, 50%, 60%, 70%, 80% 90%, 95%, 99%) fewer stutter artifacts compared to a DNA polymerase comprising the DNA polymerase domain in the absence of the TBD and/or TRX. In some embodiments, the DNA polymerase system comprises the DNA polymerase domain conjugated to the TBD and/or TRX. In some embodiments, the DNA polymerase system comprises the DNA polymerase domain genetically fused to one or both of the TBD and/or TRX. In some embodiments, the DNA polymerase system comprises a genetic fusion of the DNA polymerase domain, TBD, and TRX. In some embodiments, one of the TBD and TRX are not conjugated to the other components of the system. In some embodiments, the system comprises a free TRX and a DNA polymerase domain conjugated or genetically fused to a TBD. In some embodiments, the system comprises a free TBD and a DNA polymerase domain conjugated or genetically fused to a TRX. In some embodiments, the system comprises a DNA polymerase domain, TRX, and TBD conjugated or genetically fused together.

In some embodiments, provided herein are chimeric DNA polymerases with reduced stutter proclivity (e.g., at least 10% (e.g., 10%, 15%, 20%, 25%, 30%, 35%, 40%, 45%, 50%, 60%, 70%, 80% 90%, 95%, 99%), the chimeric DNA polymerase comprising a genetic fusion of: (a) a DNA polymerase domain; (b) a thioredoxin binding domain (TBD), wherein the TBD has non-cysteine amino acids at positions corresponding to positions 14 and 52 of SEQ ID NO: 15; and (c) a thioredoxin (TRX) domain, wherein the TRX domain has a non-cysteine amino acid at a position corresponding to position 35 of SEQ ID NO: 16. In some embodiments, the amino acid at the position corresponding to position 14 of SEQ ID NO: 15 is a leucine. In some embodiments, the amino acid at the position corresponding to position 52 of SEQ ID NO: 15 is a valine. In some embodiments, the amino acid at the position corresponding to position 35 of SEQ ID NO: 16 is a serine. In some embodiments, the TBD has a substitution at a position corresponding to position 66 of SEQ ID NO: 15. In some embodiments, the amino acid at a position corresponding to position 66 of SEQ ID NO: 15 is threonine, valine, or cysteine. In some embodiments, the TBD has a substitution at a position corresponding to position 32 of SEQ ID NO: 16. In some embodiments, the amino acid at a position corresponding to position 32 of SEQ ID NO: 16 is alanine, cysteine, aspartic acid, glycine, proline, or serine. In some embodiments, the DNA polymerase domain is thermophilic. In some embodiments, the DNA polymerase domain is derived from a native thermophilic DNA polymerase. In some embodiments, the native thermophilic DNA polymerase is selected from the group consisting of the Thermus aquaticus DNA polymerase, Thermus thermophilus DNA polymerase, Thermus flavus DNA polymerase, Thermotoga neapolitana polymerase, and Geobacillus stearothermophilus DNA polymerase. In some embodiments, the DNA polymerase domain is derived from a Family A DNA polymerase. In some embodiments, the DNA polymerase domain comprises at least 40% (e.g., 40%, 45%, 50%, 55%, 60%, 65%, 70%, 75%, 80%, 85%, 90%, 95%, 99%, 100%, or ranges therebetween) sequence identity with SEQ ID NO: 1. In some embodiments, the DNA polymerase domain further comprises an internal amino acid sequence insertion. In some embodiments, the DNA polymerase domain comprises an N-terminal portion with at least 40% (e.g., 40%, 45%, 50%, 55%, 60%, 65%, 70%, 75%, 80%, 85%, 90%, 95%, 99%, 100%, or ranges therebetween) sequence identity to SEQ ID NO: 14 and a C-terminal portion with 40% (e.g., 40%, 45%, 50%, 55%, 60%, 65%, 70%, 75%, 80%, 85%, 90%, 95%, 99%, 100%, or ranges therebetween) sequence identity to SEQ ID NO: 12, wherein the N-terminal portion and the C-terminal portion are separated by the internal amino acid sequence insertion. In some embodiments, the internal amino acid sequence insertion comprises the TBD. In some embodiments, the TBD is derived from the thioredoxin binding domain of a T3 or T7 bacteriophage DNA polymerase. In some embodiments, the TBD comprises at least 40% (e.g., 40%, 45%, 50%, 55%, 60%, 65%, 70%, 75%, 80%, 85%, 90%, 95%, 99%, 100%, or ranges therebetween) sequence identity with SEQ ID NO: 199. In some embodiments, the TBD is derived from the thioredoxin binding domain of a Klebsiella pneumoniae, Salmonella enterica, or Aeromonas hydrophila phage DNA polymerase. In some embodiments, the TBD comprises at least 50% (e.g., 50%, 55%, 60%, 65%, 70%, 75%, 80%, 85%, 90%, 95%, 99%, 100%, or ranges therebetween) sequence identity with one of SEQ ID NOS: 101-103. In some embodiments, the TBD sequence resides internally within the DNA polymerase domain sequence. In some embodiments, the TRX domain is derived from Escherichia coli thioredoxin. In some embodiments, the TRX domain comprises at least 50% (e.g., 50%, 55%, 60%, 65%, 70%, 75%, 80%, 85%, 90%, 95%, 99%, 100%, or ranges therebetween) sequence identity with SEQ ID NOS: 16, 17, or 107. In some embodiments, the TRX domain is derived from Alishwanella jeotgali or Thiococcus pfennigii thioredoxin. In some embodiments, the TRX domain comprises at least 50% (e.g., 50%, 55%, 60%, 65%, 70%, 75%, 80%, 85%, 90%, 95%, 99%, 100%, or ranges therebetween) sequence identity with one of SEQ ID NOS: 93 or 94. In some embodiments, the TRX domain comprises at least 50% (e.g., 50%, 55%, 60%, 65%, 70%, 75%, 80%, 85%, 90%, 95%, 99%, 100%, or ranges therebetween) sequence identity with one of SEQ ID NOS: 51-53. In some embodiments, the TRX sequence is fused to the N- or C-terminus of the DNA polymerase domain. In some embodiments, TRX sequence is fused to the DNA polymerase domain by a linker of 1-300 (e.g., 1, 2, 5, 10, 15, 20, 25, 30, 40, 50, 60, 70, 80, 90, 100, 125, 150, 175, 200, 250, 300, or more, or ranges or lengths therebetween) amino acids. In some embodiments, the linker is a flexible linker. In some embodiments, the linker is 50-100% (e.g., 50%, 60%, 70%, 80%, 90%, 100%, or ranges therebetween) glycine and serine residues. For example, a linker may comprise one or more repeating GS units, one or more repeating GSAT units, etc. In some embodiments, the linker comprises a rigid linker segment. In some embodiments, the rigid segment comprises one or more EAAAK peptide segments. In some embodiments, the DNA polymerase comprises at least 40% (e.g., 40%, 45%, 50%, 55%, 60%, 65%, 70%, 75%, 80%, 85%, 90%, 95%, 99%, 100%, or ranges therebetween) sequence identity to one of SEQ ID NOS: 22-49. In some embodiments, a linker comprises a sequence of Table 13 of U.S. patent application Ser. No. 18/595,339 (incorporated by reference in its entirety), -GS (24)-CASSIDYKRISRMPSKIMDAVIDTLNICKLANCE-GS(24), GS(24)-CASSIDYKRISRMPAVLADAVIDTLNICKLANCE-GS(24), etc.

In some embodiments, provided herein are compositions comprising a DNA polymerase domain conjugated to a thioredoxin binding domain (TBD), wherein the TBD has non-cysteine amino acids at positions corresponding to positions 14 and 52 of SEQ ID NO: 15. In some embodiments, the amino acid at the position corresponding to position 14 of SEQ ID NO: 15 is a leucine. In some embodiments, the amino acid at the position corresponding to position 52 of SEQ ID NO: 15 is a valine. In some embodiments, the TBD has a substitution at a position corresponding to position 66 of SEQ ID NO: 15. In some embodiments, the amino acid at a position corresponding to position 66 of SEQ ID NO: 15 is threonine, valine, or cysteine. In some embodiments, the DNA polymerase domain is genetically fused to the thioredoxin binding domain (TBD). In some embodiments, the DNA polymerase domain is derived from a Family A DNA polymerase (e.g., Taq polymerase, Tne polymerase, etc.). In some embodiments, the DNA polymerase domain is thermophilic. In some embodiments, the DNA polymerase domain is derived from a native thermophilic DNA polymerase. In some embodiments, the native thermophilic DNA polymerase is selected from the group consisting of the Thermus aquaticus DNA polymerase, Thermus thermophilus DNA polymerase, Thermus flavus DNA polymerase, Thermotoga neapolitana polymerase, and Geobacillus stearothermophilus DNA polymerase. In some embodiments, the DNA polymerase domain comprises at least 40% (e.g., 40%, 45%, 50%, 55%, 60%, 65%, 70%, 75%, 80%, 85%, 90%, 95%, 99%, 100%, or ranges therebetween) sequence identity with SEQ ID NO: 1. In some embodiments, the DNA polymerase domain comprises at least 40% (e.g., 40%, 45%, 50%, 55%, 60%, 65%, 70%, 75%, 80%, 85%, 90%, 95%, 99%, 100%, or ranges therebetween) sequence identity with one or more (e.g., 1, 2, 3, 4, 5, 6, 7, 8, 9, 10, or 11, in any ordered combination) of SEQ ID NOS: 2-12. In some embodiments, the DNA polymerase domain comprises at least 40% (e.g., 40%, 45%, 50%, 55%, 60%, 65%, 70%, 75%, 80%, 85%, 90%, 95%, 99%, 100%, or ranges therebetween) sequence identity with SEQ ID NOS: 14 and 12. In some embodiments, the DNA polymerase domain comprises a portion with at least 40% (e.g., 40%, 45%, 50%, 55%, 60%, 65%, 70%, 75%, 80%, 85%, 90%, 95%, 99%, 100%, or ranges therebetween) sequence identity with SEQ ID NOS: 14, excluding SEQ ID NO: 13. In some embodiments, the TBD sequence resides internally within the DNA polymerase domain sequence. In some embodiments, the DNA polymerase domain comprises an N-terminal portion with at least 40% (e.g., 40%, 45%, 50%, 55%, 60%, 65%, 70%, 75%, 80%, 85%, 90%, 95%, 99%, 100%, or ranges therebetween) sequence identity to SEQ ID NO: 14 and a C-terminal portion with at least 40% (e.g., 40%, 45%, 50%, 55%, 60%, 65%, 70%, 75%, 80%, 85%, 90%, 95%, 99%, 100%, or ranges therebetween) sequence identity to SEQ ID NO: 12, wherein the N-terminal portion and the C-terminal portion are separated by the TBD. In some embodiments, the TBD is derived from the thioredoxin binding domain of a T3 or T7 bacteriophage DNA polymerase. In some embodiments, the TBD comprises at least 40% (e.g., 40%, 45%, 50%, 55%, 60%, 65%, 70%, 75%, 80%, 85%, 90%, 95%, 99%, 100%, or ranges therebetween) sequence identity with SEQ ID NO: 199. In some embodiments, the TBD is derived from the thioredoxin binding domain of a Klebsiella pneumoniae, Salmonella enterica, or Aeromonas hydrophila phage DNA polymerase. In some embodiments, the TBD comprises at least 50% (e.g., 50%, 55%, 60%, 65%, 70%, 75%, 80%, 85%, 90%, 95%, 99%, 100%, or ranges therebetween) sequence identity with one of SEQ ID NOS: 101-103. In some embodiments, the composition further comprises thioredoxin (TRX), wherein the thioredoxin is present in the composition at 800 molar excess or less relative to the TBD (e.g., 5×, 10×, 20×, 30×, 40×, 50×, 60×, 70×, 80×, 90×, 100× 150×, 200× 250×, 300×, 400× 500×, 600×, 700×, 800×, or ranges therebetween). In some embodiments, the TRX domain has a non-cysteine amino acid at a position corresponding to position 35 of SEQ ID NO: 16. In some embodiments, the amino acid at the position corresponding to position 35 of SEQ ID NO: 16 is a serine. In some embodiments, the TRX has a substitution at a position corresponding to position 32 of SEQ ID NO: 16. In some embodiments, the amino acid at a position corresponding to position 32 of SEQ ID NO: 16 is alanine, cysteine, aspartic acid, glycine, proline, or serine. In some embodiments, the TRX is derived from Escherichia coli thioredoxin. In some embodiments, the TRX comprises at least 50% (e.g., 50%, 55%, 60%, 65%, 70%, 75%, 80%, 85%, 90%, 95%, 99%, 100%, or ranges therebetween) sequence identity with SEQ ID NO: 16, 17, or 107. In some embodiments, the TRX is derived from Thiococcus pfennigii thioredoxin. In some embodiments, the TRX comprises at least 50% (e.g., 50%, 55%, 60%, 65%, 70%, 75%, 80%, 85%, 90%, 95%, 99%, 100%, or ranges therebetween) sequence identity with SEQ ID NO: 93. In some embodiments, the TRX is derived from Alishwanella jeotgali thioredoxin. In some embodiments, the TRX comprises at least 50% (e.g., 50%, 55%, 60%, 65%, 70%, 75%, 80%, 85%, 90%, 95%, 99%, 100%, or ranges therebetween) sequence identity with SEQ ID NO: 94. In some embodiments, the TRX comprises at least 50% (e.g., 50%, 55%, 60%, 65%, 70%, 75%, 80%, 85%, 90%, 95%, 99%, 100%, or ranges therebetween) sequence identity with SEQ ID NO: 51-53.

In some embodiments, the TRX is a fusion with an additional polypeptide sequence. In some embodiments, the additional polypeptide sequence is a DNA binding protein, an amino acid sequence capable of binding DNA, a protein associated with a DNA replication site, a TBD, and/or a DNA polymerase. In some embodiments, the additional polypeptide sequence is fused to the TRX by a linker peptide or polypeptide. In some embodiments, the linker peptide or polypeptide is 1-300 amino acids in length (e.g., 1, 2, 5, 10, 20, 50, 100, 150, 200, 250, 300, or ranges or values therebetween). In some embodiments, any linkers described herein may find use in such embodiments.

In some embodiments, the thioredoxin is present in the composition at a TRX:TBD ratio of 0.1 to 2000 (e.g., 0.1, 0.2, 0.3, 0.4, 0.5, 0.6, 0.7, 0.8, 0.9, 1, 3, 4, 5, 6, 7, 8, 9, 10, 20, 30, 40, 60, 70, 80, 90, 100, 200, 300, 400, 500, 600, 700. 800, 900, 1000, 1500, 2000, or ranges therebetween (e.g., 0.1 to 800, 0.6 to 600)). In some embodiments, a fusion protein is provided having at least 40% (e.g., 40%, 45%, 50%, 55%, 60%, 65%, 70%, 75%, 80%, 85%, 90%, 95%, 99%, 100%, or ranges therebetween) sequence identity to one of SEQ ID NO: 28-34.

In some embodiments, provided herein are DNA polymerases comprising a DNA polymerase domain corresponding to SEQ ID NO: 1 and comprising: (a) segments having at least 40% (e.g., 40%, 45%, 50%, 55%, 60%, 65%, 70%, 75%, 80%, 85%, 90%, 95%, 99%, 100%, or ranges therebetween) sequence identity to SEQ ID NOS: 2, 4, 6, 8, 10, and 12; (b) segments having (i) at least 40% (e.g., 40%, 45%, 50%, 55%, 60%, 65%, 70%, 75%, 80%, 85%, 90%, 95%, 99%, 100%, or ranges therebetween) sequence identity to SEQ ID NOS: 3, 5, 7, 9, and 11, or (ii) wherein all or a portion of the sequences in SEQ ID NO: 1 corresponding to one or more of SEQ ID NOS: 3, 5, 7, 9, and 11 are substituted for a heterologous sequence selected from a TBD, TRX, and TIS (TBD/TRX interacting sequence); wherein the TBD has non-cysteine amino acids at positions corresponding to positions 14 and 52 of SEQ ID NO: 15; and wherein the TRX domain has a non-cysteine amino acid at a position corresponding to position 35 of SEQ ID NO: 16. In some embodiments, the amino acid at the position corresponding to position 14 of SEQ ID NO: 15 is a leucine. In some embodiments, the amino acid at the position corresponding to position 52 of SEQ ID NO: 15 is a valine. In some embodiments, the amino acid at the position corresponding to position 35 of SEQ ID NO: 16 is a serine. In some embodiments, the TBD has a substitution at a position corresponding to position 66 of SEQ ID NO: 15. In some embodiments, the amino acid at a position corresponding to position 66 of SEQ ID NO: 15 is threonine, valine, or cysteine. In some embodiments, the TRX has a substitution at a position corresponding to position 32 of SEQ ID NO: 16. In some embodiments, the amino acid at a position corresponding to position 32 of SEQ ID NO: 16 is alanine, cysteine, aspartic acid, glycine, proline, or serine. In some embodiments, a DNA polymerase herein comprises a TBD having at least 50% (e.g., 50%, 55%, 60%, 65%, 70%, 75%, 80%, 85%, 90%, 95%, 99%, 100%, or ranges therebetween) sequence identity with SEQ ID NO: 199. In some embodiments, the TBD is located at the C-terminus, N-terminus, inserted within one of SEQ ID NOS: 3, 5, 7, 9, and 11, and/or substituted for all or a portion one of SEQ ID NOS: 3, 5, 7, 9, and 11. In some embodiments, a DNA polymerase herein comprises a TRX having at least 50% (e.g., 50%, 55%, 60%, 65%, 70%, 75%, 80%, 85%, 90%, 95%, 99%, 100%, or ranges therebetween) sequence identity with SEQ ID NO: 16, 17, or 107. In some embodiments, the TRX is located at the C-terminus, N-terminus, inserted within one of SEQ ID NOS: 3, 5, 7, 9, and 11, and/or substituted for all or a portion one of SEQ ID NOS: 3, 5, 7, 9, and 11. In some embodiments, a DNA polymerase herein comprises a TIS having at least 40% (e.g., 40%, 45%, 50%, 55%, 60%, 65%, 70%, 75%, 80%, 85%, 90%, 95%, 99%, 100%, or ranges therebetween) sequence identity with one of SEQ ID NOS: 18-21. In some embodiments, the TIS is located at the C-terminus, N-terminus, inserted within one of SEQ ID NOS: 3, 5, 7, 9, and 11, and/or substituted for all or a portion of SEQ ID NOS: 3, 5, 7, 9, and 11. In some embodiments, an exonuclease domain of SEQ ID NO: 13 is deleted from the sequence corresponding to SEQ ID NO: 1.

In some embodiments, provided herein are DNA polymerases comprising a sequence having at least 40% (e.g., 40%, 45%, 50%, 55%, 60%, 65%, 70%, 75%, 80%, 85%, 90%, 95%, 99%, 100%, or ranges therebetween) sequence identity to one of:
(a) (SEQ ID NO: 2)-(SEQ ID NO: 3)-(SEQ ID NO: 4)-(SEQ ID NO: 5)-(SEQ ID NO: 6)-(SEQ ID NO: 7)-(SEQ ID NO: 8)-(SEQ ID NO: 9)-(SEQ ID NO: 10)-(SEQ ID NO: 199)-(SEQ ID NO: 12);
(b) (SEQ ID NO: 2)-(one of SEQ ID NOS: 18-21)-(SEQ ID NO: 4)-(SEQ ID NO: 5)-(SEQ ID NO: 6)-(SEQ ID NO: 7)-(SEQ ID NO: 8)-(SEQ ID NO: 9)-(SEQ ID NO: 10)-(SEQ ID NO: 199)-(SEQ ID NO: 12);
(c) (SEQ ID NO: 2)-(SEQ ID NO: 3)-(SEQ ID NO: 4)-(one of SEQ ID NOS: 18-21)-(SEQ ID NO: 6)-(SEQ ID NO: 7)-(SEQ ID NO: 8)-(SEQ ID NO: 9)-(SEQ ID NO: 10)-(SEQ ID NO: 199)-(SEQ ID NO: 12);
(d) (SEQ ID NO: 2)-(SEQ ID NO: 3)-(SEQ ID NO: 4)-(SEQ ID NO: 5)-(SEQ ID NO: 6)-(one of SEQ ID NOS: 18-21)-(SEQ ID NO: 8)-(SEQ ID NO: 9)-(SEQ ID NO: 10)-(SEQ ID NO: 199)-(SEQ ID NO: 12);
(e) (SEQ ID NO: 2)-(SEQ ID NO: 3)-(SEQ ID NO: 4)-(SEQ ID NO: 5)-(SEQ ID NO: 6)-(SEQ ID NO: 7)-(SEQ ID NO: 8)-(one of SEQ ID NOS: 18-21)-(SEQ ID NO: 10)-(SEQ ID NO: 199)-(SEQ ID NO: 12);
(f) (SEQ ID NO: 2)-(SEQ ID NO: 3)-(SEQ ID NO: 4)-(SEQ ID NO: 5)-(SEQ ID NO: 6)-(SEQ ID NO: 7)-(SEQ ID NO: 8)-(SEQ ID NO: 9)-(SEQ ID NO: 10)-(SEQ ID NO: 199)-(SEQ ID NO: 12)-(SEQ ID NO: 200);
(g) (SEQ ID NO: 2)-(one of SEQ ID NOS: 18-21)-(SEQ ID NO: 4)-(SEQ ID NO: 5)-(SEQ ID NO: 6)-(SEQ ID NO: 7)-(SEQ ID NO: 8)-(SEQ ID NO: 9)-(SEQ ID NO: 10)-(SEQ ID NO: 199)-(SEQ ID NO: 12)-(SEQ ID NO: 200);
(h) (SEQ ID NO: 2)-(SEQ ID NO: 3)-(SEQ ID NO: 4)-(one of SEQ ID NOS: 18-21)-(SEQ ID NO: 6)-(SEQ ID NO: 7)-(SEQ ID NO: 8)-(SEQ ID NO: 9)-(SEQ ID NO: 10)-(SEQ ID NO: 199)-(SEQ ID NO: 12)-(SEQ ID NO: 200);
(i) (SEQ ID NO: 2)-(SEQ ID NO: 3)-(SEQ ID NO: 4)-(SEQ ID NO: 5)-(SEQ ID NO: 6)-(one of SEQ ID NOS: 18-21)-(SEQ ID NO: 8)-(SEQ ID NO: 9)-(SEQ ID NO: 10)-(SEQ ID NO: 199)-(SEQ ID NO: 12)-(SEQ ID NO: 200);
(j) (SEQ ID NO: 2)-(SEQ ID NO: 3)-(SEQ ID NO: 4)-(SEQ ID NO: 5)-(SEQ ID NO: 6)-(SEQ ID NO: 7)-(SEQ ID NO: 8)-(one of SEQ ID NOS: 18-21)-(SEQ ID NO: 10)-(SEQ ID NO: 199)-(SEQ ID NO: 12)-(SEQ ID NO: 200);
(k) (SQ ID NO: 200)-(SEQ ID NO: 2)-(SEQ ID NO: 3)-(SEQ ID NO: 4)-(SEQ ID NO: 5)-(SEQ ID NO: 6)-(SEQ ID NO: 7)-(SEQ ID NO: 8)-(SEQ ID NO: 9)-(SEQ ID NO: 10)-(SEQ ID NO: 15)-(SEQ ID NO: 12);
(l) (SQ ID NO: 200)-(SEQ ID NO: 2)-(one of SEQ ID NOS: 18-21)-(SEQ ID NO: 4)-(SEQ ID NO: 5)-(SEQ ID NO: 6)-(SEQ ID NO: 7)-(SEQ ID NO: 8)-(SEQ ID NO: 9)-(SEQ ID NO: 10)-(SEQ ID NO: 199)-(SEQ ID NO: 12);
(m) (SQ ID NO: 200)-(SEQ ID NO: 2)-(SEQ ID NO: 3)-(SEQ ID NO: 4)-(one of SEQ ID NOS: 18-21)-(SEQ ID NO: 6)-(SEQ ID NO: 7)-(SEQ ID NO: 8)-(SEQ ID NO: 9)-(SEQ ID NO: 10)-(SEQ ID NO: 199)-(SEQ ID NO: 12);
(n) (SQ ID NO: 200)-(SEQ ID NO: 2)-(SEQ ID NO: 3)-(SEQ ID NO: 4)-(SEQ ID NO: 5)-(SEQ ID NO: 6)-(one of SEQ ID NOS: 18-21)-(SEQ ID NO: 8)-(SEQ ID NO: 9)-(SEQ ID NO: 10)-(SEQ ID NO: 199)-(SEQ ID NO: 12); and
(o) (SQ ID NO: 200)-(SEQ ID NO: 2)-(SEQ ID NO: 3)-(SEQ ID NO: 4)-(SEQ ID NO: 5)-(SEQ ID NO: 6)-(SEQ ID NO: 7)-(SEQ ID NO: 8)-(one of SEQ ID NOS: 18-21)-(SEQ ID NO: 10)-(SEQ ID NO: 199)-(SEQ ID NO: 12).

In some embodiments, provided herein are DNA polymerases comprising a sequence having at least 40% (e.g., 40%, 45%, 50%, 55%, 60%, 65%, 70%, 75%, 80%, 85%, 90%, 95%, 99%, 100%, or ranges therebetween) sequence identity to one of SEQ ID NOS: 22-49.

In some embodiments, provided herein are DNA polymerases comprising:
(a) one DNA polymerase domain, one TBD, and one TRX;
(b) one DNA polymerase domain, one TBD, and two or more TRXs;
(c) one DNA polymerase domain, two or more TBDs, and one TRX;
(d) one exonuclease-deficient DNA polymerase domain, one TBD, and one TRX;
(e) one exonuclease-deficient DNA polymerase domain, one TBD, and two or more TRXs;
(f) one exonuclease-deficient DNA polymerase domain, two or more TBDs, and one TRX;
(g) one DNA polymerase domain, one TBD, one TRX, and one TIS;
(h) one DNA polymerase domain, one TBD, two or more TRXs, and one TIS;
(i) one DNA polymerase domain, two or more TBDs, one TRX, and one TIS;
(j) one exonuclease-deficient DNA polymerase domain, one TBD, one TRX, and one TIS;
(k) one exonuclease-deficient DNA polymerase domain, one TBD, two or more TRXs, and one TIS; or
(l) one exonuclease-deficient DNA polymerase domain, two or more TBDs, one TRX, and one TIS;
wherein the TBD has non-cysteine amino acids at positions corresponding to positions 14 and 52 of SEQ ID NO: 15; and wherein the TRX domain has a non-cysteine amino acid at a position corresponding to position 35 of SEQ ID NO: 16. In some embodiments, the amino acid at the position corresponding to position 14 of SEQ ID NO: 15 is a leucine. In some embodiments, the amino acid at the position corresponding to position 52 of SEQ ID NO: 15 is a valine. In some embodiments, the amino acid at the position corresponding to position 35 of SEQ ID NO: 16 is a serine. In some embodiments, the TBD has a substitution at a position corresponding to position 66 of SEQ ID NO: 15. In some embodiments, the amino acid at a position corresponding to position 66 of SEQ ID NO: 15 is threonine, valine, or cysteine. In some embodiments, the TBD has a substitution at a position corresponding to position 32 of SEQ ID NO: 16. In some embodiments, the amino acid at a position corresponding to position 32 of SEQ ID NO: 16 is alanine, cysteine, aspartic acid, glycine, proline, or serine. In some embodiments, the DNA polymerase domain has at least 40% (e.g., 40%, 45%, 50%, 55%, 60%, 65%, 70%, 75%, 80%, 85%, 90%, 95%, 99%, 100%, or ranges therebetween) sequence identity to SEQ ID NO: 1 or an ordered combination of 8 or more (e.g., 8, 9, 10, 11) of SEQ ID NOS 2-12. In some embodiments, the TBD has at least 50% (e.g., 50%, 55%, 60%, 65%, 70%, 75%, 80%, 85%, 90%, 95%, 99%, 100%, or ranges therebetween) sequence identity to SEQ ID NO: 199. In some embodiments, the TRX has at least 50% (e.g., 50%, 55%, 60%, 65%, 70%, 75%, 80%, 85%, 90%, 95%, 99%, 100%, or ranges therebetween) sequence identity to SEQ ID NO: 200. In some embodiments, the TIS has at least 40% (e.g., 40%, 45%, 50%, 55%, 60%, 65%, 70%, 75%, 80%, 85%, 90%, 95%, 99%, 100%, or ranges therebetween) sequence identity to one of SEQ ID NOS: 18-21. In some embodiments, the exonuclease-deficient DNA polymerase domain lacks all or a portion of SEQ ID NO: 13.

In some embodiments, provided herein are reaction mixtures comprising a composition, DNA polymerase, or DNA polymerase system, and amplification reagents sufficient to amplify a DNA target sequence. In some embodiments, the amplification reagents comprise one or more of oligonucleotide primers, deoxynucleotide triphosphates, magnesium chloride, buffer, water, and a template DNA comprising the DNA target sequence. In some embodiments, the DNA target sequence comprises one or more short tandem repeats (STRs). In some embodiments, the STR comprises a repetitive unit of 1-50 nucleotides extending 10-500 nucleotides in length. In some embodiments, reaction mixtures further comprise a reducing agent. In some embodiments, the reducing agent is a thiol reductant or non-thiol reductant. In some embodiments, the reducing agent is dithiothreitol (DTT) or tris (2-carboxyethyl) phosphine (TCEP). In some embodiments, reaction mixtures do not comprise a reducing agent. In some embodiments, reaction mixtures do not comprise a thiol reductant or non-thiol reductant. In some embodiments, reaction mixtures do not comprise dithiothreitol (DTT) or tris (2-carboxyethyl) phosphine (TCEP).

In some embodiments, provided herein are methods of amplifying a DNA target sequence comprising exposing the reaction mixture described herein to polymerase chain reaction thermal cycling conditions.

In some embodiments, provided herein are thioredoxin (TRX) polypeptides comprising at least 70% (e.g., 70%, 75%, 80%, 85%, 90%, 95%, 100%) sequence similarity relative to SEQ ID NO: 200 at positions 29-37, 60-77, and 89-98, and wherein the TRX polypeptide is capable of binding to a TRX binding domain (TBD) having an amino acid sequence of SEQ ID NO: 199. In some embodiments, the TRX polypeptide comprises at least 70% (e.g., 70%, 65%, 80%, 85%, 90%, 95%, 100%) sequence identity to SEQ ID NO: 200 at positions 29-37, 60-77, and 89-98. In some embodiments, the TRX polypeptide comprises 100% sequence identity to SEQ ID NO: 200 at positions 29-37, 60-77, and 89-98. In some embodiments, the TRX polypeptide comprises at least 40% (e.g., 40%, 45%, 50%, 55%, 60%, 65%, 70%, 75%, 80%, 85%, 90%, 95%, 99%, 100%, or ranges therebetween) sequence identity with SEQ ID NO: 200. In some embodiments, the TRX polypeptide comprises 50-60% sequence identity with SEQ ID NO: 200. In some embodiments, the TRX polypeptide is 100-120 amino acids in length. In some embodiments, the TRX polypeptide has a 3D fold threshold of 0.8 or greater (e.g., 0.8, 0.85, 0.90, 0.95. 1.0, or ranges therebetween) relative to a TRX of protein database model 6N7W. In some embodiments, the TRX polypeptide has an instability score of less than 40 (e.g., <35, <30, <25, <20, etc.).

In some embodiments, provided herein are thioredoxin (TRX) polypeptides capable of binding to a TRX binding domain (TBD) and having (i) a 3D fold threshold of 0.8 or greater (e.g., 0.8, 0.85, 0.90, 0.95. 1.0, or ranges therebetween) relative to a TRX of protein database model 6N7W, and/or (ii) an instability score of less than 40 (e.g., <35, <30, <25, <20, etc.). In some embodiments, the TRX polypeptide comprises 100% sequence similarity relative to SEQ ID NO: 200 at positions 29-37, 60-77, and 89-98. In some embodiments, the TRX polypeptide is 100-120 amino acids in length. In some embodiments, the TRX is greater than 120 amino acids in length (e.g., 125, 130, 140, 150, 175, 200, 250, 300, 400, 500, or more).

In some embodiments, provided herein are thioredoxin (TRX) polypeptides capable of binding to a TRX binding domain (TBD), wherein for a 3D molecular structure of the TRX polypeptide (e.g., calculated by ESMFold) a root mean squared deviation (RMSD) calculated for alpha carbons of at least 70% (e.g., 70%, 75%, 80%, 85%, 90%, 95%, 100%, or ranges therebetween) of the amino acid residues corresponding amino acids 29-73, 60-77, and 89-98 of SEQ ID NO: 200 is 3.0 Å or less (e.g., 3.0 Å, 2.8 Å, 2.6 Å, 2.4 Å, 2.2 Å, 2.0 Å, 1.8 Å, 1.6 Å, 1.4 Å, 1.2 Å, 1.0 Å, 0.8 Å, 0.6 Å, 0.4 Å, 0.2 Å, or less, or ranges or values therebetween) relative to a TRX of protein database model 6N7W. In some embodiments, the TBD interaction residues of the TRX have an alpha carbon RMSD relative to protein database model 6N7W of 3.0 Å or less. In some embodiments, the TBD interaction residues of the TRX have at least 70% (e.g., 70%, 75%, 80%, 85%, 90%, 95%, 100%, or ranges therebetween) sequence similarity to SEQ ID NO: 200. In some embodiments, the TBD interaction residues of the TRX have at least 70% (e.g., 70%, 75%, 80%, 85%, 90%, 95%, 100%, or ranges therebetween) sequence identity to SEQ ID NO: 200.

In some embodiments, provided herein are A DNA polymerase systems comprising: (a) a DNA polymerase domain comprising at least 40% sequence identity with a Family A DNA polymerase; (b) a thioredoxin binding domain (TBD) having at least 50% sequence identity to a natural phage-derived TBD, wherein the TBD has non-cysteine amino acids at positions corresponding to positions 14 and 52 of SEQ ID NO: 15; and (c) a thioredoxin (TRX) domain capable of binding to the TBD (e.g., a TRX described herein). In some embodiments, the amino acid at the position corresponding to position 14 of SEQ ID NO: 15 is a leucine. In some embodiments, the amino acid at the position corresponding to position 52 of SEQ ID NO: 15 is a valine. In some embodiments, the TBD has a substitution at a position corresponding to position 66 of SEQ ID NO: 15. In some embodiments, the amino acid at a position corresponding to position 66 of SEQ ID NO: 15 is threonine, valine, or cysteine.

In some embodiments, provided herein are DNA polymerase systems comprising: (a) a first polypeptide comprising: (i) a DNA polymerase domain; (ii) a thioredoxin binding domain (TBD); and (iii) a thioredoxin (TRX) domain; and (b) a second polypeptide comprising: (i) a DNA polymerase domain; and (ii) a TBD; wherein the TRX domain has a non-cysteine amino acid at a position corresponding to position 35 of SEQ ID NO: 16; and wherein the TBD of the first polypeptide and/or the TBD of the second polypeptide has non-cysteine amino acids at positions corresponding to positions 14 and 52 of SEQ ID NO: 15. In some embodiments, the amino acid at the position corresponding to position 14 of SEQ ID NO: 15 is a leucine. In some embodiments, the amino acid at the position corresponding to position 52 of SEQ ID NO: 15 is a valine. In some embodiments, the amino acid at the position corresponding to position 35 of SEQ ID NO: 16 is a serine. In some embodiments, the TBD has a substitution at a position corresponding to position 66 of SEQ ID NO: 15. In some embodiments, the amino acid at a position corresponding to position 66 of SEQ ID NO: 15 is threonine, valine, or cysteine. In some embodiments, the TRX has a substitution at a position corresponding to position 32 of SEQ ID NO: 16. In some embodiments, the amino acid at a position corresponding to position 32 of SEQ ID NO: 16 is threonine, valine, or cysteine.

### BRIEF DESCRIPTION OF THE DRAWINGS

FIG. 1. Location of cysteine residues introduced into exemplary reduced stutter polymerase (e.g., construct 8100; SEQ ID NO: 108).

FIG. 2. Average amplicon peak heights (top) and Stutter percentage (bottom) for six constructs combining mutations at the C36, C683, and C721 positions of RSP polymerase construct 8100 (SEQ ID NO: 108); (C36S, C493L, C531K)=8412; (C36S, C493V, C531K)=8413; (C36S, C493K, C531K)=8414; (C36S, C493K, C531V)-8415; (C36S, C493L, C531V)=8416; and (C36S, C493V, C531V)-8417. The six constructs were then evaluated for the ability to amplify an STR duplex in the presence of a reducing agent (DTT) using the lysate stutter assay described in described in U.S. patent application Ser. No. 18/595,339; incorporated by reference in its entirety.

FIG. 3. Average amplicon peak heights (top) and Stutter percentage (bottom) for six constructs combining mutations at the C36, C683, and C721 positions of RSP polymerase construct 8100 (SEQ ID NO: 108); (C36S, C493L, C531K)-8412; (C36S, C493V, C531K)=8413; (C36S, C493K, C531K)=8414; (C36S, C493K, C531V)=8415; (C36S, C493L, C531V)-8416; and (C36S, C493V, C531V)=8417. The six constructs were then evaluated for the ability to amplify an STR duplex in the absence of a reducing agent (DTT) using the lysate stutter assay described in described in U.S. patent application Ser. No. 18/595,339; incorporated by reference in its entirety.

FIG. 4A-B. (A) Exemplary electropherograms of Taq controls (top) and a modified RSP polymerase construct containing the T735C mutation (bottom; SEQ ID NO: 204) amplified in the absence of any reducing agent. (B) Quantification of average stutter percentage (n=3 per condition) demonstrating a reduction in stutter for amplifications mediated by the indicated RSP construct compared to amplifications mediated by Taq.

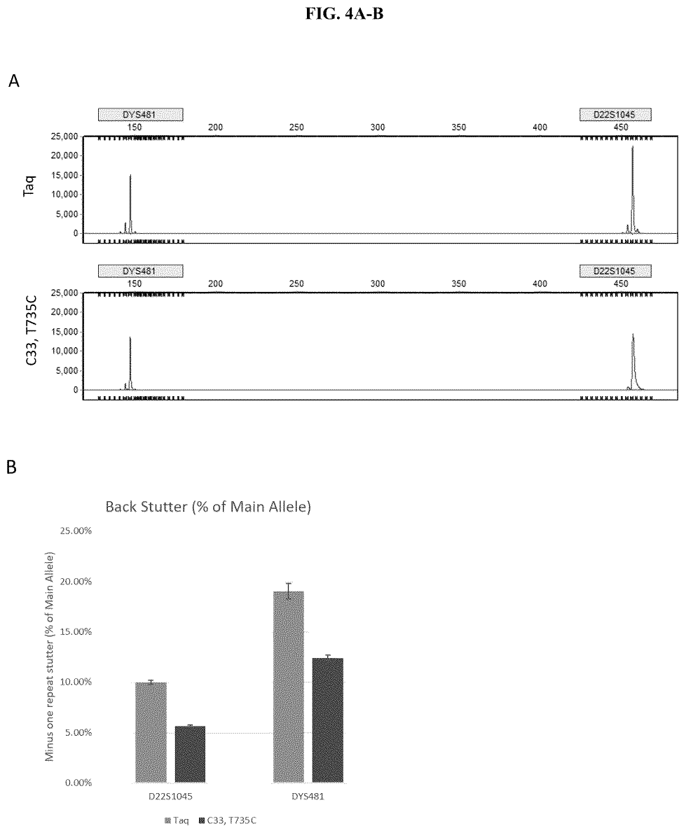

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies, or protocols herein described, as these may vary in accordance with routine experimentation and optimization. It is also to be understood that the terminology used in the description is for the purpose of describing the particular versions or embodiments only and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a,” “an,” and “the” include plural reference unless the context clearly dictates otherwise. Thus, for example, reference to “a domain” is a reference to one or more domains and equivalents thereof known to those skilled in the art, and so forth.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

As used herein, the term “comprise” and linguistic variations thereof denote the presence of recited feature(s), element(s), method step(s), etc., without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily associated impurities. The phrase “consisting essentially of” denotes the recited feature(s), element(s), method step(s), etc. and any additional feature(s), element(s), method step(s), etc., that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “system” refers to a collection of compositions grouped together in any suitable manner (e.g., physically associated, within the same fluid (e.g., reaction mixture, cell lysate, etc.), body (e.g., cell), packaged together (e.g., in a kit), etc.) for a particular purpose.

As used herein, the term “sample” is used in its broadest sense. In one sense, it is meant to include a specimen or culture obtained from any source as well as biological and environmental samples. Biological samples may be obtained from animals (including humans) and encompass fluids, solids, tissues, and gases. Biological samples include blood products such as plasma, serum, and the like. Sample may also refer to cell lysates or purified forms of the enzymes, peptides, and/or polypeptides described herein. Cell lysates may include cells that have been lysed with a lysing agent or lysates such as rabbit reticulocyte or wheat germ lysates. Sample may also include cell-free expression systems. Environmental samples include environmental material such as surface matter, soil, water, crystals, and industrial samples. Such examples are not however to be construed as limiting the sample types applicable to the present invention.

As used herein, the term “reducing agent” refers to a reagent which is capable of reducing another compound in an oxidation-reduction reaction. Examples of reducing agent within the scope herein include tris (2-carboxyethyl) phosphine (TCEP), dithiothreitol (DTT), mercaptoethanol, cysteine, thioglycerol, thioglycolic acid, cysteamine, glutathione, N-acetylcysteine (NAC), β-mercaptoethanol, sodium sulfite, ammonium sulfamate, sodium bisulfite, dithionite metabisulfite sulfur dioxide, vitamin C, ascorbic acid, thiourea, and dimethylsulfoxide (DMSO).

As used herein, the term “DNA polymerase” refers to an enzyme capable of catalyzing the synthesis of a DNA molecule from nucleoside triphosphate building blocks using a DNA template molecule to guide the sequence of the types of nucleotides added. Native DNA polymerases have highly conserved structures among polymerases within the same classes, with the “DNA polymerase domain” or “catalytic domain” varying very little between species. The DNA polymerase domain resembles a right hand and contains “thumb,” “finger,” and “palm” subdomains. DNA polymerases may also contain additional domains that impart various functionalities (e.g., exonuclease domain(s), thioredoxin binding domain, TIS, etc.). DNA polymerases are divided into seven families based on their sequence homology and tertiary structures. These include families A, B, C, D, X, Y, and RT. Polymerase family A includes Pol I (encoded by the polA gene), which is the most abundant and ubiquitous DNA polymerase among prokaryotes, for example, various thermostable DNA polymerase, such as Thermus aquaticus DNA polymerase, Thermus thermophilus DNA polymerase, Thermus flavus DNA polymerase, Thermotoga neapolitana DNA polymerase, and Geobacillus stearothermophilus DNA polymerase, and certain bacteriophage DNA polymerases, such as T7 bacteriophage DNA polymerase and T3 bacteriophage DNA polymerase. In addition to the catalytic domain, Family A polymerases comprise a 3′ to 5′ exonuclease domain.

The terms “DNA polymerase activity,” “synthesis activity,” and “polymerase activity” are used interchangeably and refer to the ability of a DNA polymerase to synthesize new DNA strands by the incorporation of deoxynucleotide triphosphates.

As used herein, the term “Taq DNA polymerase” or “Taq” refers to a DNA polymerase of SEQ ID NO: 1, unless otherwise indicated.

The term “genomic DNA” as used herein refers to any DNA ultimately derived from the DNA of a genome. The term includes, for example, cloned DNA in a heterologous organism, whole genomic DNA, and partial genomic DNA (e.g., the DNA of a single isolated chromosome). The DNA detected, analyzed, isolated, etc., according to embodiments herein can be single-stranded or double-stranded. For example, single-stranded DNA can be obtained from bacteriophage, bacteria, or fragments of genomic DNA. Double-stranded DNA can be obtained from any one of a number of different sources, for example, DNA with tandem repeat sequences, including phage libraries, cosmid libraries, and bacterial genomic or plasmid DNA, and DNA isolated from any eukaryotic organism, including human genomic DNA. In some embodiments, DNA is obtained from human genomic DNA. Any one of a number of different sources of human genomic DNA can be used, including medical or forensic samples, such as blood, semen, vaginal swabs, tissue, hair, saliva, urine, and mixtures of bodily fluids. Such samples can be fresh, old, dried, and/or partially degraded. The samples can be collected from evidence at the scene of a crime.

As used herein, the term “slipped strand mispairing,” “slippage,” and “stutter” refer to the skipping or re-reading by a DNA polymerase of several nucleotides (e.g., 1-8 nucleotides) in the template DNA strand, resulting in the deletion or duplication of nucleotides in the resulting complementary product strand. Forward stutter results in several nucleotides (e.g., 1-8 nucleotides) in the template strand being read twice by the polymerase and the resulting product strand containing a duplication of the sequence complementary to the re-read nucleotides. Backwards stutter results in several nucleotides (e.g., 1-8 nucleotides) in the template strand being skipped and the resulting product strand containing a deletion of the sequence complementary to the skipped nucleotides. Stutter typically occurs at a very low rate on most template sequences, but more commonly occurs when the template strand contains repeated sequences of 1-8 nucleotides (e.g., a tandem repeat).

As used herein, the term “tandem repeat” (a “simple tandem repeat”) refers to a DNA sequence pattern in which a sequence of one or more nucleotides is repeated and the repetitions are directly adjacent to each other. Although typically a short repeating sequence (e.g., 1-8 nucleotides) spanning a 10-500 nucleotide length DNA segment (e.g., 10, 20, 50, 100, 200, 300, 400, 500, or ranges therebetween), tandem repeats may be longer (e.g., 9-50 nucleotides) spanning a DNA segment of 500, 750, 1000 nucleotides or longer. Repetition of a short sequence (e.g., 1-8 nucleotide may be referred to herein as a “short tandem repeat” (“STR”) or a “microsatellite”. Repetition of a single nucleotide is referred to as a “mononucleotide repeat” (for example, “AAAAA”), repetition of two nucleotides is referred to as a “dinucleotide repeat” (for example, “ACACACAC”), repetition of three nucleotides is referred to as a “trinucleotide repeat” (for example, “AGCAGCAGCAGC”), and so on.

As used herein, the term “compound repeat” refers to two or more adjacent simple repeats (i.e., simple tandem repeats with difference sequences).

As used herein, the term “complex repeat” refers to several repeat blocks of variable unit length as well as variable intervening sequences.

As used herein, the term “complex hypervariable repeats” contain numerous non-consensus alleles that can differ in both size and sequence (e.g., SE33)

STR types (e.g., simple, compound, complex, complex hypervariable, etc.) are described, for example, in Chapter 5 (p. 100) of “Advanced Topics in Forensic DNA Typing: Methodology” by John M. Butler (2012), Academic Press; incorporated by reference in its entirety.

Tandem repeats used in forensic analysis (“forensic STRs”) may be simple or complex repeats. In some embodiments, during forensic analysis, the type of STR (simple or complex) is not distinguished.

The term “stutter artifact”, as used herein, refers to the DNA product having an insertion or deletion of a nucleotide or series of nucleotides as the result of a stutter. In an analysis of the DNA product, the stutter artifact will typically appear as a minor signal (e.g., having the insertion or deletion) paired with the major signal (e.g., produced without stutter). Stutter artifacts have been attributed to slipped-strand mispairing during replication of DNA, both in vivo and in vitro (See, e.g., Levinson and Gutman (1987), Mol. Biol. Evol, 4 (3): 203-221; and Schlotterer and Tautz (1992), Nucleic Acids Research 20 (2): 211-215; incorporated by reference in their entireties). Such artifacts are particularly apparent when DNA containing any such repeat sequence is amplified in vitro, using a method of amplification such as the polymerase chain reaction (PCR), as any minor fragment present in a sample or produced during polymerization is amplified along with the major fragments.

As used herein, the term “back stutter” refers to a stutter artifact that occurs at exactly minus one repeat unit.

As used herein, the term “stutter proclivity” refers to the likelihood that a given set of reaction conditions will give rise to stutter and/or stutter artifacts. For example, if a particular DNA polymerase produces fewer stutter artifacts than a control, then the DNA polymerase has a reduced stutter proclivity. If a particular template sequence (e.g., a tandem repeat) gives rise to higher incidences of stutter, then that template increases the stutter proclivity.

As used herein, the terms “template strand” or “template DNA” refer to a sequence of DNA that is read by the DNA polymerase during DNA replication or synthesis. The terms “product strand” or “product DNA” refer to the sequence of DNA that is synthesized during DNA replication. If stutter occurs when duplicating a template strand, the resulting stutter artifact will be present in the product strand.

As used herein, the term “primer” refers to an oligonucleotide capable of hybridizing to a template DNA and serving as an initiation point for DNA synthesis by a DNA polymerase. A primer may be single-stranded or double-stranded. A primer may be perfectly complementary to a sequence within the template DNA or may have one or more mismatches or non-Watson-Crick pairings, provided the primer is capable of hybridizing to the template under amplification conditions. A primer is said to be “capable of hybridizing to a DNA molecule” if that primer is capable of annealing to the DNA molecule; that is the primer shares a degree of complementarity with the DNA molecule. The degree of complementarity can be, but need not be, complete (i.e., the primer need not be 100% complementary to the DNA molecule). Any primer which can anneal to and support primer extension along a template DNA molecule under the reaction conditions employed is capable of hybridizing to a DNA molecule.

As used herein, the terms “complementary” or “complementarity” are used in reference to a sequence of nucleotides related by the base-pairing rules. For example, for the sequence 5′ “A-G-T”3′, is complementary to the sequence 3′ “T-C-A” 5′. Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods which depend upon hybridization of nucleic acids.

As used herein, the term “polymerase chain reaction” (“PCR”) refers to the method described in, for example, U.S. Pat. Nos. 4,683,195, 4,889,818, and 4,683,202, all of which are hereby incorporated by reference. These patents describe methods for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase (e.g., Taq polymerase). The two primers are complementary to their respective strands of the double stranded target sequence. To effect amplification, the mixture is denatured, and the primers then annealed to their complementary sequences within the target molecule. Following annealing, the primers are extended with a polymerase to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

With PCR, it is possible to amplify a single copy of a specific target sequence in genomic DNA to a level detectable by several different methodologies (i.e., hybridization with a labeled probe; incorporation of biotinylated primers followed by avidin-enzyme conjugate detection; incorporation of labeled deoxynucleotide triphosphates, etc.). In addition to genomic DNA, any oligonucleotide sequence can be amplified with the appropriate set of primer molecules. In particular, the amplified segments created by the PCR process itself are, themselves, efficient templates for subsequent PCR amplifications.

As used herein, the term “fusion protein” refers to a chimeric protein comprising two or more peptide/polypeptide portions originating or derived from different sources.

As used herein, the term “modifier” refers to any peptide or polypeptide sequence fused to a peptide, polypeptide, protein of interest to impart a functionality. Non-limiting examples of modifiers include His tags, HaloTag, streptavidin, an antibody, an epitope, a FLAG tag, etc.

As used therein, the terms “conjugated,” “linked,” or linguistic variations thereof refer to the connecting of two moieties via covalent or non-covalent connection. Conjugation or linking can involve a direct covalent bond, or may employ any suitable linking agents, such as peptide linkers, non-peptide linkers, chemical cross-linking agents, etc.

As used herein, the term “peptide” refers to a short polymer of amino acids linked together by peptide bonds. In contrast to other amino acid polymers (e.g., proteins, polypeptides, etc.), peptides are of about 50 amino acids or less in length. A peptide may comprise natural amino acids, non-natural amino acids, amino acid analogs, and/or modified amino acids. A peptide may be a subsequence of naturally occurring protein or a non-natural (artificial) sequence.

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another: 1) Alanine (A) and Glycine (G); 2) Aspartic acid (D) and Glutamic acid (E); 3) Asparagine (N) and Glutamine (Q); 4) Arginine (R) and Lysine (K); 5) Isoleucine (I), Leucine (L), Methionine (M), and Valine (V); 6) Phenylalanine (F), Tyrosine (Y), and Tryptophan (W); 7) Serine(S) and Threonine (T); and 8) Cysteine (C) and Methionine (M).

Naturally occurring residues may be divided into classes based on common side chain properties, for example: polar positive (histidine (H), lysine (K), and arginine (R)); polar negative (aspartic acid (D), glutamic acid (E)); polar neutral (serine(S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs.

Non-conservative substitutions may involve the exchange of a member of one class for a member from another class.

As used herein, the term “sequence identity” refers to the degree to which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Any peptides described herein as having a particular percent sequence identity or similarity (e.g., at least 70%) with a reference sequence, may also be expressed as having a maximum number of substitutions (or terminal deletions) with respect to that reference sequence. For example, a sequence “having at least 70% sequence identity with SEQ ID NO:X” may have up to 3 substitutions relative to SEQ ID NO:X (when SEQ ID NO: X is 10 amino acids in length) and may therefore also be expressed as “having 3 or fewer substitutions relative to SEQ ID NO: X.” Further, a sequence “having at least 80% sequence similarity with SEQ ID NO:X” may have 0, 1, or 2 non-conservative substitutions relative to SEQ ID NO:X, and may therefore also be expressed as “having 2 or fewer non-conservative substitutions relative to SEQ ID NO:X.”

As used herein, the term “root mean squared deviation” (RMSD″) refers to a commonly used quantitative measure of the similarity between pairs of superimposed atomic coordinates. RMSD values are presented in angstroms (Å) and calculated by:

$$RMSD = \sqrt{\frac{1}{N} {\sum }_{i = 1}^{N} {δ}_{i}^{2}}$$

Kufareval and Abagyan. Methods Mol Biol. 2012; 857:231-257; incorporated by reference in its entirety). For calculation of RMSDs for polypeptides herein, 3D molecular structures may be calculated using ESMFold (Zeming Lin et al., Evolutionary-scale prediction of atomic-level protein structure with a language model. Science 379, 1123-1130 (2023); incorporated by reference in its entirety).

As used herein, the term “closely homologous 3D structures” refers to a pair of polypeptides, or a domain or subdomain thereof, that have an alpha carbon RMSD of less the 3 Å between the two.

As used herein, the term “3D fold threshold” refers to a TM-Score calculated using TMAlign v 20170708 (https://bioweb.pasteur.fr/packages/pack@TM-align@20170708; Y. Zhang, J. Skolnick, TM-align-A protein structure alignment algorithm based on TM-score, Nucleic Acids Research, 33 2302-2309 (2005); incorporated by reference in its entirety). In some embodiments, a 3D fold threshold above 0.8 (e.g., 0.85, 0.90, 0.91, 0.92, 0.93, 0.94, 0.95, 0.96, 0.97, 0.98, 0.99, or greater) indicates a high degree of 3D structural identity. For calculation of 3D fold thresholds for polypeptides herein, 3D molecular structures may be calculated using ESMFold (Zeming Lin et al., Evolutionary-scale prediction of atomic-level protein structure with a language model. Science 379, 1123-1130 (2023); incorporated by reference in its entirety).

As used herein, the term “instability score” refers to a quantitative prediction of in vivo stability of a protein based on its primary sequence (Guruprasad et al. Protein Engineering, Design and Selection, Volume 4, Issue 2, December 1990, Pages 155-161; incorporated by reference in its entirety).

### DETAILED DESCRIPTION

Provided herein are compositions and systems comprising a DNA polymerase domain, a thioredoxin binding domain (TBD), and thioredoxin (TRX) engineered to synthesize DNA with reduced stutter artifacts in the absence of a reducing agent. Kits comprising the DNA polymerase/TBD/TRX compositions and systems herein and methods of use thereof in the absence of a reducing agent are also within the scope herein.

In some embodiments, the DNA polymerases and DNA polymerase systems herein are provided in the absence of a reducing agent, for example, in the absence of one or more (e.g., all) of tris (2-carboxyethyl) phosphine (TCEP), dithiothreitol (DTT), mercaptoethanol, cysteine, thioglycerol, thioglycolic acid, cysteamine, glutathione, N-acetylcysteine (NAC), β-mercaptoethanol, sodium sulfite, thiourea, and dimethylsulfoxide (DMSO).

T3 and T7 bacteriophage DNA polymerases are structurally similar to Taq DNA polymerase, however, these phage polymerases contain an additional domain referred to as the “thioredoxin binding domain” (TBD). Binding of host thioredoxin (TRX) to the TBD is required for phage propagation and greatly enhances the processivity of these phage polymerases. Previous publications have described grafting the T3 TBD onto thermostable Taq polymerase (Davidson et al., 2003; incorporated by reference in its entirety), forming a functional chimeric polymerase. This chimera was shown to have increased processivity in the presence of an extremely high concentration of TRX, similar to studies performed with T7 DNA polymerase. Increases in processivity do not typically translate into reduced stutter formation (Verheij, S., Harteveld, J., and Sijen, T. (2012) Forensic Science International: genetics, Vol. 6, pp. 167-175; incorporated by reference in its entirety). The need for a large molar excess of TRX relative to the chimeric Taq-TBD (Davidson et al.; incorporated by reference in its entirety) greatly restricts the utility of this approach when applied to traditional PCR techniques. Specifically, the solubility and volume restrictions to achieve this molar ratio while maintaining stability throughout thermal cycling limit the commercial utility and practical applications of this approach. Experiments were conducted during development of embodiments herein to overcome this limitation through multiple approaches, such as by genetically fusing one or more thioredoxins with a TBD-modified Taq polymerase (e.g., internally or at the N- or C-terminus). These modifications ameliorate the need for excess, exogenous thioredoxin for reduced stutter and robust polymerase activity. An STR multiplex amplified with these genetic fusion constructs exhibit ˜10-50% of the stutter artifacts compared to multiplexes amplified by unmodified Taq, with the precise stutter formation being dependent on the specific locus being amplified. 0.6-160 molar fold excess of free thioredoxin in the presence of a Taq-TBD (without genetically fused thioredoxin) is sufficient to amplify an STR multiplex with the same reduction in stutter as the genetic fusions. This amount of thioredoxin can be delivered in a sufficiently concentrated stock solution that is compatible with traditional PCR approaches. Additional experiments have demonstrated the utility of other Taq, TBD, and/or TRX constructs that find use in, for example, reducing stutter.

In addition to a TBD, T3 and T7 DNA polymerases have a “TBD/TRX interacting sequence” (TIS) that is contemplated to interact with one or more of the TBD, TRX, catalytic domain, and/or DNA template to enhance aspects of DNA synthesis. In some embodiments, all (e.g., SEQ ID NO: 18) or a portion (e.g., one or SEQ ID NOS: 19-21 or a portion of SEQ ID NO: 18) is fused to or inserted within a polymerase as described herein to enhance one or more aspects of DNA synthesis.

U.S. patent application Ser. No. 18/595,339 (incorporated by reference in its entirety) describes engineered DNA polymerases and DNA polymerase systems with reduced artifact formation. Provided herein are engineered DNA polymerases and DNA polymerase systems comprising substitutions with respect the DNA polymerases and DNA polymerase systems of U.S. patent application Ser. No. 18/595,339 that reduce or eliminate the need for reducing agent(s) in DNA synthesis reactions. In some embodiments, the polymerases herein (or polymerase-containing systems) comprise substitutions in the TRX domain and/or TBD relative to the DNA polymerases and DNA polymerase systems U.S. patent application Ser. No. 18/595,339 that reduce or eliminate the need for reducing agent(s) in DNA synthesis reactions. In some embodiments, the polymerases herein (or polymerase-containing systems) provide reduced formation of stutter products when amplifying highly repetitive sequences (e.g., STR multiplexes) in the absence of reducing agents (e.g., DTT, TCEP, etc.). In some embodiments, polymerases herein (or polymerase-containing systems) produce reduced stutter artifacts (e.g., due to a reduced stutter proclivity for the polymerases or systems herein relative to Taq or other polymerases) in the absence of reducing agents (e.g., DTT, TCEP, etc.). For example, in some embodiments, polymerases herein (or polymerase-containing systems) produce reduced stutter artifacts (e.g., have reduced stutter proclivity) compared to a polymerase comprising the DNA polymerase domain only (e.g., a Taq polymerase of SEQ ID NO: 1). In some embodiments, the polymerases herein (or polymerase-containing systems) produce fewer stutter artifacts (e.g., 5% fewer, 10% fewer, 15% fewer, 20% fewer, 25% fewer, 30% fewer, 35% fewer, 40% fewer, 45% fewer, 50% fewer, 65% fewer, 70% fewer, 75% fewer, 80% fewer, 85% fewer, 90% fewer, 95% fewer, 99% fewer, or ranges therebetween) compared to a polymerase comprising the DNA polymerase domain only (e.g., a Taq polymerase of SEQ ID NO: 1). In some embodiments, the polymerases herein (or polymerase-containing systems) have a reduced stutter proclivity (e.g., 5% reduced, 10% reduced, 15% reduced, 20% reduced, 25% reduced, 30% reduced, 35% reduced, 40% reduced, 45% reduced, 50% reduced, 65% reduced, 70% reduced, 75% reduced, 80% reduced, 85% reduced, 90% reduced, 95% reduced, 99% reduced, or ranges therebetween) compared to a polymerase comprising the DNA polymerase domain only (e.g., a Taq polymerase of SEQ ID NO: 1). In some embodiments, the polymerases herein (or polymerase-containing systems) produced fewer stutter artifacts, for example, when amplifying highly repetitive sequences (e.g., STR multiplexes, mononucleotide repeats, etc.). In some embodiments, the polymerases herein (or polymerase-containing systems) are capable of amplifying DNA comprising highly repetitive sequences (e.g., STR multiplexes, mononucleotide repeats, etc.) in the absence of reducing agent (e.g., DTT, TCEP, etc.).

In some embodiments, provided herein are systems and compositions comprising a DNA polymerase domain, a thioredoxin (TRX), and a thioredoxin binding domain (TBD). In some embodiments, a TRX comprises the substitutions of SEQ ID NO: 200 relative to SEQ ID NO: 16 or 17. In some embodiments, a TBD comprises the substitutions of SEQ ID NO: 199 relative to SEQ ID NO: 15. In some embodiments, systems and compositions herein further comprise one or more additional components, such as linkers to all or a portion of a heterologous polymerase domain (e.g., capable of interacting with TBD and/or TRX). In some embodiments, systems and compositions herein further comprise portions of heterologous polymerases (e.g., T3, T7, etc.), for example, portions of the T3 or T7 exonuclease domain (e.g., all or a portion of the TIS (e.g., SEQ ID NOS: 18-21).

In some embodiments, the two or more of the various components of the compositions and systems herein are provided as a fusion (e.g., a single polypeptide). In some embodiments, all of the components of a composition herein (e.g., polymerase domain, TRX (e.g., comprising the substitutions of SEQ ID NO: 200 relative to SEQ ID NO: 16 or 17), TBD (e.g., comprising the substitutions of SEQ ID NO: 199 relative to SEQ ID NO: 15), etc.) are provided as a single fusion polypeptide. In some embodiments, one or more of the various components of the compositions and systems herein are provided as a separate polypeptide (e.g., not fused to one or more of the other components). In some embodiments, either the TBD (e.g., comprising the substitutions of SEQ ID NO: 199 relative to SEQ ID NO: 15) or TRX (e.g., comprising the substitutions of SEQ ID NO: 200 relative to SEQ ID NO: 16 or 17) (or both) are fused or otherwise conjugated to the DNA polymerase domain. In some embodiments, the components of a system herein may be provided as 2, 3, or more different polypeptides. In some embodiments, the components of a composition herein may be provided as a single polypeptide.

### DNA Polymerase Domain

In some embodiments, the polymerases herein comprise a DNA polymerase domain. As defined herein, the DNA polymerase domain is a polypeptide capable of catalyzing DNA synthesis under appropriate conditions. In some embodiments, the DNA polymerase domain of a composition or system herein comprises sequence homology with all or a portion (e.g., 40%, 45%, 50%, 55%, 60%, 65%, 70%, 75%, 80%, 85%, 90%, 95%, 100%) of a DNA polymerase enzyme (e.g., a Family A DNA polymerase (e.g., Taq polymerase, Tne polymerase, etc.), etc.). In some embodiments, a composition (or component of a system) herein comprise a DNA polymerase domain having sequence homology to all or a portion of a DNA polymerase enzyme, with various other components (e.g., TRX, TBD, TIS or portion thereof, linkers, etc.) inserted within the sequence of the DNA polymerase enzyme, replacing a portion of the sequence of the DNA polymerase enzyme (e.g., 1-50 amino acids (e.g., 1, 2, 5, 10, 15, 20, 25, 30, 35, 40, 45, 50, or ranges therebetween)), or fused (directly or via one or more linkers) to the N-terminus or C-terminus of the DNA polymerase enzyme. In certain embodiments, such as with an exonuclease-deficient polymerase, regions of the polymerase domain as large as 100-300 amino acids (e.g., 235 amino acids) may be deleted or replaced with alternative domains or components. In some embodiments, homology modeling and tertiary structure analysis are utilized to identify regions of a DNA polymerase enzyme sequence that are suitable sites of insertion of components of the compositions herein (e.g., TRX, TBD, TIS, linkers, etc.) or replacement by components of the compositions herein (e.g., TRX, TBD, TIS, linkers, etc.).

In some embodiments, a jFATCAT pairwise structure alignment between pdb files 1TAQ and IT7P was used to determine suitable sites of insertion of components of the compositions herein (e.g., TRX, TBD, TIS, linkers, etc.) or replacement by components of the compositions herein (e.g., TRX, TBD, TIS, linkers, etc.). In some embodiments, primary sequence homology within alpha helices or flexible domains of Taq DNA polymerase and the T7 DNA polymerase was used to determine suitable sites of insertion of components of the compositions herein (e.g., TRX, TBD, TIS, linkers, etc.) or replacement by components of the compositions herein (e.g., TRX, TBD, TIS, linkers, etc.).

In some embodiments, the polymerases herein comprise a DNA polymerase domain that is derived from a natural or previously known DNA polymerase. In some embodiments, the DNA polymerase domain is derived from a Family A DNA polymerase, such as the Thermus aquaticus DNA polymerase (SEQ ID NO: 1), T7 DNA polymerase, DNA polymerase I, DNA polymerase y, Tne polymerase, and DNA polymerase 0. In some embodiments, a DNA polymerase domain is a chimera of two or more different Family A DNA polymerases. In some embodiments, the DNA polymerase domain is derived from a native thermophilic DNA polymerase. In some embodiments, the native thermophilic DNA polymerase is selected from the group consisting of the Thermus aquaticus DNA polymerase, Thermus thermophilus DNA polymerase, Thermus flavus DNA polymerase, Thermotoga neapolitana polymerase, and Geobacillus stearothermophilus DNA polymerase. In some embodiments, the DNA polymerase domain comprises at least 40% (e.g., 40%, 45%, 50%, 55%, 60%, 65%, 70%, 75%, 80%, 85%, 90%, 95%, 98%, 99%, 100%, or ranges therebetween) sequence identity with SEQ ID NO: 1; however, in other embodiments, a functional DNA polymerase domain may comprise less than 70% (e.g., <60%, <50%, <40%, or less) sequence identity with SEQ ID NO: 1. In some embodiments, the DNA polymerase domain maintains the DNA synthesis functionality as well as one or more additional characteristics (e.g., thermostability) of the DNA polymerase from which they are derived. In some embodiments, a polymerase with proof-reading activity, a polymerase without (or with negligible) proof-reading activity, with exonuclease activity (e.g., 3′ to 5′, 5′ to 3′, etc.), without exonuclease activity, hot start polymerase, a non-hot start polymerase, etc., is used as the basis for the DNA polymerase domain. Examples of DNA polymerases from which a DNA polymerase domain is derived include a HotStarTaq DNA polymerase (QIAGEN catalog No. 203203), AmpliTaq Gold® DNA Polymerase (Applied Biosystems catalog No._N8080241), KAPA Taq DNA Polymerase, KAPA Taq HotStart DNA Polymerase (KAPA BIOSYSTEMS catalog No. BK1000), Pfu DNA polymerase (Thermo Scientific catalog No._EP0501), Klentaq1 (DNA POLYMERASE TECHNOLOGY, Inc, St. Louis, Mo., catalog No._100), a PHUSION DNA polymerase, such as PHUSION High Fidelity DNA polymerase (M0530S, New England BioLabs, Inc.) or PHUSION Hot Start Flex DNA polymerase (M0535S, New England BioLabs, Inc), a Q5R DNA Polymerase, such as Q5® High-Fidelity DNA Polymerase (M0491S, New England BioLabs, Inc.) or Q5@ Hot Start High-Fidelity DNA Polymerase (M0493S, New England BioLabs, Inc.), a T4 DNA polymerase (M0203S, New England BioLabs, Inc.), Sequenase Version 2.0 DNA polymerase (ThermoFisher Scientific catalog No. 70775Y200UN), etc.

In some embodiments, a DNA polymerase domain herein is defined with reference to a Taq DNA polymerase sequence of SEQ ID NO: 1. In certain embodiments, the DNA polymerase domain of a DNA polymerase herein has at least 40% (e.g., 40%, 45%, 50%, 55%, 60%, 65%, 70%, 75%, 80%, 85%, 90%, 95%, 99%, or more, or ranges therebetween) sequence identity to SEQ ID NO: 1. In some embodiments, the DNA polymerase domain comprises a C-terminal and/or N-terminal truncation of 1-50 amino acids (e.g., 1, 2, 3, 4, 5, 6, 7, 8, 9, 10, 11, 12, 13, 14, 15, 16, 17, 18, 19, 20, 25, 30, 35, 40, 45, 50, or ranges therebetween) relative to SEQ ID NO: 1. In some embodiments, the DNA polymerase domain comprises conservative or nonconservative substitutions relative to SEQ ID NO: 1. In some embodiments, the DNA polymerase domain of a DNA polymerase herein has at least 40% (e.g., 40%, 45%, 50%, 55%, 60%, 65%, 70%, 75%, 80%, 85%, 90%, 95%, 99%, or more, or ranges therebetween) sequence identity to a portion of SEQ ID NO: 1.

In some embodiments, a DNA polymerase may comprise at least 40% (e.g., 40%, 45%, 50%, 55%, 60%, 65%, 70%, 75%, 80%, 85%, 90%, 95%, 99%, or more, or ranges therebetween) sequence identity with one or more of SEQ ID NOS: 2, 3, 4, 5, 6, 7, 8, 9, 10, 11, and 12. In some embodiments, a DNA polymerase comprises at least 40% (e.g., 40%, 45%, 50%, 55%, 60%, 65%, 70%, 75%, 80%, 85%, 90%, 95%, 99%, or more, or ranges therebetween) sequence identity with each of SEQ ID NOS: 2, 4, 6, 8, 10, and 12 (in order, but allowing for one or more of SEQ ID NOS: 3, 5, 7, 9, 11, and/or other sequences inserted between). In some embodiments, a DNA polymerase comprises at least 40% (e.g., 40%, 45%, 50%, 55%, 60%, 65%, 70%, 75%, 80%, 85%, 90%, 95%, 99%, or more, or ranges therebetween) sequence identity with each of SEQ ID NOS: 2, 4, 6, 8, 10, and 12 (in order), and one or more of SEQ ID NOS: 3, 5, 7, 9, and 11 (positioned in order).

In some embodiments, a DNA polymerase comprises at least 40% (e.g., 40%, 45%, 50%, 55%, 60%, 65%, 70%, 75%, 80%, 85%, 90%, 95%, 99%, or more, or ranges therebetween) sequence identity with each of SEQ ID NOS: 2, 3, 4, 5, 6, 7, 8, 9, 10, and 12 (in order). In some embodiments, a heterologous amino acid sequence (e.g., TIS, TRX, TBD, etc.) is inserted between SEQ ID NOS: 10 and 12.

In some embodiments, a DNA polymerase comprises at least 40% (e.g., 40%, 45%, 50%, 55%, 60%, 65%, 70%, 75%, 80%, 85%, 90%, 95%, 99%, or more, or ranges therebetween) sequence identity with each of SEQ ID NOS: 2, 4, 5, 6, 7, 8, 9, 10, and 12 (in order). In some embodiments, a heterologous amino acid sequence (e.g., TIS, TRX, TBD, etc.) is inserted between SEQ ID NOS: 2 and 4 and/or SEQ ID NOS: 10 and 12.

In some embodiments, a DNA polymerase comprises at least 40% (e.g., 40%, 45%, 50%, 55%, 60%, 65%, 70%, 75%, 80%, 85%, 90%, 95%, 99%, or more, or ranges therebetween) sequence identity with each of SEQ ID NOS: 2, 3, 4, 6, 7, 8, 9, 10, and 12 (in order). In some embodiments, a heterologous amino acid sequence (e.g., TIS, TRX, TBD, etc.) is inserted between SEQ ID NOS: 4 and 6 and/or SEQ ID NOS: 10 and 12.

In some embodiments, a DNA polymerase comprises at least 40% (e.g., 40%, 45%, 50%, 55%, 60%, 65%, 70%, 75%, 80%, 85%, 90%, 95%, 99%, or more, or ranges therebetween) sequence identity with each of SEQ ID NOS: 2, 3, 4, 5, 6, 8, 9, 10, and 12 (in order). In some embodiments, a heterologous amino acid sequence (e.g., TIS, TRX, TBD, etc.) is inserted between SEQ ID NOS: 6 and 8 and/or SEQ ID NOS: 10 and 12.

In some embodiments, a DNA polymerase comprises at least 40% (e.g., 40%, 45%, 50%, 55%, 60%, 65%, 70%, 75%, 80%, 85%, 90%, 95%, 99%, or more, or ranges therebetween) sequence identity with each of SEQ ID NOS: 2, 3, 4, 5, 6, 7, 8, 10, and 12 (in order). In some embodiments, a heterologous amino acid sequence (e.g., TIS, TRX, TBD, etc.) is inserted between SEQ ID NOS: 8 and 10 and/or SEQ ID NOS: 10 and 12.

In some embodiments, a heterologous amino acid sequence (e.g., TIS, TRX, TBD, etc.) is inserted within SEQ ID NO: 3. In some embodiments, a heterologous amino acid sequence (e.g., TIS, TRX, TBD, etc.) is inserted within SEQ ID NO: 5. In some embodiments, a heterologous amino acid sequence (e.g., TIS, TRX, TBD, etc.) is inserted within SEQ ID NO: 7. In some embodiments, a heterologous amino acid sequence (e.g., TIS, TRX, TBD, etc.) is inserted within SEQ ID NO: 9. In some embodiments, a heterologous amino acid sequence (e.g., TIS, TRX, TBD, etc.) is inserted within SEQ ID NO: 11.

In some embodiments, the DNA polymerase domain of a DNA polymerase herein has overall homology to SEQ ID NO: 1 (as described in the preceding paragraph), but all or a portion of one or more of SEQ ID NOS: 3, 5, 7, 9, and 11 are replaced by a heterologous insertion sequence. For example, the positions corresponding to SEQ ID NOS: 3, 5, 7, and/or 9 (or portions thereof) may be replaced by all or a portion of a TIS of a DNA polymerase (e.g., T7 polymerase TIS (e.g., SEQ ID NOS: 18-21 or portions or variants thereof, T3 polymerase TIS, etc.)). In some embodiments, the positions corresponding to SEQ ID NO: 11 may be replaced by all or a portion of a TBD (e.g., SEQ ID NO: 15 or portions or variants thereof) or a TRX (e.g., SEQ ID NO: 16 or 17 or portions or variants thereof). In some embodiments, other sequences within the DNA binding domain may be deleted or replaced by heterologous sequences (e.g., a TBD, a TRX, a TIS, other portions of other polymerases, etc.) provided that the DNA polymerase domain maintains a catalytic DNA synthesis activity. In some embodiments, only a portion of one or more of SEQ ID NOS: 3, 5, 7, 9, and 11 are replaced by a heterologous insertion sequence. In such embodiments, a portion(s) of one or more of SEQ ID NOS: 3, 5, 7, 9, and 11 remain in place in the DNA polymerase domain.

In some embodiments, the DNA polymerase domain comprises an internal amino acid sequence insertion. In some embodiments, the DNA polymerase domain comprises an N-terminal portion with at least 40% sequence identity (e.g., 40%, 45%, 50%, 55%, 60%, 65%, 70%, 75%, 80%, 85%, 90%, 95%, 98%, 99%, 100%, or ranges therebetween) to SEQ ID NO: 14 and a C-terminal portion with at least 40% sequence identity (e.g., 40%, 45%, 50%, 55%, 60%, 65%, 70%, 75%, 80%, 85%, 90%, 95%, 98%, 99%, 100%, or ranges therebetween) to SEQ ID NO: 12, wherein the N-terminal portion and the C-terminal portion are separated by the internal amino acid sequence insertion. In some embodiments, the internal amino acid sequence insertion comprises the TBD.

In some embodiments, a DNA polymerase domain (e.g., SEQ ID NO: 1) of a DNA polymerase (or polymerase-containing system) herein comprises an exonuclease domain (SEQ ID NO: 13). In some embodiments, a DNA polymerase domain is truncated by deletion of the exonuclease domain (SEQ ID NO: 13).

In some embodiments, a DNA polymerase domain herein comprises one or more substitutions relative to a reference DNA polymerase sequence. For example, using Taq DNA polymerase as a base sequence for a DNA polymerase domain, the DNA polymerase domain may comprise one or more substitutions (e.g., 1, 2, 3, 4, 5, 6, 7, 8, 9, 10, 15, 20, 25, 30, 35, 40, 45, 50, 60, 70, 80, 90, 100, 125, 150, 175, 200, 225, 250, 275, 300, 325, or ranges or values therebetween) relative to SEQ ID NO: 1. Exemplary substitutions include the H914 substitutions (position 914 relative to SEQ ID NO: 35) of Table 1 of U.S. patent application Ser. No. 18/595,339 (incorporated by reference in its entirety), A913 substitutions (position 913 relative to SEQ ID NO: 35) of Table 2 of U.S. patent application Ser. No. 18/595,339 (incorporated by reference in its entirety), R915 substitutions (position 915 relative to SEQ ID NO: 35) of Table 3 of U.S. patent application Ser. No. 18/595,339 (incorporated by reference in its entirety), and the various mutations of the Taq DNA polymerase of Table 4 of U.S. patent application Ser. No. 18/595,339 (incorporated by reference in its entirety); however, substitutions in the DNA polymerase domain relative to SEQ ID NO: 1 or another base DNA polymerase sequence are not limited to these positions or substitutions. In some embodiments, a DNA polymerase domain is based on a Tne DNA polymerase, Tfl DNA polymerase, Taq DNA polymerase, or chimeras thereof (See e.g., Tables 20 and 21). Experiments conducted during development of embodiments herein have demonstrated that Family A DNA polymerases with divergent sequences and substitutions at a wide variety of locations throughout the sequence find use as a DNA polymerase domain in the embodiments herein. The DNA polymerase domains of the construct herein are not limited to the sequence of a particular DNA polymerase.

### Thioredoxin Binding Domain (TBD)

In some embodiments, a DNA polymerase (or polymerase-containing system) herein comprises a thioredoxin binding domain. In some embodiments, a DNA polymerase domain of a DNA polymerase herein comprises a TBD (e.g., comprising the substitutions of SEQ ID NO: 199 relative to SEQ ID NO: 15) fused to the N- or C-terminus of the DNA polymerase domain (e.g., directly or via one or more linkers). In some embodiments, a DNA polymerase domain comprises a TBD (e.g., comprising the substitutions of SEQ ID NO: 199 relative to SEQ ID NO: 15) inserted internally within the DNA polymerase domain. In some embodiments, a TBD (e.g., comprising the substitutions of SEQ ID NO: 199 relative to SEQ ID NO: 15) is inserted at a position corresponding to or adjacent to amino acid positions within a sequence provided herein (e.g., SEQ ID NO: 1 or a sequence having at least 50% sequence identity thereto). In other embodiments, a TBD (e.g., comprising the substitutions of SEQ ID NO: 199 relative to SEQ ID NO: 15) is inserted within or replaces all or a portion of an amino acid sequence corresponding to all or a portion of a sequence provided herein (e.g., SEQ ID NO: 3, 5, 7, 9, 11, or any suitable region of SEQ ID NO: 1, or a sequence having at least 50% sequence identity thereto) is replaced by a TBD (e.g., comprising the substitutions of SEQ ID NO: 199 relative to SEQ ID NO: 15). In some embodiments, the TBD (e.g., comprising the substitutions of SEQ ID NO: 199 relative to SEQ ID NO: 15) is fused or inserted at a location of the DNA polymerase domain that maintains all or a portion of the catalytic function or other functional characteristics of the DNA polymerase domain. In some embodiments, the TBD (e.g., comprising the substitutions of SEQ ID NO: 199 relative to SEQ ID NO: 15) is inserted within the thumb domain (e.g., SEQ ID NO: 11) of a DNA polymerase domain (e.g., SEQ ID NO: 1). In some embodiments, all or a portion of the thumb domain (e.g., SEQ ID NO: 11) of a DNA polymerase domain (e.g., SEQ ID NO: 1) herein is replaced by a TBD (e.g., comprising the substitutions of SEQ ID NO: 199 relative to SEQ ID NO: 15). In some embodiments, a system comprises a TBD (e.g., comprising the substitutions of SEQ ID NO: 199 relative to SEQ ID NO: 15) that is not fused or otherwise conjugated to a DNA polymerase domain (e.g., in a binary system in which a TRX (e.g., comprising the substitutions of SEQ ID NO: 200 relative to SEQ ID NO: 16 or 17) is fused/conjugated to a DNA polymerase domain).

In embodiments in which a DNA polymerase domain corresponds to a Family A DNA polymerase without a high degree of sequence identity to SEQ ID NO: 1, the TBD (e.g., comprising the substitutions of SEQ ID NO: 199 relative to SEQ ID NO: 15) is inserted at a location that retains all or a portion of the catalytic activity of a DNA polymerase.

In some embodiments, a system herein comprises a TBD (e.g., comprising the substitutions of SEQ ID NO: 199 relative to SEQ ID NO: 15) that is not fused to a DNA polymerase domain. In such embodiments, the DNA polymerase domain is fused or otherwise conjugated to at least one TRX (e.g., comprising the substitutions of SEQ ID NO: 200 relative to SEQ ID NO: 16 or 17). In some embodiments, the presence of the TBD (e.g., comprising the substitutions of SEQ ID NO: 199 relative to SEQ ID NO: 15) within the same system as a DNA polymerase domain fused/conjugated to a TRX (e.g., comprising the substitutions of SEQ ID NO: 200 relative to SEQ ID NO: 16 or 17) results in an active polymerase or polymerase system in the absence of reducing agent and reduced stutter proclivity for the DNA polymerase domain relative to a system lacking the TBD and/or TRX. In some embodiments, the TRX (e.g., comprising the substitutions of SEQ ID NO: 200 relative to SEQ ID NO: 16 or 17) is fused or otherwise conjugated to the DNA polymerase domain. In some embodiments, the TRX (e.g., comprising the substitutions of SEQ ID NO: 200 relative to SEQ ID NO: 16 or 17) is fused or otherwise conjugated to the TBD. In some embodiments, the TBD (e.g., comprising the substitutions of SEQ ID NO: 199 relative to SEQ ID NO: 15) is conjugated (e.g., covalently or non-covalently) but not fused to the DNA polymerase domain.

In some embodiments, a TBD of a DNA polymerase (or system comprising a DNA polymerase) herein is derived from the thioredoxin binding domain of a T3 or T7 bacteriophage DNA polymerase. In some embodiments, the TBD comprises at least 50% (e.g., 50%, 55%, 60%, 65%, 70%, 75%, 80%, 85%, 90%, 95%, 98%, 99%, 100%, or ranges therebetween) sequence identity with SEQ ID NO: 15. In some embodiments, a TBD comprises a C-terminal and/or N-terminal truncation relative to SEQ ID NO: 15 of 1-20 amino acids (e.g., 1, 2, 3, 4, 5, 6, 7, 8, 9, 10, 11, 12, 13, 14, 15, 16, 17, 18, 19, 20, or ranges therebetween). In some embodiments, a TBD comprises up to 30 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or ranges therebetween) substitutions (e.g., conservative or nonconservative) relative to SEQ ID NO: 15.

In some embodiments, a TBD of a DNA polymerase (or system comprising a DNA polymerase) herein comprises at least 50% (e.g., 50%, 55%, 60%, 65%, 70%, 75%, 80%, 85%, 90%, 95%, 98%, 99%, 100%, or ranges therebetween) sequence identity with SEQ ID NO: 199. In some embodiments, a TBD of a DNA polymerase (or system comprising a DNA polymerase) herein comprises substitutions relative to SEQ ID NO: 15 at one or more of positions 14, 52, and 66 of SEQ ID NO: 15. In some embodiments, a TBD of a DNA polymerase (or system comprising a DNA polymerase) herein comprises a non-cysteine amino acid at position 14 of SEQ ID NO: 15 (e.g., a sequence of SEQ ID NO: 199). In some embodiments, a TBD of a DNA polymerase (or system comprising a DNA polymerase) herein comprises a non-cysteine amino acid at position 52 of SEQ ID NO: 15 (e.g., a sequence of SEQ ID NO: 199). In some embodiments, a TBD of a DNA polymerase (or system comprising a DNA polymerase) herein comprises a alanine, cysteine, aspartic acid, glycine, proline, or serine at position 66 of SEQ ID NO: 15 (e.g., a sequence of SEQ ID NO: 199).

In some embodiments, a TBD of a DNA polymerase (or system comprising a DNA polymerase) herein (e.g., a sequence derived from a T3 or T7 TBD) may comprise substitutions relative to the reference sequences herein (e.g., SEQ ID NO: 15 or 199), such as the exemplary substitutions of Table 6 and Table 7 of U.S. patent application Ser. No. 18/595,339 (incorporated by reference in its entirety). In some embodiments, a TBD comprises substitutions at one or more of T489, R506, T535, E537, E548, and S555 (relative to SEQ ID NO: 35), such as those listed in Table 8 of U.S. patent application Ser. No. 18/595,339 (incorporated by reference in its entirety). Other substitutions relative to a reference TBD (e.g., SEQ ID NO: 15) are within the scope herein. In some embodiments, a TBD of a DNA polymerase (or system comprising a DNA polymerase) herein comprises a non-cysteine amino acid at position 14 of SEQ ID NO: 15, a non-cysteine amino acid at position 52 of SEQ ID NO: 15, and a alanine, cysteine, aspartic acid, glycine, proline, or serine at position 66 of SEQ ID NO: 15 (e.g., a sequence of SEQ ID NO: 199).

In some embodiments, a TBD of a DNA polymerase (or system comprising a DNA polymerase) herein comprises a non-cysteine amino acid at position 14 of SEQ ID NO: 15. In some embodiments, the non-cysteine amino acid at position 14 of SEQ ID NO: 15 is a leucine.

In some embodiments, a TBD of a DNA polymerase (or system comprising a DNA polymerase) herein comprises a non-cysteine amino acid at position 52 of SEQ ID NO: 15. In some embodiments, the non-cysteine amino acid at position 52 of SEQ ID NO: 15 is a valine.

In some embodiments, a TBD of a DNA polymerase (or system comprising a DNA polymerase) herein comprises an alanine, cysteine, aspartic acid, glycine, proline, or serine at position 66 of SEQ ID NO: 15. In some embodiments, a TBD of a DNA polymerase (or system comprising a DNA polymerase) herein is derived from the thioredoxin binding domain of a Salmonella enterica phage DNA polymerase (e.g., and comprising the substitutions of SEQ ID NO: 199 relative to SEQ ID NO: 15). In some embodiments, the TBD comprises at least 50% (e.g., 50%, 55%, 60%, 65%, 70%, 75%, 80%, 85%, 90%, 95%, 98%, 99%, 100%, or ranges therebetween) sequence identity with SEQ ID NO: 101 (e.g., and comprising the substitutions of SEQ ID NO: 199 relative to SEQ ID NO: 15). In some embodiments, a TBD comprises a C-terminal and/or N-terminal truncation relative to SEQ ID NO: 101 of 1-20 amino acids (e.g., 1, 2, 3, 4, 5, 6, 7, 8, 9, 10, 11, 12, 13, 14, 15, 16, 17, 18, 19, 20, or ranges therebetween). In some embodiments, a TBD comprises up to 30 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or ranges therebetween) substitutions (e.g., conservative or nonconservative) relative to SEQ ID NO: 101.

In some embodiments, a TBD of a DNA polymerase (or system comprising a DNA polymerase) herein is derived from the thioredoxin binding domain of an Aeromonas hydrophila phage DNA polymerase (e.g., and comprising the substitutions of SEQ ID NO: 199 relative to SEQ ID NO: 15). In some embodiments, the TBD comprises at least 50% (e.g., 50%, 55%, 60%, 65%, 70%, 75%, 80%, 85%, 90%, 95%, 98%, 99%, 100%, or ranges therebetween) sequence identity with SEQ ID NO: 102 (e.g., and comprising the substitutions of SEQ ID NO: 199 relative to SEQ ID NO: 15). In some embodiments, a TBD comprises a C-terminal and/or N-terminal truncation relative to SEQ ID NO: 102 of 1-20 amino acids (e.g., 1, 2, 3, 4, 5, 6, 7, 8, 9, 10, 11, 12, 13, 14, 15, 16, 17, 18, 19, 20, or ranges therebetween). In some embodiments, a TBD comprises up to 30 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or ranges therebetween) substitutions relative to SEQ ID NO: 102.

In some embodiments, a TBD of a DNA polymerase (or system comprising a DNA polymerase) herein is derived from the thioredoxin binding domain of a Klebsiella pneumoniae phage DNA polymerase (e.g., and comprising the substitutions of SEQ ID NO: 199 relative to SEQ ID NO: 15). In some embodiments, the TBD comprises at least 50% (e.g., 50%, 55%, 60%, 65%, 70%, 75%, 80%, 85%, 90%, 95%, 98%, 99%, 100%, or ranges therebetween) sequence identity with SEQ ID NO: 103 (e.g., and comprising the substitutions of SEQ ID NO: 199 relative to SEQ ID NO: 15). In some embodiments, a TBD comprises a C-terminal and/or N-terminal truncation relative to SEQ ID NO: 103 of 1-20 amino acids (e.g., 1, 2, 3, 4, 5, 6, 7, 8, 9, 10, 11, 12, 13, 14, 15, 16, 17, 18, 19, 20, or ranges therebetween). In some embodiments, a TBD comprises up to 30 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or ranges therebetween) substitutions relative to SEQ ID NO: 103.

In some embodiments, a DNA polymerase (or system comprising a DNA polymerase) herein may comprise two or more TBDs (e.g., 2, 3, 4, 5, or more). In some embodiments, at least one of the TBDs in a DNA polymerase (or system comprising a DNA polymerase) herein comprises the substitutions described herein to promote activity in the absence of a reducing agent (e.g., DTT, TCEP, etc.). In some embodiments, the TBDs are fused or conjugated to different locations on the DNA polymerase domain. In some embodiments, two or more TBD sequences (e.g., identical TBD sequences (e.g., comprising the substitutions of SEQ ID NO: 199 relative to SEQ ID NO: 15)), different TBD sequences) are fused or conjugated to a DNA polymerase domain in series (e.g., one after another). In some embodiments, two or more TBDs are included in a monomeric DNA polymerase polypeptide. In other embodiments, two or more TBDs are included in separate polypeptides in a binary DNA polymerase system (e.g., TBD/Pol-TRX, TBD-Pol-TRX/TBD, TBD-Pol-TRX/TBD-Pol, etc.).

In some embodiments, a TBD is fused or conjugated to a TRX. In some embodiments, a system comprises a TBD and a TRX are conjugated or fused in a manner (e.g., directly, via one more linkers, through interaction partners, etc.) to facilitate binding of the TBD to the TRX (and subsequently to reduce stutter proclivity of an associated (e.g., bound to one or both of the TBD or TRX, within the same system, etc.) DNA polymerase domain. In embodiments in which a TRX and TBD are fused or otherwise conjugated (e.g., directly or via a linker), one or both of the TBD and/or TRX is fused or otherwise conjugated (e.g., directly or via a linker) to the DNA polymerase domain.

In some embodiments, a free TBD is provided (e.g., in a binary system comprising a DNA polymerase domain fused/conjugated to a TRX). In some embodiments, a free TBD is not fused or conjugated to a DNA polymerase domain or a TRX. In some embodiments, addition of a free TBD to a system comprising a suitable DNA polymerase domain fused or otherwise linked to a TRX results in reduced stutter relative to the DNA polymerase domain in the absence of TRX and/or the free TBD. In some embodiments, a binary system comprises a first polypeptide comprising a TBD (e.g., TBD, TBD-Pol, TBD-Pol-TRX, etc.) and a second polypeptide comprising a TRX (e.g., TRX, TRX-Pol, TBD-Pol-TRX, etc.).

### Thioredoxin (TRX)

In some embodiments, a DNA polymerase (or polymerase-containing system) herein comprises a thioredoxin (e.g., comprising the substitutions of SEQ ID NO: 200 relative to SEQ ID NO: 16 or 17). In some embodiments, a DNA polymerase domain of a polymerase herein comprises a thioredoxin (TRX) fused to the N- or C-terminus or inserted internally within the DNA polymerase domain. In some embodiments, the TRX (e.g., comprising the substitutions of SEQ ID NO: 200 relative to SEQ ID NO: 16 or 17) is fused or inserted at a location of the DNA polymerase domain that allows for maintenance of all or a portion of the catalytic activity or other functional characteristics of the DNA polymerase or the TRX. In some embodiments, a DNA polymerase domain comprises a TRX (e.g., comprising the substitutions of SEQ ID NO: 200 relative to SEQ ID NO: 16 or 17) inserted internally within the DNA polymerase domain. In some embodiments, a TRX (e.g., comprising the substitutions of SEQ ID NO: 200 relative to SEQ ID NO: 16 or 17) is inserted at a position corresponding to or adjacent to amino acid positions within a sequence provided herein (e.g., SEQ ID NO: 1 or a sequence having at least 50% sequence identity thereto). In other embodiments, a TRX (e.g., comprising the substitutions of SEQ ID NO: 200 relative to SEQ ID NO: 16 or 17) is inserted within or replaces all or a portion of an amino acid sequence corresponding to all or a portion of a sequence provided herein (e.g., SEQ ID NO: 3, 5, 7, 9, 11, or any suitable region of SEQ ID NO: 1, or a sequence having at least 50% sequence identity thereto).

In some embodiments, a TRX of a DNA polymerase (or system comprising a DNA polymerase) herein is derived from E. coli thioredoxin (e.g., comprising the substitutions of SEQ ID NO: 200 relative to SEQ ID NO: 16 or 17). In some embodiments, the TRX domain comprises at least 50% (e.g., 50%, 55%, 60%, 65%, 70%, 75%, 80%, 85%, 90%, 95%, 98%, 99%, 100%, or ranges therebetween) sequence identity with SEQ ID NO: 200 (e.g., and comprising the substitutions of SEQ ID NO: 200 relative to SEQ ID NO: 16 or 17). In some embodiments, a TRX comprises a C-terminal and/or N-terminal truncation relative to SEQ ID NO: 200 of 1-20 amino acids (e.g., 1, 2, 3, 4, 5, 6, 7, 8, 9, 10, 11, 12, 13, 14, 15, 16, 17, 18, 19, 20, or ranges therebetween). In some embodiments, a TRX comprises up to 30 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or ranges therebetween) substitutions relative to SEQ ID NO: 200. In some embodiments, a TRX of a DNA polymerase (or system comprising a DNA polymerase) herein (e.g., a sequence derived from an E. coli TRX) may comprise substitutions relative to the reference sequence (e.g., SEQ ID NO: 16, 17, or 200), such as the exemplary substitutions of Table 9 and Table 10 of U.S. patent application Ser. No. 18/595,339 (incorporated by reference in its entirety). In some embodiments, a TRX comprises substitutions at E31 (relative to SEQ ID NO: 35), such as those listed in Table 11 of U.S. patent application Ser. No. 18/595,339 (incorporated by reference in its entirety). Other substitutions relative to a reference TRX (e.g., SEQ ID NO: 16, 17, 51-53, 93, 94, etc.) are within the scope herein. In some embodiments, a TRX comprises one or more substitutions of SEQ ID NO: 200 relative to SEQ ID NO: 16 or 17.

In some embodiments, a TRX of a DNA polymerase (or system comprising a DNA polymerase) herein is derived from Alishwanella jeotgali thioredoxin (e.g., comprising substitutions of SEQ ID NO: 200 relative to SEQ ID NO: 16 or 17). In some embodiments, the TRX domain comprises at least 50% (e.g., 50%, 55%, 60%, 65%, 70%, 75%, 80%, 85%, 90%, 95%, 98%, 99%, 100%, or ranges therebetween) sequence identity with SEQ ID NO: 94 (e.g., and comprising substitutions of SEQ ID NO: 200 relative to SEQ ID NO: 16 or 17). In some embodiments, a TRX comprises a C-terminal and/or N-terminal truncation relative to SEQ ID NO: 94 of 1-20 amino acids (e.g., 1, 2, 3, 4, 5, 6, 7, 8, 9, 10, 11, 12, 13, 14, 15, 16, 17, 18, 19, 20, or ranges therebetween). In some embodiments, a TRX comprises up to 30 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or ranges therebetween) substitutions relative to SEQ ID NO: 94 (e.g., and comprising substitutions of SEQ ID NO: 200 relative to SEQ ID NO: 16 or 17).

In some embodiments, a TRX of a DNA polymerase (or system comprising a DNA polymerase) herein is derived from Thiococcus pfennigii thioredoxin (e.g., comprising substitutions of SEQ ID NO: 200 relative to SEQ ID NO: 16 or 17). In some embodiments, the TRX domain comprises at least 50% (e.g., 50%, 55%, 60%, 65%, 70%, 75%, 80%, 85%, 90%, 95%, 98%, 99%, 100%, or ranges therebetween) sequence identity with SEQ ID NO: 93 (e.g., and comprising substitutions of SEQ ID NO: 200 relative to SEQ ID NO: 16 or 17). In some embodiments, a TRX comprises a C-terminal and/or N-terminal truncation relative to SEQ ID NO: 93 of 1-20 amino acids (e.g., 1, 2, 3, 4, 5, 6, 7, 8, 9, 10, 11, 12, 13, 14, 15, 16, 17, 18, 19, 20, or ranges therebetween). In some embodiments, a TRX comprises up to 30 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or ranges therebetween) substitutions relative to SEQ ID NO: 93 (e.g., and comprising substitutions of SEQ ID NO: 200 relative to SEQ ID NO: 16 or 17).

In some embodiments, a TRX of a polymerase and/or polymerase system herein comprises an engineered TRX that is functionally and/or structurally based on known TRX polypeptide(s) but has a divergent sequence with low sequence identity, such as the exemplary engineered TRX sequences of Table 25 of U.S. patent application Ser. No. 18/595,339 (incorporated by reference in its entirety). In some embodiments, a TRX is engineered via traditional methods of random mutagenesis, directed mutagenesis and other techniques for altering the amino acid sequence in a directed (e.g., rational) or undirected (e.g., random) manner. In other embodiments, engineered TRXs are generated by maintaining the 3D structure of all or a portion of a reference TRX (e.g., SEQ ID NO: 16). For example, the 3D structure of the portion of a TRX that contacts the TBD (e.g., in PDB 6N7W).

TRX from E. coli, T. pfennigii, and A. jeotgali are all capable of functioning to reduce stutter in DNA polymerase systems described herein. These TRX exhibit overall sequence identities of 69-76% between each other, but higher sequence identities of 77.8% to 100% between their TBD interaction subdomains:
TBD interaction subdomain 1 of SEQ ID NO: 16 has 100% identity to E. coli TRX, 100% identity to T. pfennigii TRX, and 88.9% identity to A. jeotgali TRX;
TBD interaction subdomain 2 of SEQ ID NO: 16 has 100% identity to E. coli TRX, 77.8% identity to T. pfennigii TRX, and 83.3% identity to A. jeotgali TRX; and
TBD interaction subdomain 3 has 100% identity to E. coli TRX, 80% identity T. pfennigii TRX, and 90% identity to A. jeotgali TRX.

In some embodiments, a TRX derived from E. coli, T. pfennigii, and A. jeotgali and comprising one or more substitutions of SEQ ID NO: 200 relative to SEQ ID NO: 16 or 17 is provided herein.

TRX polypeptides have been engineered using AI-assisted protein sequence design, protein structure prediction, and protein structure alignment software and methods. The identity and 3D structural fold of the TBD interaction residues (selected residues in the putative TBD-TRX binding interface; residues 29-73 (TBD interaction subdomain 1), 60-77 (TBD interaction subdomain 2), and 89-98 (TBD interaction subdomain 3), were fixed and candidate sequences were generated that were predicted to fold to present the TBD interaction residues in the same 3D configuration. Of 1000 candidate TRX molecules generated, the alpha carbon RMSDs between 3D models of those sequences and 6N7W for the TBD interaction residues was between 0.86 Å and 2.82 Å, with a mean of 1.15 Å and 1.10 Å. Three TRXs engineered by this process were tested for the capacity to function to reduce stutter. Despite having less than 55% sequence identity to E. coli, these three TRX sequences (SEQ ID NOS: 51-53), were capable of reducing stutter in a test system. These experiments indicate that structurally similar presentation of the TBD interacting residues is sufficient to confer the reduced stutter interaction between TRX and the TBD.

3D molecular structures were calculated for SEQ ID NOS: 51-53 using ESMFold (Zeming Lin et al., Evolutionary-scale prediction of atomic-level protein structure with a language model. Science 379, 1123-1130(2023); incorporated by reference in its entirety). The RMSDs were calculated for the “TBD interaction residues” (Residues 29-73, 60-77, and 89-98 relative to SEQ ID NO: 16) in each of the 3D molecular structures calculated for SEQ ID NOS: 51-53 using ESMFold with the molecular structure of PDB entry 6N7W (Gao et al. (2019) Science 363(6429); incorporated by reference in its entirety), and the resulting RMSDs for the TBD interaction residues were between 1.0 Å and 1.1 Å for the three engineered TRXs. RMSDs were calculated using the “superimpose Proteins” plugin tool (docs.nanome.ai/plugins/superimpose.html #instructions; incorporated by reference in its entirety) on Nanome Version 1.24 (Bennie S, Maritan M, Gast J, Loschen M, Gruffat D, Bartolotta R, Hessenauer S, Leija E, McCloskey S. A Virtual and Mixed Reality Platform for Molecular Design & Drug Discovery-Nanome Version 1.24. 5th Workshop on Molecular Graphics and Visual Analysis of Molecular Data, 2023; 2023 Jun. 12, The Eurographics Association; incorporated by reference in its entirety).

In some embodiments, provided herein are TRX polypeptides with predicted 3D molecular structures (e.g., predicted using ESMFold (Zeming Lin et al., Evolutionary-scale prediction of atomic-level protein structure with a language model. Science 379, 1123-1130 (2023); incorporated by reference in its entirety) in which the TBD interaction residues (Residues 29-73, 60-77, and 89-98 relative to SEQ ID NO: 16) have an alpha carbon RMSD relative to PDB 6N7W of 3 Å or less (e.g., 3 Å, 2.8 Å, 2.6 Å, 2.4 Å. 2.2 Å, 2.0 Å, 1.8 Å, 1.6 Å, 1.4 Å. 1.2 Å, 1.0 Å, 0.8 Å, 0.6 Å, 0.4 Å. 0.2 Å, or less, or values or ranges therebetween) (e.g., and comprising one or more substitutions of SEQ ID NO: 200 relative to SEQ ID NO: 16 or 17). In some embodiments, a group of at least 70% (e.g., 70%, 75%, 80%, 85%, 90%, 95%, 100%) of the TBD interaction residues (Residues 29-73, 60-77, and 89-98 relative to SEQ ID NO: 16) have an alpha carbon RMSD relative to PDB 6N7W of 3 Å or less (e.g., 3 Å, 2.8 Å, 2.6 Å, 2.4 Å. 2.2 Å, 2.0 Å, 1.8 Å, 1.6 Å, 1.4 Å. 1.2 Å, 1.0 Å, 0.8 Å, 0.6 Å, 0.4 Å. 0.2 Å, or less, or values or ranges therebetween) (e.g., and comprising one or more substitutions of SEQ ID NO: 200 relative to SEQ ID NO: 16 or 17). In some embodiments, TBD interaction subdomain 1 (Residues 29-73 relative to SEQ ID NO: 16) has an alpha carbon RMSD relative to PDB 6N7W of 3 Å or less (e.g., 3 Å, 2.8 Å, 2.6 Å, 2.4 Å. 2.2 Å, 2.0 Å, 1.8 Å, 1.6 Å, 1.4 Å. 1.2 Å, 1.0 Å, 0.8 Å, 0.6 Å, 0.4 Å. 0.2 Å, or less, or values or ranges therebetween) (e.g., and comprising one or more substitutions of SEQ ID NO: 200 relative to SEQ ID NO: 16 or 17). In some embodiments, TBD interaction subdomain 2 (Residues 60-77 relative to SEQ ID NO: 16) has an alpha carbon RMSD relative to PDB 6N7W of 3 Å or less (e.g., 3 Å, 2.8 Å, 2.6 Å, 2.4 Å. 2.2 Å, 2.0 Å, 1.8 Å, 1.6 Å, 1.4 Å. 1.2 Å, 1.0 Å, 0.8 Å, 0.6 Å, 0.4 Å. 0.2 Å, or less, or values or ranges therebetween) (e.g., and comprising one or more substitutions of SEQ ID NO: 200 relative to SEQ ID NO: 16 or 17). In some embodiments, TBD interaction subdomain 3 (Residues 89-98 relative to SEQ ID NO: 16) has an alpha carbon RMSD relative to PDB 6N7W of 3 Å or less (e.g., 3 Å, 2.8 Å, 2.6 Å, 2.4 Å. 2.2 Å, 2.0 Å, 1.8 Å, 1.6 Å, 1.4 Å. 1.2 Å, 1.0 Å, 0.8 Å, 0.6 Å, 0.4 Å. 0.2 Å, or less, or values or ranges therebetween) (e.g., and comprising one or more substitutions of SEQ ID NO: 200 relative to SEQ ID NO: 16 or 17).

In some embodiments, the TBD interaction residues (Residues 29-73, 60-77, and 89-98 relative to SEQ ID NO: 16) of a TRX have at least 70% (e.g., 70%, 75%, 80%, 85%, 90%, 95%, 100%) sequence similarity with the TBD interaction residues of SEQ ID NO: 200 (e.g., and comprising one or more substitutions of SEQ ID NO: 200 relative to SEQ ID NO: 16 or 17). In some embodiments, the TBD interaction residues (Residues 29-73, 60-77, and 89-98 relative to SEQ ID NO: 16) of a TRX have at least 70% (e.g., 70%, 75%, 80%, 85%, 90%, 95%, 100%) sequence identity with the TBD interaction residues of SEQ ID NO: 200 (e.g., and comprising one or more substitutions of SEQ ID NO: 200 relative to SEQ ID NO: 16 or 17).

In some embodiments, the TBD interaction subdomain 1 (Residues 29-73 relative to SEQ ID NO: 16) of a TRX has at least 70% (e.g., 70%, 75%, 80%, 85%, 90%, 95%, 100%) sequence similarity with the TBD interaction residues of SEQ ID NO: 200 (e.g., and comprising one or more substitutions of SEQ ID NO: 200 relative to SEQ ID NO: 16 or 17). In some embodiments, the TBD interaction subdomain 1 (Residues 29-73 relative to SEQ ID NO: 16) of a TRX has at least 70% (e.g., 70%, 75%, 80%, 85%, 90%, 95%, 100%) sequence identity with the TBD interaction residues of SEQ ID NO: 200 (e.g., and comprising one or more substitutions of SEQ ID NO: 200 relative to SEQ ID NO: 16 or 17).

In some embodiments, the TBD interaction subdomain 2 (Residues 60-77 relative to SEQ ID NO: 16) of a TRX has at least 70% (e.g., 70%, 75%, 80%, 85%, 90%, 95%, 100%) sequence similarity with the TBD interaction residues of SEQ ID NO: 200 (e.g., and comprising one or more substitutions of SEQ ID NO: 200 relative to SEQ ID NO: 16 or 17). In some embodiments, the TBD interaction subdomain 2 (Residues 60-77 relative to SEQ ID NO: 16) of a TRX has at least 70% (e.g., 70%, 75%, 80%, 85%, 90%, 95%, 100%) sequence identity with the TBD interaction residues of SEQ ID NO: 200 (e.g., and comprising one or more substitutions of SEQ ID NO: 200 relative to SEQ ID NO: 16 or 17).

In some embodiments, the TBD interaction subdomain 3 (Residues 89-98 relative to SEQ ID NO: 16) of a TRX has at least 70% (e.g., 70%, 75%, 80%, 85%, 90%, 95%, 100%) sequence similarity with the TBD interaction residues of SEQ ID NO: 200 (e.g., and comprising one or more substitutions of SEQ ID NO: 200 relative to SEQ ID NO: 16 or 17). In some embodiments, the TBD interaction subdomain 3 (Residues 89-98 relative to SEQ ID NO: 16) of a TRX has at least 70% (e.g., 70%, 75%, 80%, 85%, 90%, 95%, 100%) sequence identity with the TBD interaction residues of SEQ ID NO: 200 (e.g., and comprising one or more substitutions of SEQ ID NO: 200 relative to SEQ ID NO: 16 or 17).

In some embodiments, an engineered TRX may be shorter or longer than a TRX of SEQ ID NO: 200, provided that TBD interaction residues (e.g., residues having structural and/or sequence identity or similarity to a TRX of SEQ ID NO: 16) are closely homologous 3D structures (e.g., and comprising one or more substitutions of SEQ ID NO: 200 relative to SEQ ID NO: 16 or 17). A TRX may be between about 75 and 500 (or more residues in length (e.g., 75, 100, 125, 150, 175, 200, 250, 300, 400, 500, or more).

In some embodiments, an engineered TRX comprises a 3D fold threshold relative to PDB 6N7W above 0.8 (e.g., 0.85, 0.90, 0.91, 0.92, 0.93, 0.94, 0.95, 0.96, 0.97, 0.98, 0.99, or greater) indicating a high degree of 3D structural identity.

In some embodiments, a TRX of a polymerase or system herein comprises at least 50% (e.g., 50%, 55%, 60%, 65%, 70%, 75%, 80%, 85%, 90%, 95%, 100%) sequence identity to one of SEQ ID NOS: 51, 52, or 53 (e.g., and comprising one or more substitutions of SEQ ID NO: 200 relative to SEQ ID NO: 16 or 17). In some embodiments, a TRX comprises the structural elements of a TRX and/or the capability to reduce stutter proclivity in a polymerase system.

In some embodiments, the TRX sequence (e.g., comprising one or more substitutions of SEQ ID NO: 200 relative to SEQ ID NO: 16 or 17) is fused to the N- or C-terminus of the DNA polymerase domain. In some embodiments, the TRX sequence (e.g., comprising one or more substitutions of SEQ ID NO: 200 relative to SEQ ID NO: 16 or 17) is fused to the DNA polymerase domain by a linker of 1-300 amino acids (e.g., 1, 2, 3, 4, 5, 6, 7, 8, 9, 10, 15, 20, 25, 30, 35, 40, 45, 50, 55, 60, 65, 70, 75, 80, 85, 90, 100, 125, 150, 175, 200, 250, 300 or ranges therebetween (e.g., 30-70 amino acids in length, etc.)). A linker may be of any suitable peptide/polypeptide sequence, including, but not limited to those of Table 13 of U.S. patent application Ser. No. 18/595,339 (incorporated by reference in its entirety). In some embodiments, a linker is a flexible linker. For example, in some embodiments, the linker is 50-100% (e.g., 50%, 55%, 60%, 65%, 70%, 75%, 80%, 85%, 90%, 95%, 100%, or ranges therebetween) glycine and serine residues, but linkers may be of any suitable amino acid makeup. In some embodiments, a linker comprises a sequence having at least 40% sequence identity to an exemplary linker in Tables 13 or 14. In some embodiments, a linker is a rigid linker and/or comprises a rigid segment. For example, a linker may comprise one or more (e.g., 1, 2, 3, 4, 5, 6, 7, 8, 9, 10, 12, 15, 20, 25, 30, or more) EAAK peptide segments or other peptides capable of introducing rigidity into the linker. Certain embodiments herein are not limited by the identity of the linker.

In some embodiments, the TRX sequence (e.g., comprising one or more substitutions of SEQ ID NO: 200 relative to SEQ ID NO: 16 or 17) is not fused to the DNA polymerase and/or TBD. In some embodiments, a free TRX (e.g., comprising one or more substitutions of SEQ ID NO: 200 relative to SEQ ID NO: 16 or 17) may be fused to one or more peptide or polypeptide modifiers of 1-100 amino acids (e.g., 1, 2, 3, 4, 5, 6, 7, 8, 9, 10,15, 20, 25, 30, 35, 40, 45, 50, 55, 60, 65, 70, 75, 80, 85, 90, 95, 100, or ranges therebetween). In some embodiments, a free TRX (e.g., comprising one or more substitutions of SEQ ID NO: 200 relative to SEQ ID NO: 16 or 17) comprises a peptide or polypeptide modifier fused to the C- or N-terminus of the TRX sequence (e.g., comprising one or more substitutions of SEQ ID NO: 200 relative to SEQ ID NO: 16 or 17). Examples of modifiers include, but are not limited to, a His tag, HaloTag, streptavidin, an antibody, an epitope, a FLAG tag, etc. In some embodiments, a free TRX (e.g., and comprising one or more substitutions of SEQ ID NO: 200 relative to SEQ ID NO: 16 or 17) is conjugated (e.g., non-genetically linked) to a peptide, polypeptide, or non-peptide (e.g., small molecule, solid surface, etc.) by any suitable conjugation method, such as, click chemistry, thiol-maleimide linkage, cysteine-maleimide-cysteine conjugation, etc.

### Conjugation and Linkers

Provided herein are systems comprising various components (e.g., DNA polymerase domain(s), TBD(s), TRX(s), TIS, etc.). In some embodiments, two or more components (one of which is a DNA polymerase domain) are conjugated, fused, or otherwise physically connected together. For example, in certain embodiments herein, a DNA polymerase domain is genetically fused to a TBD and/or TRX to form a chimeric DNA polymerase. In some embodiments, any of the components described herein may be fused in a manner consistent with this disclosure to yield a DNA polymerase and/or polymerase system within the scope herein. However, the disclosure is not limited to the genetic fusion of the components (e.g., including a DNA polymerase domain) into a single polypeptide. In some embodiments, components may be conjugated or linked (e.g., directly or via one or more linkers), covalently or non-covalently, via any suitable conjugation systems.

In the case of fusion of two or more peptide or polypeptide components, the components (e.g., DNA polymerase domain(s), TBD(s), TRX(s), TIS(s), etc.) may be fused directly (e.g., one component inserted within the other, the C-terminus of one component fused to the N-terminus of a second component, one component substituting a portion of the other, etc.) or indirectly (e.g., via a linker segment). For example, in some embodiments, the DNA polymerase domain and the TBD are connected by a linker. In some embodiments, the DNA polymerase domain and the TRX are connected by a linker. In some embodiments, the TBD and the TRX are connected by a linker. In some embodiments, a component herein (e.g., DNA polymerase domain(s), TBD(s), TRX(s), TIS(s), etc.) is connected to an additional element (e.g., antibody, affinity molecule, DNA binding protein, etc.) by a linker. In some embodiments involving genetic fusion of two or more components, a linker is a peptide or polypeptide linker. In some embodiments, the linker is of a suitable length to allow the components to appropriately interact with one another, to increase the local concentration of one component relative to another, and/or to allow the components to retain their activity or function (e.g., to allow a TBD to function within a chimeric polymerase in a manner similar to that of a TBD of T3 or T7 DNA polymerase). In some embodiments, a TBD sequence is fused to a DNA polymerase domain by a linker of, for example, 1-300 amino acids (e.g., 1, 2, 3, 4, 5, 6, 7, 8, 9, 10, 15, 20, 25, 30, 35, 40, 45, 50, 55, 60, 65, 70, 75, 80, 85, 90, 100, 125, 150, 175, 200, 250, 300, or values or ranges therebetween (e.g., 4-10 amino acids, 30-70 amino acids in length, etc.)). In some embodiments, a TRX sequence is fused to a DNA polymerase domain by a linker of, for example, 1-300 amino acids (e.g., 1, 2, 3, 4, 5, 6, 7, 8, 9, 10, 15, 20, 25, 30, 35, 40, 45, 50, 55, 60, 65, 70, 75, 80, 85, 90, 100, 125, 150, 175, 200, 250, 300, or values or ranges therebetween (e.g., 4-10 amino acids, 30-70 amino acids in length, etc.)). In some embodiments, a TBD sequence is fused to a TRX by a linker of, for example, 1-300 amino acids (e.g., 1, 2, 3, 4, 5, 6, 7, 8, 9, 10, 15, 20, 25, 30, 35, 40, 45, 50, 55, 60, 65, 70, 75, 80, 85, 90, 100, 125, 150, 175, 200, 250, 300, or values or ranges therebetween (e.g., 4-10 amino acids, 30-70 amino acids in length, etc.)). In some embodiments, two tandem elements (e.g., two TRXs, two TBDs, etc.) are fused by a linker of, for example, 1-300 amino acids (e.g., 1, 2, 3, 4, 5, 6, 7, 8, 9, 10, 15, 20, 25, 30, 35, 40, 45, 50, 55, 60, 65, 70, 75, 80, 85, 90, 100, 125, 150, 175, 200, 250, 300, or values or ranges therebetween (e.g., 4-10 amino acids, 30-70 amino acids in length, etc.)). In some embodiments, a component herein (e.g., DNA polymerase domain(s), TBD(s), TRX(s), TIS(s), etc.) and an additional element (e.g., antibody, affinity molecule, DNA binding protein, etc.) are fused by a linker of, for example, 1-300 amino acids (e.g., 1, 2, 3, 4, 5, 6, 7, 8, 9, 10, 15, 20, 25, 30, 35, 40, 45, 50, 55, 60, 65, 70, 75, 80, 85, 90, 100, 125, 150, 175, 200, 250, 300, or values or ranges therebetween (e.g., 4-10 amino acids, 30-70 amino acids in length, etc.)). In some embodiments, two or more linker segments are provided within a polypeptide herein and/or linking two components.

Exemplary linkers for connecting any suitable elements described herein are provided in Tables 12-14. In some embodiments, linkers having at least 60% identity (e.g., 60%, 65%, 70%, 75%, 80%, 85%, 90%, 95%, 100%) with an exemplary linker of Tables 12-14 are provided connecting two components herein (e.g., DNA polymerase domain(s), TBD(s), TRX(s), TIS(s), etc.).

In some embodiments, two components of the DNA polymerases and/or DNA polymerase systems herein are conjugated by disulfide bond formation between components (e.g., TRX and TBD), chemical linkage (e.g., via click chemistry), through the use of protein and/or chemical tags, etc.

Two components (e.g., DNA polymerase domain, TBD(s), TRX(s), TIS, etc.) can be joined by any means known in the art, including covalent and non-covalent interactions. In some embodiments, a first component may be joined to a second component enzymatically or chemically. In some embodiments, a first component may be joined to a second component via ligation. In other embodiments, a first component may be joined to a second component via affinity binding pairs (e.g., biotin and streptavidin). In some cases, a first component may be joined to a second component via an unnatural amino acid, such as via a covalent interaction with an unnatural amino acid.

In some embodiments, a first component may be joined to a second component via SpyCatcher-SpyTag interaction. The SpyTag peptide forms an irreversible covalent bond to the SpyCatcher protein via a spontaneous isopeptide linkage, thereby offering a genetically encoded way to create peptide interactions that resist force and harsh conditions (Zakeri et al., 2012, Proc. Natl. Acad. Sci. 109: E690-697; Li et al., 2014, J. Mol. Biol. 426:309-317). A binding agent may be expressed as a fusion protein comprising the SpyCatcher protein. In some embodiments, the SpyCatcher protein is appended on the N-terminus or C-terminus of the component of the DNA polymerase systems herein. The SpyTag peptide can be coupled to a second component using standard conjugation chemistries (Hermanson, Bioconjugate Techniques, (2013) Academic Press).

In some embodiments, an enzyme-based strategy is used to join a first component to a second component. For example, the first component may be joined to a second component using a formylglycine (FGly)-generating enzyme (FGE). In one example, a protein, e.g., SpyLigase, is used to join the first components to a second component (Fierer et al., Proc Natl Acad Sci USA. 2014; 111(13): E1176-E1181).

In other embodiments, a first components may be joined to a second component via SnoopTag-SnoopCatcher peptide-protein interaction. The SnoopTag peptide forms an isopeptide bond with the SnoopCatcher protein (Veggiani et al., Proc. Natl. Acad. Sci. USA, 2016, 113:1202-1207). A first component may be expressed as a fusion protein comprising the SnoopCatcher protein. In some embodiments, the SnoopCatcher protein is appended on the N-terminus or C-terminus of a component. The SnoopTag peptide can be coupled to the second component using standard conjugation chemistries.

In yet other embodiments, a first component may be joined to a second component via the HaloTag® protein fusion tag and its chemical ligand. HaloTag is a modified haloalkane dehalogenase designed to covalently bind to synthetic ligands (HaloTag® ligands) (Los et al., 2008, ACS Chem. Biol. 3:373-382). The synthetic ligands comprise a chloroalkane linker attached to a variety of molecules. A covalent bond forms between the HaloTag and the chloroalkane linker that is highly specific, occurs rapidly under physiological conditions, and is essentially irreversible.

In some cases, a first component may be joined to a second component by attaching (conjugating) using an enzyme, such as sortase-mediated labeling (See e.g., Antos et al., Curr Protoc Protein Sci. (2009) CHAPTER 15: Unit-15.3; International Patent Publication No. WO2013003555). The sortase enzyme catalyzes a transpeptidation reaction (See e.g., Falck et al, Antibodies (2018) 7(4):1-19). In some aspects, the first component is modified with or attached to one or more N-terminal or C-terminal glycine residues.

In some embodiments, a first component may be joined to a second component using a cysteine bioconjugation method. In some embodiments, a first component is joined to a second component using x-TIS-mediated cysteine bioconjugation (See e.g., Zhang et al., Nat Chem. (2016) 8(2):120-128). In some cases, a first component may be joined to a second component using 3-arylpropiolonitriles (APN)-mediated tagging (e.g., Koniev et al., Bioconjug Chem. 2014; 25(2):202-206).

Other mechanisms of joining the components (e.g., DNA polymerase domain, TBD(s), TRX(s), TIS, etc.) of the systems herein (e.g., click chemistry, antibody conjugation, etc.) are within the scope of this disclosure.

### Chimeras, Systems, and Methods

In some embodiments, provided herein are chimeric DNA polymerases comprising a first DNA polymerase domain fused to a second heterologous (e.g., not native to the DNA polymerase domain) sequence. In some embodiments, the DNA polymerase domain may be fused (or otherwise conjugated) to two or more heterologous sequences. In some embodiments, one or more heterologous sequences may be inserted within the DNA polymerase domain or may replace amino acid segments of the sequence upon which the DNA polymerase domain is based (e.g., SEQ ID NO: 1).

In some embodiments, provided herein are compositions comprising a chimeric DNA polymerase with reduced stutter proclivity, the chimeric DNA polymerase comprising: (a) a DNA polymerase domain; (b) a thioredoxin binding domain (TBD), wherein the TBD has non-cysteine amino acids at positions corresponding to positions 14 and 52 of SEQ ID NO: 15; and (c) a thioredoxin (TRX) domain, wherein the TRX domain has a non-cysteine amino acid at a position corresponding to position 35 of SEQ ID NO: 16. In some embodiments, the chimeric DNA polymerase with reduced stutter proclivity and activity in the absence of a reducing agent (e.g., DTT, TCEP, etc.) comprises a sequence having at least 60% (e.g., 60%, 65%, 70%, 75%, 80%, 85%, 90%, 95%, 98%, 99%, 100%, or ranges therebetween) sequence identity to one of SEQ ID NOS: 22-27 and 35-49.

Some embodiments herein involve a chimeric DNA polymerase comprising a DNA polymerase domain (e.g., based on the DNA polymerase domain of SEQ ID NO: 1) with one or more insertions, substitutions, N- or C-terminal additions, or deletions. For reference, the DNA polymerase domain sequence of SEQ ID NO: 1 can be divided into 11 segments: N-terminal segment (SEQ ID NO 2), insertion site A (SEQ ID NO 3), internal segment 1 (SEQ ID NO 4), insertion site B (SEQ ID NO 5), internal segment 2 (SEQ ID NO 6), insertion site C (SEQ ID NO 7), internal segment 3 (SEQ ID NO 8), insertion site D (SEQ ID NO 9), internal segment 4 (SEQ ID NO 10), thumb insertion site (SEQ ID NO 11), and C-terminal segment (SEQ ID NO 12). Each of the insertion sites (A-D and thumb) represent a portion of the DNA polymerase domain that, in certain embodiments, is substituted for a heterologous sequence (e.g., TIS, TBD, TRX) or is the site of insertion of a heterologous sequence (e.g., TIS, TBD, TRX). All or a portion of the insertion site may be replaced by the heterologous sequence. Alternatively, the entire insertion site may remain with the heterologous sequence inserted between two amino acids of the insertion site. Each of the internal segments (C-terminal, 1-4, and N-terminal) represents a portion of the DNA polymerase domain that, in certain embodiments, remain without insertion or substitution of a heterologous segment therein. In some embodiments, the internal segments may be the locations of various substitutions, deletions, additions, etc., for the purpose of enhancing a characteristic of the polymerase. Any of the above sequences or combinations thereof may comprise various substitutions to enhance one or more characteristics of the systems herein.

In particular embodiments, insertion sites A-D are locations for insertion of or substitution with a TIS described herein. In some embodiments, a DNA polymerase is provided with one or more of insertion sites A-D containing the insertion or substitution (of all or a portion of the insertion site) with a TIS (e.g., a sequence having greater than 50% (e.g., 50%, 55%, 60%, 65%, 70%, 75%, 80%, 85%, 90%, 95%, 99%, 100%, or ranges therebetween) sequence identity with one of SEQ ID NOS: 18-21). In some embodiments, insertion sites A-D are locations for insertion of or substitution with a TBD or TRX described herein. In some embodiments, a DNA polymerase is provided with one or more of insertion sites A-D containing the insertion or substitution (of all or a portion of the insertion site) with a TBD or TRX (e.g., a sequence having greater than 50% (e.g., 50%, 55%, 60%, 65%, 70%, 75%, 80%, 85%, 90%, 95%, 99%, 100%, or ranges therebetween) sequence identity with one of SEQ ID NOS: 199-200.

In some embodiments, provided herein is a DNA polymerase having activity in the absence of reducing agent and having at least 50% (e.g., 50%, 55%, 60%, 65%, 70%, 75%, 80%, 85%, 90%, 95%, 99%, 100%, or ranges therebetween) sequence identity with one of SEQ ID NOS: 201-209, and comprising TBD and/or TRX substitutions described herein to facilitate activity in the absence of reducing agent.

In some embodiments, the thumb insertion site is a location for insertion of or substitution with a TIS described herein. In some embodiments, the thumb insertion site is a location for insertion of or substitution with a TBD or TRX described herein (e.g., comprising TBD and/or TRX substitutions described herein to facilitate activity in the absence of reducing agent). In some embodiments, a DNA polymerase is provided with the thumb insertion site containing the insertion or substitution (of all or a portion of the insertion site) with a TBD or TRX (e.g., a sequence having greater than 50% (e.g., 50%, 55%, 60%, 65%, 70%, 75%, 80%, 85%, 90%, 95%, 99%, 100%, or ranges therebetween) sequence identity with one of SEQ ID NOS: 199-200). In some embodiments, a DNA polymerase is provided with a thumb insertion site containing the insertion or substitution (of all or a portion of the insertion site) with a TIS (e.g., a sequence having greater than 50% (e.g., 50%, 55%, 60%, 65%, 70%, 75%, 80%, 85%, 90%, 95%, 99%, 100%, or ranges therebetween) sequence identity with one of SEQ ID NOS: 18-21).

In some embodiments, provided herein are DNA polymerase systems comprising a first DNA polymerase domain and second heterologous (e.g., not native to the DNA polymerase domain) sequence, wherein the DNA polymerase domain and the heterologous sequence are not fused as a single polypeptide.

In some embodiments, a TRX sequence is fused to a DNA polymerase domain or TBD via a linker that allows both intramolecular interactions between the TRX and the TBD on the same protein monomer, and intermolecular interactions between the TRX and the TBD on different protein monomers. In other embodiments, the TRX sequence is fused to the DNA polymerase domain or TBD via a linker that only allows intramolecular interactions between the TRX and the TBD on the same protein monomer. In some embodiments, the TRX sequence is fused to the DNA polymerase domain or TBD via a linker that only allows intermolecular interactions between the TRX and the TBD on different protein monomers.

In some embodiments, a TBD sequence is fused to a DNA polymerase domain or TRX via a linker that allows both intramolecular interactions between the TBD and the TRX on the same protein monomer, and intermolecular interactions between the TBD and the TRX on different protein monomers. In other embodiments, the TBD sequence is fused to the DNA polymerase domain or TRX via a linker that only allows intramolecular interactions between the TBD and the TRX on the same protein monomer. In some embodiments, the TBD sequence is fused to the DNA polymerase domain or TRX via a linker that only allows intermolecular interactions between the TBD and the TRX on different protein monomers.

In some embodiments, the TRX sequence is not fused to the DNA polymerase domain or TBD. In some embodiments, the TRX sequence is fused to another protein or peptide that interacts with DNA, Pol-TBD, and/or TRX-Pol-TBD. In some embodiments, the TRX sequence is fused to another protein or peptide. In some embodiments, a free TRX may be fused to one or more peptide or polypeptide modifiers of 1-200 amino acids (e.g., 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 160, 165, 170, 180, 185, 190, 195, 200 or ranges therebetween). In some embodiments, a free TRX comprises a peptide or polypeptide modifier fused to the C- or N-terminus of the TRX sequence. Examples of modifiers include, but are not limited to, a His tag, HaloTag, streptavidin, an antibody, an epitope, a FLAG tag, etc. In some embodiments, a free TRX is conjugated (e.g., non-genetically linked) to a peptide, polypeptide, or non-peptide (e.g., small molecule, solid surface, etc.) by any suitable conjugation method, such as, click chemistry, thiol-maleimide linkage, cysteinemaleimide-cysteine conjugation, etc. In some embodiments, chemistries are utilized that increase the local concentration of TRX relative to the TBD than could otherwise be achieved in a purely binary system.

In some embodiments, the TBD sequence is not fused to the DNA polymerase domain or TRX. In some embodiments, the TBD sequence is fused to another protein or peptide that interacts with DNA, Pol-TRX, and/or TRX-Pol-TBD. In some embodiments, the TBD sequence is fused to another protein or peptide. In some embodiments, a free TBD may be fused to one or more peptide or polypeptide modifiers of 1-200 amino acids (e.g., 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 160, 165, 170, 180, 185, 190, 195, 200 or ranges therebetween). In some embodiments, a free TBD comprises a peptide or polypeptide modifier fused to the C- or N-terminus of the TBD sequence. Examples of modifiers include, but are not limited to, a His tag, HaloTag, streptavidin, an antibody, an epitope, a FLAG tag, etc. In some embodiments, a free TRX is conjugated (e.g., non-genetically linked) to a peptide, polypeptide, or non-peptide (e.g., small molecule, solid surface, etc.) by any suitable conjugation method, such as, click chemistry, thiol-maleimide linkage, cysteinemaleimide-cysteine conjugation, etc. In some embodiments, chemistries are utilized that increase the local concentration of TBD relative to the TRX than could otherwise be achieved in a purely binary system.

In some embodiments, provided herein are compositions comprising: (a) a fusion protein comprising: (i) a DNA polymerase domain, and (ii) a thioredoxin binding domain (TBD); and (b) free thioredoxin. In some embodiments, the free thioredoxin is present in the composition at a TRX:TBD ratio of 0.1 to 2000 (e.g., 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700. 800, 900, 1000, 1500, 2000, or ranges therebetween (e.g., 0.1 to 800, 0.6 to 600, etc.)). In some embodiments, the fusion protein comprises a sequence having at least 60% (e.g., 60%, 65%, 70%, 75%, 80%, 85%, 90%, 95%, 98%, 99%, 100%, or ranges therebetween) sequence identity to SEQ ID NOS: 28-34.

In some embodiments, provided herein are compositions comprising: (a) a fusion protein comprising: (i) a DNA polymerase domain, (ii) a thioredoxin binding domain (TBD), and (iii) a thioredoxin (TRX); and (b) (i) free thioredoxin or (ii) a TRX and DNA polymerase fusion. In some embodiments, the (a) and (b) are present in the composition at ratio of between 1:100 and 100:1 (e.g., 1:100, 1:80, 1:60, 1:40, 1:20, 1:10, 1:5, 1:4, 1:3, 1:2, 1:1, 2:1, 3:1, 4:1, 5:1, 10:1, 20:1: 40:1, 60:1, 80:1, 100:1).

In some embodiments, provided herein are compositions comprising: (a) a fusion protein comprising: (i) a DNA polymerase domain, (ii) a thioredoxin binding domain (TBD), and (iii) a thioredoxin (TRX); and (b) (i) a free TBD or (ii) a TBD and DNA polymerase fusion. In some embodiments, the (a) and (b) are present in the composition at ratio of between 1:100 and 100:1 (e.g., 1:100, 1:80, 1:60, 1:40, 1:20, 1:10, 1:5, 1:4, 1:3, 1:2, 1:1, 2:1, 3:1, 4:1, 5:1, 10:1, 20:1: 40:1, 60:1, 80:1, 100:1).

In some embodiments, a DNA polymerase domain herein is a fusion of portions of two or more DNA polymerases (e.g., natural sequences (e.g., portions of Taq and Tfl DNA polymerases), engineered sequences, etc.). In some embodiments, a DNA polymerase domain is a chimeric DNA polymerase.

In some embodiments, the polymerases (or polymerase-containing systems) herein find use in any systems (e.g., amplification reactions) in which a DNA polymerase (e.g., thermostable DNA polymerase (e.g., Taq polymerase, etc.), etc.) would otherwise find use. In some embodiments, the polymerases (or polymerase-containing systems) herein find use in PCR reactions, multiplex amplifications, STR amplification, sequencing applications (e.g., Sanger, NGS), MSI-related technologies, etc.

In some embodiments, any PCR conditions disclosed herein, or any standard PCR conditions, can be used with the polymerases and subsystems described herein. Any PCR conditions may be used in any of the methods herein to amplify a target nucleic acid. In some embodiments, provided herein are kits or reaction mixtures comprising the chimeric DNA polymerase or fusion protein herein, and amplification reagents sufficient to amplify a DNA target sequence. In some embodiments, the amplification reagents comprise one or more of oligonucleotide primers, deoxynucleotide triphosphates, magnesium, ethylenediaminetetraacetic acid (EDTA), buffer, water, and a template DNA comprising the DNA target sequence. In some embodiments, the kits or reaction mixtures further comprise a reducing agent. In some embodiments, the reducing agent is a thiol reductant or non-thiol reductant. In some embodiments, the reducing agent is dithiothreitol (DTT) or tris (2-carboxyethyl) phosphine (TCEP).

In some embodiments, the DNA target sequence comprises one or more short tandem repeats (STRs). In some embodiments, the STR comprises a repetitive unit of 1-8 nucleotides (e.g., 1, 2, 3, 4, 5, 6, 7, 8, or ranges therebetween) extending 10-500 nucleotides in length (e.g., 10, 15, 20, 25, 30, 35, 40, 45, 50, 55, 60, 65, 70, 75, 80, 85, 90, 95, 100, 200, 300, 400, 500, or ranges therebetween). In some embodiments, the tandem repeat comprises a repetitive unit of 1-50 nucleotides (e.g., 1, 2, 3, 4, 5, 6, 7, 8, 9, 10,15, 20, 25, 30, 35, 40, 45, 50, or ranges therebetween) extending up to 1000 nucleotides in length.

In some embodiments, the reaction volume includes ethylenediaminetetraacetic acid (EDTA), magnesium, tetramethyl ammonium chloride (TMAC), or any combination thereof. In some embodiments, the concentration of TMAC is between 20 and 80 mM, such as between 25 and 70 mM, 30 and 60 mM, 30 and 40 mM, 40 and 50 mM, 50 and 60 mM, or 60 and 70 mM, inclusive. In some embodiments, the concentration of magnesium (such as magnesium from magnesium chloride) is between 1 and 10 mM, such as between 1 and 8 mM, 1 and 5 mM, 1 and 3 mM, 3 and 5 mM, 3 and 6 mM, or 5 and 8 mM, inclusive. In some embodiments, the concentration of available magnesium (the concentration of magnesium that is assumed to be available for binding the polymerase and not bound to molecules other than the polymerase), such as the magnesium that is not bound by phosphate groups on dNTPs, primers, or nucleic acid templates, or carboxylic acid groups on magnetic or other beads, if present, is between 0.5 to 10 mM, such as between 1 and 8 mM, 1 and 5 mM, 1 and 3 mM, 3 and 5 mM, 3 and 6 mM, 4 and 6 mM, or 5 and 8 mM, inclusive. In some embodiments, EDTA is used to decrease the amount of magnesium available as a cofactor for the polymerase since high concentrations of magnesium can result in PCR errors, such as amplification of non-target nucleic acids. In some embodiments, the concentration of EDTA reduces the amount of available magnesium to between 1 and 5 mM (such as between 3 and 5 mM).

In some embodiments, the pH is between 6.0 and 9.0 such as between 6.0 and 6.8, 6.8 and 7.5, 7.5 and 8.8, 8 and 8.3, or 8.3 and 8.5, inclusive. In some embodiments, Tris is used at, for example, a concentration of between 10 and 100 mM, such as between 10 and 25 mM, 25 and 50 mM, 50 and 75 mM, or 25 and 75 mM, inclusive. In some embodiments, any of these concentrations of Tris are used at a pH between 7.5 and 8.5.

In some embodiments, a combination of KCl and (NH₄)₂SO₄ is used, such as between 50 and 150 mM KCl and between 10 and 90 mM (NH₄)₂SO₄, inclusive. In some embodiments, the concentration of KCl is between 0 and 30 mM, between 50 and 100 mM, or between 100 and 150 mM, inclusive. In some embodiments, the concentration of (NH₄)₂SO₄ is between 10 and 50 mM, 50 and 90 mM, 10 and 20 mM, 20 and 40 mM, 40 mM and 60, or 60 mM and 80 mM (NH₄)₂SO₄, inclusive. In some embodiments, the ammonium [NH₄.⁺] concentration is between 0 and 160 mM, such as between 0 to 50, 50 to 100, or 100 to 160 mM, inclusive.

In some embodiments, a crowding agent is used, such as polyethylene glycol (PEG, such as PEG 8,000) or glycerol. In some embodiments, the amount of PEG (such as PEG 8,000) is between 0.1 to 20%, such as between 0.5 to 15%, 1 to 10%, 2 to 8%, or 4 to 8%, inclusive. In some embodiments, the amount of glycerol is between 0.1 to 20%, such as between 0.5 to 15%, 1 to 10%, 2 to 8%, or 4 to 8%, inclusive. In some embodiments, a crowding agent allows either a low polymerase concentration and/or a shorter annealing time to be used. In some embodiments, a crowding agent improves the uniformity of the direct oxide reduction (DOR) and/or reduces dropouts (undetected alleles).

In some embodiment, between 5 and 2000 Units/mL (Units per 1 mL of reaction volume) of polymerase is used, such as between 5 to 100, 100 to 200, 200 to 300, 300 to 400, 400 to 500, 500 to 600, 600 to 700, 700 to 800, 800 to 900, 900 to 1000, 1000 to 1500, or 1500 to 2000 Units/mL, inclusive. One unit is defined as the amount of enzyme required to catalyze the incorporation of 10 nanomoles of dNTPs into acid-insoluble material in 30 minutes at 74° C.

In some embodiments, hot-start PCR is used to reduce or prevent polymerization prior to PCR thermocycling. Exemplary hot-start PCR methods include initial inhibition of the DNA polymerase, or physical separation of reaction components until the reaction mixture reaches the higher temperatures. In some embodiments, the enzyme is spatially separated from the reaction mixture by wax that melts when the reaction reaches high temperature. In some embodiments, slow release of magnesium is used. DNA polymerase requires magnesium ions for activity, so the magnesium is chemically separated from the reaction by binding to a chemical compound, and is released into the solution only at high temperature. In some embodiments, non-covalent binding of an inhibitor is used. In this method a peptide, antibody, or aptamer are non-covalently bound to the enzyme at low temperature and inhibit its activity. After incubation at elevated temperature, the inhibitor is released, and the reaction starts. In some embodiments, a cold-sensitive Taq polymerase is used, such as a modified DNA polymerase with almost no activity at low temperature. In some embodiments, chemical modification is used. In this method, a molecule is covalently bound to the side chain of an amino acid in the active site of the DNA polymerase. The molecule is released from the enzyme by incubation of the reaction mixture at elevated temperature. Once the molecule is released, the enzyme is activated. In some embodiments, the amount of template nucleic acids (such as an RNA or DNA sample) is between 20 and 5,000 ng, such as between 20 to 200, 200 to 400, 400 to 600, 600 to 1,000; 1,000 to 1,500; or 2,000 to 3,000 ng, inclusive. In some embodiments, a reaction comprises 0.2 ng/ml to 2 μg/mL (e.g., 0.2 ng/ml, 0.5 ng/ml, 1 ng/ml, 2 ng/ml, 5 ng/ml, 10 ng/ml, 20 ng/ml, 50 ng/mL, 100 ng/mL, 500 ng/mL, 1 μg/mL, 2 μg/mL, or ranges therebetween) of template DNA.

In some embodiments, provided herein are methods of amplifying a DNA target sequence comprising exposing a reaction mixture comprising chimeric DNA polymerase or fusion protein herein, and amplification reagents to PCR thermal cycling conditions.

In some embodiments, exemplary PCR thermocycling conditions include 95° C. for 10 minutes (hot start); 20 cycles of 96° C. for 30 seconds; 65° C. for 15 seconds; and 72° C. for 30 seconds; followed by 72° C. for 2 minutes (final extension); and then a 4° C. hold. In some embodiments, the PCR thermocycling conditions include 95° C. for 10 minutes (hot start); 25 cycles of 96° C. for 30 seconds; 65° C. for 20 seconds; and 72° C. for 30 seconds); followed by 72° C. for 2 minutes (final extension); and then a 4° C. hold. In some embodiments, an exemplary set of PCR thermocycling conditions includes 95° C. for 10 minutes, 15 cycles of 95° C. for 30 seconds, 65° C. for 1 minute, 60° C. for 5 minutes, 65° C. for 5 minutes and 72° C. for 30 seconds; and then 72° C. for 2 minutes. In some embodiments, an exemplary set of PCR thermocycling conditions includes 96° C. for 1 minute, 30 cycles of 94° C. for 10 seconds, 59° C. for 30 seconds, 72° C. for 1 minute, and finally 60° C. for 10 minutes and 4° C. hold. In other embodiments, an exemplary set of PCR thermocycling conditions includes 96° C. for 1 minute, 30 cycles of 94° C. for 10 seconds, 59° C. for 30 seconds, and finally 60° C. for 10 minutes and 4° C. hold. In some embodiments, PCR thermocycling is used with the following reaction exemplary conditions: 100 mM KCl, 50 mM (NH₄)₂SO₄, 3 mM MgCl₂, 7.5 nM of each primer in the library, 50 mM TMAC, and 7 ul DNA template in a 20 ul final volume at pH 8.1. In some embodiments, other reaction conditions understood in the field are utilized.

### Experimental

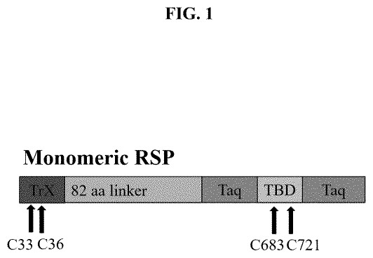

It was contemplated that the reducing agent requirement of a reduced stutter polymerase and/or polymerase systems was to maintain a thiol (sulfhydryl) group on cysteine residues that were introduced during its development of such polymerase/systems, which ensures proper interactions with neighboring residues and prevents disulfide bond formation with other cysteines. In total, four cysteine residues were introduced into the enzyme: two from the thioredoxin domain, and two from the Thioredoxin Binding Domain (FIG. 1).

Experiments conducted during the development of reduced stutter polymerases have demonstrated that three (C36, C683, and C721 of SEQ ID NO: 108) of the four cysteine residues could be independently mutated to other residues without impairing enzyme activity. On the other hand, all mutations attempted at the C33 (of SEQ ID NO: 108) residue failed to result in a functional enzyme. Therefore, six constructs were created that combined the best mutations at the C36, C683, and C721 positions while fixing the C33:
(C36S, C683L, C721K)=8412
(C36S, C683V, C721K)=8413
(C36S, C683K, C721K)=8414
(C36S, C683K, C721V)=8415
(C36S, C683L, C721V)=8416
(C36S, C683V, C721V)=8417
*substitutions relative to polymerase 8100 (SEQ ID NO: 108).

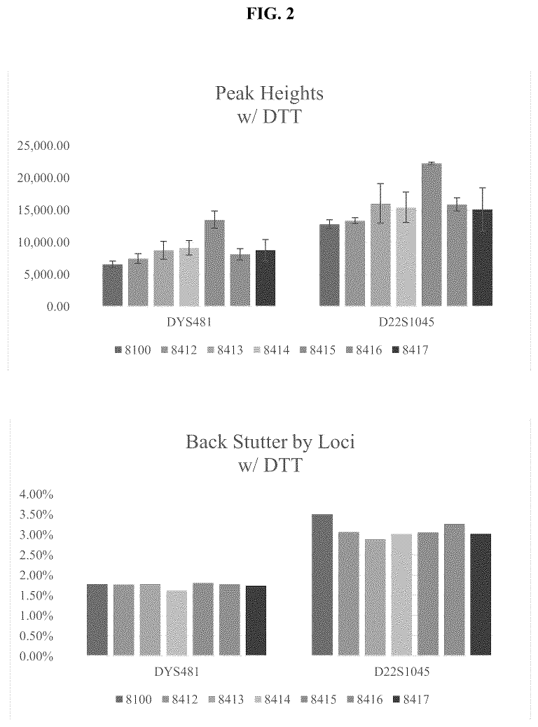

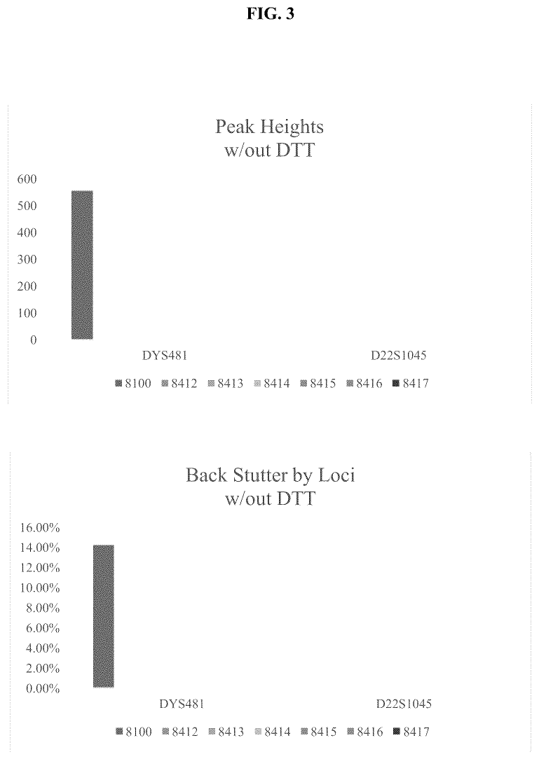

The six constructs were then evaluated for the ability to amplify an STR duplex in the presence and absence of a reducing agent (DTT) using the lysate stutter assay described in U.S. patent application Ser. No. 18/595,339; incorporated by reference in its entirety. The results in the presence of DTT are presented in FIG. 2. All six constructs amplified both allelic peaks with comparably low stutter compared to 8100. The results in the absence of DTT are presented in FIG. 3. In the absence of DTT, only 8100 was capable of amplifying a small peak at DYS481, which also had a high stutter peak. The other enzyme variants were unable to amplify either allelic peak. These variants contain only one cysteine residue at position C33 (of SEQ ID NO: 108). In the crystal structure of T7 DNA polymerase, C33 makes a hydrogen bond with T735 (of SEQ ID NO: 108). Given the important role C33 plays in polymerase activity and stutter, a series of 167 mutations at C33 and/or T735 were evaluated using the lysate stutter assay without reducing agent, including site saturation at C33. These mutations were made in one construct (8416; SEQ ID NO: 201), which was selected from the six triple cysteine point mutants. These results are listed in Table 1.

TABLE 1
Stutter assay results for C33/T735 variants
(relative to 8416; SEQ ID NO: 201)

| C33 | T735 | | | |
|---|---|---|---|---|
| Residue | Residue | D22S1045 Stutter | DYS481 Stutter | Trials |
| | Taq | 10.46 ± 0.6% | 19.75 ± 0.27% | 3 |
| C | T | No allelic peaks | | 3 |
| A | A | 8.56 ± 0.66% | 18.76 ± 0.81% | 3 |
| A | F | 10.86 ± 0.24% | 19.06 ± 0.71% | 3 |
| A | I | 8.9 ± 0.26% | 17.26 ± 0.44% | 3 |
| A | L | 8.84 ± 0.29% | 17.96 ± 0.23% | 3 |
| A | M | 8.86 ± 0.07% | 18.08 ± 0.38% | 3 |
| A | P | 11.8 ± 0.43% | 19.99 ± 0.78% | 3 |
| A | T | 7.36 ± 0.27% | 16.2 ± 0.24% | 3 |
| A | V | 6.5 ± 0.14% | 15.92 ± 0.33% | 3 |
| A | W | 10.08 ± 0.83% | 18.13 ± 0.38% | 3 |
| C | C | 5.09 ± 0.38% | 11.93 ± 0.16% | 3 |
| C | G | 6.3%^{†} | 17.85 ± 0.3% | 3 |
| D | D | 11.13 ± 0.44% | 18.95 ± 0.23% | 3 |
| D | E | 11.08 ± 0.31% | 19.16 ± 0.46% | 3 |
| D | H | 11.34 ± 0.12% | 19.5 ± 0.39% | 3 |
| D | K | 11.77 ± 0.2% | 19.41 ± 0.16% | 3 |
| D | N | 11.09 ± 0.17% | 18.86 ± 0.35% | 3 |
| D | R | 11.09 ± 0.21% | 19.18 ± 0.38% | 3 |
| D | T | 5.05 ± 0.1% | 13.28 ± 0.55% | 3 |
| E | E | 9.09 ± 0.22% | 18.48 ± 0.24% | 3 |
| E | R | 11.21 ± 2.98% | 18.54 ± 0.71% | 3 |
| G | G | 10.06 ± 0.21% | 19.18 ± 0.26% | 3 |
| G | T | 4.22 ± 0.21% | 11.21 ± 0.57% | 3 |
| H | E | 8.31 ± 0.55% | 18.47 ± 0.38% | 3 |
| H | Q | 10.99 ± 0.65% | 18.84 ± 0.26% | 2 |
| H | R | 11.07 ± 0.1% | 18.96 ± 0.51% | 3 |
| H | S | 10.64 ± 0.42% | 18.61 ± 0.67% | 3 |
| H | Y | 9.63 ± 0.85% | 18.75 ± 0.51% | 3 |
| K | R | 11.15 ± 0.13% | 19.22 ± 0.43% | 3 |
| M | T | 8.62 ± 2.52% | 18.29 ± 0.28% | 3 |
| M | V | 9.59 ± 0.6% | 18.97 ± 0.41% | 3 |
| N | D | 11.02 ± 0.02% | 18.57 ± 0.26% | 3 |
| N | S | 9.6 ± 0.35% | 18.68 ± 0.67% | 3 |
| N | T | 8.18 ± 0.34% | 17.25 ± 0.55% | 3 |
| P | A | 10.55 ± 0.41% | 19 ± 0.51% | 3 |
| P | G | 10.99 ± 0.27% | 18.96 ± 0.31% | 3 |
| P | P | 11.25 ± 0.38% | 18.98 ± 0.22% | 3 |
| P | T | 6.46 ± 0.35% | 15.79 ± 1.25% | 3 |
| P | V | 7.16 ± 1.83% | 17.39 ± 0.31% | 3 |
| Q | T | 10.08 ± 0.79% | 18.65 ± 0.31% | 3 |
| R | E | 9.67 ± 0.03% | 19.51 ± 0.31% | 3 |
| R | R | 9.44 ± 0.73% | 18.7 ± 0.11% | 3 |
| S | D | 10.62 ± 0.22% | 19.19 ± 0.57% | 3 |
| S | E | 10.64 ± 0.14% | 18.53 ± 0.81% | 3 |
| S | G | 11.52 ± 0.61% | 18.91 ± 0.71% | 3 |
| S | H | 11.34 ± 0.34% | 19.81 ± 0.87% | 3 |
| S | K | 11.69 ± 0.22% | 19.63 ± 0.14% | 3 |
| S | N | 7.24 ± 0.04% | 15.94 ± 0.24% | 3 |
| S | Q | 10.67 ± 0.37% | 18.64 ± 0.7% | 3 |
| S | R | 11.69 ± 0.55% | 19.96 ± 0.88% | 3 |
| S | S | 9.01 ± 0.53% | 17.6 ± 0.2% | 3 |
| S | T | 5.82 ± 0.23% | 15.03 ± 0.3% | 3 |
| S | Y | 11.48 ± 0.56% | 18.67 ± 0.41% | 3 |
| T | G | 9.85 ± 0.08% | 18.52 ± 0.55% | 3 |
| T | K | 11 ± 0.3% | 19.34 ± 0.71% | 3 |
| T | Q | 9.27 ± 0.27% | 18.61 ± 0.11% | 3 |
| T | R | 10.91 ± 0.63% | 19.2 ± 1.62% | 2 |
| V | A | 10.86 ± 0.09% | 18.02 ± 0.24% | 3 |
| V | T | 9.75 ± 1.48% | 18.06 ± 0.51% | 3 |
| Y | Y | 9.4 ± 0.67% | 18.61 ± 0.44% | 3 |
| C | D | No allelic peak | 17.7 ± 1.3% | 3 |
| C | H | No allelic peak | 17.89 ± 0.38% | 3 |
| C | K | No allelic peak | 18.6 ± 1.16% | 3 |
| C | N | No allelic peak | 17.9 ± 0.54% | 3 |
| C | Q | No allelic peak | 18.19 ± 0.52% | 3 |
| C | R | No allelic peak | 18.3 ± 0.49% | 3 |
| C | Y | No allelic peak | 17.64 ± 0.68% | 3 |
| E | D | No allelic peak | 17.38 ± 0.3% | 3 |
| E | H | No allelic peak | 18.07 ± 0.34% | 3 |
| E | T | No allelic peak | 16.01 ± 0.07% | 3 |
| F | A | No allelic peak | 18.41 ± 0.63% | 3 |
| F | F | No allelic peak | 17.86 ± 0.39% | 3 |
| F | I | No allelic peak | 18.53 ± 0.13% | 3 |
| F | L | No allelic peak | 17.74 ± 0.22% | 3 |
| F | M | No allelic peak | 17.25 ± 1.04% | 3 |
| F | T | No allelic peak | 17.42 ± 0.23% | 3 |
| F | V | No allelic peak | 17.62 ± 0.62% | 3 |
| F | W | No allelic peak | 17.12 ± 0.61% | 3 |
| G | F | No allelic peak | 16.06 ± 0.85% | 3 |
| G | H | No allelic peak | 15.73 ± 0.19% | 3 |
| G | P | No allelic peak | 17.19 ± 0.28% | 3 |
| G | S | No allelic peak | 17.27 ± 0.25% | 3 |
| G | W | No allelic peak | 17.61 ± 0.78% | 3 |
| G | Y | No allelic peak | 17.95 ± 0.71% | 3 |
| H | D | No allelic peak | 17.89 ± 0.54% | 3 |
| H | K | No allelic peak | 15.91 ± 0.63% | 3 |
| H | T | No allelic peak | 18.52 ± 0.56% | 3 |
| I | A | No allelic peak | 18.53 ± 0.86% | 3 |
| I | F | No allelic peak | 17.48 ± 0.78% | 3 |
| I | I | No allelic peak | 18.25 ± 0.86% | 3 |
| I | L | No allelic peak | 17.68 ± 0.29% | 3 |
| I | M | No allelic peak | 17.59 ± 1.45% | 3 |
| I | T | No allelic peak | 17.31 ± 0.34% | 3 |
| I | V | No allelic peak | 18.48 ± 0.7% | 3 |
| I | W | No allelic peak | 18.32 ± 0.33% | 3 |
| K | D | No allelic peak | 13.39 ± 1.28% | 3 |
| K | H | No allelic peak | 14.62 ± 1.29% | 3 |
| K | K | No allelic peak | 13.24 ± 0.36% | 3 |
| K | S | No allelic peak | 17.44 ± 0.56% | 3 |
| K | T | No allelic peak | 17.31 ± 0.1% | 3 |
| L | A | No allelic peak | 19.1 ± 5.43% | 3 |
| L | F | No allelic peak | 16.75 ± 0.38% | 3 |
| L | I | No allelic peak | 17.16%^{†} | 2 |
| L | L | No allelic peak | 16.7 ± 0.21% | 3 |
| L | M | No allelic peak | 14.38 ± 0.82% | 3 |
| L | T | No allelic peak | 17.87 ± 0.6% | 3 |
| L | V | No allelic peak | 14.09 ± 0.03% | 3 |
| M | A | No allelic peak | 17.31 ± 0.29% | 3 |
| M | F | No allelic peak | 15.31 ± 0.29% | 3 |
| M | I | No allelic peak | 12.64 ± 0.77% | 3 |
| M | M | No allelic peak | 18.91 ± 0.59% | 3 |
| M | W | No allelic peak | 12.78 ± 0.89% | 3 |
| N | N | No allelic peak | 13.49 ± 0.3% | 3 |
| N | Q | No allelic peak | 17.94 ± 0.39% | 3 |
| N | Y | No allelic peak | 17.96 ± 0.71% | 3 |
| Q | H | No allelic peak | 14.74 ± 1.11% | 3 |
| Q | Q | No allelic peak | 15.88 ± 8.45% | 3 |
| Q | S | No allelic peak | 17.29 ± 0.18% | 3 |
| R | S | No allelic peak | 18.19 ± 0.16% | 3 |
| R | T | No allelic peak | 17.63 ± 0.35% | 3 |
| R | Y | No allelic peak | 18.87 ± 0.22% | 3 |
| T | S | No allelic peak | 18.12 ± 0.59% | 3 |
| T | T | No allelic peak | 18.74 ± 0.46% | 3 |
| T | Y | No allelic peak | 18.65 ± 0.5% | 3 |
| V | F | No allelic peak | 14.97 ± 0.17% | 3 |
| V | I | No allelic peak | 16.04 ± 0.66% | 3 |
| V | L | No allelic peak | 14.42 ± 0.23% | 3 |
| V | M | No allelic peak | 14.9 ± 0.26% | 3 |
| V | V | No allelic peak | 15.92 ± 0.69% | 3 |
| W | A | No allelic peak | 17.28 ± 0.18% | 3 |
| W | F | No allelic peak | 15.44 ± 0.25% | 3 |
| W | I | No allelic peak | 16.14 ± 0.34% | 3 |
| W | L | No allelic peak | 14.22 ± 0.29% | 3 |
| W | M | No allelic peak | 14.81 ± 0.6% | 3 |
| W | T | No allelic peak | 18.92 ± 0.94% | 3 |
| W | V | No allelic peak | 13.66 ± 0.24% | 3 |
| Y | H | No allelic peak | 16.81 ± 0.28% | 3 |
| Y | K | No allelic peak | 18.86 ± 0.22% | 3 |
| Y | N | No allelic peak | 17.39 ± 0.08% | 2 |
| Y | Q | No allelic peak | 17.71 ± 0.33% | 3 |
| Y | R | No allelic peak | 18.48 ± 0.37% | 3 |
| Y | S | No allelic peak | 17.6 ± 0.55% | 3 |
| Y | T | No allelic peak | 17.74 ± 0.28% | 3 |
| R | D | No allelic peaks | | 3 |
| Q | N | | | 3 |
| L | W | | | 3 |
| D | C | | | 2 |
| Q | C | | | 3 |
| K | C | | | 3 |
| C | S | | | 3 |
| K | E | | | 3 |
| S | C | | | 3 |
| R | H | | | 3 |
| R | K | | | 3 |
| R | C | | | 3 |
| M | L | | | 3 |
| Q | Y | | | 3 |
| K | Y | | | 3 |
| E | K | | | 3 |
| G | C | | | 3 |
| H | C | | | 3 |
| H | H | | | 3 |
| N | C | | | 3 |
| T | C | | | 3 |
| T | N | | | 3 |
| V | W | | | 3 |
| W | W | | | 2 |
| Y | C | | | 3 |

In total, 59 mutants were capable of amplifying both allelic peaks in the absence of reducing agent, 83 amplified only the DYS481 allele, and 26 mutants were unable to amplify either allelic peak (this includes the parental 8416 construct). At these loci, native Taq has a stutter frequency of approximately 10.5% and 20% at D22S1045 and DYS481, respectively, for an average stutter frequency of ˜15%. If only combinations that resulted in a 20% or greater reduction in stutter are considered (12% or less average stutter frequency), then a total of 8 combinations exist capable of reducing stutter without a reducing agent requirement, which are C33A, C33A/T735V, T735C, C33D, C33G, C33P, C33S/T735N, and C33S.

### Sequences

Below are sequences provided herein identified by SEQ ID NOS. In addition to modifications described and allowed within the embodiments herein, any sequences herein may additionally be provided with or without sequences intended for purification or other related purposes, such as a His tag or other purification tags that are understood in the field but may or may not be present in the sequences provided herein.

Unless otherwise specified “X” residues present in the following sequences may be any amino acid residue, or may be absent. Sequences encompassing any residue at an X position below (or without a residue at position X) are within the scope herein. For example, “X1-X10” refers to up to 10 amino acids of any identity.

In the sequences below and throughout the specification, if a position number is provided without reference to a specific base sequence, the assumed reference sequences are SEQ ID NO: 1 for a DNA polymerase, SEQ ID NO: 15 for TBD, SEQ ID NO: 16 for TRX, and SEQ ID NO: 35 for a TRX-Taq-TBD construct. For example, H914 refers to the histidine at the 914th position of a TRX-Taq-TBD with a 60 amino acid linker. When H914 is referred to without other context for a TRX-Taq-TBD construct with a different length linker, it refers to the histidine at the position in the construct corresponding to the H914 position in SEQ ID NO: 35. Likewise, mutations made within the TBD (T489, R506, T535, E537, E548, and S555) are assigned based on their location within SEQ ID NO:50 (pATG6979), which encodes Taq-TBD without a fused thioredoxin. When these residues are referred to without other context for a TRX-Taq-TBD construct, it refers to their positions in SEQ ID NO:50. Similarly, mutations of specific cysteine and threonine residues in TBD (C683, C721, and T735) are assigned based on their location within SEQ ID NO:108 (pATG8100), which encodes a TRX-Taq-TBD with an 82 amino acid linker. When these residues are referred to without other context for a TRX-Taq-TBD construct, it refers to their positions in SEQ ID NO:108.

SEQ ID NO: 1-Taq DNA polymerase
RGMLPLFEPKGRVLLVDGHHLAYRTFHALKGLTTSRGEPVQAVYGFAKSLLKALKEDG
DAVIVVFDAKAPSFRHEAYGGYKAGRAPTPEDFPRQLALIKELVDLLGLARLEVPGYEA
DDVLASLAKKAEKEGYEVRILTADKDLYQLLSDRIHVLHPEGYLITPAWLWEKYGLRPD
QWADYRALTGDESDNLPGVKGIGEKTARKLLEEWGSLEALLKNLDRLKPAIREKILAHM
DDLKLSWDLAKVRTDLPLEVDFAKRREPDRERLRAFLERLEFGSLLHEFGLLESPKALEE
APWPPPEGAFVGFVLSRKEPMWADLLALAAARGGRVHRAPEPYKALRDLKEARGLLA
KDLSVLALREGLGLPPGDDPMLLAYLLDPSNTTPEGVARRYGGEWTEEAGERAALSERL
FANLWGRLEGEERLLWLYREVERPLSAVLAHMEATGVRLDVAYLRALSLEVAEEIARL
EAEVFRLAGHPFNLNSRDQLERVLFDELGLPAIGKTEKTGKRSTSAAVLEALREAHPIVE
KILQYRELTKLKSTYIDPLPDLIHPRTGRLHTRFNQTATATGRLSSSDPNLQNIPVRTPLGQ
RIRRAFIAEEGWLLVALDYSQIELRVLAHLSGDENLIRVFQEGRDIHTETASWMFGVPRE
AVDPLMRRAAKTINFGVLYGMSAHRLSQELAIPYEEAQAFIERYFQSFPKVRAWIEKTLE
EGRRRGYVETLFGRRRYVPDLEARVKSVREAAERMAFNMPVQGTAADLMKLAMVKLF
PRLEEMGARMLLQVHDELVLEAPKERAEAVARLAKEVMEGVYPLAVPLEVEVGIGED
WLSAKGD
SEQ ID NO: 2-Taq DNA polymerase N-terminal segment
RGMLPLFEPKGRVLLVDGHHLAYRTFHALKG
SEQ ID NO: 3-Taq DNA polymerase insertion site A
LTTSRGEPVQAVYGFAKSLLKALKE
SEQ ID NO: 4-Taq DNA polymerase internal segment 1
DGDAVIVVFDA
SEQ ID NO: 5-Taq DNA polymerase insertion site B
KAPSFRHEAYGGYKAG
SEQ ID NO: 6-Taq DNA polymerase internal segment 2
RAPTPEDFPRQLALIKELVDLLGLARLEVPGYEADDVLASLAKKAEKEGYEVRILTADK
DLYQLLSDRIHVLHPEGYLITPAWLWEKYGLRPDQWADYRALTGDESDNLPGVKGIGE
KTARKLLEEWGSLEALLKNLDRLKPAIREKILAHMDDLKLSWDLAKVRTDLPLEVDFAK
RREPDRERL
SEQ ID NO: 7-Taq DNA polymerase insertion site C
RAFLER
SEQ ID NO: 8-Taq DNA polymerase internal segment 3
LEFGSLLHEFGLLESPKALEEAPWPPPEGAFVGFVLSRKEPMWADLLALAAARGGRVHR
APEPYKALRDLKEARGLLAKDLSVLALREGLGLPPGDDPMLLAYLLDPSNT
SEQ ID NO: 9-Taq DNA polymerase insertion site D
TPEGVARRYGGEWTEE
SEQ ID NO: 10-Taq DNA polymerase internal segment 4
AGERAALSERLFANLWGRLEGEERLLWLYREVERPLSAVLAHMEATGVRLDVAYLRAL
SLEVAEEIARLEAEVFRLAG
SEQ ID NO: 11-Taq DNA polymerase thumb insertion site
HPFNLN
SEQ ID NO: 12-Taq DNA polymerase C-terminal segment
SRDQLERVLFDELGLPAIGKTEKTGKRSTSAAVLEALREAHPIVEKILQYRELTKLKSTYI
DPLPDLIHPRTGRLHTRFNQTATATGRLSSSDPNLQNIPVRTPLGQRIRRAFIAEEGWLLV
ALDYSQIELRVLAHLSGDENLIRVFQEGRDIHTETASWMFGVPREAVDPLMRRAAKTINF
GVLYGMSAHRLSQELAIPYEEAQAFIERYFQSFPKVRAWIEKTLEEGRRRGYVETLFGRR
RYVPDLEARVKSVREAAERMAFNMPVQGTAADLMKLAMVKLFPRLEEMGARMLLQV
HDELVLEAPKERAEAVARLAKEVMEGVYPLAVPLEVEVGIGEDWLSAKGD
SEQ ID NO: 13-Taq DNA polymerase exonuclease domain
RGMLPLFEPKGRVLLVDGHHLAYRTFHALKGLTTSRGEPVQAVYGFAKSLLKALKEDG
DAVIVVFDAKAPSFRHEAYGGYKAGRAPTPEDFPRQLALIKELVDLLGLARLEVPGYEA
DDVLASLAKKAEKEGYEVRILTADKDLYQLLSDRIHVLHPEGYLITPAWLWEKYGLRPD
QWADYRALTGDESDNLPGVKGIGEKTARKLLEEWGSLEALLKNLDRLKPAIREKILAH
SEQ ID NO: 14-Taq DNA polymerase, Large N-terminal portion
RGMLPLFEPKGRVLLVDGHHLAYRTFHALKGLTTSRGEPVQAVYGFAKSLLKALKEDG
DAVIVVFDAKAPSFRHEAYGGYKAGRAPTPEDFPRQLALIKELVDLLGLARLEVPGYEA
DDVLASLAKKAEKEGYEVRILTADKDLYQLLSDRIHVLHPEGYLITPAWLWEKYGLRPD
QWADYRALTGDESDNLPGVKGIGEKTARKLLEEWGSLEALLKNLDRLKPAIREKILAHM
DDLKLSWDLAKVRTDLPLEVDFAKRREPDRERLRAFLERLEFGSLLHEFGLLESPKALEE
APWPPPEGAFVGFVLSRKEPMWADLLALAAARGGRVHRAPEPYKALRDLKEARGLLA
KDLSVLALREGLGLPPGDDPMLLAYLLDPSNTTPEGVARRYGGEWTEEAGERAALSERL
FANLWGRLEGEERLLWLYREVERPLSAVLAHMEATGVRLDVAYLRALSLEVAEEIARL
EAEVFRLAG
SEQ ID NO: 15-Thioredoxin binding domain (TBD)
GSWYQPKGGTEMFCHPRTGKPLPKYPRIKIPKVGGIFKKPKNKAQREGREPCELDTREY
VAGAPYTPVEHVVENPS
SEQ ID NO: 16-Thioredoxin (TRX)
SDKIIHLTDDSFDTDVLKADGAILVDFWAEWCGPCKMIAPILDEIADEYQGKLTVAKLNI
DQNPGTAPKYGIRGIPTLLLFKNGEVAATKVGALSKGQLKEFLDANLA
SEQ ID NO: 17-Thioredoxin C36S (TRX-C36S)
SDKIIHLTDDSFDTDVLKADGAILVDFWAEWCGPSKMIAPILDEIADEYQGKLTVAKLNI
DQNPGTAPKYGIRGIPTLLLFKNGEVAATKVGALSKGQLKEFLDANLA
SEQ ID NO: 18-T7 TBD/TRX interacting sequence (TIS)
DTDMGLLRSGKLPGKRFGSHALEAWGYRLGEMKGEYKDDFKRMLEEQGEEYVDGME
WWNFNEEMMDY
SEQ ID NO: 19-T7 TIS truncation A
AWGYRLGEMKGEYKDDFKRMLEEQGEEYVDG
SEQ ID NO: 20-T7 TIS truncation B
GEMKGEYKDDFKRMLEEQGEEYVDG
SEQ ID NO: 21-T7 TIS truncation C
EYKDDFKRMLEEQGEEYV
SEQ ID NO: 22-Exemplary DNA polymerase/TBD/TRX chimera (N-terminal TRX)
X1-
X10SDKIIHLTDDSFDTDVLKADGAILVDFWAEWCGPCKMIAPILDEIADEYQGKLTVAK
LNIDQNPGTAPKYGIRGIPTLLLFKNGEVAATKVGALSKGQLKEFLDANLAX20-
X300RGMLPLFEPKGRVLLVDGHHLAYRTFHALKGLTTSRGEPVQAVYGFAKSLLKALK
EDGDAVIVVFDAKAPSFRHEAYGGYKAGRAPTPEDFPRQLALIKELVDLLGLARLEVPG
YEADDVLASLAKKAEKEGYEVRILTADKDLYQLLSDRIHVLHPEGYLITPAWLWEKYGL
RPDQWADYRALTGDESDNLPGVKGIGEKTARKLLEEWGSLEALLKNLDRLKPAIREKIL
AHMDDLKLSWDLAKVRTDLPLEVDFAKRREPDRERLRAFLERLEFGSLLHEFGLLESPK
ALEEAPWPPPEGAFVGFVLSRKEPMWADLLALAAARGGRVHRAPEPYKALRDLKEARG
LLAKDLSVLALREGLGLPPGDDPMLLAYLLDPSNTTPEGVARRYGGEWTEEAGERAALS
ERLFANLWGRLEGEERLLWLYREVERPLSAVLAHMEATGVRLDVAYLRALSLEVAEEI
ARLEAEVFRLAGGSWYQPKGGTEMFCHPRTGKPLPKYPRIKIPKVGGIFKKPKNKAQRE
GREPCELDTREYVAGAPYTPVEHVVFNPSSRDQLERVLFDELGLPAIGKTEKTGKRSTSA
AVLEALREAHPIVEKILQYRELTKLKSTYIDPLPDLIHPRTGRLHTRFNQTATATGRLSSSD
PNLQNIPVRTPLGQRIRRAFIAEEGWLLVALDYSQIELRVLAHLSGDENLIRVFQEGRDIH
TETASWMFGVPREAVDPLMRRAAKTINFGVLYGMSAHRLSQELAIPYEEAQAFIERYFQ
SFPKVRAWIEKTLEEGRRRGYVETLFGRRRYVPDLEARVKSVREAAERMAFNMPVQGT
AADLMKLAMVKLFPRLEEMGARMLLQVHDELVLEAPKERAEAVARLAKEVMEGVYPL
AVPLEVEVGIGEDWLSAKX101-X110
SEQ ID NO: 23-Exemplary DNA polymerase/TBD/TRX chimera (N-terminal TRX) with 60
residue linker
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGGGGSG
GGSGSSGGSGSSGGSSGGGGSGGGSGSSGGSGSSGSGSSGSSGGGGSGGSSRGMLPLFEP
KGRVLLVDGHHLAYRTFHALKGLTTSRGEPVQAVYGFAKSLLKALKEDGDAVIVVFDA
KAPSFRHEAYGGYKAGRAPTPEDFPRQLALIKELVDLLGLARLEVPGYEADDVLASLAK
KAEKEGYEVRILTADKDLYQLLSDRIHVLHPEGYLITPAWLWEKYGLRPDQWADYRAL
TGDESDNLPGVKGIGEKTARKLLEEWGSLEALLKNLDRLKPAIREKILAHMDDLKLSWD
LAKVRTDLPLEVDFAKRREPDRERLRAFLERLEFGSLLHEFGLLESPKALEEAPWPPPEG
AFVGFVLSRKEPMWADLLALAAARGGRVHRAPEPYKALRDLKEARGLLAKDLSVLAL
REGLGLPPGDDPMLLAYLLDPSNTTPEGVARRYGGEWTEEAGERAALSERLFANLWGR
LEGEERLLWLYREVERPLSAVLAHMEATGVRLDVAYLRALSLEVAEEIARLEAEVFRLA
GGSWYQPKGGTEMFCHPRTGKPLPKYPRIKIPKVGGIFKKPKNKAQREGREPCELDTRE
YVAGAPYTPVEHVVFNPSSRDQLERVLFDELGLPAIGKTEKTGKRSTSAAVLEALREAHP
IVEKILQYRELTKLKSTYIDPLPDLIHPRTGRLHTRFNQTATATGRLSSSDPNLQNIPVRTP
LGQRIRRAFIAEEGWLLVALDYSQIELRVLAHLSGDENLIRVFQEGRDIHTETASWMFGV
PREAVDPLMRRAAKTINFGVLYGMSAHRLSQELAIPYEEAQAFIERYFQSFPKVRAWIEK
TLEEGRRRGYVETLFGRRRYVPDLEARVKSVREAAERMAFNMPVQGTAADLMKLAMV
KLFPRLEEMGARMLLQVHDELVLEAPKERAEAVARLAKEVMEGVYPLAVPLEVEVGIG
EDWLSAKGD
SEQ ID NO: 24-Exemplary DNA polymerase/TBD/TRX chimera (C-terminal TRX)
X1-
X10RGMLPLFEPKGRVLLVDGHHLAYRTFHALKGLTTSRGEPVQAVYGFAKSLLKALKE
DGDAVIVVFDAKAPSFRHEAYGGYKAGRAPTPEDFPRQLALIKELVDLLGLARLEVPGY
EADDVLASLAKKAEKEGYEVRILTADKDLYQLLSDRIHVLHPEGYLITPAWLWEKYGLR
PDQWADYRALTGDESDNLPGVKGIGEKTARKLLEEWGSLEALLKNLDRLKPAIREKILA
HMDDLKLSWDLAKVRTDLPLEVDFAKRREPDRERLRAFLERLEFGSLLHEFGLLESPKA
LEEAPWPPPEGAFVGFVLSRKEPMWADLLALAAARGGRVHRAPEPYKALRDLKEARGL
LAKDLSVLALREGLGLPPGDDPMLLAYLLDPSNTTPEGVARRYGGEWTEEAGERAALSE
RLFANLWGRLEGEERLLWLYREVERPLSAVLAHMEATGVRLDVAYLRALSLEVAEEIA
RLEAEVFRLAGGSWYQPKGGTEMFCHPRTGKPLPKYPRIKIPKVGGIFKKPKNKAQREG
REPCELDTREYVAGAPYTPVEHVVFNPSSRDQLERVLFDELGLPAIGKTEKTGKRSTSAA
VLEALREAHPIVEKILQYRELTKLKSTYIDPLPDLIHPRTGRLHTRFNQTATATGRLSSSDP
NLQNIPVRTPLGQRIRRAFIAEEGWLLVALDYSQIELRVLAHLSGDENLIRVFQEGRDIHT
ETASWMFGVPREAVDPLMRRAAKTINFGVLYGMSAHRLSQELAIPYEEAQAFIERYFQS
FPKVRAWIEKTLEEGRRRGYVETLFGRRRYVPDLEARVKSVREAAERMAFNMPVQGTA
ADLMKLAMVKLFPRLEEMGARMLLQVHDELVLEAPKERAEAVARLAKEVMEGVYPLA
VPLEVEVGIGEDWLSAKX11-
X100SDKIIHLTDDSFDTDVLKADGAILVDFWAEWCGPCKMIAPILDEIADEYQGKLTVA
KLNIDQNPGTAPKYGIRGIPTLLLFKNGEVAATKVGALSKGQLKEFLDANLAX101-X110
SEQ ID NO: 25-Exemplary DNA polymerase/TBD/TRX chimera (C-terminal TRX) with 40
residue linker
MRGMLPLFEPKGRVLLVDGHHLAYRTFHALKGLTTSRGEPVQAVYGFAKSLLKALKED
GDAVIVVFDAKAPSFRHEAYGGYKAGRAPTPEDFPRQLALIKELVDLLGLARLEVPGYE
ADDVLASLAKKAEKEGYEVRILTADKDLYQLLSDRIHVLHPEGYLITPAWLWEKYGLRP
DQWADYRALTGDESDNLPGVKGIGEKTARKLLEEWGSLEALLKNLDRLKPAIREKILAH
MDDLKLSWDLAKVRTDLPLEVDFAKRREPDRERLRAFLERLEFGSLLHEFGLLESPKAL
EEAPWPPPEGAFVGFVLSRKEPMWADLLALAAARGGRVHRAPEPYKALRDLKEARGLL
AKDLSVLALREGLGLPPGDDPMLLAYLLDPSNTTPEGVARRYGGEWTEEAGERAALSER
LFANLWGRLEGEERLLWLYREVERPLSAVLAHMEATGVRLDVAYLRALSLEVAEEIAR
LEAEVFRLAGGSWYQPKGGTEMFCHPRTGKPLPKYPRIKIPKVGGIFKKPKNKAQREGR
EPCELDTREYVAGAPYTPVEHVVFNPSSRDQLERVLFDELGLPAIGKTEKTGKRSTSAAV
LEALREAHPIVEKILQYRELTKLKSTYIDPLPDLIHPRTGRLHTRFNQTATATGRLSSSDPN
LONIPVRTPLGQRIRRAFIAEEGWLLVALDYSQIELRVLAHLSGDENLIRVFQEGRDIHTE
TASWMFGVPREAVDPLMRRAAKTINFGVLYGMSAHRLSQELAIPYEEAQAFIERYFQSF
PKVRAWIEKTLEEGRRRGYVETLFGRRRYVPDLEARVKSVREAAERMAFNMPVQGTAA
DLMKLAMVKLFPRLEEMGARMLLQVHDELVLEAPKERAEAVARLAKEVMEGVYPLAV
PLEVEVGIGEDWLSAKGDGGSSGSGGGSSGGSGSSGGGGSGGGSGSGSSGSGSSGSSGSD
KIIHLTDDSFDTDVLKADGAILVDFWAEWCGPCKMIAPILDEIADEYQGKLTVAKLNIDQ
NPGTAPKYGIRGIPTLLLFKNGEVAATKVGALSKGQLKEFLDANLA
SEQ ID NO: 26-Exemplary DNA polymerase/TBD/TRX chimera (dual TRX)
X1-
X10SDKIIHLTDDSFDTDVLKADGAILVDFWAEWCGPCKMIAPILDEIADEYQGKLTVAK
LNIDQNPGTAPKYGIRGIPTLLLFKNGEVAATKVGALSKGQLKEFLDANLAX11-
X100RGMLPLFEPKGRVLLVDGHHLAYRTFHALKGLTTSRGEPVQAVYGFAKSLLKALK
EDGDAVIVVFDAKAPSFRHEAYGGYKAGRAPTPEDFPRQLALIKELVDLLGLARLEVPG
YEADDVLASLAKKAEKEGYEVRILTADKDLYQLLSDRIHVLHPEGYLITPAWLWEKYGL
RPDQWADYRALTGDESDNLPGVKGIGEKTARKLLEEWGSLEALLKNLDRLKPAIREKIL
AHMDDLKLSWDLAKVRTDLPLEVDFAKRREPDRERLRAFLERLEFGSLLHEFGLLESPK
ALEEAPWPPPEGAFVGFVLSRKEPMWADLLALAAARGGRVHRAPEPYKALRDLKEARG
LLAKDLSVLALREGLGLPPGDDPMLLAYLLDPSNTTPEGVARRYGGEWTEEAGERAALS
ERLFANLWGRLEGEERLLWLYREVERPLSAVLAHMEATGVRLDVAYLRALSLEVAEEI
ARLEAEVFRLAGGSWYQPKGGTEMFCHPRTGKPLPKYPRIKIPKVGGIFKKPKNKAQRE
GREPCELDTREYVAGAPYTPVEHVVFNPSSRDQLERVLFDELGLPAIGKTEKTGKRSTSA
AVLEALREAHPIVEKILQYRELTKLKSTYIDPLPDLIHPRTGRLHTRFNQTATATGRLSSSD
PNLQNIPVRTPLGQRIRRAFIAEEGWLLVALDYSQIELRVLAHLSGDENLIRVFQEGRDIH
TETASWMFGVPREAVDPLMRRAAKTINFGVLYGMSAHRLSQELAIPYEEAQAFIERYFQ
SFPKVRAWIEKTLEEGRRRGYVETLFGRRRYVPDLEARVKSVREAAERMAFNMPVQGT
AADLMKLAMVKLFPRLEEMGARMLLQVHDELVLEAPKERAEAVARLAKEVMEGVYPL
AVPLEVEVGIGEDWLSAKX101-
X200DKIIHLTDDSFDTDVLKADGAILVDFWAEWCGPCKMIAPILDEIADEYQGKLTVAK
LNIDQNPGTAPKYGIRGIPTLLLFKNGEVAATKVGALSKGQLKEFLDANLAX201 X220
SEQ ID NO: 27-Exemplary DNA polymerase/TBD/TRX chimera (dual TRX)
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGGGGSG
GGSGSSGGSGSSGGSSGGGGSGSGSGSSGGSGSSGSGSSGSSGGGGSGGSSRGMLPLFEP
KGRVLLVDGHHLAYRTFHALKGLTTSRGEPVQAVYGFAKSLLKALKEDGDAVIVVFDA
KAPSFRHEAYGGYKAGRAPTPEDFPRQLALIKELVDLLGLARLEVPGYEADDVLASLAK
KAEKEGYEVRILTADKDLYQLLSDRIHVLHPEGYLITPAWLWEKYGLRPDQWADYRAL
TGDESDNLPGVKGIGEKTARKLLEEWGSLEALLKNLDRLKPAIREKILAHMDDLKLSWD
LAKVRTDLPLEVDFAKRREPDRERLRAFLERLEFGSLLHEFGLLESPKALEEAPWPPPEG
AFVGFVLSRKEPMWADLLALAAARGGRVHRAPEPYKALRDLKEARGLLAKDLSVLAL
REGLGLPPGDDPMLLAYLLDPSNTTPEGVARRYGGEWTEEAGERAALSERLFANLWGR
LEGEERLLWLYREVERPLSAVLAHMEATGVRLDVAYLRALSLEVAEEIARLEAEVFRLA
GGSWYQPKGGTEMFCHPRTGKPLPKYPRIKIPKVGGIFKKPKNKAQREGREPCELDTRE
YVAGAPYTPVEHVVFNPSSRDQLERVLFDELGLPAIGKTEKTGKRSTSAAVLEALREAHP
IVEKILQYRELTKLKSTYIDPLPDLIHPRTGRLHTRFNQTATATGRLSSSDPNLQNIPVRTP
LGQRIRRAFIAEEGWLLVALDYSQIELRVLAHLSGDENLIRVFQEGRDIHTETASWMFGV
PREAVDPLMRRAAKTINFGVLYGMSAHRLSQELAIPYEEAQAFIERYFQSFPKVRAWIEK
TLEEGRRRGYVETLFGRRRYVPDLEARVKSVREAAERMAFNMPVQGTAADLMKLAMV
KLFPRLEEMGARMLLQVHDELVLEAPKERAEAVARLAKEVMEGVYPLAVPLEVEVGIG
EDWLSAKGDGGSSGSGGGSSGGSGSSGGGGSGGGSGSGSSGSGSSGSSGSDKIIHLTDDS
FDTDVLKADGAILVDFWAEWCGPCKMIAPILDEIADEYQGKLTVAKLNIDQNPGTAPKY
GIRGIPTLLLFKNGEVAATKVGALSKGQLKEFLDANLA
SEQ ID NO: 28-Exemplary DNA polymerase/TBD chimera
X1-
X10RGMLPLFEPKGRVLLVDGHHLAYRTFHALKGLTTSRGEPVQAVYGFAKSLLKALKE
DGDAVIVVFDAKAPSFRHEAYGGYKAGRAPTPEDFPRQLALIKELVDLLGLARLEVPGY
EADDVLASLAKKAEKEGYEVRILTADKDLYQLLSDRIHVLHPEGYLITPAWLWEKYGLR
PDQWADYRALTGDESDNLPGVKGIGEKTARKLLEEWGSLEALLKNLDRLKPAIREKILA
HMDDLKLSWDLAKVRTDLPLEVDFAKRREPDRERLRAFLERLEFGSLLHEFGLLESPKA
LEEAPWPPPEGAFVGFVLSRKEPMWADLLALAAARGGRVHRAPEPYKALRDLKEARGL
LAKDLSVLALREGLGLPPGDDPMLLAYLLDPSNTTPEGVARRYGGEWTEEAGERAALSE
RLFANLWGRLEGEERLLWLYREVERPLSAVLAHMEATGVRLDVAYLRALSLEVAEEIA
RLEAEVFRLAGGSWYQPKGGTEMFCHPRTGKPLPKYPRIKIPKVGGIFKKPKNKAQREG
REPCELDTREYVAGAPYTPVEHVVFNPSSRDQLERVLFDELGLPAIGKTEKTGKRSTSAA
VLEALREAHPIVEKILQYRELTKLKSTYIDPLPDLIHPRTGRLHTRFNQTATATGRLSSSDP
NLQNIPVRTPLGQRIRRAFIAEEGWLLVALDYSQIELRVLAHLSGDENLIRVFQEGRDIHT
ETASWMFGVPREAVDPLMRRAAKTINFGVLYGMSAHRLSQELAIPYEEAQAFIERYFOS
FPKVRAWIEKTLEEGRRRGYVETLFGRRRYVPDLEARVKSVREAAERMAFNMPVQGTA
ADLMKLAMVKLFPRLEEMGARMLLQVHDELVLEAPKERAEAVARLAKEVMEGVYPLA
VPLEVEVGIGEDWLSAKX11-X20
SEQ ID NO: 29-Exemplary DNA polymerase/TBD chimera
MKHHHHHHMRGMLPLFEPKGRVLLVDGHHLAYRTFHALKGLTTSRGEPVQAVYGFAK
SLLKALKEDGDAVIVVFDAKAPSFRHEAYGGYKAGRAPTPEDFPRQLALIKELVDLLGL
ARLEVPGYEADDVLASLAKKAEKEGYEVRILTADKDLYQLLSDRIHVLHPEGYLITPAW
LWEKYGLRPDQWADYRALTGDESDNLPGVKGIGEKTARKLLEEWGSLEALLKNLDRLK
PAIREKILAHMDDLKLSWDLAKVRTDLPLEVDFAKRREPDRERLRAFLERLEFGSLLHEF
GLLESPKALEEAPWPPPEGAFVGFVLSRKEPMWADLLALAAARGGRVHRAPEPYKALR
DLKEARGLLAKDLSVLALREGLGLPPGDDPMLLAYLLDPSNTTPEGVARRYGGEWTEE
AGERAALSERLFANLWGRLEGEERLLWLYREVERPLSAVLAHMEATGVRLDVAYLRAL
SLEVAEEIARLEAEVFRLAGGSWYQPKGGTEMFCHPRTGKPLPKYPRIKIPKVGGIFKKP
KNKAQREGREPCELDTREYVAGAPYTPVEHVVFNPSSRDQLERVLFDELGLPAIGKTEK
TGKRSTSAAVLEALREAHPIVEKILQYRELTKLKSTYIDPLPDLIHPRTGRLHTRFNQTAT
ATGRLSSSDPNLQNIPVRTPLGQRIRRAFIAEEGWLLVALDYSQIELRVLAHLSGDENLIR
VFQEGRDIHTETASWMFGVPREAVDPLMRRAAKTINFGVLYGMSAHRLSQELAIPYEEA
QAFIERYFQSFPKVRAWIEKTLEEGRRRGYVETLFGRRRYVPDLEARVKSVREAAERMA
FNMPVQGTAADLMKLAMVKLFPRLEEMGARMLLQVHDELVLEAPKERAEAVARLAKE
VMEGVYPLAVPLEVEVGIGEDWLSAKGD
SEQ ID NO: 30-Exemplary Taq-TBD(pATG6370)
MKHHHHHHMRGMLPLFEPKGRVLLVDGHHLAYRTFHALKGLTTSRGEPVQAVYGFAK
SLLKALKEDGDAVIVVFDAKAPSFRHEAYGGYKAGRAPTPEDFPRQLALIKELVDLLGL
ARLEVPGYEADDVLASLAKKAEKEGYEVRILTADKDLYQLLSDRIHVLHPEGYLITPAW
LWEKYGLRPDQWADYRALTGDESDNLPGVKGIGEKTARKLLEEWGSLEALLKNLDRLK
PAIREKILAHMDDLKLSWDLAKVRTDLPLEVDFAKRREPDRERLRAFLERLEFGSLLHEF
GLLESPKALEEAPWPPPEGAFVGFVLSRKEPMWADLLALAAARGGRVHRAPEPYKALR
DLKEARGLLAKDLSVLALREGLGLPPGDDPMLLAYLLDPSNTTPEGVARRYGGEWTEE
AGERAALSERLFANLWGRLEGEERLLWLYREVERPLSAVLAHMEATGVRLDVAYLRAL
SLEVAEEIARLEAEVFRLAGGSWYQPKGGTEMFCHPRTGKPLPKYPRIKIPKVGGIFKKP
KNKAQREGREPCELDTREYVAGAPYTPVEHVVFNPSSRDQLERVLFDELGLPAIGKTEK
TGKRSTSAAVLEALREAHPIVEKILQYRELTKLKSTYIDPLPDLIHPRTGRLHTRFNQTAT
ATGRLSSSDPNLQNIPVRTPLGQRIRRAFIAEEGWLLVALDYSQIELRVLAHLSGDENLIR
VFQEGRDIHTETASWMFGVPREAVDPLMRRAAKTINFGVLYGMSAHRLSQELAIPYEEA
QAFIERYFQSFPKVRAWIEKTLEEGRRRGYVETLFGRRRYVPDLEARVKSVREAAERMA
FNMPVQGTAADLMKLAMVKLFPRLEEMGARMLLQVHDELVLEAPKERAEAVARLAKE
VMEGVYPLAVPLEVEVGIGEDWLSAKGD
SEQ ID NO: 31-Exemplary Taq-TBD Δ235Exo (pATG7221)
MDDLKLSWDLAKVRTDLPLEVDFAKRREPDRERLRAFLERLEFGSLLHEFGLLESPKAL
EEAPWPPPEGAFVGFVLSRKEPMWADLLALAAARGGRVHRAPEPYKALRDLKEARGLL
AKDLSVLALREGLGLPPGDDPMLLAYLLDPSNTTPEGVARRYGGEWTEEAGERAALSER
LFANLWGRLEGEERLLWLYREVERPLSAVLAHMEATGVRLDVAYLRALSLEVAEEIAR
LEAEVFRLAGGSWYQPKGGTEMFCHPRTGKPLPKYPRIKIPKVGGIFKKPKNKAQREGR
EPCELDTREYVAGAPYTPVEHVVFNPSSRDQLERVLFDELGLPAIGKTEKTGKRSTSAAV
LEALREAHPIVEKILQYRELTKLKSTYIDPLPDLIHPRTGRLHTRFNQTATATGRLSSSDPN
LONIPVRTPLGQRIRRAFIAEEGWLLVALDYSQIELRVLAHLSGDENLIRVFQEGRDIHTE
TASWMFGVPREAVDPLMRRAAKTINFGVLYGMSAHRLSQELAIPYEEAQAFIERYFQSF
PKVRAWIEKTLEEGRRRGYVETLFGRRRYVPDLEARVKSVREAAERMAFNMPVQGTAA
DLMKLAMVKLFPRLEEMGARMLLQVHDELVLEAPKERAEAVARLAKEVMEGVYPLAV
PLEVEVGIGEDWLSAKGD
SEQ ID NO: 32-Exemplary Taq-TBD (C493S), pATG7354
MRGMLPLFEPKGRVLLVDGHHLAYRTFHALKGLTTSRGEPVQAVYGFAKSLLKALKED
GDAVIVVFDAKAPSFRHEAYGGYKAGRAPTPEDFPRQLALIKELVDLLGLARLEVPGYE
ADDVLASLAKKAEKEGYEVRILTADKDLYQLLSDRIHVLHPEGYLITPAWLWEKYGLRP
DQWADYRALTGDESDNLPGVKGIGEKTARKLLEEWGSLEALLKNLDRLKPAIREKILAH
MDDLKLSWDLAKVRTDLPLEVDFAKRREPDRERLRAFLERLEFGSLLHEFGLLESPKAL
EEAPWPPPEGAFVGFVLSRKEPMWADLLALAAARGGRVHRAPEPYKALRDLKEARGLL
AKDLSVLALREGLGLPPGDDPMLLAYLLDPSNTTPEGVARRYGGEWTEEAGERAALSER
LFANLWGRLEGEERLLWLYREVERPLSAVLAHMEATGVRLDVAYLRALSLEVAEEIAR
LEAEVFRLAGGSWYQPKGGTEMFSHPRTGKPLPKYPRIKIPKVGGIFKKPKNKAQREGR
EPCELDTREYVAGAPYTPVEHVVFNPSSRDQLERVLFDELGLPAIGKTEKTGKRSTSAAV
LEALREAHPIVEKILQYRELTKLKSTYIDPLPDLIHPRTGRLHTRFNQTATATGRLSSSDPN
LONIPVRTPLGQRIRRAFIAEEGWLLVALDYSQIELRVLAHLSGDENLIRVFQEGRDIHTE
TASWMFGVPREAVDPLMRRAAKTINFGVLYGMSAHRLSQELAIPYEEAQAFIERYFQSF
PKVRAWIEKTLEEGRRRGYVETLFGRRRYVPDLEARVKSVREAAERMAFNMPVQGTAA
DLMKLAMVKLFPRLEEMGARMLLQVHDELVLEAPKERAEAVARLAKEVMEGVYPLAV
PLEVEVGIGEDWLSAKGD
SEQ ID NO: 33-Exemplary Taq-TBD (C531S), pATG7355
MRGMLPLFEPKGRVLLVDGHHLAYRTFHALKGLTTSRGEPVQAVYGFAKSLLKALKED
GDAVIVVFDAKAPSFRHEAYGGYKAGRAPTPEDFPRQLALIKELVDLLGLARLEVPGYE
ADDVLASLAKKAEKEGYEVRILTADKDLYQLLSDRIHVLHPEGYLITPAWLWEKYGLRP
DQWADYRALTGDESDNLPGVKGIGEKTARKLLEEWGSLEALLKNLDRLKPAIREKILAH
MDDLKLSWDLAKVRTDLPLEVDFAKRREPDRERLRAFLERLEFGSLLHEFGLLESPKAL
EEAPWPPPEGAFVGFVLSRKEPMWADLLALAAARGGRVHRAPEPYKALRDLKEARGLL
AKDLSVLALREGLGLPPGDDPMLLAYLLDPSNTTPEGVARRYGGEWTEEAGERAALSER
LFANLWGRLEGEERLLWLYREVERPLSAVLAHMEATGVRLDVAYLRALSLEVAEEIAR
LEAEVFRLAGGSWYQPKGGTEMFCHPRTGKPLPKYPRIKIPKVGGIFKKPKNKAQREGR
EPSELDTREYVAGAPYTPVEHVVFNPSSRDQLERVLFDELGLPAIGKTEKTGKRSTSAAV
LEALREAHPIVEKILQYRELTKLKSTYIDPLPDLIHPRTGRLHTRFNQTATATGRLSSSDPN
LONIPVRTPLGQRIRRAFIAEEGWLLVALDYSQIELRVLAHLSGDENLIRVFQEGRDIHTE
TASWMFGVPREAVDPLMRRAAKTINFGVLYGMSAHRLSQELAIPYEEAQAFIERYFQSF
PKVRAWIEKTLEEGRRRGYVETLFGRRRYVPDLEARVKSVREAAERMAFNMPVQGTAA
DLMKLAMVKLFPRLEEMGARMLLQVHDELVLEAPKERAEAVARLAKEVMEGVYPLAV
PLEVEVGIGEDWLSAKGD
SEQ ID NO: 34-Exemplary Taq-TBD (C493S/C531S), pATG7356
MRGMLPLFEPKGRVLLVDGHHLAYRTFHALKGLTTSRGEPVQAVYGFAKSLLKALKED
GDAVIVVFDAKAPSFRHEAYGGYKAGRAPTPEDFPRQLALIKELVDLLGLARLEVPGYE
ADDVLASLAKKAEKEGYEVRILTADKDLYQLLSDRIHVLHPEGYLITPAWLWEKYGLRP
DQWADYRALTGDESDNLPGVKGIGEKTARKLLEEWGSLEALLKNLDRLKPAIREKILAH
MDDLKLSWDLAKVRTDLPLEVDFAKRREPDRERLRAFLERLEFGSLLHEFGLLESPKAL
EEAPWPPPEGAFVGFVLSRKEPMWADLLALAAARGGRVHRAPEPYKALRDLKEARGLL
AKDLSVLALREGLGLPPGDDPMLLAYLLDPSNTTPEGVARRYGGEWTEEAGERAALSER
LFANLWGRLEGEERLLWLYREVERPLSAVLAHMEATGVRLDVAYLRALSLEVAEEIAR
LEAEVFRLAGGSWYQPKGGTEMFSHPRTGKPLPKYPRIKIPKVGGIFKKPKNKAQREGR
EPSELDTREYVAGAPYTPVEHVVFNPSSRDQLERVLFDELGLPAIGKTEKTGKRSTSAAV
LEALREAHPIVEKILQYRELTKLKSTYIDPLPDLIHPRTGRLHTRFNQTATATGRLSSSDPN
LONIPVRTPLGQRIRRAFIAEEGWLLVALDYSQIELRVLAHLSGDENLIRVFQEGRDIHTE
TASWMFGVPREAVDPLMRRAAKTINFGVLYGMSAHRLSQELAIPYEEAQAFIERYFQSF
PKVRAWIEKTLEEGRRRGYVETLFGRRRYVPDLEARVKSVREAAERMAFNMPVQGTAA
DLMKLAMVKLFPRLEEMGARMLLQVHDELVLEAPKERAEAVARLAKEVMEGVYPLAV
PLEVEVGIGEDWLSAKGD
SEQ ID NO: 35-Exemplary TRX-Taq-TBD, N-terminal TRX with 60 residue linker,
(pATG7346)
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGGGGSG
GGSGSSGGSGSSGGSSGGGGSGSGSGSSGGSGSSGSGSSGSSGGGGSGGSSRGMLPLFEP
KGRVLLVDGHHLAYRTFHALKGLTTSRGEPVQAVYGFAKSLLKALKEDGDAVIVVFDA
KAPSFRHEAYGGYKAGRAPTPEDFPRQLALIKELVDLLGLARLEVPGYEADDVLASLAK
KAEKEGYEVRILTADKDLYQLLSDRIHVLHPEGYLITPAWLWEKYGLRPDQWADYRAL
TGDESDNLPGVKGIGEKTARKLLEEWGSLEALLKNLDRLKPAIREKILAHMDDLKLSWD
LAKVRTDLPLEVDFAKRREPDRERLRAFLERLEFGSLLHEFGLLESPKALEEAPWPPPEG
AFVGFVLSRKEPMWADLLALAAARGGRVHRAPEPYKALRDLKEARGLLAKDLSVLAL
REGLGLPPGDDPMLLAYLLDPSNTTPEGVARRYGGEWTEEAGERAALSERLFANLWGR
LEGEERLLWLYREVERPLSAVLAHMEATGVRLDVAYLRALSLEVAEEIARLEAEVFRLA
GGSWYQPKGGTEMFCHPRTGKPLPKYPRIKIPKVGGIFKKPKNKAQREGREPCELDTRE
YVAGAPYTPVEHVVFNPSSRDQLERVLFDELGLPAIGKTEKTGKRSTSAAVLEALREAHP
IVEKILQYRELTKLKSTYIDPLPDLIHPRTGRLHTRFNQTATATGRLSSSDPNLQNIPVRTP
LGQRIRRAFIAEEGWLLVALDYSQIELRVLAHLSGDENLIRVFQEGRDIHTETASWMFGV
PREAVDPLMRRAAKTINFGVLYGMSAHRLSQELAIPYEEAQAFIERYFQSFPKVRAWIEK
TLEEGRRRGYVETLFGRRRYVPDLEARVKSVREAAERMAFNMPVQGTAADLMKLAMV
KLFPRLEEMGARMLLQVHDELVLEAPKERAEAVARLAKEVMEGVYPLAVPLEVEVGIG
EDWLSAKGD
SEQ ID NO: 36-Exemplary TRX-Taq-TBD, N-terminal TRX with 60 residue linker, I677T
(pATG7435)
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGGGGSG
GGSGSSGGSGSSGGSSGGGGSGGGSGSSGGSGSSGSGSSGSSGGGGSGGSSRGMLPLFEP
KGRVLLVDGHHLAYRTFHALKGLTTSRGEPVQAVYGFAKSLLKALKEDGDAVIVVEDA
KAPSFRHEAYGGYKAGRAPTPEDFPRQLALIKELVDLLGLARLEVPGYEADDVLASLAK
KAEKEGYEVRILTADKDLYQLLSDRIHVLHPEGYLITPAWLWEKYGLRPDQWADYRAL
TGDESDNLPGVKGIGEKTARKLLEEWGSLEALLKNLDRLKPAIREKILAHMDDLKLSWD
LAKVRTDLPLEVDFAKRREPDRERLRAFLERLEFGSLLHEFGLLESPKALEEAPWPPPEG
AFVGFVLSRKEPMWADLLALAAARGGRVHRAPEPYKALRDLKEARGLLAKDLSVLAL
REGLGLPPGDDPMLLAYLLDPSNTTPEGVARRYGGEWTEEAGERAALSERLFANLWGR
LEGEERLLWLYREVERPLSAVLAHMEATGVRLDVAYLRALSLEVAEEIARLEAEVFRLA
GGSWYQPKGGTEMFCHPRTGKPLPKYPRIKTPKVGGIFKKPKNKAQREGREPCELDTRE
YVAGAPYTPVEHVVFNPSSRDQLERVLFDELGLPAIGKTEKTGKRSTSAAVLEALREAHP
IVEKILQYRELTKLKSTYIDPLPDLIHPRTGRLHTRFNQTATATGRLSSSDPNLQNIPVRTP
LGQRIRRAFIAEEGWLLVALDYSQIELRVLAHLSGDENLIRVFQEGRDIHTETASWMFGV
PREAVDPLMRRAAKTINFGVLYGMSAHRLSQELAIPYEEAQAFIERYFQSFPKVRAWIEK
TLEEGRRRGYVETLFGRRRYVPDLEARVKSVREAAERMAFNMPVQGTAADLMKLAMV
KLFPRLEEMGARMLLQVHDELVLEAPKERAEAVARLAKEVMEGVYPLAVPLEVEVGIG
EDWLSAKGD
SEQ ID NO: 37-Exemplary TRX-Taq-TBD, N-terminal TRX with 55 residue linker,
(pATG7360)
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGGGSGSS
GGSGSSGGSSGGGGSGSGSGSSGGSGSSGSGSSGSSGGGGSGGSSRGMLPLFEPKGRVLL
VDGHHLAYRTFHALKGLTTSRGEPVQAVYGFAKSLLKALKEDGDAVIVVFDAKAPSFR
HEAYGGYKAGRAPTPEDFPRQLALIKELVDLLGLARLEVPGYEADDVLASLAKKAEKEG
YEVRILTADKDLYQLLSDRIHVLHPEGYLITPAWLWEKYGLRPDQWADYRALTGDESD
NLPGVKGIGEKTARKLLEEWGSLEALLKNLDRLKPAIREKILAHMDDLKLSWDLAKVRT
DLPLEVDFAKRREPDRERLRAFLERLEFGSLLHEFGLLESPKALEEAPWPPPEGAFVGFVL
SRKEPMWADLLALAAARGGRVHRAPEPYKALRDLKEARGLLAKDLSVLALREGLGLPP
GDDPMLLAYLLDPSNTTPEGVARRYGGEWTEEAGERAALSERLFANLWGRLEGEERLL
WLYREVERPLSAVLAHMEATGVRLDVAYLRALSLEVAEEIARLEAEVERLAGGSWYQP
KGGTEMFCHPRTGKPLPKYPRIKIPKVGGIFKKPKNKAQREGREPCELDTREYVAGAPYT
PVEHVVFNPSSRDQLERVLFDELGLPAIGKTEKTGKRSTSAAVLEALREAHPIVEKILQYR
ELTKLKSTYIDPLPDLIHPRTGRLHTRFNQTATATGRLSSSDPNLQNIPVRTPLGQRIRRAF
IAEEGWLLVALDYSQIELRVLAHLSGDENLIRVFQEGRDIHTETASWMFGVPREAVDPL
MRRAAKTINFGVLYGMSAHRLSQELAIPYEEAQAFIERYFQSFPKVRAWIEKTLEEGRRR
GYVETLFGRRRYVPDLEARVKSVREAAERMAFNMPVQGTAADLMKLAMVKLFPRLEE
MGARMLLQVHDELVLEAPKERAEAVARLAKEVMEGVYPLAVPLEVEVGIGEDWLSAK
GD
SEQ ID NO: 38-Exemplary TRX-Taq-TBD, N-terminal TRX with 50 residue linker,
(pATG7376)
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGSGGSGS
SGGSSGGGGSGSGSGSSGGSGSSGSGSSGSSGGGGSGGSSRGMLPLFEPKGRVLLVDGH
HLAYRTFHALKGLTTSRGEPVQAVYGFAKSLLKALKEDGDAVIVVFDAKAPSFRHEAY
GGYKAGRAPTPEDFPRQLALIKELVDLLGLARLEVPGYEADDVLASLAKKAEKEGYEVR
ILTADKDLYQLLSDRIHVLHPEGYLITPAWLWEKYGLRPDQWADYRALTGDESDNLPGV
KGIGEKTARKLLEEWGSLEALLKNLDRLKPAIREKILAHMDDLKLSWDLAKVRTDLPLE
VDFAKRREPDRERLRAFLERLEFGSLLHEFGLLESPKALEEAPWPPPEGAFVGFVLSRKEP
MWADLLALAAARGGRVHRAPEPYKALRDLKEARGLLAKDLSVLALREGLGLPPGDDP
MLLAYLLDPSNTTPEGVARRYGGEWTEEAGERAALSERLFANLWGRLEGEERLLWLYR
EVERPLSAVLAHMEATGVRLDVAYLRALSLEVAEEIARLEAEVFRLAGGSWYQPKGGT
EMFCHPRTGKPLPKYPRIKIPKVGGIFKKPKNKAQREGREPCELDTREYVAGAPYTPVEH
VVFNPSSRDQLERVLFDELGLPAIGKTEKTGKRSTSAAVLEALREAHPIVEKILQYRELTK
LKSTYIDPLPDLIHPRTGRLHTRFNQTATATGRLSSSDPNLQNIPVRTPLGQRIRRAFIAEE
GWLLVALDYSQIELRVLAHLSGDENLIRVFQEGRDIHTETASWMFGVPREAVDPLMRRA
AKTINFGVLYGMSAHRLSQELAIPYEEAQAFIERYFQSFPKVRAWIEKTLEEGRRRGYVE
TLFGRRRYVPDLEARVKSVREAAERMAFNMPVQGTAADLMKLAMVKLFPRLEEMGAR
MLLQVHDELVLEAPKERAEAVARLAKEVMEGVYPLAVPLEVEVGIGEDWLSAKGD
SEQ ID NO: 39-Exemplary Taq-TBD-TRX, C-terminal TRX with 45 residue linker,
(pATG7358)
MRGMLPLFEPKGRVLLVDGHHLAYRTFHALKGLTTSRGEPVQAVYGFAKSLLKALKED
GDAVIVVFDAKAPSFRHEAYGGYKAGRAPTPEDFPRQLALIKELVDLLGLARLEVPGYE
ADDVLASLAKKAEKEGYEVRILTADKDLYQLLSDRIHVLHPEGYLITPAWLWEKYGLRP
DQWADYRALTGDESDNLPGVKGIGEKTARKLLEEWGSLEALLKNLDRLKPAIREKILAH
MDDLKLSWDLAKVRTDLPLEVDFAKRREPDRERLRAFLERLEFGSLLHEFGLLESPKAL
EEAPWPPPEGAFVGFVLSRKEPMWADLLALAAARGGRVHRAPEPYKALRDLKEARGLL
AKDLSVLALREGLGLPPGDDPMLLAYLLDPSNTTPEGVARRYGGEWTEEAGERAALSER
LFANLWGRLEGEERLLWLYREVERPLSAVLAHMEATGVRLDVAYLRALSLEVAEEIAR
LEAEVFRLAGGSWYQPKGGTEMFCHPRTGKPLPKYPRIKIPKVGGIFKKPKNKAQREGR
EPCELDTREYVAGAPYTPVEHVVFNPSSRDQLERVLFDELGLPAIGKTEKTGKRSTSAAV
LEALREAHPIVEKILQYRELTKLKSTYIDPLPDLIHPRTGRLHTRFNQTATATGRLSSSDPN
LONIPVRTPLGQRIRRAFIAEEGWLLVALDYSQIELRVLAHLSGDENLIRVFQEGRDIHTE
TASWMFGVPREAVDPLMRRAAKTINFGVLYGMSAHRLSQELAIPYEEAQAFIERYFQSF
PKVRAWIEKTLEEGRRRGYVETLFGRRRYVPDLEARVKSVREAAERMAFNMPVQGTAA
DLMKLAMVKLFPRLEEMGARMLLQVHDELVLEAPKERAEAVARLAKEVMEGVYPLAV
PLEVEVGIGEDWLSAKGDGGSSGSGGGSSGGSGSSGGGGSGGGSGSGSSGSGSSGGSSG
GSSGSDKIIHLTDDSFDTDVLKADGAILVDFWAEWCGPCKMIAPILDEIADEYQGKLTVA
KLNIDQNPGTAPKYGIRGIPTLLLFKNGEVAATKVGALSKGQLKEFLDANLA
SEQ ID NO: 40-Exemplary Taq-TBD-TRX, C-terminal TRX with 40 residue linker,
(pATG7347)
MRGMLPLFEPKGRVLLVDGHHLAYRTFHALKGLTTSRGEPVQAVYGFAKSLLKALKED
GDAVIVVFDAKAPSFRHEAYGGYKAGRAPTPEDFPRQLALIKELVDLLGLARLEVPGYE
ADDVLASLAKKAEKEGYEVRILTADKDLYQLLSDRIHVLHPEGYLITPAWLWEKYGLRP
DQWADYRALTGDESDNLPGVKGIGEKTARKLLEEWGSLEALLKNLDRLKPAIREKILAH
MDDLKLSWDLAKVRTDLPLEVDFAKRREPDRERLRAFLERLEFGSLLHEFGLLESPKAL
EEAPWPPPEGAFVGFVLSRKEPMWADLLALAAARGGRVHRAPEPYKALRDLKEARGLL
AKDLSVLALREGLGLPPGDDPMLLAYLLDPSNTTPEGVARRYGGEWTEEAGERAALSER
LFANLWGRLEGEERLLWLYREVERPLSAVLAHMEATGVRLDVAYLRALSLEVAEEIAR
LEAEVFRLAGGSWYQPKGGTEMFCHPRTGKPLPKYPRIKIPKVGGIFKKPKNKAQREGR
EPCELDTREYVAGAPYTPVEHVVFNPSSRDQLERVLFDELGLPAIGKTEKTGKRSTSAAV
LEALREAHPIVEKILQYRELTKLKSTYIDPLPDLIHPRTGRLHTRFNQTATATGRLSSSDPN
LONIPVRTPLGQRIRRAFIAEEGWLLVALDYSQIELRVLAHLSGDENLIRVFQEGRDIHTE
TASWMFGVPREAVDPLMRRAAKTINFGVLYGMSAHRLSQELAIPYEEAQAFIERYFQSF
PKVRAWIEKTLEEGRRRGYVETLFGRRRYVPDLEARVKSVREAAERMAFNMPVQGTAA
DLMKLAMVKLFPRLEEMGARMLLQVHDELVLEAPKERAEAVARLAKEVMEGVYPLAV
PLEVEVGIGEDWLSAKGDGGSSGSGGGSSGGSGSSGGGGSGGGSGSGSSGSGSSGSSGSD
KIIHLTDDSFDTDVLKADGAILVDFWAEWCGPCKMIAPILDEIADEYQGKLTVAKLNIDQ
NPGTAPKYGIRGIPTLLLFKNGEVAATKVGALSKGQLKEFLDANLA
SEQ ID NO: 41-Exemplary Taq-TBD-TRX, C-terminal TRX with 35 residue linker,
(pATG7357)
MRGMLPLFEPKGRVLLVDGHHLAYRTFHALKGLTTSRGEPVQAVYGFAKSLLKALKED
GDAVIVVFDAKAPSFRHEAYGGYKAGRAPTPEDFPRQLALIKELVDLLGLARLEVPGYE
ADDVLASLAKKAEKEGYEVRILTADKDLYQLLSDRIHVLHPEGYLITPAWLWEKYGLRP
DQWADYRALTGDESDNLPGVKGIGEKTARKLLEEWGSLEALLKNLDRLKPAIREKILAH
MDDLKLSWDLAKVRTDLPLEVDFAKRREPDRERLRAFLERLEFGSLLHEFGLLESPKAL
EEAPWPPPEGAFVGFVLSRKEPMWADLLALAAARGGRVHRAPEPYKALRDLKEARGLL
AKDLSVLALREGLGLPPGDDPMLLAYLLDPSNTTPEGVARRYGGEWTEEAGERAALSER
LFANLWGRLEGEERLLWLYREVERPLSAVLAHMEATGVRLDVAYLRALSLEVAEEIAR
LEAEVFRLAGGSWYQPKGGTEMFCHPRTGKPLPKYPRIKIPKVGGIFKKPKNKAQREGR
EPCELDTREYVAGAPYTPVEHVVFNPSSRDQLERVLFDELGLPAIGKTEKTGKRSTSAAV
LEALREAHPIVEKILQYRELTKLKSTYIDPLPDLIHPRTGRLHTRFNQTATATGRLSSSDPN
LQNIPVRTPLGQRIRRAFIAEEGWLLVALDYSQIELRVLAHLSGDENLIRVFQEGRDIHTE
TASWMFGVPREAVDPLMRRAAKTINFGVLYGMSAHRLSQELAIPYEEAQAFIERYFQSF
PKVRAWIEKTLEEGRRRGYVETLFGRRRYVPDLEARVKSVREAAERMAFNMPVQGTAA
DLMKLAMVKLFPRLEEMGARMLLQVHDELVLEAPKERAEAVARLAKEVMEGVYPLAV
PLEVEVGIGEDWLSAKGDGGSSGSGGGSSGGSGSSGGGGSGGGSGSGSSGSSGSDKIIHL
TDDSFDTDVLKADGAILVDFWAEWCGPCKMIAPILDEIADEYQGKLTVAKLNIDQNPGT
APKYGIRGIPTLLLFKNGEVAATKVGALSKGQLKEFLDANLA
SEQ ID NO: 42-Exemplary TRX-Taq-TBD-TRX, dually fused TRX with 60 and 40 residue
linkers, (pATG7350)
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGGGGSG
GGSGSSGGSGSSGGSSGGGGSGSGSGSSGGSGSSGSGSSGSSGGGGSGGSSRGMLPLFEP
KGRVLLVDGHHLAYRTFHALKGLTTSRGEPVQAVYGFAKSLLKALKEDGDAVIVVFDA
KAPSFRHEAYGGYKAGRAPTPEDFPRQLALIKELVDLLGLARLEVPGYEADDVLASLAK
KAEKEGYEVRILTADKDLYQLLSDRIHVLHPEGYLITPAWLWEKYGLRPDQWADYRAL
TGDESDNLPGVKGIGEKTARKLLEEWGSLEALLKNLDRLKPAIREKILAHMDDLKLSWD
LAKVRTDLPLEVDFAKRREPDRERLRAFLERLEFGSLLHEFGLLESPKALEEAPWPPPEG
AFVGFVLSRKEPMWADLLALAAARGGRVHRAPEPYKALRDLKEARGLLAKDLSVLAL
REGLGLPPGDDPMLLAYLLDPSNTTPEGVARRYGGEWTEEAGERAALSERLFANLWGR
LEGEERLLWLYREVERPLSAVLAHMEATGVRLDVAYLRALSLEVAEEIARLEAEVFRLA
GGSWYQPKGGTEMFCHPRTGKPLPKYPRIKIPKVGGIFKKPKNKAQREGREPCELDTRE
YVAGAPYTPVEHVVFNPSSRDQLERVLFDELGLPAIGKTEKTGKRSTSAAVLEALREAHP
IVEKILQYRELTKLKSTYIDPLPDLIHPRTGRLHTRFNQTATATGRLSSSDPNLQNIPVRTP
LGQRIRRAFIAEEGWLLVALDYSQIELRVLAHLSGDENLIRVFQEGRDIHTETASWMFGV
PREAVDPLMRRAAKTINFGVLYGMSAHRLSQELAIPYEEAQAFIERYFQSFPKVRAWIEK
TLEEGRRRGYVETLFGRRRYVPDLEARVKSVREAAERMAFNMPVQGTAADLMKLAMV
KLFPRLEEMGARMLLQVHDELVLEAPKERAEAVARLAKEVMEGVYPLAVPLEVEVGIG
EDWLSAKGDGGSSGSGGGSSGGSGSSGGGGSGGGSGSGSSGSGSSGSSGSDKIIHLTDDS
FDTDVLKADGAILVDFWAEWCGPCKMIAPILDEIADEYQGKLTVAKLNIDQNPGTAPKY
GIRGIPTLLLFKNGEVAATKVGALSKGQLKEFLDANLA
SEQ ID NO: 43-Exemplary 2x TRX-Taq-TBD (pATG7427)
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGSGSDKII
HLTDDSFDTDVLKADGAILVDFWAEWCGPCKMIAPILDEIADEYQGKLTVAKLNIDQNP
GTAPKYGIRGIPTLLLFKNGEVAATKVGALSKGQLKEFLDANLASSGGGGGSGGGSGSS
GGSGSSGGSSGGGGSGSGSGSSGGSGSSGSGSSGSSGGGGSGGSSRGMLPLFEPKGRVLL
VDGHHLAYRTFHALKGLTTSRGEPVQAVYGFAKSLLKALKEDGDAVIVVFDAKAPSFR
HEAYGGYKAGRAPTPEDFPRQLALIKELVDLLGLARLEVPGYEADDVLASLAKKAEKEG
YEVRILTADKDLYQLLSDRIHVLHPEGYLITPAWLWEKYGLRPDQWADYRALTGDESD
NLPGVKGIGEKTARKLLEEWGSLEALLKNLDRLKPAIREKILAHMDDLKLSWDLAKVRT
DLPLEVDFAKRREPDRERLRAFLERLEFGSLLHEFGLLESPKALEEAPWPPPEGAFVGFVL
SRKEPMWADLLALAAARGGRVHRAPEPYKALRDLKEARGLLAKDLSVLALREGLGLPP
GDDPMLLAYLLDPSNTTPEGVARRYGGEWTEEAGERAALSERLFANLWGRLEGEERLL
WLYREVERPLSAVLAHMEATGVRLDVAYLRALSLEVAEEIARLEAEVFRLAGGSWYQP
KGGTEMFCHPRTGKPLPKYPRIKIPKVGGIFKKPKNKAQREGREPCELDTREYVAGAPYT
PVEHVVFNPSSRDQLERVLFDELGLPAIGKTEKTGKRSTSAAVLEALREAHPIVEKILQYR
ELTKLKSTYIDPLPDLIHPRTGRLHTRFNQTATATGRLSSSDPNLQNIPVRTPLGQRIRRAF
IAEEGWLLVALDYSQIELRVLAHLSGDENLIRVFQEGRDIHTETASWMFGVPREAVDPL
MRRAAKTINFGVLYGMSAHRLSQELAIPYEEAQAFIERYFQSFPKVRAWIEKTLEEGRRR
GYVETLFGRRRYVPDLEARVKSVREAAERMAFNMPVQGTAADLMKLAMVKLFPRLEE
MGARMLLQVHDELVLEAPKERAEAVARLAKEVMEGVYPLAVPLEVEVGIGEDWLSAK
GD
SEQ ID NO: 44-Exemplary 3xTRX-Taq-TBD (pATG7426)
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGGGSDKI
IHLTDDSFDTDVLKADGAILVDFWAEWCGPCKMIAPILDEIADEYQGKLTVAKLNIDQNP
GTAPKYGIRGIPTLLLFKNGEVAATKVGALSKGQLKEFLDANLAGSSGSGSDKIIHLTDD
SFDTDVLKADGAILVDFWAEWCGPCKMIAPILDEIADEYQGKLTVAKLNIDQNPGTAPK
YGIRGIPTLLLFKNGEVAATKVGALSKGQLKEFLDANLASSGGGGGSGGGSGSSGGSGSS
GGSSGGGGSGSGSGSSGGSGSSGSGSSGSSGGGGSGGSSRGMLPLFEPKGRVLLVDGHH
LAYRTFHALKGLTTSRGEPVQAVYGFAKSLLKALKEDGDAVIVVFDAKAPSFRHEAYG
GYKAGRAPTPEDFPRQLALIKELVDLLGLARLEVPGYEADDVLASLAKKAEKEGYEVRI
LTADKDLYQLLSDRIHVLHPEGYLITPAWLWEKYGLRPDQWADYRALTGDESDNLPGV
KGIGEKTARKLLEEWGSLEALLKNLDRLKPAIREKILAHMDDLKLSWDLAKVRTDLPLE
VDFAKRREPDRERLRAFLERLEFGSLLHEFGLLESPKALEEAPWPPPEGAFVGFVLSRKEP
MWADLLALAAARGGRVHRAPEPYKALRDLKEARGLLAKDLSVLALREGLGLPPGDDP
MLLAYLLDPSNTTPEGVARRYGGEWTEEAGERAALSERLFANLWGRLEGEERLLWLYR
EVERPLSAVLAHMEATGVRLDVAYLRALSLEVAEEIARLEAEVFRLAGGSWYQPKGGT
EMFCHPRTGKPLPKYPRIKIPKVGGIFKKPKNKAQREGREPCELDTREYVAGAPYTPVEH
VVFNPSSRDQLERVLFDELGLPAIGKTEKTGKRSTSAAVLEALREAHPIVEKILQYRELTK
LKSTYIDPLPDLIHPRTGRLHTRFNQTATATGRLSSSDPNLQNIPVRTPLGQRIRRAFIAEE
GWLLVALDYSQIELRVLAHLSGDENLIRVFQEGRDIHTETASWMFGVPREAVDPLMRRA
AKTINFGVLYGMSAHRLSQELAIPYEEAQAFIERYFQSFPKVRAWIEKTLEEGRRRGYVE
TLFGRRRYVPDLEARVKSVREAAERMAFNMPVQGTAADLMKLAMVKLFPRLEEMGAR
MLLQVHDELVLEAPKERAEAVARLAKEVMEGVYPLAVPLEVEVGIGEDWLSAKGD
SEQ ID NO: 45-TRX-Taq-TBD with 60 residue linker and T7 TIS sequence at insertion
site D
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGGGGSG
GGSGSSGGSGSSGGSSGGGGSGGGSGSSGGSGSSGSGSSGSSGGGGSGGSSRGMLPLFEP
KGRVLLVDGHHLAYRTFHALKGLTTSRGEPVQAVYGFAKSLLKALKEDGDAVIVVFDA
KAPSFRHEAYGGYKAGRAPTPEDFPRQLALIKELVDLLGLARLEVPGYEADDVLASLAK
KAEKEGYEVRILTADKDLYQLLSDRIHVLHPEGYLITPAWLWEKYGLRPDQWADYRAL
TGDESDNLPGVKGIGEKTARKLLEEWGSLEALLKNLDRLKPAIREKILAHMDDLKLSWD
LAKVRTDLPLEVDFAKRREPDRERLRAFLERLEFGSLLHEFGLLESPKALEEAPWPPPEG
AFVGFVLSRKEPMWADLLALAAARGGRVHRAPEPYKALRDLKEARGLLAKDLSVLAL
REGLGLPPGDDPMLLAYLLDPSNTDTDMGLLRSGKLPGKRFGSHALEAWGYRLGEMKG
EYKDDFKRMLEEQGEEYVDGMEWWNFNEEMMDYAGERAALSERLFANLWGRLEGEE
RLLWLYREVERPLSAVLAHMEATGVRLDVAYLRALSLEVAEEIARLEAEVFRLAGGSW
YQPKGGTEMFCHPRTGKPLPKYPRIKIPKVGGIFKKPKNKAQREGREPCELDTREYVAG
APYTPVEHVVFNPSSRDQLERVLFDELGLPAIGKTEKTGKRSTSAAVLEALREAHPIVEKI
LQYRELTKLKSTYIDPLPDLIHPRTGRLHTRFNQTATATGRLSSSDPNLQNIPVRTPLGQRI
RRAFIAEEGWLLVALDYSQIELRVLAHLSGDENLIRVFQEGRDIHTETASWMFGVPREAV
DPLMRRAAKTINFGVLYGMSAHRLSQELAIPYEEAQAFIERYFQSFPKVRAWIEKTLEEG
RRRGYVETLFGRRRYVPDLEARVKSVREAAERMAFNMPVQGTAADLMKLAMVKLFPR
LEEMGARMLLQVHDELVLEAPKERAEAVARLAKEVMEGVYPLAVPLEVEVGIGEDWLS
AKGD
SEQ ID NO: 46-Exonuclease deficient TRX-Taq-TBD with 60 residue linker and T7 TIS
sequence at insertion site D
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGGGGSG
GGSGSSGGSGSSGGSSGGGGSGGGSGSSGGSGSSGSGSSGSSGGGGSGGSSMDDLKLSW
DLAKVRTDLPLEVDFAKRREPDRERLRAFLERLEFGSLLHEFGLLESPKALEEAPWPPPE
GAFVGFVLSRKEPMWADLLALAAARGGRVHRAPEPYKALRDLKEARGLLAKDLSVLA
LREGLGLPPGDDPMLLAYLLDPSNTDTDMGLLRSGKLPGKRFGSHALEAWGYRLGEMK
GEYKDDFKRMLEEQGEEYVDGMEWWNFNEEMMDYAGERAALSERLFANLWGRLEGE
ERLLWLYREVERPLSAVLAHMEATGVRLDVAYLRALSLEVAEEIARLEAEVFRLAGGS
WYQPKGGTEMFCHPRTGKPLPKYPRIKIPKVGGIFKKPKNKAQREGREPCELDTREYVA
GAPYTPVEHVVFNPSSRDQLERVLFDELGLPAIGKTEKTGKRSTSAAVLEALREAHPIVE
KILQYRELTKLKSTYIDPLPDLIHPRTGRLHTRFNQTATATGRLSSSDPNLQNIPVRTPLGQ
RIRRAFIAEEGWLLVALDYSQIELRVLAHLSGDENLIRVFQEGRDIHTETASWMFGVPRE
AVDPLMRRAAKTINFGVLYGMSAHRLSQELAIPYEEAQAFIERYFQSFPKVRAWIEKTLE
EGRRRGYVETLFGRRRYVPDLEARVKSVREAAERMAFNMPVQGTAADLMKLAMVKLF
PRLEEMGARMLLQVHDELVLEAPKERAEAVARLAKEVMEGVYPLAVPLEVEVGIGED
WLSAKGD
SEQ ID NO: 47-TRX-Taq-TBD with 60 residue linker and T7 TIS truncation A at
insertion site B
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGGGGSG
GGSGSSGGSGSSGGSSGGGGSGGGSGSSGGSGSSGSGSSGSSGGGGSGGSSRGMLPLFEP
KGRVLLVDGHHLAYRTFHALKGLTTSRGEPVQAVYGFAKSLLKALKEDGDAVIVVFDA
AWGYRLGEMKGEYKDDFKRMLEEQGEEYVDGRAPTPEDFPRQLALIKELVDLLGLARL
EVPGYEADDVLASLAKKAEKEGYEVRILTADKDLYQLLSDRIHVLHPEGYLITPAWLWE
KYGLRPDQWADYRALTGDESDNLPGVKGIGEKTARKLLEEWGSLEALLKNLDRLKPAI
REKILAHMDDLKLSWDLAKVRTDLPLEVDFAKRREPDRERLRAFLERLEFGSLLHEFGL
LESPKALEEAPWPPPEGAFVGFVLSRKEPMWADLLALAAARGGRVHRAPEPYKALRDL
KEARGLLAKDLSVLALREGLGLPPGDDPMLLAYLLDPSNTTPEGVARRYGGEWTEEAG
ERAALSERLFANLWGRLEGEERLLWLYREVERPLSAVLAHMEATGVRLDVAYLRALSL
EVAEEIARLEAEVFRLAGGSWYQPKGGTEMFCHPRTGKPLPKYPRIKIPKVGGIFKKPKN
KAQREGREPCELDTREYVAGAPYTPVEHVVFNPSSRDQLERVLFDELGLPAIGKTEKTG
KRSTSAAVLEALREAHPIVEKILQYRELTKLKSTYIDPLPDLIHPRTGRLHTRFNQTATAT
GRLSSSDPNLQNIPVRTPLGQRIRRAFIAEEGWLLVALDYSQIELRVLAHLSGDENLIRVF
QEGRDIHTETASWMFGVPREAVDPLMRRAAKTINFGVLYGMSAHRLSQELAIPYEEAQA
FIERYFQSFPKVRAWIEKTLEEGRRRGYVETLFGRRRYVPDLEARVKSVREAAERMAFN
MPVQGTAADLMKLAMVKLFPRLEEMGARMLLQVHDELVLEAPKERAEAVARLAKEV
MEGVYPLAVPLEVEVGIGEDWLSAKGD
SEQ ID NO: 48-TRX-Taq-TBD with 60 residue linker and T7 TIS truncation B at
insertion site C
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGGGGSG
GGSGSSGGSGSSGGSSGGGGSGGGSGSSGGSGSSGSGSSGSSGGGGSGGSSRGMLPLFEP
KGRVLLVDGHHLAYRTFHALKGLTTSRGEPVQAVYGFAKSLLKALKEDGDA VIVVFDA
KAPSFRHEAYGGYKAGRAPTPEDFPRQLALIKELVDLLGLARLEVPGYEADDVLASLAK
KAEKEGYEVRILTADKDLYQLLSDRIHVLHPEGYLITPAWLWEKYGLRPDQWADYRAL
TGDESDNLPGVKGIGEKTARKLLEEWGSLEALLKNLDRLKPAIREKILAHMDDLKLSWD
LAKVRTDLPLEVDFAKRREPDRERLGEMKGEYKDDFKRMLEEQGEEYVDGLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTREYVAGAPYTPVEHVVFNPSSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAHRLSQELAIPYE
EAQAFIERYFQSFPKVRAWIEKTLEEGRRRGYVETLFGRRRYVPDLEARVKSVREAAER
MAFNMPVQGTAADLMKLAMVKLFPRLEEMGARMLLQVHDELVLEAPKERAEAVARL
AKEVMEGVYPLAVPLEVEVGIGEDWLSAKGD
SEQ ID NO: 49-TRX-Taq-TBD with 60 residue linker and T7 TIS truncation C at
insertion site A
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGGGGSG
GGSGSSGGSGSSGGSSGGGGSGGGSGSSGGSGSSGSGSSGSSGGGGSGGSSRGMLPLFEP
KGRVLLVDGHHLAYRTFHALKGEYKDDFKRMLEEQGEEYVDGDAVIVVFDAKAPSFR
HEAYGGYKAGRAPTPEDFPRQLALIKELVDLLGLARLEVPGYEADDVLASLAKKAEKEG
YEVRILTADKDLYQLLSDRIHVLHPEGYLITPAWLWEKYGLRPDQWADYRALTGDESD
NLPGVKGIGEKTARKLLEEWGSLEALLKNLDRLKPAIREKILAHMDDLKLSWDLAKVRT
DLPLEVDFAKRREPDRERLRAFLERLEFGSLLHEFGLLESPKALEEAPWPPPEGAFVGFVL
SRKEPMWADLLALAAARGGRVHRAPEPYKALRDLKEARGLLAKDLSVLALREGLGLPP
GDDPMLLAYLLDPSNTTPEGVARRYGGEWTEEAGERAALSERLFANLWGRLEGEERLL
WLYREVERPLSAVLAHMEATGVRLDVAYLRALSLEVAEEIARLEAEVFRLAGGSWYQP
KGGTEMFCHPRTGKPLPKYPRIKIPKVGGIFKKPKNKAQREGREPCELDTREYVAGAPYT
PVEHVVFNPSSRDQLERVLFDELGLPAIGKTEKTGKRSTSAAVLEALREAHPIVEKILQYR
ELTKLKSTYIDPLPDLIHPRTGRLHTRFNQTATATGRLSSSDPNLQNIPVRTPLGQRIRRAF
IAEEGWLLVALDYSQIELRVLAHLSGDENLIRVFQEGRDIHTETASWMFGVPREAVDPL
MRRAAKTINFGVLYGMSAHRLSQELAIPYEEAQAFIERYFQSFPKVRAWIEKTLEEGRRR
GYVETLFGRRRYVPDLEARVKSVREAAERMAFNMPVQGTAADLMKLAMVKLFPRLEE
MGARMLLQVHDELVLEAPKERAEAVARLAKEVMEGVYPLAVPLEVEVGIGEDWLSAK
GD
SEQ ID NO: 50-Taq-TBD, pATG6979
MRGMLPLFEPKGRVLLVDGHHLAYRTFHALKGLTTSRGEPVQAVYGFAKSLLKALKED
GDAVIVVFDAKAPSFRHEAYGGYKAGRAPTPEDFPRQLALIKELVDLLGLARLEVPGYE
ADDVLASLAKKAEKEGYEVRILTADKDLYQLLSDRIHVLHPEGYLITPAWLWEKYGLRP
DQWADYRALTGDESDNLPGVKGIGEKTARKLLEEWGSLEALLKNLDRLKPAIREKILAH
MDDLKLSWDLAKVRTDLPLEVDFAKRREPDRERLRAFLERLEFGSLLHEFGLLESPKAL
EEAPWPPPEGAFVGFVLSRKEPMWADLLALAAARGGRVHRAPEPYKALRDLKEARGLL
AKDLSVLALREGLGLPPGDDPMLLAYLLDPSNTTPEGVARRYGGEWTEEAGERAALSER
LFANLWGRLEGEERLLWLYREVERPLSAVLAHMEATGVRLDVAYLRALSLEVAEEIAR
LEAEVFRLAGGSWYQPKGGTEMFCHPRTGKPLPKYPRIKIPKVGGIFKKPKNKAQREGR
EPCELDTREYVAGAPYTPVEHVVFNPSSRDQLERVLFDELGLPAIGKTEKTGKRSTSAAV
LEALREAHPIVEKILQYRELTKLKSTYIDPLPDLIHPRTGRLHTRFNQTATATGRLSSSDPN
LONIPVRTPLGQRIRRAFIAEEGWLLVALDYSQIELRVLAHLSGDENLIRVFQEGRDIHTE
TASWMFGVPREAVDPLMRRAAKTINFGVLYGMSAHRLSQELAIPYEEAQAFIERYFQSF
PKVRAWIEKTLEEGRRRGYVETLFGRRRYVPDLEARVKSVREAAERMAFNMPVQGTAA
DLMKLAMVKLFPRLEEMGARMLLQVHDELVLEAPKERAEAVARLAKEVMEGVYPLAV
PLEVEVGIGEDWLSAKGD
SEQ ID NO: 51-AI generated TRX Sequence no. 1, pATG8280
MKKIKTVKTDEELEKILEESNKYTIVLFGAEWCGPCKMFLQENGETLEKISKEKGFEVLL
VNIDQNPGTAPKYGIRGIPTVVVFKNGKKIATKVGALSKGQLLEFADAV
SEQ ID NO: 52-AI generated TRX Sequence no. 2, pATG8279
MVKKVTEEELEKLIEEAKKKGEKIMVIFSAEWCGPCKMLLKELEEIEDELKALGIKEVLE
LNIDQNPGTAPKYGIRGIPTIMFIDANGIVYTKVGALSKGQILELAKAV
SEQ ID NO: 53-AI generated TRX Sequence no. 3, pATG8281
MIKEVNGEELDKIIKEESPKRKIIIDFGAEWCGPCKMLKAELEKIAKELEEKYGYDIYLLNI
DQNPGTAPKYGIRGIPTLIILTPSGKKLTKVGALSKGQILQLVKAA
SEQ ID NO: 54-TRX-90GS linker-Taq-TBD, pATG7580
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSGSSGGSGSRGMLPLFEPKGRVLLVDGHHLAYRTFHALKGLTTSRGEP
VQAVYGFAKSLLKALKEDGDAVIVVFDAKAPSFRHEAYGGYKAGRAPTPEDFPRQLALI
KELVDLLGLARLEVPGYEADDVLASLAKKAEKEGYEVRILTADKDLYQLLSDRIHVLHP
EGYLITPAWLWEKYGLRPDQWADYRALTGDESDNLPGVKGIGEKTARKLLEEWGSLEA
LLKNLDRLKPAIREKILAHMDDLKLSWDLAKVRTDLPLEVDFAKRREPDRERLRAFLER
LEFGSLLHEFGLLESPKALEEAPWPPPEGAFVGFVLSRKEPMWADLLALAAARGGRVHR
APEPYKALRDLKEARGLLAKDLSVLALREGLGLPPGDDPMLLAYLLDPSNTTPEGVARR
YGGEWTEEAGERAALSERLFANLWGRLEGEERLLWLYREVERPLSAVLAHMEATGVRL
DVAYLRALSLEVAEEIARLEAEVFRLAGGSWYQPKGGTEMFCHPRTGKPLPKYPRIKIPK
VGGIFKKPKNKAQREGREPCELDTREYVAGAPYTPVEHVVFNPSSRDQLERVLFDELGL
PAIGKTEKTGKRSTSAAVLEALREAHPIVEKILQYRELTKLKSTYIDPLPDLIHPRTGRLHT
RFNQTATATGRLSSSDPNLQNIPVRTPLGQRIRRAFIAEEGWLLVALDYSQIELRVLAHLS
GDENLIRVFQEGRDIHTETASWMFGVPREAVDPLMRRAAKTINFGVLYGMSAHRLSQEL
AIPYEEAQAFIERYFQSFPKVRAWIEKTLEEGRRRGYVETLFGRRRYVPDLEARVKSVRE
AAERMAFNMPVQGTAADLMKLAMVKLFPRLEEMGARMLLQVHDELVLEAPKERAEA
VARLAKEVMEGVYPLAVPLEVEVGIGEDWLSAKGD
SEQ ID NO: 55-TRX (E31P)-82GS linker-Taq-TBD H914F, pATG7646
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSGSGSS
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTREYVAGAPYTPVEHVVFNPSSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD
SEQ ID NO: 56-TRX-82GS linker-Taq-TBD H914F, pATG7620
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTREYVAGAPYTPVEHVVFNPSSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD
Seq. ID No: 57-TRX (E31P)-82GS linker-Taq-TBD E537V/S555N (H914F), pATG7860
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVERLAGGSWYQPKGGTEMFCHPRTGKPLPKYPRIKIPKVGGIFK
KPKNKAQREGREPCELDTRVYVAGAPYTPVEHVVFNPNSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD
Seq. ID No: 58-TRX-TaqTBD with no linker, pATG8388
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLARGMLPLFEPK
GRVLLVDGHHLAYRTFHALKGLTTSRGEPVQAVYGFAKSLLKALKEDGDAVIVVFDAK
APSFRHEAYGGYKAGRAPTPEDFPRQLALIKELVDLLGLARLEVPGYEADDVLASLAKK
AEKEGYEVRILTADKDLYQLLSDRIHVLHPEGYLITPAWLWEKYGLRPDQWADYRALT
GDESDNLPGVKGIGEKTARKLLEEWGSLEALLKNLDRLKPAIREKILAHMDDLKLSWDL
AKVRTDLPLEVDFAKRREPDRERLRAFLERLEFGSLLHEFGLLESPKALEEAPWPPPEGA
FVGFVLSRKEPMWADLLALAAARGGRVHRAPEPYKALRDLKEARGLLAKDLSVLALRE
GLGLPPGDDPMLLAYLLDPSNTTPEGVARRYGGEWTEEAGERAALSERLFANLWGRLE
GEERLLWLYREVERPLSAVLAHMEATGVRLDVAYLRALSLEVAEEIARLEAEVFRLAGG
SWYQPKGGTEMFCHPRTGKPLPKYPRIKIPKVGGIFKKPKNKAQREGREPCELDTREYV
AGAPYTPVEHVVFNPSSRDQLERVLFDELGLPAIGKTEKTGKRSTSAAVLEALREAHPIV
EKILQYRELTKLKSTYIDPLPDLIHPRTGRLHTRFNQTATATGRLSSSDPNLQNIPVRTPLG
QRIRRAFIAEEGWLLVALDYSQIELRVLAHLSGDENLIRVFQEGRDIHTETASWMFGVPR
EAVDPLMRRAAKTINFGVLYGMSAHRLSQELAIPYEEAQAFIERYFQSFPKVRAWIEKTL
EEGRRRGYVETLFGRRRYVPDLEARVKSVREAAERMAFNMPVQGTAADLMKLAMVKL
FPRLEEMGARMLLQVHDELVLEAPKERAEAVARLAKEVMEGVYPLAVPLEVEVGIGED
WLSAKGD
Seq. ID No: 59-TRX-5GS linker-TaqTBD, pATG8387
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLASGGSS
RGMLPLFEPKGRVLLVDGHHLAYRTFHALKGLTTSRGEPVQAVYGFAKSLLKALKEDG
DAVIVVFDAKAPSFRHEAYGGYKAGRAPTPEDFPRQLALIKELVDLLGLARLEVPGYEA
DDVLASLAKKAEKEGYEVRILTADKDLYQLLSDRIHVLHPEGYLITPAWLWEKYGLRPD
QWADYRALTGDESDNLPGVKGIGEKTARKLLEEWGSLEALLKNLDRLKPAIREKILAHM
DDLKLSWDLAKVRTDLPLEVDFAKRREPDRERLRAFLERLEFGSLLHEFGLLESPKALEE
APWPPPEGAFVGFVLSRKEPMWADLLALAAARGGRVHRAPEPYKALRDLKEARGLLA
KDLSVLALREGLGLPPGDDPMLLAYLLDPSNTTPEGVARRYGGEWTEEAGERAALSERL
FANLWGRLEGEERLLWLYREVERPLSAVLAHMEATGVRLDVAYLRALSLEVAEEIARL
EAEVFRLAGGSWYQPKGGTEMFCHPRTGKPLPKYPRIKIPKVGGIFKKPKNKAQREGRE
PCELDTREYVAGAPYTPVEHVVFNPSSRDQLERVLFDELGLPAIGKTEKTGKRSTSAAVL
EALREAHPIVEKILQYRELTKLKSTYIDPLPDLIHPRTGRLHTRFNQTATATGRLSSSDPNL
QNIPVRTPLGQRIRRAFIAEEGWLLVALDYSQIELRVLAHLSGDENLIRVFQEGRDIHTET
ASWMFGVPREAVDPLMRRAAKTINFGVLYGMSAHRLSQELAIPYEEAQAFIERYFQSFP
KVRAWIEKTLEEGRRRGYVETLFGRRRYVPDLEARVKSVREAAERMAFNMPVQGTAA
DLMKLAMVKLFPRLEEMGARMLLQVHDELVLEAPKERAEAVARLAKEVMEGVYPLAV
PLEVEVGIGEDWLSAKGD
Seq. ID No: 60-TRX-10GS linker-TaqTBD, pATG8386
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLA
SGGGGSGGSS
RGMLPLFEPKGRVLLVDGHHLAYRTFHALKGLTTSRGEPVQAVYGFAKSLLKALKEDG
DAVIVVFDAKAPSFRHEAYGGYKAGRAPTPEDFPRQLALIKELVDLLGLARLEVPGYEA
DDVLASLAKKAEKEGYEVRILTADKDLYQLLSDRIHVLHPEGYLITPAWLWEKYGLRPD
QWADYRALTGDESDNLPGVKGIGEKTARKLLEEWGSLEALLKNLDRLKPAIREKILAHM
DDLKLSWDLAKVRTDLPLEVDFAKRREPDRERLRAFLERLEFGSLLHEFGLLESPKALEE
APWPPPEGAFVGFVLSRKEPMWADLLALAAARGGRVHRAPEPYKALRDLKEARGLLA
KDLSVLALREGLGLPPGDDPMLLAYLLDPSNTTPEGVARRYGGEWTEEAGERAALSERL
FANLWGRLEGEERLLWLYREVERPLSAVLAHMEATGVRLDVAYLRALSLEVAEEIARL
EAEVFRLAGGSWYQPKGGTEMFCHPRTGKPLPKYPRIKIPKVGGIFKKPKNKAQREGRE
PCELDTREYVAGAPYTPVEHVVFNPSSRDQLERVLFDELGLPAIGKTEKTGKRSTSAAVL
EALREAHPIVEKILQYRELTKLKSTYIDPLPDLIHPRTGRLHTRFNQTATATGRLSSSDPNL
QNIPVRTPLGQRIRRAFIAEEGWLLVALDYSQIELRVLAHLSGDENLIRVFQEGRDIHTET
ASWMFGVPREAVDPLMRRAAKTINFGVLYGMSAHRLSQELAIPYEEAQAFIERYFQSFP
KVRAWIEKTLEEGRRRGYVETLFGRRRYVPDLEARVKSVREAAERMAFNMPVQGTAA
DLMKLAMVKLFPRLEEMGARMLLQVHDELVLEAPKERAEAVARLAKEVMEGVYPLAV
PLEVEVGIGEDWLSAKGD
Seq. ID No: 61-TRX-15GS linker-TaqTBD, pATG8385
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGSSGGGG
SGGSSRGMLPLFEPKGRVLLVDGHHLAYRTFHALKGLTTSRGEPVQAVYGFAKSLLKAL
KEDGDAVIVVFDAKAPSFRHEAYGGYKAGRAPTPEDFPRQLALIKELVDLLGLARLEVP
GYEADDVLASLAKKAEKEGYEVRILTADKDLYQLLSDRIHVLHPEGYLITPAWLWEKY
GLRPDQWADYRALTGDESDNLPGVKGIGEKTARKLLEEWGSLEALLKNLDRLKPAIREK
ILAHMDDLKLSWDLAKVRTDLPLEVDFAKRREPDRERLRAFLERLEFGSLLHEFGLLESP
KALEEAPWPPPEGAFVGFVLSRKEPMWADLLALAAARGGRVHRAPEPYKALRDLKEAR
GLLAKDLSVLALREGLGLPPGDDPMLLAYLLDPSNTTPEGVARRYGGEWTEEAGERAA
LSERLFANLWGRLEGEERLLWLYREVERPLSAVLAHMEATGVRLDVAYLRALSLEVAE
EIARLEAEVFRLAGGSWYQPKGGTEMFCHPRTGKPLPKYPRIKIPKVGGIFKKPKNKAQR
EGREPCELDTREYVAGAPYTPVEHVVFNPSSRDQLERVLFDELGLPAIGKTEKTGKRSTS
AAVLEALREAHPIVEKILQYRELTKLKSTYIDPLPDLIHPRTGRLHTRFNQTATATGRLSSS
DPNLQNIPVRTPLGQRIRRAFIAEEGWLLVALDYSQIELRVLAHLSGDENLIRVFQEGRDI
HTETASWMFGVPREAVDPLMRRAAKTINFGVLYGMSAHRLSQELAIPYEEAQAFIERYF
QSFPKVRAWIEKTLEEGRRRGYVETLFGRRRYVPDLEARVKSVREAAERMAFNMPVQG
TAADLMKLAMVKLFPRLEEMGARMLLQVHDELVLEAPKERAEAVARLAKEVMEGVYP
LAVPLEVEVGIGEDWLSAKGD
Seq. ID No: 62-TRX-20GS linker-TaqTBD, pATG8384
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLA
GSSGSGSSGSSGGGGSGGSS
RGMLPLFEPKGRVLLVDGHHLAYRTFHALKGLTTSRGEPVQAVYGFAKSLLKALKEDG
DAVIVVFDAKAPSFRHEAYGGYKAGRAPTPEDFPRQLALIKELVDLLGLARLEVPGYEA
DDVLASLAKKAEKEGYEVRILTADKDLYQLLSDRIHVLHPEGYLITPAWLWEKYGLRPD
QWADYRALTGDESDNLPGVKGIGEKTARKLLEEWGSLEALLKNLDRLKPAIREKILAHM
DDLKLSWDLAKVRTDLPLEVDFAKRREPDRERLRAFLERLEFGSLLHEFGLLESPKALEE
APWPPPEGAFVGFVLSRKEPMWADLLALAAARGGRVHRAPEPYKALRDLKEARGLLA
KDLSVLALREGLGLPPGDDPMLLAYLLDPSNTTPEGVARRYGGEWTEEAGERAALSERL
FANLWGRLEGEERLLWLYREVERPLSAVLAHMEATGVRLDVAYLRALSLEVAEEIARL
EAEVFRLAGGSWYQPKGGTEMFCHPRTGKPLPKYPRIKIPKVGGIFKKPKNKAQREGRE
PCELDTREYVAGAPYTPVEHVVFNPSSRDQLERVLFDELGLPAIGKTEKTGKRSTSAAVL
EALREAHPIVEKILQYRELTKLKSTYIDPLPDLIHPRTGRLHTRFNQTATATGRLSSSDPNL
QNIPVRTPLGQRIRRAFIAEEGWLLVALDYSQIELRVLAHLSGDENLIRVFQEGRDIHTET
ASWMFGVPREAVDPLMRRAAKTINFGVLYGMSAHRLSQELAIPYEEAQAFIERYFQSFP
KVRAWIEKTLEEGRRRGYVETLFGRRRYVPDLEARVKSVREAAERMAFNMPVQGTAA
DLMKLAMVKLFPRLEEMGARMLLQVHDELVLEAPKERAEAVARLAKEVMEGVYPLAV
PLEVEVGIGEDWLSAKGD
Seq. ID No: 63-TRX-25GS linker-TaqTBD, pATG8383
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLA
GSSGSGSSGSGSSGSSGGGGSGGSS
RGMLPLFEPKGRVLLVDGHHLAYRTFHALKGLTTSRGEPVQAVYGFAKSLLKALKEDG
DAVIVVFDAKAPSFRHEAYGGYKAGRAPTPEDFPRQLALIKELVDLLGLARLEVPGYEA
DDVLASLAKKAEKEGYEVRILTADKDLYQLLSDRIHVLHPEGYLITPAWLWEKYGLRPD
QWADYRALTGDESDNLPGVKGIGEKTARKLLEEWGSLEALLKNLDRLKPAIREKILAHM
DDLKLSWDLAKVRTDLPLEVDFAKRREPDRERLRAFLERLEFGSLLHEFGLLESPKALEE
APWPPPEGAFVGFVLSRKEPMWADLLALAAARGGRVHRAPEPYKALRDLKEARGLLA
KDLSVLALREGLGLPPGDDPMLLAYLLDPSNTTPEGVARRYGGEWTEEAGERAALSERL
FANLWGRLEGEERLLWLYREVERPLSAVLAHMEATGVRLDVAYLRALSLEVAEEIARL
EAEVFRLAGGSWYQPKGGTEMFCHPRTGKPLPKYPRIKIPKVGGIFKKPKNKAQREGRE
PCELDTREYVAGAPYTPVEHVVFNPSSRDQLERVLFDELGLPAIGKTEKTGKRSTSAAVL
EALREAHPIVEKILQYRELTKLKSTYIDPLPDLIHPRTGRLHTRFNQTATATGRLSSSDPNL
QNIPVRTPLGQRIRRAFIAEEGWLLVALDYSQIELRVLAHLSGDENLIRVFQEGRDIHTET
ASWMFGVPREAVDPLMRRAAKTINFGVLYGMSAHRLSQELAIPYEEAQAFIERYFQSFP
KVRAWIEKTLEEGRRRGYVETLFGRRRYVPDLEARVKSVREAAERMAFNMPVQGTAA
DLMKLAMVKLFPRLEEMGARMLLQVHDELVLEAPKERAEAVARLAKEVMEGVYPLAV
PLEVEVGIGEDWLSAKGD
Seq. ID No: 64-TRX-30GS linker-TaqTBD, pATG8382
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLA
GSSGSSGGSSGGGGSGSGSGSSGGSGSSGS
RGMLPLFEPKGRVLLVDGHHLAYRTFHALKGLTTSRGEPVQAVYGFAKSLLKALKEDG
DAVIVVFDAKAPSFRHEAYGGYKAGRAPTPEDFPRQLALIKELVDLLGLARLEVPGYEA
DDVLASLAKKAEKEGYEVRILTADKDLYQLLSDRIHVLHPEGYLITPAWLWEKYGLRPD
QWADYRALTGDESDNLPGVKGIGEKTARKLLEEWGSLEALLKNLDRLKPAIREKILAHM
DDLKLSWDLAKVRTDLPLEVDFAKRREPDRERLRAFLERLEFGSLLHEFGLLESPKALEE
APWPPPEGAFVGFVLSRKEPMWADLLALAAARGGRVHRAPEPYKALRDLKEARGLLA
KDLSVLALREGLGLPPGDDPMLLAYLLDPSNTTPEGVARRYGGEWTEEAGERAALSERL
FANLWGRLEGEERLLWLYREVERPLSAVLAHMEATGVRLDVAYLRALSLEVAEEIARL
EAEVFRLAGGSWYQPKGGTEMFCHPRTGKPLPKYPRIKIPKVGGIFKKPKNKAQREGRE
PCELDTREYVAGAPYTPVEHVVFNPSSRDQLERVLFDELGLPAIGKTEKTGKRSTSAAVL
EALREAHPIVEKILQYRELTKLKSTYIDPLPDLIHPRTGRLHTRFNQTATATGRLSSSDPNL
QNIPVRTPLGQRIRRAFIAEEGWLLVALDYSQIELRVLAHLSGDENLIRVFQEGRDIHTET
ASWMFGVPREAVDPLMRRAAKTINFGVLYGMSAHRLSQELAIPYEEAQAFIERYFQSFP
KVRAWIEKTLEEGRRRGYVETLFGRRRYVPDLEARVKSVREAAERMAFNMPVQGTAA
DLMKLAMVKLFPRLEEMGARMLLQVHDELVLEAPKERAEAVARLAKEVMEGVYPLAV
PLEVEVGIGEDWLSAKGD
SEQ. ID No: 65-TRX-35GS linker-TaqTBD, pATG8381
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLA
GSSGSSGGSSGGGGSGSGSGSSGGSGSSGSSGGSS
RGMLPLFEPKGRVLLVDGHHLAYRTFHALKGLTTSRGEPVQAVYGFAKSLLKALKEDG
DAVIVVFDAKAPSFRHEAYGGYKAGRAPTPEDFPRQLALIKELVDLLGLARLEVPGYEA
DDVLASLAKKAEKEGYEVRILTADKDLYQLLSDRIHVLHPEGYLITPAWLWEKYGLRPD
QWADYRALTGDESDNLPGVKGIGEKTARKLLEEWGSLEALLKNLDRLKPAIREKILAHM
DDLKLSWDLAKVRTDLPLEVDFAKRREPDRERLRAFLERLEFGSLLHEFGLLESPKALEE
APWPPPEGAFVGFVLSRKEPMWADLLALAAARGGRVHRAPEPYKALRDLKEARGLLA
KDLSVLALREGLGLPPGDDPMLLAYLLDPSNTTPEGVARRYGGEWTEEAGERAALSERL
FANLWGRLEGEERLLWLYREVERPLSAVLAHMEATGVRLDVAYLRALSLEVAEEIARL
EAEVFRLAGGSWYQPKGGTEMFCHPRTGKPLPKYPRIKIPKVGGIFKKPKNKAQREGRE
PCELDTREYVAGAPYTPVEHVVFNPSSRDQLERVLFDELGLPAIGKTEKTGKRSTSAAVL
EALREAHPIVEKILQYRELTKLKSTYIDPLPDLIHPRTGRLHTRFNQTATATGRLSSSDPNL
QNIPVRTPLGQRIRRAFIAEEGWLLVALDYSQIELRVLAHLSGDENLIRVFQEGRDIHTET
ASWMFGVPREAVDPLMRRAAKTINFGVLYGMSAHRLSQELAIPYEEAQAFIERYFQSFP
KVRAWIEKTLEEGRRRGYVETLFGRRRYVPDLEARVKSVREAAERMAFNMPVQGTAA
DLMKLAMVKLFPRLEEMGARMLLQVHDELVLEAPKERAEAVARLAKEVMEGVYPLAV
PLEVEVGIGEDWLSAKGD
Seq. ID No: 66-TRX-40GS linker-TaqTBD, pATG8380
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLA
GSSGSSGGSSGGGGSGSGSGSSGGSGSSGSGSSGSSGGSS
RGMLPLFEPKGRVLLVDGHHLAYRTFHALKGLTTSRGEPVQAVYGFAKSLLKALKEDG
DAVIVVFDAKAPSFRHEAYGGYKAGRAPTPEDFPRQLALIKELVDLLGLARLEVPGYEA
DDVLASLAKKAEKEGYEVRILTADKDLYQLLSDRIHVLHPEGYLITPAWLWEKYGLRPD
QWADYRALTGDESDNLPGVKGIGEKTARKLLEEWGSLEALLKNLDRLKPAIREKILAHM
DDLKLSWDLAKVRTDLPLEVDFAKRREPDRERLRAFLERLEFGSLLHEFGLLESPKALEE
APWPPPEGAFVGFVLSRKEPMWADLLALAAARGGRVHRAPEPYKALRDLKEARGLLA
KDLSVLALREGLGLPPGDDPMLLAYLLDPSNTTPEGVARRYGGEWTEEAGERAALSERL
FANLWGRLEGEERLLWLYREVERPLSAVLAHMEATGVRLDVAYLRALSLEVAEEIARL
EAEVFRLAGGSWYQPKGGTEMFCHPRTGKPLPKYPRIKIPKVGGIFKKPKNKAQREGRE
PCELDTREYVAGAPYTPVEHVVFNPSSRDQLERVLFDELGLPAIGKTEKTGKRSTSAAVL
EALREAHPIVEKILQYRELTKLKSTYIDPLPDLIHPRTGRLHTRFNQTATATGRLSSSDPNL
QNIPVRTPLGQRIRRAFIAEEGWLLVALDYSQIELRVLAHLSGDENLIRVFQEGRDIHTET
ASWMFGVPREAVDPLMRRAAKTINFGVLYGMSAHRLSQELAIPYEEAQAFIERYFQSFP
KVRAWIEKTLEEGRRRGYVETLFGRRRYVPDLEARVKSVREAAERMAFNMPVQGTAA
DLMKLAMVKLFPRLEEMGARMLLQVHDELVLEAPKERAEAVARLAKEVMEGVYPLAV
PLEVEVGIGEDWLSAKGD
Seq. ID No: 67-TRX-73GS linker-TaqTBD, pATG7584
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLA
GSSGSSGSSGGGSGGGSGSSGGSGSSGGSSGGGGSGSGSGSSGGSGSSGSGSSGSSGGGG
SGGSSGSSGGSGS
RGMLPLFEPKGRVLLVDGHHLAYRTFHALKGLTTSRGEPVQAVYGFAKSLLKALKEDG
DAVIVVFDAKAPSFRHEAYGGYKAGRAPTPEDFPRQLALIKELVDLLGLARLEVPGYEA
DDVLASLAKKAEKEGYEVRILTADKDLYQLLSDRIHVLHPEGYLITPAWLWEKYGLRPD
QWADYRALTGDESDNLPGVKGIGEKTARKLLEEWGSLEALLKNLDRLKPAIREKILAHM
DDLKLSWDLAKVRTDLPLEVDFAKRREPDRERLRAFLERLEFGSLLHEFGLLESPKALEE
APWPPPEGAFVGFVLSRKEPMWADLLALAAARGGRVHRAPEPYKALRDLKEARGLLA
KDLSVLALREGLGLPPGDDPMLLAYLLDPSNTTPEGVARRYGGEWTEEAGERAALSERL
FANLWGRLEGEERLLWLYREVERPLSAVLAHMEATGVRLDVAYLRALSLEVAEEIARL
EAEVFRLAGGSWYQPKGGTEMFCHPRTGKPLPKYPRIKIPKVGGIFKKPKNKAQREGRE
PCELDTREYVAGAPYTPVEHVVFNPSSRDQLERVLFDELGLPAIGKTEKTGKRSTSAAVL
EALREAHPIVEKILQYRELTKLKSTYIDPLPDLIHPRTGRLHTRFNQTATATGRLSSSDPNL
QNIPVRTPLGQRIRRAFIAEEGWLLVALDYSQIELRVLAHLSGDENLIRVFQEGRDIHTET
ASWMFGVPREAVDPLMRRAAKTINFGVLYGMSAHRLSQELAIPYEEAQAFIERYFQSFP
KVRAWIEKTLEEGRRRGYVETLFGRRRYVPDLEARVKSVREAAERMAFNMPVQGTAA
DLMKLAMVKLFPRLEEMGARMLLQVHDELVLEAPKERAEAVARLAKEVMEGVYPLAV
PLEVEVGIGEDWLSAKGD
Seq. ID No: 68-TRX-100GS linker-TaqTBD, pATG7585
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLA
GSSGSSGSSGGGSGGGSGSSGSSGSSGGGSGGGSGSSGGSGSSGGSSGGGGSGSGSGSSG
GSGSSGSGSSGSSGGGGSGGSSGSSSGGSSSGGSSGGSGSRGMLPLFEPKGRVLLVDGHH
LAYRTFHALKGLTTSRGEPVQAVYGFAKSLLKALKEDGDAVIVVFDAKAPSFRHEAYG
GYKAGRAPTPEDFPRQLALIKELVDLLGLARLEVPGYEADDVLASLAKKAEKEGYEVRI
LTADKDLYQLLSDRIHVLHPEGYLITPAWLWEKYGLRPDQWADYRALTGDESDNLPGV
KGIGEKTARKLLEEWGSLEALLKNLDRLKPAIREKILAHMDDLKLSWDLAKVRTDLPLE
VDFAKRREPDRERLRAFLERLEFGSLLHEFGLLESPKALEEAPWPPPEGAFVGFVLSRKEP
MWADLLALAAARGGRVHRAPEPYKALRDLKEARGLLAKDLSVLALREGLGLPPGDDP
MLLAYLLDPSNTTPEGVARRYGGEWTEEAGERAALSERLFANLWGRLEGEERLLWLYR
EVERPLSAVLAHMEATGVRLDVAYLRALSLEVAEEIARLEAEVFRLAGGSWYQPKGGT
EMFCHPRTGKPLPKYPRIKIPKVGGIFKKPKNKAQREGREPCELDTREYVAGAPYTPVEH
VVFNPSSRDQLERVLFDELGLPAIGKTEKTGKRSTSAAVLEALREAHPIVEKILQYRELTK
LKSTYIDPLPDLIHPRTGRLHTRFNQTATATGRLSSSDPNLQNIPVRTPLGQRIRRAFIAEE
GWLLVALDYSQIELRVLAHLSGDENLIRVFQEGRDIHTETASWMFGVPREAVDPLMRRA
AKTINFGVLYGMSAHRLSQELAIPYEEAQAFIERYFQSFPKVRAWIEKTLEEGRRRGYVE
TLFGRRRYVPDLEARVKSVREAAERMAFNMPVQGTAADLMKLAMVKLFPRLEEMGAR
MLLQVHDELVLEAPKERAEAVARLAKEVMEGVYPLAVPLEVEVGIGEDWLSAKGD
Seq. ID No: 69-TRX-110GS linker-TaqTBD, pATG8345
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLA
GSSGSSGSSGGGSGGGSGSSGSSGSSGGGSGGGGSSGSGGSGGGSGSSGSSGSSGGGSGG
GSGSSGGSGSSGGSSGGGGSGSGSGSSGGSGSSGSGSSGSSGGGGSGGSS
RGMLPLFEPKGRVLLVDGHHLAYRTFHALKGLTTSRGEPVQAVYGFAKSLLKALKEDG
DAVIVVFDAKAPSFRHEAYGGYKAGRAPTPEDFPRQLALIKELVDLLGLARLEVPGYEA
DDVLASLAKKAEKEGYEVRILTADKDLYQLLSDRIHVLHPEGYLITPAWLWEKYGLRPD
QWADYRALTGDESDNLPGVKGIGEKTARKLLEEWGSLEALLKNLDRLKPAIREKILAHM
DDLKLSWDLAKVRTDLPLEVDFAKRREPDRERLRAFLERLEFGSLLHEFGLLESPKALEE
APWPPPEGAFVGFVLSRKEPMWADLLALAAARGGRVHRAPEPYKALRDLKEARGLLA
KDLSVLALREGLGLPPGDDPMLLAYLLDPSNTTPEGVARRYGGEWTEEAGERAALSERL
FANLWGRLEGEERLLWLYREVERPLSAVLAHMEATGVRLDVAYLRALSLEVAEEIARL
EAEVFRLAGGSWYQPKGGTEMFCHPRTGKPLPKYPRIKIPKVGGIFKKPKNKAQREGRE
PCELDTREYVAGAPYTPVEHVVFNPSSRDQLERVLFDELGLPAIGKTEKTGKRSTSAAVL
EALREAHPIVEKILQYRELTKLKSTYIDPLPDLIHPRTGRLHTRFNQTATATGRLSSSDPNL
QNIPVRTPLGQRIRRAFIAEEGWLLVALDYSQIELRVLAHLSGDENLIRVFQEGRDIHTET
ASWMFGVPREAVDPLMRRAAKTINFGVLYGMSAHRLSQELAIPYEEAQAFIERYFQSFP
KVRAWIEKTLEEGRRRGYVETLFGRRRYVPDLEARVKSVREAAERMAFNMPVQGTAA
DLMKLAMVKLFPRLEEMGARMLLQVHDELVLEAPKERAEAVARLAKEVMEGVYPLAV
PLEVEVGIGEDWLSAKGD
Seq. ID No: 70-TRX-130GS linker-TaqTBD, pATG8351
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLA
GSSGSSGSSGGGSGGGSGSSGSSGSSGGGSGGGSGSSGGSGGSSSSGGSSGGGGSSGSGG
SGGGSGSSGSSGSSGGGSGGGSGSSGGSGSSGGSSGGGGSGSGSGSSGGSGSSGSGSSGSS
GGGGSGGSSRGMLPLFEPKGRVLLVDGHHLAYRTFHALKGLTTSRGEPVQAVYGFAKS
LLKALKEDGDAVIVVFDAKAPSFRHEAYGGYKAGRAPTPEDFPRQLALIKELVDLLGLA
RLEVPGYEADDVLASLAKKAEKEGYEVRILTADKDLYQLLSDRIHVLHPEGYLITPAWL
WEKYGLRPDQWADYRALTGDESDNLPGVKGIGEKTARKLLEEWGSLEALLKNLDRLKP
AIREKILAHMDDLKLSWDLAKVRTDLPLEVDFAKRREPDRERLRAFLERLEFGSLLHEFG
LLESPKALEEAPWPPPEGAFVGFVLSRKEPMWADLLALAAARGGRVHRAPEPYKALRD
LKEARGLLAKDLSVLALREGLGLPPGDDPMLLAYLLDPSNTTPEGVARRYGGEWTEEA
GERAALSERLFANLWGRLEGEERLLWLYREVERPLSAVLAHMEATGVRLDVAYLRALS
LEVAEEIARLEAEVFRLAGGSWYQPKGGTEMFCHPRTGKPLPKYPRIKIPKVGGIFKKPK
NKAQREGREPCELDTREYVAGAPYTPVEHVVFNPSSRDQLERVLFDELGLPAIGKTEKT
GKRSTSAAVLEALREAHPIVEKILQYRELTKLKSTYIDPLPDLIHPRTGRLHTRFNQTATA
TGRLSSSDPNLQNIPVRTPLGQRIRRAFIAEEGWLLVALDYSQIELRVLAHLSGDENLIRV
FQEGRDIHTETASWMFGVPREAVDPLMRRAAKTINFGVLYGMSAHRLSQELAIPYEEAQ
AFIERYFQSFPKVRAWIEKTLEEGRRRGYVETLFGRRRYVPDLEARVKSVREAAERMAF
NMPVQGTAADLMKLAMVKLFPRLEEMGARMLLQVHDELVLEAPKERAEAVARLAKE
VMEGVYPLAVPLEVEVGIGEDWLSAKGD
Seq. ID No: 71-TRX-150GS linker-TaqTBD, pATG8346
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLA
GSSGSSGSSGGGSGGGSGSSGSSGSSGGGSGGGSGSSGGSGGSSSSGGSSGGGGSGSGSG
SSGGSGSSGSGSSGSSGSGGSGGGSGSSGSSGSSGGGSGGGSGSSGGSGSSGGSSGGGGS
GSGSGSSGGSGSSGSGSSGSSGGGGSGGSSRGMLPLFEPKGRVLLVDGHHLAYRTFHAL
KGLTTSRGEPVQAVYGFAKSLLKALKEDGDAVIVVFDAKAPSFRHEAYGGYKAGRAPT
PEDFPRQLALIKELVDLLGLARLEVPGYEADDVLASLAKKAEKEGYEVRILTADKDLYQ
LLSDRIHVLHPEGYLITPAWLWEKYGLRPDQWADYRALTGDESDNLPGVKGIGEKTAR
KLLEEWGSLEALLKNLDRLKPAIREKILAHMDDLKLSWDLAKVRTDLPLEVDFAKRREP
DRERLRAFLERLEFGSLLHEFGLLESPKALEEAPWPPPEGAFVGFVLSRKEPMWADLLAL
AAARGGRVHRAPEPYKALRDLKEARGLLAKDLSVLALREGLGLPPGDDPMLLAYLLDP
SNTTPEGVARRYGGEWTEEAGERAALSERLFANLWGRLEGEERLLWLYREVERPLSAV
LAHMEATGVRLDVAYLRALSLEVAEEIARLEAEVFRLAGGSWYQPKGGTEMFCHPRTG
KPLPKYPRIKIPKVGGIFKKPKNKAQREGREPCELDTREYVAGAPYTPVEHVVFNPSSRD
QLERVLFDELGLPAIGKTEKTGKRSTSAAVLEALREAHPIVEKILQYRELTKLKSTYIDPL
PDLIHPRTGRLHTRFNQTATATGRLSSSDPNLQNIPVRTPLGQRIRRAFIAEEGWLLVALD
YSQIELRVLAHLSGDENLIRVFQEGRDIHTETASWMFGVPREAVDPLMRRAAKTINFGVL
YGMSAHRLSQELAIPYEEAQAFIERYFQSFPKVRAWIEKTLEEGRRRGYVETLFGRRRYV
PDLEARVKSVREAAERMAFNMPVQGTAADLMKLAMVKLFPRLEEMGARMLLQVHDE
LVLEAPKERAEAVARLAKEVMEGVYPLAVPLEVEVGIGEDWLSAKGD
Seq. ID No: 72-TRX-170GS linker-TaqTBD, pATG8347
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLA
GSSGSSGSSGGGSGGGSGSSGSSGSSGGGSGGGSGSSGGSGGSSSSGGSSGGGGSGSGSG
SSGGSGSSGSGSSGSSGGGGSGGSSGSSGGSGSGSSGSGGSGGGSGSSGSSGSSGGGSGG
GSGSSGGSGSSGGSSGGGGSGSGSGSSGGSGSSGSGSSGSSGGGGSGGSSRGMLPLFEPK
GRVLLVDGHHLAYRTFHALKGLTTSRGEPVQAVYGFAKSLLKALKEDGDAVIVVFDAK
APSFRHEAYGGYKAGRAPTPEDFPRQLALIKELVDLLGLARLEVPGYEADDVLASLAKK
AEKEGYEVRILTADKDLYQLLSDRIHVLHPEGYLITPAWLWEKYGLRPDQWADYRALT
GDESDNLPGVKGIGEKTARKLLEEWGSLEALLKNLDRLKPAIREKILAHMDDLKLSWDL
AKVRTDLPLEVDFAKRREPDRERLRAFLERLEFGSLLHEFGLLESPKALEEAPWPPPEGA
FVGFVLSRKEPMWADLLALAAARGGRVHRAPEPYKALRDLKEARGLLAKDLSVLALRE
GLGLPPGDDPMLLAYLLDPSNTTPEGVARRYGGEWTEEAGERAALSERLFANLWGRLE
GEERLLWLYREVERPLSAVLAHMEATGVRLDVAYLRALSLEVAEEIARLEAEVFRLAGG
SWYQPKGGTEMFCHPRTGKPLPKYPRIKIPKVGGIFKKPKNKAQREGREPCELDTREYV
AGAPYTPVEHVVFNPSSRDQLERVLFDELGLPAIGKTEKTGKRSTSAAVLEALREAHPIV
EKILQYRELTKLKSTYIDPLPDLIHPRTGRLHTRFNQTATATGRLSSSDPNLQNIPVRTPLG
QRIRRAFIAEEGWLLVALDYSQIELRVLAHLSGDENLIRVFQEGRDIHTETASWMFGVPR
EAVDPLMRRAAKTINFGVLYGMSAHRLSQELAIPYEEAQAFIERYFQSFPKVRAWIEKTL
EEGRRRGYVETLFGRRRYVPDLEARVKSVREAAERMAFNMPVQGTAADLMKLAMVKL
FPRLEEMGARMLLQVHDELVLEAPKERAEAVARLAKEVMEGVYPLAVPLEVEVGIGED
WLSAKGD
Seq. ID No: 73-TRX-200GS linker-TaqTBD, pATG8348
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLA
GSSGSSGSSGGGSGGGSGSSGSSGSSGGGSGGGSGSSGGSGGSSSSGGSSGGGGSGSGSG
SSGGSGSSGSGSSGSSGGGGSGGSSGSSGGSGSSSGSSGSSGGGSGGGSGSSGGSGSSGGS
SGGSSGSGGSGGGSGSSGSSGSSGGGSGGGSGSSGGSGSSGGSSGGGGSGSGSGSSGGSG
SSGSGSSGSSGGGGSGGSSRGMLPLFEPKGRVLLVDGHHLAYRTFHALKGLTTSRGEPV
QAVYGFAKSLLKALKEDGDAVIVVFDAKAPSFRHEAYGGYKAGRAPTPEDFPRQLALIK
ELVDLLGLARLEVPGYEADDVLASLAKKAEKEGYEVRILTADKDLYQLLSDRIHVLHPE
GYLITPAWLWEKYGLRPDQWADYRALTGDESDNLPGVKGIGEKTARKLLEEWGSLEAL
LKNLDRLKPAIREKILAHMDDLKLSWDLAKVRTDLPLEVDFAKRREPDRERLRAFLERL
EFGSLLHEFGLLESPKALEEAPWPPPEGAFVGFVLSRKEPMWADLLALAAARGGRVHRA
PEPYKALRDLKEARGLLAKDLSVLALREGLGLPPGDDPMLLAYLLDPSNTTPEGVARRY
GGEWTEEAGERAALSERLFANLWGRLEGEERLLWLYREVERPLSAVLAHMEATGVRLD
VAYLRALSLEVAEEIARLEAEVFRLAGGSWYQPKGGTEMFCHPRTGKPLPKYPRIKIPKV
GGIFKKPKNKAQREGREPCELDTREYVAGAPYTPVEHVVFNPSSRDQLERVLFDELGLP
AIGKTEKTGKRSTSAAVLEALREAHPIVEKILQYRELTKLKSTYIDPLPDLIHPRTGRLHTR
FNQTATATGRLSSSDPNLQNIPVRTPLGQRIRRAFIAEEGWLLVALDYSQIELRVLAHLSG
DENLIRVFQEGRDIHTETASWMFGVPREAVDPLMRRAAKTINFGVLYGMSAHRLSQELA
IPYEEAQAFIERYFQSFPKVRAWIEKTLEEGRRRGYVETLFGRRRYVPDLEARVKSVREA
AERMAFNMPVQGTAADLMKLAMVKLFPRLEEMGARMLLQVHDELVLEAPKERAEAV
ARLAKEVMEGVYPLAVPLEVEVGIGEDWLSAKGD
Seq. ID No: 74-TRX E31P-18GS(EAAAK46)18GS linker-TaqTBD [537V 555N] (H914F),
pATG8264
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGAEAAAKEAAAKEAAAKEAAAKALEAEAAAKEAAAKEAAAKEAAAKASG
SGSSGSSGGGGSGGSSRGMLPLFEPKGRVLLVDGHHLAYRTFHALKGLTTSRGEPVQAV
YGFAKSLLKALKEDGDAVIVVFDAKAPSFRHEAYGGYKAGRAPTPEDFPRQLALIKELV
DLLGLARLEVPGYEADDVLASLAKKAEKEGYEVRILTADKDLYQLLSDRIHVLHPEGYL
ITPAWLWEKYGLRPDQWADYRALTGDESDNLPGVKGIGEKTARKLLEEWGSLEALLKN
LDRLKPAIREKILAHMDDLKLSWDLAKVRTDLPLEVDFAKRREPDRERLRAFLERLEFGS
LLHEFGLLESPKALEEAPWPPPEGAFVGFVLSRKEPMWADLLALAAARGGRVHRAPEPY
KALRDLKEARGLLAKDLSVLALREGLGLPPGDDPMLLAYLLDPSNTTPEGVARRYGGE
WTEEAGERAALSERLFANLWGRLEGEERLLWLYREVERPLSAVLAHMEATGVRLDVA
YLRALSLEVAEEIARLEAEVFRLAGGSWYQPKGGTEMFCHPRTGKPLPKYPRIKIPKVGG
IFKKPKNKAQREGREPCELDTRVYVAGAPYTPVEHVVFNPNSRDQLERVLFDELGLPAIG
KTEKTGKRSTSAAVLEALREAHPIVEKILQYRELTKLKSTYIDPLPDLIHPRTGRLHTRFN
QTATATGRLSSSDPNLQNIPVRTPLGQRIRRAFIAEEGWLLVALDYSQIELRVLAHLSGDE
NLIRVFQEGRDIHTETASWMFGVPREAVDPLMRRAAKTINFGVLYGMSAFRLSQELAIP
YEEAQAFIERYFQSFPKVRAWIEKTLEEGRRRGYVETLFGRRRYVPDLEARVKSVREAA
ERMAFNMPVQGTAADLMKLAMVKLFPRLEEMGARMLLQVHDELVLEAPKERAEAVA
RLAKEVMEGVYPLAVPLEVEVGIGEDWLSAKGD
Seq. ID No: 75-TRX E31P-18GS(EAAAK repeat)18GS linker-TaqTBD [537V 555N]
(H914A), pATG8292
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGAEAAAKEAAAKEAAAKEAAAKALEAEAAAKEAAAKEAAAKEAAAKASG
SGSSGSSGGGGSGGSSRGMLPLFEPKGRVLLVDGHHLAYRTFHALKGLTTSRGEPVQAV
YGFAKSLLKALKEDGDAVIVVFDAKAPSFRHEAYGGYKAGRAPTPEDFPRQLALIKELV
DLLGLARLEVPGYEADDVLASLAKKAEKEGYEVRILTADKDLYQLLSDRIHVLHPEGYL
ITPAWLWEKYGLRPDQWADYRALTGDESDNLPGVKGIGEKTARKLLEEWGSLEALLKN
LDRLKPAIREKILAHMDDLKLSWDLAKVRTDLPLEVDFAKRREPDRERLRAFLERLEFGS
LLHEFGLLESPKALEEAPWPPPEGAFVGFVLSRKEPMWADLLALAAARGGRVHRAPEPY
KALRDLKEARGLLAKDLSVLALREGLGLPPGDDPMLLAYLLDPSNTTPEGVARRYGGE
WTEEAGERAALSERLFANLWGRLEGEERLLWLYREVERPLSAVLAHMEATGVRLDVA
YLRALSLEVAEEIARLEAEVFRLAGGSWYQPKGGTEMFCHPRTGKPLPKYPRIKIPKVGG
IFKKPKNKAQREGREPCELDTRVYVAGAPYTPVEHVVFNPNSRDQLERVLFDELGLPAIG
KTEKTGKRSTSAAVLEALREAHPIVEKILQYRELTKLKSTYIDPLPDLIHPRTGRLHTRFN
QTATATGRLSSSDPNLQNIPVRTPLGQRIRRAFIAEEGWLLVALDYSQIELRVLAHLSGDE
NLIRVFQEGRDIHTETASWMFGVPREAVDPLMRRAAKTINFGVLYGMSAARLSQELAIP
YEEAQAFIERYFQSFPKVRAWIEKTLEEGRRRGYVETLFGRRRYVPDLEARVKSVREAA
ERMAFNMPVQGTAADLMKLAMVKLFPRLEEMGARMLLQVHDELVLEAPKERAEAVA
RLAKEVMEGVYPLAVPLEVEVGIGEDWLSAKGD
Seq. ID No: 76-TRX E31P-HaloTag linker(GS50) HaloTag linker-TaqTBD [537V 555N]
(H914F), pATG8297
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLASLEPTTEDLY
FQSDNDSGSSGSSGSSGGGSGGGSGSSGGSGSSGGSSGGGGSGSGSGSSGGSGSSGSLEPT
TEDLYFQSDNDRGMLPLFEPKGRVLLVDGHHLAYRTFHALKGLTTSRGEPVQAVYGFA
KSLLKALKEDGDAVIVVFDAKAPSFRHEAYGGYKAGRAPTPEDFPRQLALIKELVDLLG
LARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTRVYVAGAPYTPVEHVVFNPNSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD
Seq. ID No: 77-TRX E31P-GSAT linker(82)-TaqTBD [537V 555N] (H914F), pATG8285
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSTAAGAAT
AAGSTGAAGAATAAGGSAGGTGSGSATGSSGASGTGTAGGTGAGSGTGSGAAGAATA
AGTSGAAGAATAATSGRGMLPLFEPKGRVLLVDGHHLAYRTFHALKGLTTSRGEPVQA
VYGFAKSLLKALKEDGDAVIVVFDAKAPSFRHEAYGGYKAGRAPTPEDFPRQLALIKEL
VDLLGLARLEVPGYEADDVLASLAKKAEKEGYEVRILTADKDLYQLLSDRIHVLHPEGY
LITPAWLWEKYGLRPDQWADYRALTGDESDNLPGVKGIGEKTARKLLEEWGSLEALLK
NLDRLKPAIREKILAHMDDLKLSWDLAKVRTDLPLEVDFAKRREPDRERLRAFLERLEF
GSLLHEFGLLESPKALEEAPWPPPEGAFVGFVLSRKEPMWADLLALAAARGGRVHRAPE
PYKALRDLKEARGLLAKDLSVLALREGLGLPPGDDPMLLAYLLDPSNTTPEGVARRYG
GEWTEEAGERAALSERLFANLWGRLEGEERLLWLYREVERPLSAVLAHMEATGVRLDV
AYLRALSLEVAEEIARLEAEVFRLAGGSWYQPKGGTEMFCHPRTGKPLPKYPRIKIPKVG
GIFKKPKNKAQREGREPCELDTRVYVAGAPYTPVEHVVFNPNSRDQLERVLFDELGLPAI
GKTEKTGKRSTSAAVLEALREAHPIVEKILQYRELTKLKSTYIDPLPDLIHPRTGRLHTRF
NOTATATGRLSSSDPNLQNIPVRTPLGQRIRRAFIAEEGWLLVALDYSQIELRVLAHLSGD
ENLIRVFQEGRDIHTETASWMFGVPREAVDPLMRRAAKTINFGVLYGMSAFRLSQELAIP
YEEAQAFIERYFQSFPKVRAWIEKTLEEGRRRGYVETLFGRRRYVPDLEARVKSVREAA
ERMAFNMPVQGTAADLMKLAMVKLFPRLEEMGARMLLQVHDELVLEAPKERAEAVA
RLAKEVMEGVYPLAVPLEVEVGIGEDWLSAKGD
Seq. ID No: 78-TRX (E31P)-24GS linker (Novelty sequence 1) 24GS linker-TaqSTBD
[537V 555N] H914F, pATG8298
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSGGCASSIDYKRISRMPSKIMDAVIDTLNICKLANCESGGSGSSGSGSSG
SSGGGGSGGSSRGMLPLFEPKGRVLLVDGHHLAYRTFHALKGLTTSRGEPVQAVYGFA
KSLLKALKEDGDAVIVVFDAKAPSFRHEAYGGYKAGRAPTPEDFPRQLALIKELVDLLG
LARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTRVYVAGAPYTPVEHVVFNPNSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD
Seq. ID No: 79-TRX (E31P)-24GS linker (Novelty sequence 2) 24GS linker-TaqSTBD
[537V 555N] H914F, pATG 8286
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSGGCASSIDYKRISRMPAVLADAVIDTLNICKLANCESGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITP
AWLWEKYGLRPDQWADYRALTGDESDNLPGVKGIGEKTARKLLEEWGSLEALLKNLD
RLKPAIREKILAHMDDLKLSWDLAKVRTDLPLEVDFAKRREPDRERLRAFLERLEFGSLL
HEFGLLESPKALEEAPWPPPEGAFVGFVLSRKEPMWADLLALAAARGGRVHRAPEPYK
ALRDLKEARGLLAKDLSVLALREGLGLPPGDDPMLLAYLLDPSNTTPEGVARRYGGEW
TEEAGERAALSERLFANLWGRLEGEERLLWLYREVERPLSAVLAHMEATGVRLDVAYL
RALSLEVAEEIARLEAEVFRLAGGSWYQPKGGTEMFCHPRTGKPLPKYPRIKIPKVGGIF
KKPKNKAQREGREPCELDTRVYVAGAPYTPVEHVVFNPNSRDQLERVLFDELGLPAIGK
TEKTGKRSTSAAVLEALREAHPIVEKILQYRELTKLKSTYIDPLPDLIHPRTGRLHTRFNQ
TATATGRLSSSDPNLQNIPVRTPLGQRIRRAFIAEEGWLLVALDYSQIELRVLAHLSGDEN
LIRVFQEGRDIHTETASWMFGVPREAVDPLMRRAAKTINFGVLYGMSAFRLSQELAIPYE
EAQAFIERYFQSFPKVRAWIEKTLEEGRRRGYVETLFGRRRYVPDLEARVKSVREAAER
MAFNMPVQGTAADLMKLAMVKLFPRLEEMGARMLLQVHDELVLEAPKERAEAVARL
AKEVMEGVYPLAVPLEVEVGIGEDWLSAKGD
Seq. ID No: 80-TaqTBD-55GS linker-TRX, pATG7589
MRGMLPLFEPKGRVLLVDGHHLAYRTFHALKGLTTSRGEPVQAVYGFAKSLLKALKED
GDAVIVVFDAKAPSFRHEAYGGYKAGRAPTPEDFPRQLALIKELVDLLGLARLEVPGYE
ADDVLASLAKKAEKEGYEVRILTADKDLYQLLSDRIHVLHPEGYLITPAWLWEKYGLRP
DQWADYRALTGDESDNLPGVKGIGEKTARKLLEEWGSLEALLKNLDRLKPAIREKILAH
MDDLKLSWDLAKVRTDLPLEVDFAKRREPDRERLRAFLERLEFGSLLHEFGLLESPKAL
EEAPWPPPEGAFVGFVLSRKEPMWADLLALAAARGGRVHRAPEPYKALRDLKEARGLL
AKDLSVLALREGLGLPPGDDPMLLAYLLDPSNTTPEGVARRYGGEWTEEAGERAALSER
LFANLWGRLEGEERLLWLYREVERPLSAVLAHMEATGVRLDVAYLRALSLEVAEEIAR
LEAEVFRLAGGSWYQPKGGTEMFCHPRTGKPLPKYPRIKIPKVGGIFKKPKNKAQREGR
EPCELDTREYVAGAPYTPVEHVVFNPSSRDQLERVLFDELGLPAIGKTEKTGKRSTSAAV
LEALREAHPIVEKILQYRELTKLKSTYIDPLPDLIHPRTGRLHTRFNQTATATGRLSSSDPN
LQNIPVRTPLGQRIRRAFIAEEGWLLVALDYSQIELRVLAHLSGDENLIRVFQEGRDIHTE
TASWMFGVPREAVDPLMRRAAKTINFGVLYGMSAHRLSQELAIPYEEAQAFIERYFQSF
PKVRAWIEKTLEEGRRRGYVETLFGRRRYVPDLEARVKSVREAAERMAFNMPVQGTAA
DLMKLAMVKLFPRLEEMGARMLLQVHDELVLEAPKERAEAVARLAKEVMEGVYPLAV
PLEVEVGIGEDWLSAKGDGGSSGGSSGGSGSSGSGGGSSGGSGSSGGGGSGGGSGSGSS
GSGSSGGSSGGSSGSDKIIHLTDDSFDTDVLKADGAILVDFWAEWCGPCKMIAPILDEIA
DEYQGKLTVAKLNIDQNPGTAPKYGIRGIPTLLLFKNGEVAATKVGALSKGQLKEFLDA
NLA
Seq. ID No: 81-TaqTBD-50GS linker-TRX, pATG7588
MRGMLPLFEPKGRVLLVDGHHLAYRTFHALKGLTTSRGEPVQAVYGFAKSLLKALKED
GDAVIVVFDAKAPSFRHEAYGGYKAGRAPTPEDFPRQLALIKELVDLLGLARLEVPGYE
ADDVLASLAKKAEKEGYEVRILTADKDLYQLLSDRIHVLHPEGYLITPAWLWEKYGLRP
DQWADYRALTGDESDNLPGVKGIGEKTARKLLEEWGSLEALLKNLDRLKPAIREKILAH
MDDLKLSWDLAKVRTDLPLEVDFAKRREPDRERLRAFLERLEFGSLLHEFGLLESPKAL
EEAPWPPPEGAFVGFVLSRKEPMWADLLALAAARGGRVHRAPEPYKALRDLKEARGLL
AKDLSVLALREGLGLPPGDDPMLLAYLLDPSNTTPEGVARRYGGEWTEEAGERAALSER
LFANLWGRLEGEERLLWLYREVERPLSAVLAHMEATGVRLDVAYLRALSLEVAEEIAR
LEAEVFRLAGGSWYQPKGGTEMFCHPRTGKPLPKYPRIKIPKVGGIFKKPKNKAQREGR
EPCELDTREYVAGAPYTPVEHVVFNPSSRDQLERVLFDELGLPAIGKTEKTGKRSTSAAV
LEALREAHPIVEKILQYRELTKLKSTYIDPLPDLIHPRTGRLHTRFNQTATATGRLSSSDPN
LONIPVRTPLGQRIRRAFIAEEGWLLVALDYSQIELRVLAHLSGDENLIRVFQEGRDIHTE
TASWMFGVPREAVDPLMRRAAKTINFGVLYGMSAHRLSQELAIPYEEAQAFIERYFQSF
PKVRAWIEKTLEEGRRRGYVETLFGRRRYVPDLEARVKSVREAAERMAFNMPVQGTAA
DLMKLAMVKLFPRLEEMGARMLLQVHDELVLEAPKERAEAVARLAKEVMEGVYPLAV
PLEVEVGIGEDWLSAKGDGGSSGSGSSGSGGGSSGGSGSSGGGGSGGGSGSGSSGSGSSG
GSSGGSSGSDKIIHLTDDSFDTDVLKADGAILVDFWAEWCGPCKMIAPILDEIADEYQGK
LTVAKLNIDQNPGTAPKYGIRGIPTLLLFKNGEVAATKVGALSKGQLKEFLDANLA
Seq. ID No: 82-TRX-45GS linker-TaqTBD, pATG7375
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGSSGGSS
GGGGSGSGSGSSGGSGSSGSGSSGSSGGGGSGGSSRGMLPLFEPKGRVLLVDGHHLAYR
TFHALKGLTTSRGEPVQAVYGFAKSLLKALKEDGDAVIVVFDAKAPSFRHEAYGGYKA
GRAPTPEDFPRQLALIKELVDLLGLARLEVPGYEADDVLASLAKKAEKEGYEVRILTAD
KDLYQLLSDRIHVLHPEGYLITPAWLWEKYGLRPDQWADYRALTGDESDNLPGVKGIG
EKTARKLLEEWGSLEALLKNLDRLKPAIREKILAHMDDLKLSWDLAKVRTDLPLEVDFA
KRREPDRERLRAFLERLEFGSLLHEFGLLESPKALEEAPWPPPEGAFVGFVLSRKEPMWA
DLLALAAARGGRVHRAPEPYKALRDLKEARGLLAKDLSVLALREGLGLPPGDDPMLLA
YLLDPSNTTPEGVARRYGGEWTEEAGERAALSERLFANLWGRLEGEERLLWLYREVER
PLSAVLAHMEATGVRLDVAYLRALSLEVAEEIARLEAEVFRLAGGSWYQPKGGTEMFC
HPRTGKPLPKYPRIKIPKVGGIFKKPKNKAQREGREPCELDTREYVAGAPYTPVEHVVFN
PSSRDQLERVLFDELGLPAIGKTEKTGKRSTSAAVLEALREAHPIVEKILQYRELTKLKST
YIDPLPDLIHPRTGRLHTRFNQTATATGRLSSSDPNLQNIPVRTPLGQRIRRAFIAEEGWLL
VALDYSQIELRVLAHLSGDENLIRVFQEGRDIHTETASWMFGVPREAVDPLMRRAAKTI
NFGVLYGMSAHRLSQELAIPYEEAQAFIERYFQSFPKVRAWIEKTLEEGRRRGYVETLFG
RRRYVPDLEARVKSVREAAERMAFNMPVQGTAADLMKLAMVKLFPRLEEMGARMLL
QVHDELVLEAPKERAEAVARLAKEVMEGVYPLAVPLEVEVGIGEDWLSAKGD.
Seq. ID No: 83-TaqSTBD-30GS linker-TRX, pATG7389
MRGMLPLFEPKGRVLLVDGHHLAYRTFHALKGLTTSRGEPVQAVYGFAKSLLKALKED
GDAVIVVFDAKAPSFRHEAYGGYKAGRAPTPEDFPRQLALIKELVDLLGLARLEVPGYE
ADDVLASLAKKAEKEGYEVRILTADKDLYQLLSDRIHVLHPEGYLITPAWLWEKYGLRP
DQWADYRALTGDESDNLPGVKGIGEKTARKLLEEWGSLEALLKNLDRLKPAIREKILAH
MDDLKLSWDLAKVRTDLPLEVDFAKRREPDRERLRAFLERLEFGSLLHEFGLLESPKAL
EEAPWPPPEGAFVGFVLSRKEPMWADLLALAAARGGRVHRAPEPYKALRDLKEARGLL
AKDLSVLALREGLGLPPGDDPMLLAYLLDPSNTTPEGVARRYGGEWTEEAGERAALSER
LFANLWGRLEGEERLLWLYREVERPLSAVLAHMEATGVRLDVAYLRALSLEVAEEIAR
LEAEVFRLAGGSWYQPKGGTEMFCHPRTGKPLPKYPRIKIPKVGGIFKKPKNKAQREGR
EPCELDTREYVAGAPYTPVEHVVFNPSSRDQLERVLFDELGLPAIGKTEKTGKRSTSAAV
LEALREAHPIVEKILQYRELTKLKSTYIDPLPDLIHPRTGRLHTRFNQTATATGRLSSSDPN
LQNIPVRTPLGQRIRRAFIAEEGWLLVALDYSQIELRVLAHLSGDENLIRVFQEGRDIHTE
TASWMFGVPREAVDPLMRRAAKTINFGVLYGMSAHRLSQELAIPYEEAQAFIERYFQSF
PKVRAWIEKTLEEGRRRGYVETLFGRRRYVPDLEARVKSVREAAERMAFNMPVQGTAA
DLMKLAMVKLFPRLEEMGARMLLQVHDELVLEAPKERAEAVARLAKEVMEGVYPLAV
PLEVEVGIGEDWLSAKGDGGSSGSGGGSSGGSGSSGGGGSGGSSGSSGSDKIIHLTDDSF
DTDVLKADGAILVDFWAEWCGPCKMIAPILDEIADEYQGKLTVAKLNIDQNPGTAPKYG
IRGIPTLLLFKNGEVAATKVGALSKGQLKEFLDANLA
Seq. ID No: 84-TaqTBD-25GS linker-TRX, pATG7428
MRGMLPLFEPKGRVLLVDGHHLAYRTFHALKGLTTSRGEPVQAVYGFAKSLLKALKED
GDAVIVVFDAKAPSFRHEAYGGYKAGRAPTPEDFPRQLALIKELVDLLGLARLEVPGYE
ADDVLASLAKKAEKEGYEVRILTADKDLYQLLSDRIHVLHPEGYLITPAWLWEKYGLRP
DQWADYRALTGDESDNLPGVKGIGEKTARKLLEEWGSLEALLKNLDRLKPAIREKILAH
MDDLKLSWDLAKVRTDLPLEVDFAKRREPDRERLRAFLERLEFGSLLHEFGLLESPKAL
EEAPWPPPEGAFVGFVLSRKEPMWADLLALAAARGGRVHRAPEPYKALRDLKEARGLL
AKDLSVLALREGLGLPPGDDPMLLAYLLDPSNTTPEGVARRYGGEWTEEAGERAALSER
LFANLWGRLEGEERLLWLYREVERPLSAVLAHMEATGVRLDVAYLRALSLEVAEEIAR
LEAEVFRLAGGSWYQPKGGTEMFCHPRTGKPLPKYPRIKIPKVGGIFKKPKNKAQREGR
EPCELDTREYVAGAPYTPVEHVVFNPSSRDQLERVLFDELGLPAIGKTEKTGKRSTSAAV
LEALREAHPIVEKILQYRELTKLKSTYIDPLPDLIHPRTGRLHTRFNQTATATGRLSSSDPN
LQNIPVRTPLGQRIRRAFIAEEGWLLVALDYSQIELRVLAHLSGDENLIRVFQEGRDIHTE
TASWMFGVPREAVDPLMRRAAKTINFGVLYGMSAHRLSQELAIPYEEAQAFIERYFQSF
PKVRAWIEKTLEEGRRRGYVETLFGRRRYVPDLEARVKSVREAAERMAFNMPVQGTAA
DLMKLAMVKLFPRLEEMGARMLLQVHDELVLEAPKERAEAVARLAKEVMEGVYPLAV
PLEVEVGIGEDWLSAKGDGGSSGSGGGSSGGSGSSGGSSGSSGSDKIIHLTDDSFDTDVL
KADGAILVDFWAEWCGPCKMIAPILDEIADEYQGKLTVAKLNIDQNPGTAPKYGIRGIPT
LLLFKNGEVAATKVGALSKGQLKEFLDANLA
Seq. ID No: 85-Alishwanella jeotgali TRX-60GS linker-Taq-TBD, pATG8151
MSEHILQVSDDSFETDVLKAEAPVLVDFWAEWCGPCKMIAPILDDVAAEYAGKVTVAK
VNIDQNPNTPPKFGIRGIPTLLLFKNGQVAATKVGALSKTQLKQFLDSNIGSSGSGGSGG
GSGSSGGSGSSGGSSGGGGSGSGSGSSGGSGSSGSGSSGSSGGGGSGGSSRGMLPLFEPK
GRVLLVDGHHLAYRTFHALKGLTTSRGEPVQAVYGFAKSLLKALKEDGDAVIVVFDAK
APSFRHEAYGGYKAGRAPTPEDFPRQLALIKELVDLLGLARLEVPGYEADDVLASLAKK
AEKEGYEVRILTADKDLYQLLSDRIHVLHPEGYLITPAWLWEKYGLRPDQWADYRALT
GDESDNLPGVKGIGEKTARKLLEEWGSLEALLKNLDRLKPAIREKILAHMDDLKLSWDL
AKVRTDLPLEVDFAKRREPDRERLRAFLERLEFGSLLHEFGLLESPKALEEAPWPPPEGA
FVGFVLSRKEPMWADLLALAAARGGRVHRAPEPYKALRDLKEARGLLAKDLSVLALRE
GLGLPPGDDPMLLAYLLDPSNTTPEGVARRYGGEWTEEAGERAALSERLFANLWGRLE
GEERLLWLYREVERPLSAVLAHMEATGVRLDVAYLRALSLEVAEEIARLEAEVFRLAGG
SWYQPKGGTEMFCHPRTGKPLPKYPRIKIPKVGGIFKKPKNKAQREGREPCELDTREYV
AGAPYTPVEHVVFNPSSRDQLERVLFDELGLPAIGKTEKTGKRSTSAAVLEALREAHPIV
EKILQYRELTKLKSTYIDPLPDLIHPRTGRLHTRFNQTATATGRLSSSDPNLQNIPVRTPLG
QRIRRAFIAEEGWLLVALDYSQIELRVLAHLSGDENLIRVFQEGRDIHTETASWMFGVPR
EAVDPLMRRAAKTINFGVLYGMSAHRLSQELAIPYEEAQAFIERYFQSFPKVRAWIEKTL
EEGRRRGYVETLFGRRRYVPDLEARVKSVREAAERMAFNMPVQGTAADLMKLAMVKL
FPRLEEMGARMLLQVHDELVLEAPKERAEAVARLAKEVMEGVYPLAVPLEVEVGIGED
WLSAKGD
Seq. ID No: 86-Alishwanella jeotgali TRX-82GS linker-Taq-Klebsiella Phage Kp_GWPB35
TBD, pATG8258
MSEHILQVSDDSFETDVLKAEAPVLVDFWAEWCGPCKMIAPILDDVAAEYAGKVTVAK
VNIDQNPNTPPKFGIRGIPTLLLFKNGQVAATKVGALSKTQLKQFLDSNIGSSGSSGSSGG
GSGGGSGSSGSSGSSGGGSGGGSGSSGGSGSSGGSSGGGGSGSGSGSSGGSGSSGSGSSG
SSGGGGSGGSSRGMLPLFEPKGRVLLVDGHHLAYRTFHALKGLTTSRGEPVQAVYGFA
KSLLKALKEDGDAVIVVFDAKAPSFRHEAYGGYKAGRAPTPEDFPRQLALIKELVDLLG
LARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGTWYQPKGGTELFLHPRTGKPLGKYPRVKYPKQGGIY
KKPKNKAQREGREPCDLDTRDYVEGAPYTPVEHVVFNPSSRDQLERVLFDELGLPAIGK
TEKTGKRSTSAAVLEALREAHPIVEKILQYRELTKLKSTYIDPLPDLIHPRTGRLHTRFNQ
TATATGRLSSSDPNLQNIPVRTPLGQRIRRAFIAEEGWLLVALDYSQIELRVLAHLSGDEN
LIRVFQEGRDIHTETASWMFGVPREAVDPLMRRAAKTINFGVLYGMSAHRLSQELAIPY
EEAQAFIERYFQSFPKVRAWIEKTLEEGRRRGYVETLFGRRRYVPDLEARVKSVREAAE
RMAFNMPVQGTAADLMKLAMVKLFPRLEEMGARMLLQVHDELVLEAPKERAEAVAR
LAKEVMEGVYPLAVPLEVEVGIGEDWLSAKGD
Seq. ID No: 87-Alishwanella jeotgali TRX-82GS linker-Taq-Salmonella enterica Phage
TBD, pATG8261
MSEHILQVSDDSFETDVLKAEAPVLVDFWAEWCGPCKMIAPILDDVAAEYAGKVTVAK
VNIDQNPNTPPKFGIRGIPTLLLFKNGQVAATKVGALSKTQLKQFLDSNIGSSGSSGSSGG
GSGGGSGSSGSSGSSGGGSGGGSGSSGGSGSSGGSSGGGGSGSGSGSSGGSGSSGSGSSG
SSGGGGSGGSSRGMLPLFEPKGRVLLVDGHHLAYRTFHALKGLTTSRGEPVQAVYGFA
KSLLKALKEDGDAVIVVFDAKAPSFRHEAYGGYKAGRAPTPEDFPRQLALIKELVDLLG
LARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYAPKGGKEFFRHPRTGKDLPKYPRVVYPKVGGIF
KKPKNKAQRLGLEPCERDTRDTMEGAPFTPITYVEFNPGSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAHRLSQELAIPYE
EAQAFIERYFQSFPKVRAWIEKTLEEGRRRGYVETLFGRRRYVPDLEARVKSVREAAER
MAFNMPVQGTAADLMKLAMVKLFPRLEEMGARMLLQVHDELVLEAPKERAEAVARL
AKEVMEGVYPLAVPLEVEVGIGEDWLSAKGD
Seq. ID No: 88-Alishwanella jeotgali TRX-82GS linker-Taq-Aeromonas phage PZL-Ah152
TBD, pATG8262
MSEHILQVSDDSFETDVLKAEAPVLVDFWAEWCGPCKMIAPILDDVAAEYAGKVTVAK
VNIDQNPNTPPKFGIRGIPTLLLFKNGQVAATKVGALSKTQLKQFLDSNIGSSGSSGSSGG
GSGGGSGSSGSSGSSGGGSGGGSGSSGGSGSSGGSSGGGGSGSGSGSSGGSGSSGSGSSG
SSGGGGSGGSSRGMLPLFEPKGRVLLVDGHHLAYRTFHALKGLTTSRGEPVQAVYGFA
KSLLKALKEDGDAVIVVFDAKAPSFRHEAYGGYKAGRAPTPEDFPRQLALIKELVDLLG
LARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGSSWYRPKGGKAFFRHPVTGKDLTNYPRVIYPKAGEIYT
KGGKLAKTLYCKDRPFTPIEYTVFNPGSRDQLERVLFDELGLPAIGKTEKTGKRSTSAAV
LEALREAHPIVEKILQYRELTKLKSTYIDPLPDLIHPRTGRLHTRFNQTATATGRLSSSDPN
LQNIPVRTPLGQRIRRAFIAEEGWLLVALDYSQIELRVLAHLSGDENLIRVFQEGRDIHTE
TASWMFGVPREAVDPLMRRAAKTINFGVLYGMSAHRLSQELAIPYEEAQAFIERYFQSF
PKVRAWIEKTLEEGRRRGYVETLFGRRRYVPDLEARVKSVREAAERMAFNMPVQGTAA
DLMKLAMVKLFPRLEEMGARMLLQVHDELVLEAPKERAEAVARLAKEVMEGVYPLAV
PLEVEVGIGEDWLSAKGD
Seq. ID No: 89-Thiococcus pfennigii TRX-60GS-Taq-TBD, pATG8156
MSDSIVHVTDDSFERDVLQSSEPVLVDYWADWCGPCKMIAPVLDEIATEYAGRIRVAKL
NIDENPNTPPRYGIRGIPTLMLFKDGEVEATKVGAVSKSQLTAFIDSNLGSSGSGGSGGGS
GSSGGSGSSGGSSGGGGSGSGSGSSGGSGSSGSGSSGSSGGGGSGGSSRGMLPLFEPKGR
VLLVDGHHLAYRTFHALKGLTTSRGEPVQAVYGFAKSLLKALKEDGDAVIVVFDAKAP
SFRHEAYGGYKAGRAPTPEDFPRQLALIKELVDLLGLARLEVPGYEADDVLASLAKKAE
KEGYEVRILTADKDLYQLLSDRIHVLHPEGYLITPAWLWEKYGLRPDQWADYRALTGD
ESDNLPGVKGIGEKTARKLLEEWGSLEALLKNLDRLKPAIREKILAHMDDLKLSWDLAK
VRTDLPLEVDFAKRREPDRERLRAFLERLEFGSLLHEFGLLESPKALEEAPWPPPEGAFV
GFVLSRKEPMWADLLALAAARGGRVHRAPEPYKALRDLKEARGLLAKDLSVLALREGL
GLPPGDDPMLLAYLLDPSNTTPEGVARRYGGEWTEEAGERAALSERLFANLWGRLEGE
ERLLWLYREVERPLSAVLAHMEATGVRLDVAYLRALSLEVAEEIARLEAEVFRLAGGS
WYQPKGGTEMFCHPRTGKPLPKYPRIKIPKVGGIFKKPKNKAQREGREPCELDTREYVA
GAPYTPVEHVVFNPSSRDQLERVLFDELGLPAIGKTEKTGKRSTSAAVLEALREAHPIVE
KILQYRELTKLKSTYIDPLPDLIHPRTGRLHTRFNQTATATGRLSSSDPNLQNIPVRTPLGQ
RIRRAFIAEEGWLLVALDYSQIELRVLAHLSGDENLIRVFQEGRDIHTETASWMFGVPRE
AVDPLMRRAAKTINFGVLYGMSAHRLSQELAIPYEEAQAFIERYFQSFPKVRAWIEKTLE
EGRRRGYVETLFGRRRYVPDLEARVKSVREAAERMAFNMPVQGTAADLMKLAMVKLF
PRLEEMGARMLLQVHDELVLEAPKERAEAVARLAKEVMEGVYPLAVPLEVEVGIGED
WLSAKGD
Seq. ID No: 90-Thiococcus pfennigii TRX-65GS-Taq-Klebsiella Phage Kp_GWPB35 TBD,
pATG8259
MSDSIVHVTDDSFERDVLQSSEPVLVDYWADWCGPCKMIAPVLDEIATEYAGRIRVAKL
NIDENPNTPPRYGIRGIPTLMLFKDGEVEATKVGAVSKSQLTAFIDSNLGSSGSSGSSGGG
SGGGSGSSGGSGSSGGSSGGGGSGSGSGSSGGSGSSGSGSSGSSGGGGSGGSSRGMLPLF
EPKGRVLLVDGHHLAYRTFHALKGLTTSRGEPVQAVYGFAKSLLKALKEDGDAVIVVF
DAKAPSFRHEAYGGYKAGRAPTPEDFPRQLALIKELVDLLGLARLEVPGYEADDVLASL
AKKAEKEGYEVRILTADKDLYQLLSDRIHVLHPEGYLITPAWLWEKYGLRPDQWADYR
ALTGDESDNLPGVKGIGEKTARKLLEEWGSLEALLKNLDRLKPAIREKILAHMDDLKLS
WDLAKVRTDLPLEVDFAKRREPDRERLRAFLERLEFGSLLHEFGLLESPKALEEAPWPPP
EGAFVGFVLSRKEPMWADLLALAAARGGRVHRAPEPYKALRDLKEARGLLAKDLSVL
ALREGLGLPPGDDPMLLAYLLDPSNTTPEGVARRYGGEWTEEAGERAALSERLFANLW
GRLEGEERLLWLYREVERPLSAVLAHMEATGVRLDVAYLRALSLEVAEEIARLEAEVFR
LAGGTWYQPKGGTELFLHPRTGKPLGKYPRVKYPKQGGIYKKPKNKAQREGREPCDLD
TRDYVEGAPYTPVEHVVFNPSSRDQLERVLFDELGLPAIGKTEKTGKRSTSAAVLEALRE
AHPIVEKILQYRELTKLKSTYIDPLPDLIHPRTGRLHTRFNQTATATGRLSSSDPNLQNIPV
RTPLGQRIRRAFIAEEGWLLVALDYSQIELRVLAHLSGDENLIRVFQEGRDIHTETASWM
FGVPREAVDPLMRRAAKTINFGVLYGMSAHRLSQELAIPYEEAQAFIERYFQSFPKVRA
WIEKTLEEGRRRGYVETLFGRRRYVPDLEARVKSVREAAERMAFNMPVQGTAADLMK
LAMVKLFPRLEEMGARMLLQVHDELVLEAPKERAEAVARLAKEVMEGVYPLAVPLEV
EVGIGEDWLSAKGD
Seq. ID No: 91-Thiococcus pfennigii TRX-65GS-Taq-Salmonella enterica Phage TBD,
pATG8260
MSDSIVHVTDDSFERDVLQSSEPVLVDYWADWCGPCKMIAPVLDEIATEYAGRIRVAKL
NIDENPNTPPRYGIRGIPTLMLFKDGEVEATKVGAVSKSQLTAFIDSNLGSSGSSGSSGGG
SGGGSGSSGGSGSSGGSSGGGGSGSGSGSSGGSGSSGSGSSGSSGGGGSGGSSRGMLPLF
EPKGRVLLVDGHHLAYRTFHALKGLTTSRGEPVQAVYGFAKSLLKALKEDGDAVIVVF
DAKAPSFRHEAYGGYKAGRAPTPEDFPRQLALIKELVDLLGLARLEVPGYEADDVLASL
AKKAEKEGYEVRILTADKDLYQLLSDRIHVLHPEGYLITPAWLWEKYGLRPDQWADYR
ALTGDESDNLPGVKGIGEKTARKLLEEWGSLEALLKNLDRLKPAIREKILAHMDDLKLS
WDLAKVRTDLPLEVDFAKRREPDRERLRAFLERLEFGSLLHEFGLLESPKALEEAPWPPP
EGAFVGFVLSRKEPMWADLLALAAARGGRVHRAPEPYKALRDLKEARGLLAKDLSVL
ALREGLGLPPGDDPMLLAYLLDPSNTTPEGVARRYGGEWTEEAGERAALSERLFANLW
GRLEGEERLLWLYREVERPLSAVLAHMEATGVRLDVAYLRALSLEVAEEIARLEAEVFR
LAGGSWYAPKGGKEFFRHPRTGKDLPKYPRVVYPKVGGIFKKPKNKAQRLGLEPCERD
TRDTMEGAPFTPITYVEFNPGSRDQLERVLFDELGLPAIGKTEKTGKRSTSAAVLEALRE
AHPIVEKILQYRELTKLKSTYIDPLPDLIHPRTGRLHTRFNQTATATGRLSSSDPNLQNIPV
RTPLGQRIRRAFIAEEGWLLVALDYSQIELRVLAHLSGDENLIRVFQEGRDIHTETASWM
FGVPREAVDPLMRRAAKTINFGVLYGMSAHRLSQELAIPYEEAQAFIERYFQSFPKVRA
WIEKTLEEGRRRGYVETLFGRRRYVPDLEARVKSVREAAERMAFNMPVQGTAADLMK
LAMVKLFPRLEEMGARMLLQVHDELVLEAPKERAEAVARLAKEVMEGVYPLAVPLEV
EVGIGEDWLSAKGD
Seq. ID No: 92-Thiococcus pfennigii TRX-82GS-Taq-Aeromonas phage PZL-Ah152 TBD,
pATG8263
MSDSIVHVTDDSFERDVLQSSEPVLVDYWADWCGPCKMIAPVLDEIATEYAGRIRVAKL
NIDENPNTPPRYGIRGIPTLMLFKDGEVEATKVGAVSKSQLTAFIDSNLGSSGSSGSSGGG
SGGGSGSSGSSGSSGGGSGGGSGSSGGSGSSGGSSGGGGSGSGSGSSGGSGSSGSGSSGSS
GGGGSGGSSRGMLPLFEPKGRVLLVDGHHLAYRTFHALKGLTTSRGEPVQAVYGFAKS
LLKALKEDGDAVIVVFDAKAPSFRHEAYGGYKAGRAPTPEDFPRQLALIKELVDLLGLA
RLEVPGYEADDVLASLAKKAEKEGYEVRILTADKDLYQLLSDRIHVLHPEGYLITPAWL
WEKYGLRPDQWADYRALTGDESDNLPGVKGIGEKTARKLLEEWGSLEALLKNLDRLKP
AIREKILAHMDDLKLSWDLAKVRTDLPLEVDFAKRREPDRERLRAFLERLEFGSLLHEFG
LLESPKALEEAPWPPPEGAFVGFVLSRKEPMWADLLALAAARGGRVHRAPEPYKALRD
LKEARGLLAKDLSVLALREGLGLPPGDDPMLLAYLLDPSNTTPEGVARRYGGEWTEEA
GERAALSERLFANLWGRLEGEERLLWLYREVERPLSAVLAHMEATGVRLDVAYLRALS
LEVAEEIARLEAEVERLAGSSWYRPKGGKAFFRHPVTGKDLTNYPRVIYPKAGEIYTKG
GKLAKTLYCKDRPFTPIEYTVFNPGSRDQLERVLFDELGLPAIGKTEKTGKRSTSAAVLE
ALREAHPIVEKILQYRELTKLKSTYIDPLPDLIHPRTGRLHTRFNQTATATGRLSSSDPNLQ
NIPVRTPLGQRIRRAFIAEEGWLLVALDYSQIELRVLAHLSGDENLIRVFQEGRDIHTETA
SWMFGVPREAVDPLMRRAAKTINFGVLYGMSAHRLSQELAIPYEEAQAFIERYFQSFPK
VRAWIEKTLEEGRRRGYVETLFGRRRYVPDLEARVKSVREAAERMAFNMPVQGTAADL
MKLAMVKLFPRLEEMGARMLLQVHDELVLEAPKERAEAVARLAKEVMEGVYPLAVPL
EVEVGIGEDWLSAKGD
Seq. ID No: 93-Thiococcus pfennigii TRX, pATG8291
MSDSIVHVTDDSFERDVLQSSEPVLVDYWADWCGPCKMIAPVLDEIATEYAGRIRVAKL
NIDENPNTPPRYGIRGIPTLMLFKDGEVEATKVGAVSKSQLTAFIDSNL
Seq. ID No: 94-Alishwanella jeotgali TRX, pATG8290
MSEHILQVSDDSFETDVLKAEAPVLVDFWAEWCGPCKMIAPILDDVAAEYAGKVTVAK
VNIDQNPNTPPKFGIRGIPTLLLFKNGQVAATKVGALSKTQLKQFLDSNI
Seq. ID No: 95-TRX-60GS linker-Taq- Salmonella enterica Phage TBD, pATG7606
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGGGGSG
GGSGSSGGSGSSGGSSGGGGSGGGSGSSGGSGSSGSGSSGSSGGGGSGGSSRGMLPLFEP
KGRVLLVDGHHLAYRTFHALKGLTTSRGEPVQAVYGFAKSLLKALKEDGDAVIVVFDA
KAPSFRHEAYGGYKAGRAPTPEDFPRQLALIKELVDLLGLARLEVPGYEADDVLASLAK
KAEKEGYEVRILTADKDLYQLLSDRIHVLHPEGYLITPAWLWEKYGLRPDQWADYRAL
TGDESDNLPGVKGIGEKTARKLLEEWGSLEALLKNLDRLKPAIREKILAHMDDLKLSWD
LAKVRTDLPLEVDFAKRREPDRERLRAFLERLEFGSLLHEFGLLESPKALEEAPWPPPEG
AFVGFVLSRKEPMWADLLALAAARGGRVHRAPEPYKALRDLKEARGLLAKDLSVLAL
REGLGLPPGDDPMLLAYLLDPSNTTPEGVARRYGGEWTEEAGERAALSERLFANLWGR
LEGEERLLWLYREVERPLSAVLAHMEATGVRLDVAYLRALSLEVAEEIARLEAEVFRLA
GGSWYAPKGGKEFFRHPRTGKDLPKYPRVVYPKVGGIFKKPKNKAQRLGLEPCERDTR
DTMEGAPFTPITYVEFNPGSRDQLERVLFDELGLPAIGKTEKTGKRSTSAAVLEALREAH
PIVEKILQYRELTKLKSTYIDPLPDLIHPRTGRLHTRFNQTATATGRLSSSDPNLQNIPVRT
PLGQRIRRAFIAEEGWLLVALDYSQIELRVLAHLSGDENLIRVFQEGRDIHTETASWMFG
VPREAVDPLMRRAAKTINFGVLYGMSAHRLSQELAIPYEEAQAFIERYFQSFPKVRAWIE
KTLEEGRRRGYVETLFGRRRYVPDLEARVKSVREAAERMAFNMPVQGTAADLMKLAM
VKLFPRLEEMGARMLLQVHDELVLEAPKERAEAVARLAKEVMEGVYPLAVPLEVEVGI
GEDWLSAKGD
Seq. ID No: 96-TRX-60GS linker-Taq-Aeromonas phage PZL-Ah152 TBD, pATG7610
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGGGGSG
GGSGSSGGSGSSGGSSGGGGSGGGSGSSGGSGSSGSGSSGSSGGGGSGGSSRGMLPLFEP
KGRVLLVDGHHLAYRTFHALKGLTTSRGEPVQAVYGFAKSLLKALKEDGDAVIVVFDA
KAPSFRHEAYGGYKAGRAPTPEDFPRQLALIKELVDLLGLARLEVPGYEADDVLASLAK
KAEKEGYEVRILTADKDLYQLLSDRIHVLHPEGYLITPAWLWEKYGLRPDQWADYRAL
TGDESDNLPGVKGIGEKTARKLLEEWGSLEALLKNLDRLKPAIREKILAHMDDLKLSWD
LAKVRTDLPLEVDFAKRREPDRERLRAFLERLEFGSLLHEFGLLESPKALEEAPWPPPEG
AFVGFVLSRKEPMWADLLALAAARGGRVHRAPEPYKALRDLKEARGLLAKDLSVLAL
REGLGLPPGDDPMLLAYLLDPSNTTPEGVARRYGGEWTEEAGERAALSERLFANLWGR
LEGEERLLWLYREVERPLSAVLAHMEATGVRLDVAYLRALSLEVAEEIARLEAEVFRLA
GSSWYRPKGGKAFFRHPVTGKDLTNYPRVIYPKAGEIYTKGGKLAKTLYCKDRPFTPIE
YTVFNPGSRDQLERVLFDELGLPAIGKTEKTGKRSTSAAVLEALREAHPIVEKILQYRELT
KLKSTYIDPLPDLIHPRTGRLHTRFNQTATATGRLSSSDPNLQNIPVRTPLGQRIRRAFIAE
EGWLLVALDYSQIELRVLAHLSGDENLIRVFQEGRDIHTETASWMFGVPREAVDPLMRR
AAKTINFGVLYGMSAHRLSQELAIPYEEAQAFIERYFQSFPKVRAWIEKTLEEGRRRGYV
ETLFGRRRYVPDLEARVKSVREAAERMAFNMPVQGTAADLMKLAMVKLFPRLEEMGA
RMLLQVHDELVLEAPKERAEAVARLAKEVMEGVYPLAVPLEVEVGIGEDWLSAKGD
Seq. ID No: 97-TRX-60GS linker-Taq-Klebsiella Phage Kp_GWPB35 TBD, pATG7602
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGGGGSG
GGSGSSGGSGSSGGSSGGGGSGGGSGSSGGSGSSGSGSSGSSGGGGSGGSSRGMLPLFEP
KGRVLLVDGHHLAYRTFHALKGLTTSRGEPVQAVYGFAKSLLKALKEDGDAVIVVFDA
KAPSFRHEAYGGYKAGRAPTPEDFPRQLALIKELVDLLGLARLEVPGYEADDVLASLAK
KAEKEGYEVRILTADKDLYQLLSDRIHVLHPEGYLITPAWLWEKYGLRPDQWADYRAL
TGDESDNLPGVKGIGEKTARKLLEEWGSLEALLKNLDRLKPAIREKILAHMDDLKLSWD
LAKVRTDLPLEVDFAKRREPDRERLRAFLERLEFGSLLHEFGLLESPKALEEAPWPPPEG
AFVGFVLSRKEPMWADLLALAAARGGRVHRAPEPYKALRDLKEARGLLAKDLSVLAL
REGLGLPPGDDPMLLAYLLDPSNTTPEGVARRYGGEWTEEAGERAALSERLFANLWGR
LEGEERLLWLYREVERPLSAVLAHMEATGVRLDVAYLRALSLEVAEEIARLEAEVFRLA
GGTWYQPKGGTELFLHPRTGKPLGKYPRVKYPKQGGIYKKPKNKAQREGREPCDLDTR
DYVEGAPYTPVEHVVFNPSSRDQLERVLFDELGLPAIGKTEKTGKRSTSAAVLEALREA
HPIVEKILQYRELTKLKSTYIDPLPDLIHPRTGRLHTRFNQTATATGRLSSSDPNLQNIPVR
TPLGQRIRRAFIAEEGWLLVALDYSQIELRVLAHLSGDENLIRVFQEGRDIHTETASWMF
GVPREAVDPLMRRAAKTINFGVLYGMSAHRLSQELAIPYEEAQAFIERYFQSFPKVRAWI
EKTLEEGRRRGYVETLFGRRRYVPDLEARVKSVREAAERMAFNMPVQGTAADLMKLA
MVKLFPRLEEMGARMLLQVHDELVLEAPKERAEAVARLAKEVMEGVYPLAVPLEVEV
GIGEDWLSAKGD
Seq. ID No: 98-Taq-Salmonella enterica Phage TBD, pATG8303
MRGMLPLFEPKGRVLLVDGHHLAYRTFHALKGLTTSRGEPVQAVYGFAKSLLKALKED
GDAVIVVFDAKAPSFRHEAYGGYKAGRAPTPEDFPRQLALIKELVDLLGLARLEVPGYE
ADDVLASLAKKAEKEGYEVRILTADKDLYQLLSDRIHVLHPEGYLITPAWLWEKYGLRP
DQWADYRALTGDESDNLPGVKGIGEKTARKLLEEWGSLEALLKNLDRLKPAIREKILAH
MDDLKLSWDLAKVRTDLPLEVDFAKRREPDRERLRAFLERLEFGSLLHEFGLLESPKAL
EEAPWPPPEGAFVGFVLSRKEPMWADLLALAAARGGRVHRAPEPYKALRDLKEARGLL
AKDLSVLALREGLGLPPGDDPMLLAYLLDPSNTTPEGVARRYGGEWTEEAGERAALSER
LFANLWGRLEGEERLLWLYREVERPLSAVLAHMEATGVRLDVAYLRALSLEVAEEIAR
LEAEVFRLAGGSWYAPKGGKEFFRHPRTGKDLPKYPRVVYPKVGGIFKKPKNKAQRLG
LEPCERDTRDTMEGAPFTPITYVEFNPGSRDQLERVLFDELGLPAIGKTEKTGKRSTSAA
VLEALREAHPIVEKILQYRELTKLKSTYIDPLPDLIHPRTGRLHTRFNQTATATGRLSSSDP
NLQNIPVRTPLGQRIRRAFIAEEGWLLVALDYSQIELRVLAHLSGDENLIRVFQEGRDIHT
ETASWMFGVPREAVDPLMRRAAKTINFGVLYGMSAHRLSQELAIPYEEAQAFIERYFQS
FPKVRAWIEKTLEEGRRRGYVETLFGRRRYVPDLEARVKSVREAAERMAFNMPVQGTA
ADLMKLAMVKLFPRLEEMGARMLLQVHDELVLEAPKERAEAVARLAKEVMEGVYPLA
VPLEVEVGIGEDWLSAKGD
Seq. ID No: 99-Taq-Aeromonas phage PZL-Ah152 TBD H914H, pATG8167
MRGMLPLFEPKGRVLLVDGHHLAYRTFHALKGLTTSRGEPVQAVYGFAKSLLKALKED
GDAVIVVFDAKAPSFRHEAYGGYKAGRAPTPEDFPRQLALIKELVDLLGLARLEVPGYE
ADDVLASLAKKAEKEGYEVRILTADKDLYQLLSDRIHVLHPEGYLITPAWLWEKYGLRP
DQWADYRALTGDESDNLPGVKGIGEKTARKLLEEWGSLEALLKNLDRLKPAIREKILAH
MDDLKLSWDLAKVRTDLPLEVDFAKRREPDRERLRAFLERLEFGSLLHEFGLLESPKAL
EEAPWPPPEGAFVGFVLSRKEPMWADLLALAAARGGRVHRAPEPYKALRDLKEARGLL
AKDLSVLALREGLGLPPGDDPMLLAYLLDPSNTTPEGVARRYGGEWTEEAGERAALSER
LFANLWGRLEGEERLLWLYREVERPLSAVLAHMEATGVRLDVAYLRALSLEVAEEIAR
LEAEVFRLAGSSWYRPKGGKAFFRHPVTGKDLTNYPRVIYPKAGEIYTKGGKLAKTLYC
KDRPFTPIEYTVFNPGSRDQLERVLFDELGLPAIGKTEKTGKRSTSAAVLEALREAHPIVE
KILQYRELTKLKSTYIDPLPDLIHPRTGRLHTRFNQTATATGRLSSSDPNLQNIPVRTPLGQ
RIRRAFIAEEGWLLVALDYSQIELRVLAHLSGDENLIRVFQEGRDIHTETASWMFGVPRE
AVDPLMRRAAKTINFGVLYGMSAHRLSQELAIPYEEAQAFIERYFQSFPKVRAWIEKTLE
EGRRRGYVETLFGRRRYVPDLEARVKSVREAAERMAFNMPVQGTAADLMKLAMVKLF
PRLEEMGARMLLQVHDELVLEAPKERAEAVARLAKEVMEGVYPLAVPLEVEVGIGED
WLSAKGD
Seq. ID No: 100-Taq-Klebsiella Phage Kp_GWPB35 TBD, pATG8304
MRGMLPLFEPKGRVLLVDGHHLAYRTFHALKGLTTSRGEPVQAVYGFAKSLLKALKED
GDAVIVVFDAKAPSFRHEAYGGYKAGRAPTPEDFPRQLALIKELVDLLGLARLEVPGYE
ADDVLASLAKKAEKEGYEVRILTADKDLYQLLSDRIHVLHPEGYLITPAWLWEKYGLRP
DQWADYRALTGDESDNLPGVKGIGEKTARKLLEEWGSLEALLKNLDRLKPAIREKILAH
MDDLKLSWDLAKVRTDLPLEVDFAKRREPDRERLRAFLERLEFGSLLHEFGLLESPKAL
EEAPWPPPEGAFVGFVLSRKEPMWADLLALAAARGGRVHRAPEPYKALRDLKEARGLL
AKDLSVLALREGLGLPPGDDPMLLAYLLDPSNTTPEGVARRYGGEWTEEAGERAALSER
LFANLWGRLEGEERLLWLYREVERPLSAVLAHMEATGVRLDVAYLRALSLEVAEEIAR
LEAEVFRLAGGTWYQPKGGTELFLHPRTGKPLGKYPRVKYPKQGGIYKKPKNKAQREG
REPCDLDTRDYVEGAPYTPVEHVVFNPSSRDQLERVLFDELGLPAIGKTEKTGKRSTSAA
VLEALREAHPIVEKILQYRELTKLKSTYIDPLPDLIHPRTGRLHTRFNQTATATGRLSSSDP
NLQNIPVRTPLGQRIRRAFIAEEGWLLVALDYSQIELRVLAHLSGDENLIRVFQEGRDIHT
ETASWMFGVPREAVDPLMRRAAKTINFGVLYGMSAHRLSQELAIPYEEAQAFIERYFQS
FPKVRAWIEKTLEEGRRRGYVETLFGRRRYVPDLEARVKSVREAAERMAFNMPVQGTA
ADLMKLAMVKLFPRLEEMGARMLLQVHDELVLEAPKERAEAVARLAKEVMEGVYPLA
VPLEVEVGIGEDWLSAKGD
Seq. ID No: 101-Salmonella enterica Phage TBD sequence
GSWYAPKGGKEFFRHPRTGKDLPKYPRVVYPKVGGIFKKPKNKAQRLGLEPCERDTRD
TMEGAPFTPITYVEFNPG
Seq. ID No: 102-Aeromonas phage PZL-Ah152 TBD sequence
SSWYRPKGGKAFFRHPVTGKDLTNYPRVIYPKAGEIYTKGGKLAKTLYCKDRPFTPIEYT
VFNPG
Seq. ID No: 103-Klebsiella Phage Kp_GWPB35 TBD sequence
GTWYQPKGGTELFLHPRTGKPLGKYPRVKYPKQGGIYKKPKNKAQREGREPCDLDTRD
YVEGAPYTPVEHVVENPS
Seq. ID No: 104-Tne Polymerase (v2), pATG8293
MKELQLYEEAEPTGYEIVKDHKTFEDLIEKLKEVPSFALALETSSLDPFNCEIVGISVSFKP
KTAYYIPLHHRNAQNLDETLVLSKLKEILEDPSSKIVGQNLKYAYKVLMVKGISPVYPHF
DTMIAAYLLEPNEKKFNLEDLSLKFLGYKMTSYQELMSFSSPLFGFSFADVPVDKAANY
SCEDADITYRLYKILSMKLHEAELENVFYRIEMPLVNVLARMELNGVYVDTEFLKKLSE
EYGKKLEELAEKIYQIAGEPFNINSPKQVSKILFEKLGIKPRGKTTKTGAYSTRIEVLEEIA
NEHEIVPLILEYRKIQKLKSTYIDTLPKLVNPKTGRIHASFHQTGTATGRLSSSDPNLQNLP
TKSEEGKEIRKAIVPQDPDWWIVSADYSQIELRILAHLSGDENLVKAFEEGIDVHTLTASR
IYNVKPEEVNEEMRRVGKMVNYSIIYGVTPYGLSVRLGIPVKEAEKMIISYFTLYPKVRS
YIQQVVAEAKEKGYVRTLFGRKRDIPQLMARDKNTQSEGERIAINTPIQGTAADIIKLAMI
DIDEELRKRNMKSRMIIQVHDELVFEVPDEEKEELVDLVKNKMTNVVKLSVPLEVDISIG
KSWS
Seq. ID No: 105-Tne Polymerase (v2)-TBD chimera, pATG8294
MKELQLYEEAEPTGYEIVKDHKTFEDLIEKLKEVPSFALALETSSLDPFNCEIVGISVSFKP
KTAYYIPLHHRNAQNLDETLVLSKLKEILEDPSSKIVGQNLKYAYKVLMVKGISPVYPHF
DTMIAAYLLEPNEKKFNLEDLSLKFLGYKMTSYQELMSFSSPLFGFSFADVPVDKAANY
SCEDADITYRLYKILSMKLHEAELENVFYRIEMPLVNVLARMELNGVYVDTEFLKKLSE
EYGKKLEELAEKIYQIAGGSWYQPKGGTEMFCHPRTGKPLPKYPRIKIPKVGGIFKKPKN
KAQREGREPCELDTREYVAGAPYTPVEHVVFNPSSPKQVSKILFEKLGIKPRGKTTKTGA
YSTRIEVLEEIANEHEIVPLILEYRKIQKLKSTYIDTLPKLVNPKTGRIHASFHQTGTATGRL
SSSDPNLQNLPTKSEEGKEIRKAIVPQDPDWWIVSADYSQIELRILAHLSGDENLVKAFEE
GIDVHTLTASRIYNVKPEEVNEEMRRVGKMVNYSIIYGVTPYGLSVRLGIPVKEAEKMIIS
YFTLYPKVRSYIQQVVAEAKEKGYVRTLFGRKRDIPQLMARDKNTQSEGERIAINTPIQG
TAADIIKLAMIDIDEELRKRNMKSRMIIQVHDELVFEVPDEEKEELVDLVKNKMTNVVK
LSVPLEVDISIGKSWS
Seq. ID No:106-TRX-60GS linker-Tfl-Taq chimera-TBD, pATG8266
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGGGGSG
GGSGSSGGSGSSGGSSGGGGSGSGSGSSGGSGSSGSGSSGSSGGGGSGGSSRGMLPLFEP
KGRVLLVDGHHLAYRTFHALKGLTTSRGEPVQAVYGFAKSLLKALKEDGDAVIVVFDA
KAPSFRHEAYGGYKAGRAPTPEDFPRQLALIKELVDLLGLARLEVPGYEADDVLASLAK
KAEKEGYEVRILTADKDLYQLLSDRIHVLHPEGYLITPAWLWEKYGLRPDQWADYRAL
TGDESDNLPGVKGIGEKTARKLLEEWGSLEALLKNLDRLKPAIREKILAHMDDLKLSWD
LAKVRTDLPLEVDFAKRREPDRERLRAFLERLEFGSLLHEFGLLESPKAAEEAPWPPPEG
AFLGFSFSRPEPMWAELLALAGAWEGRLHRAQDPLRGLRDLKGVRGILAKDLAVLALR
EGLDLFPEDDPMLLAYLLDPSNTTPEGVARRYGGEWTEDAGERALLAERLFQTLKERLK
GEERLLWLYEEVEKPLSRVLARMEATGVRLDVAYLRALSLEVAEEIARLEAEVFRLAGG
SWYQPKGGTEMFCHPRTGKPLPKYPRIKIPKVGGIFKKPKNKAQREGREPCELDTREYV
AGAPYTPVEHVVFNPSSRDQLERVLFDELGLPAIGKTEKTGKRSTSAAVLEALREAHPIV
EKILQYRELTKLKSTYIDPLPDLIHPRTGRLHTRFNQTATATGRLSSSDPNLQNIPVRTPLG
QRIRRAFIAEEGWLLVALDYSQIELRVLAHLSGDENLIRVFQEGRDIHTETASWMFGVPR
EAVDPLMRRAAKTINFGVLYGMSAHRLSQELAIPYEEAQAFIERYFQSFPKVRAWIEKTL
EEGRRRGYVETLFGRRRYVPDLEARVKSVREAAERMAFNMPVQGTAADLMKLAMVKL
FPRLEEMGARMLLQVHDELVLEAPKERAEAVARLAKEVMEGVYPLAVPLEVEVGIGED
WLSAKGD
Seq. ID No: 107-TRX E31P, pATG8143
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLA
Seq. ID No: 108-TRX E31P-82GS linker-Taq-TBD [E537V/S555N] H914A, pATG8100
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTRVYVAGAPYTPVEHVVFNPNSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAARLSQELAIPYE
EAQAFIERYFQSFPKVRAWIEKTLEEGRRRGYVETLFGRRRYVPDLEARVKSVREAAER
MAFNMPVQGTAADLMKLAMVKLFPRLEEMGARMLLQVHDELVLEAPKERAEAVARL
AKEVMEGVYPLAVPLEVEVGIGEDWLSAKGD
Seq ID No: 109-TRX V56I/A88V-60GS linker-Taq-TBD, pATG7633
MSDKIIHLTDDSFDTDVLKADGAILVDFWAEWCGPCKMIAPILDEIADEYQGKLTIAKLN
IDQNPGTAPKYGIRGIPTLLLFKNGEVVATKVGALSKGQLKEFLDANLAGSSGGGGSGG
GSGSSGGSGSSGGSSGGGGSGSGSGSSGGSGSSGSGSSGSSGGGGSGGSSRGMLPLFEPK
GRVLLVDGHHLAYRTFHALKGLTTSRGEPVQAVYGFAKSLLKALKEDGDAVIVVFDAK
APSFRHEAYGGYKAGRAPTPEDFPRQLALIKELVDLLGLARLEVPGYEADDVLASLAKK
AEKEGYEVRILTADKDLYQLLSDRIHVLHPEGYLITPAWLWEKYGLRPDQWADYRALT
GDESDNLPGVKGIGEKTARKLLEEWGSLEALLKNLDRLKPAIREKILAHMDDLKLSWDL
AKVRTDLPLEVDFAKRREPDRERLRAFLERLEFGSLLHEFGLLESPKALEEAPWPPPEGA
FVGFVLSRKEPMWADLLALAAARGGRVHRAPEPYKALRDLKEARGLLAKDLSVLALRE
GLGLPPGDDPMLLAYLLDPSNTTPEGVARRYGGEWTEEAGERAALSERLFANLWGRLE
GEERLLWLYREVERPLSAVLAHMEATGVRLDVAYLRALSLEVAEEIARLEAEVFRLAGG
SWYQPKGGTEMFCHPRTGKPLPKYPRIKIPKVGGIFKKPKNKAQREGREPCELDTREYV
AGAPYTPVEHVVFNPSSRDQLERVLFDELGLPAIGKTEKTGKRSTSAAVLEALREAHPIV
EKILQYRELTKLKSTYIDPLPDLIHPRTGRLHTRFNQTATATGRLSSSDPNLQNIPVRTPLG
QRIRRAFIAEEGWLLVALDYSQIELRVLAHLSGDENLIRVFQEGRDIHTETASWMFGVPR
EAVDPLMRRAAKTINFGVLYGMSAHRLSQELAIPYEEAQAFIERYFQSFPKVRAWIEKTL
EEGRRRGYVETLFGRRRYVPDLEARVKSVREAAERMAFNMPVQGTAADLMKLAMVKL
FPRLEEMGARMLLQVHDELVLEAPKERAEAVARLAKEVMEGVYPLAVPLEVEVGIGED
WLSAKGD
Seq ID No: 110-TRX S2M E31P-82GS linker-TaqTBD [537V,555N] +H914F, pATG7932
MMDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTRVYVAGAPYTPVEHVVFNPNSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD
Seq ID No: 111-TRXA D27E E31P-82GS linker-Taq-TBD [537V,555N] +H914F, pATG7933
MSDKIIHLTDDSFDTDVLKADGAILVEFWAPWCGPCKMIAPILDEIADEYQGKLTVAKLN
IDQNPGTAPKYGIRGIPTLLLFKNGEVAATKVGALSKGQLKEFLDANLAGSSGSSGSSGG
GSGGGSGSSGSSGSSGGGSGGGSGSSGGSGSSGGSSGGGGSGSGSGSSGGSGSSGSGSSG
SSGGGGSGGSSRGMLPLFEPKGRVLLVDGHHLAYRTFHALKGLTTSRGEPVQAVYGFA
KSLLKALKEDGDAVIVVEDAKAPSFRHEAYGGYKAGRAPTPEDFPRQLALIKELVDLLG
LARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTRVYVAGAPYTPVEHVVFNPNSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD
Seq ID No: 112-TRXA S2M D27E E31P-82GS linker-Taq-TBD [537V,555N] +H914F,
pATG7934
MMDKIIHLTDDSFDTDVLKADGAILVEFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTRVYVAGAPYTPVEHVVFNPNSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD
Seq. ID No: 113-TRX E31P-82GS linker-Taq-TBD [T535K, E537V] +H914F, pATG7840
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDKRVYVAGAPYTPVEHVVFNPSSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD
Seq. ID No: 114-TRX E31P-82GS linker-Taq-TBD [T535N, S555N] +H914F, pATG7864
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDNREYVAGAPYTPVEHVVFNPNSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD.
Seq. ID No: 115-TRX E31P-82GS linker-Taq-TBD [T535N, E537V, S555N] +H914F,
pATG7865
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDNRVYVAGAPYTPVEHVVFNPNSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD.
Seq. ID No: 116-TRX S2M E31P-82GS linker-Taq-TBD +H914F, pATG7866
MMDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSGSGSS
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTREYVAGAPYTPVEHVVFNPSSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD.
Seq. ID No: 117-TRX S2M E31P D48E-82GS linker-Taq-TBD +H914F, pATG7867
MMDKIIHLTDDSFDTDVLKADGAILVDFWAPWCGPCKMIAPILDEIAEEYQGKLTVAKL
NIDQNPGTAPKYGIRGIPTLLLFKNGEVAATKVGALSKGQLKEFLDANLAGSSGSGSGSS
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTREYVAGAPYTPVEHVVFNPSSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD.
Seq. ID No: 118-TRX S2E E31P D48E-82GS linker-Taq-TBD +H914F, pATG7868
MEDKIIHLTDDSFDTDVLKADGAILVDFWAPWCGPCKMIAPILDEIAEEYQGKLTVAKL
NIDQNPGTAPKYGIRGIPTLLLFKNGEVAATKVGALSKGQLKEFLDANLAGSSGSGSGSS
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTREYVAGAPYTPVEHVVFNPSSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD.
Seq. ID No: 119-TRX S2M E31P-82GS linker-Taq-TBD +H914F, pATG7869
MMDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSGSGSS
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVERLAGGSWYQPKGGTEMFCHPRTGKPLPKYPRIKIPKVGGIFK
KPKNKAQREGREPCELDTREYVAGAPYTPVEHVVFNPSSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD.
Seq. ID No: 120-TRX S2M E31P D48E-82GS linker-Taq-TBD [E537V] +H914F, pATG7870
MMDKIIHLTDDSFDTDVLKADGAILVDFWAPWCGPCKMIAPILDEIAEEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTRVYVAGAPYTPVEHVVFNPSSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD.
Seq. ID No: 121-TRX S2E E31P D48E-82GS linker-Taq-TBD [E537V] +H936F, pATG7871
MEDKIIHLTDDSFDTDVLKADGAILVDFWAPWCGPCKMIAPILDEIAEEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVERLAGGSWYQPKGGTEMFCHPRTGKPLPKYPRIKIPKVGGIFK
KPKNKAQREGREPCELDTRVYVAGAPYTPVEHVVFNPSSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD.
Seq. ID No: 122-TRX [D27E E31P]-82GS linker-Taq-TBD +H936F, pATG7895
MSDKIIHLTDDSFDTDVLKADGAILVEFWAPWCGPCKMIAPILDEIADEYQGKLTVAKLN
IDQNPGTAPKYGIRGIPTLLLFKNGEVAATKVGALSKGQLKEFLDANLAGSSGSGSGSSG
GSGGGSGSSGSSGSSGGGSGGGSGSSGGSGSSGGSSGGGGSGSGSGSSGGSGSSGSGSSG
SSGGGGSGGSSRGMLPLFEPKGRVLLVDGHHLAYRTFHALKGLTTSRGEPVQAVYGFA
KSLLKALKEDGDAVIVVFDAKAPSFRHEAYGGYKAGRAPTPEDFPRQLALIKELVDLLG
LARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTREYVAGAPYTPVEHVVFNPSSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD.
Seq. ID No: 123-TRX D27E E31P-82GS linker-Taq-TBD [537V] +H914F, pATG7896
MSDKIIHLTDDSFDTDVLKADGAILVEFWAPWCGPCKMIAPILDEIADEYQGKLTVAKLN
IDQNPGTAPKYGIRGIPTLLLFKNGEVAATKVGALSKGQLKEFLDANLAGSSGSSGSSGG
GSGGGSGSSGSSGSSGGGSGGGSGSSGGSGSSGGSSGGGGSGSGSGSSGGSGSSGSGSSG
SSGGGGSGGSSRGMLPLFEPKGRVLLVDGHHLAYRTFHALKGLTTSRGEPVQAVYGFA
KSLLKALKEDGDAVIVVFDAKAPSFRHEAYGGYKAGRAPTPEDFPRQLALIKELVDLLG
LARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTRVYVAGAPYTPVEHVVFNPSSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD.
Seq. ID No: 124-TRX S2E E31P-82GS linker-Taq-TBD +H914F, pATG7898
MEDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSGSGSS
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTRVYVAGAPYTPVEHVVFNPSSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD.
Seq. ID No: 125-TRX S2E E31P-82GS linker-Taq-TBD [537V] +H914F, pATG7899
MEDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVERLAGGSWYQPKGGTEMFCHPRTGKPLPKYPRIKIPKVGGIFK
KPKNKAQREGREPCELDTRVYVAGAPYTPVEHVVFNPSSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD.
Seq. ID No: 126-TRX [E31P D48E]-82GS linker-Taq-TBD +H914F, pATG7900
MSDKIIHLTDDSFDTDVLKADGAILVDFWAPWCGPCKMIAPILDEIAEEYQGKLTVAKLN
IDQNPGTAPKYGIRGIPTLLLFKNGEVAATKVGALSKGQLKEFLDANLAGSSGSGSGSSG
GSGGGSGSSGSSGSSGGGSGGGSGSSGGSGSSGGSSGGGGSGSGSGSSGGSGSSGSGSSG
SSGGGGSGGSSRGMLPLFEPKGRVLLVDGHHLAYRTFHALKGLTTSRGEPVQAVYGFA
KSLLKALKEDGDAVIVVFDAKAPSFRHEAYGGYKAGRAPTPEDFPRQLALIKELVDLLG
LARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTREYVAGAPYTPVEHVVFNPSSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD.
Seq. ID No: 127-TRX E31P D48E-82GS linker-Taq-TBD [537V] +H914F, pATG7901
MSDKIIHLTDDSFDTDVLKADGAILVDFWAPWCGPCKMIAPILDEIAEEYQGKLTVAKLN
IDQNPGTAPKYGIRGIPTLLLFKNGEVAATKVGALSKGQLKEFLDANLAGSSGSSGSSGG
GSGGGSGSSGSSGSSGGGSGGGSGSSGGSGSSGGSSGGGGSGSGSGSSGGSGSSGSGSSG
SSGGGGSGGSSRGMLPLFEPKGRVLLVDGHHLAYRTFHALKGLTTSRGEPVQAVYGFA
KSLLKALKEDGDAVIVVFDAKAPSFRHEAYGGYKAGRAPTPEDFPRQLALIKELVDLLG
LARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTRVYVAGAPYTPVEHVVFNPSSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD.
Seq. ID No: 128-TRX E31P P65Q-82GS linker-Taq-TBD +H936F, pATG7902
MSDKIIHLTDDSFDTDVLKADGAILVDFWAPWCGPCKMIAPILDEIADEYQGKLTVAKL
NIDQNQGTAPKYGIRGIPTLLLFKNGEVAATKVGALSKGQLKEFLDANLAGSSGSGSGSS
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTREYVAGAPYTPVEHVVFNPSSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD.
Seq. ID No: 129-TRX E31P-82GS linker-Taq-TBD [E537T, S555N] +H914F, pATG7903
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTRTYVAGAPYTPVEHVVFNPNSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD.
Seq. ID No: 130-TRX E31P-82GS linker-Taq-TBD [E537S, S555R] +H914F, pATG7904
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTRSYVAGAPYTPVEHVVFNPRSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD.
Seq. ID No: 131-TRX E31P-82GS linker-Taq-TBD [E537S, S555N] +H914F, pATG7905
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTRSYVAGAPYTPVEHVVFNPNSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD.
Seq. ID No: 132-TRX E31P-82GS linker-Taq-TBD [E537A, S555N] +H914F, pATG7906
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTRAYVAGAPYTPVEHVVFNPNSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD.
Seq. ID No: 133-TRX E31P-82GS linker-Taq-TBD [T535N, E537S, S555N] +H914F,
pATG7907
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDNRSYVAGAPYTPVEHVVFNPNSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD.
Seq. ID No: 134-TRX E31P-82GS linker-Taq-TBD [E537T, S555R] +H914F, pATG7908
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTRTYVAGAPYTPVEHVVFNPRSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD.
Seq. ID No: 135-TRX E31P-82GS linker-Taq-TBD [E537A, S555R] +H914F, pATG7909
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTRAYVAGAPYTPVEHVVFNPRSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD.
Seq. ID No: 136-TRX E31P-82GS linker-Taq-TBD [T535N, E537S, S555R] +H914F,
pATG7910
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDNRSYVAGAPYTPVEHVVFNPRSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD.
Seq. ID No: 137-TRX E31P-82GS linker-Taq-TBD [T535N, E537A, S555R] +H914F,
pATG7911
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDNRAYVAGAPYTPVEHVVFNPRSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD.
Seq. ID No: 138-TRX E31P-82GS linker-Taq-TBD [535N, 537A, 555N] +H914F, pATG7937
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDNRAYVAGAPYTPVEHVVFNPNSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD.
Seq. ID No: 139-TRX E31P-82GS linker-Taq-TBD [535N, 537T, 555N] +H914F, pATG7938
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDNRTYVAGAPYTPVEHVVFNPNSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD.
Seq. ID No: 140-TRX E31P-82GS linker-Taq-TBD [535N, 537T, 555R] +H914F, pATG7939
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDNRTYVAGAPYTPVEHVVFNPRSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD.
Seq. ID No: 141-TRX E31P-82GS linker-Taq-TBD [535N, 537V, 555N] +H914F, pATG7940
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDNRVYVAGAPYTPVEHVVFNPNSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD.
Seq. ID No: 142-TRX E31P-82GS linker-Taq-TBD [535N, 537V, 555R] +H914F, pATG7941
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDNRVYVAGAPYTPVEHVVFNPRSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD.
Seq. ID No: 143-TRX [D48E, P65Q] E31P-82GS linker-Taq-TBD [537V, 555N] +H914F,
pATG8041
MSDKIIHLTDDSFDTDVLKADGAILVDFWAPWCGPCKMIAPILDEIAEEYQGKLTVAKLN
IDQNQGTAPKYGIRGIPTLLLFKNGEVAATKVGALSKGQLKEFLDANLAGSSGSGSGSSG
GGSGGGSGSSSSGSSGGGSGGGSGSSGGSGSSGGSSGGGGSGSGSGSSGGSGSSGSGSSG
SSGGGGSGGSSRGMLPLFEPKGRVLLVDGHHLAYRTFHALKGLTTSRGEPVQAVYGFA
KSLLKALKEDGDAVIVVFDAKAPSFRHEAYGGYKAGRAPTPEDFPRQLALIKELVDLLG
LARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTRVYVAGAPYTPVEHVVFNPNSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD
Seq. ID No: 144-TRX [S2M, D48E, P65Q] E31P-82GS linker -Taq-TBD [537V,555N]
+H914F, pATG8042
MMDKIIHLTDDSFDTDVLKADGAILVDFWAPWCGPCKMIAPILDEIAEEYQGKLTVAKL
NIDQNQGTAPKYGIRGIPTLLLFKNGEVAATKVGALSKGQLKEFLDANLAGSSGSGSGSS
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTRVYVAGAPYTPVEHVVFNPNSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD
Seq. ID No: 145-TRX [D27E, D48E, P65Q] E31P-82GS linker-Taq-TBD [537V,555N]
+H914F, pATG8043
MSDKIIHLTDDSFDTDVLKADGAILVEFWAPWCGPCKMIAPILDEIAEEYQGKLTVAKLN
IDQNQGTAPKYGIRGIPTLLLFKNGEVAATKVGALSKGQLKEFLDANLAGSSGSGSGSSG
GGSGGGSGSSSSGSSGGGSGGGSGSSGGSGSSGGSSGGGGSGSGSGSSGGSGSSGSGSSG
SSGGGGSGGSSRGMLPLFEPKGRVLLVDGHHLAYRTFHALKGLTTSRGEPVQAVYGFA
KSLLKALKEDGDAVIVVFDAKAPSFRHEAYGGYKAGRAPTPEDFPRQLALIKELVDLLG
LARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTRVYVAGAPYTPVEHVVFNPNSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD
Seq. ID No: 146-TRX [S2M, D27E, D48E, P65Q] E31P-82GS linker-Taq-TBD [537V, 555N]
+H914F, pATG8044
MMDKIIHLTDDSFDTDVLKADGAILVEFWAPWCGPCKMIAPILDEIAEEYQGKLTVAKL
NIDQNQGTAPKYGIRGIPTLLLFKNGEVAATKVGALSKGQLKEFLDANLAGSSGSGSGSS
GGGSGGGSGSSSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTRVYVAGAPYTPVEHVVFNPNSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD
Seq. ID No: 147-TRX [S2E, D48E, P65Q] E31P-82GS linker-Taq-TBD [537V, 555N] +H914F,
pATG8045
MEDKIIHLTDDSFDTDVLKADGAILVDFWAPWCGPCKMIAPILDEIAEEYQGKLTVAKL
NIDQNQGTAPKYGIRGIPTLLLFKNGEVAATKVGALSKGQLKEFLDANLAGSSGSGSGSS
GGGSGGGSGSSSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVERLAGGSWYQPKGGTEMFCHPRTGKPLPKYPRIKIPKVGGIFK
KPKNKAQREGREPCELDTRVYVAGAPYTPVEHVVFNPNSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD
Seq. ID No: 148-TRX [S2E, D27E, D48E, P65Q] E31P-82GS linker-Taq-TBD [537V, 555N]
+H914F, pATG8046
MEDKIIHLTDDSFDTDVLKADGAILVEFWAPWCGPCKMIAPILDEIAEEYQGKLTVAKLN
IDQNQGTAPKYGIRGIPTLLLFKNGEVAATKVGALSKGQLKEFLDANLAGSSGSGSGSSG
GGSGGGSGSSSSGSSGGGSGGGSGSSGGSGSSGGSSGGGGSGSGSGSSGGSGSSGSGSSG
SSGGGGSGGSSRGMLPLFEPKGRVLLVDGHHLAYRTFHALKGLTTSRGEPVQAVYGFA
KSLLKALKEDGDAVIVVFDAKAPSFRHEAYGGYKAGRAPTPEDFPRQLALIKELVDLLG
LARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTRVYVAGAPYTPVEHVVFNPNSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD
Seq. ID No: 149-TRX E31P-82GS linker-Taq-TBD [537V/S555N], pATG8168
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTRVYVAGAPYTPVEHVVFNPNSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAHRLSQELAIPYE
EAQAFIERYFQSFPKVRAWIEKTLEEGRRRGYVETLFGRRRYVPDLEARVKSVREAAER
MAFNMPVQGTAADLMKLAMVKLFPRLEEMGARMLLQVHDELVLEAPKERAEAVARL
AKEVMEGVYPLAVPLEVEVGIGEDWLSAKGD
Seq. ID No: 150-TRX-82GS linker-Taq-TBD [537V/S555N] +H914F, pATG8170
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTRVYVAGAPYTPVEHVVFNPNSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD
Seq. ID No: 151-TRX[E31P]-60GS linker-Taq-TBD, pATG7626
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGGGGSG
GGSGSSGGSGSSGGSSGGGGSGSGSGSSGGSGSSGSGSSGSSGGGGSGGSSRGMLPLFEP
KGRVLLVDGHHLAYRTFHALKGLTTSRGEPVQAVYGFAKSLLKALKEDGDAVIVVFDA
KAPSFRHEAYGGYKAGRAPTPEDFPRQLALIKELVDLLGLARLEVPGYEADDVLASLAK
KAEKEGYEVRILTADKDLYQLLSDRIHVLHPEGYLITPAWLWEKYGLRPDQWADYRAL
TGDESDNLPGVKGIGEKTARKLLEEWGSLEALLKNLDRLKPAIREKILAHMDDLKLSWD
LAKVRTDLPLEVDFAKRREPDRERLRAFLERLEFGSLLHEFGLLESPKALEEAPWPPPEG
AFVGFVLSRKEPMWADLLALAAARGGRVHRAPEPYKALRDLKEARGLLAKDLSVLAL
REGLGLPPGDDPMLLAYLLDPSNTTPEGVARRYGGEWTEEAGERAALSERLFANLWGR
LEGEERLLWLYREVERPLSAVLAHMEATGVRLDVAYLRALSLEVAEEIARLEAEVFRLA
GGSWYQPKGGTEMFCHPRTGKPLPKYPRIKIPKVGGIFKKPKNKAQREGREPCELDTRE
YVAGAPYTPVEHVVFNPSSRDQLERVLFDELGLPAIGKTEKTGKRSTSAAVLEALREAHP
IVEKILQYRELTKLKSTYIDPLPDLIHPRTGRLHTRFNQTATATGRLSSSDPNLQNIPVRTP
LGQRIRRAFIAEEGWLLVALDYSQIELRVLAHLSGDENLIRVFQEGRDIHTETASWMFGV
PREAVDPLMRRAAKTINFGVLYGMSAHRLSQELAIPYEEAQAFIERYFQSFPKVRAWIEK
TLEEGRRRGYVETLFGRRRYVPDLEARVKSVREAAERMAFNMPVQGTAADLMKLAMV
KLFPRLEEMGARMLLQVHDELVLEAPKERAEAVARLAKEVMEGVYPLAVPLEVEVGIG
EDWLSAKGD
Seq. ID No: 152-TRX[E31P]-82GS linker-Taq-TBD, pATG7642
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSGSGSS
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTREYVAGAPYTPVEHVVFNPSSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAHRLSQELAIPYE
EAQAFIERYFQSFPKVRAWIEKTLEEGRRRGYVETLFGRRRYVPDLEARVKSVREAAER
MAFNMPVQGTAADLMKLAMVKLFPRLEEMGARMLLQVHDELVLEAPKERAEAVARL
AKEVMEGVYPLAVPLEVEVGIGEDWLSAKGD
Seq. ID No: 153-TRX[E31P]-82GS linker-Taq-TBD[S555K] +H914F, pATG7697
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTREYVAGAPYTPVEHVVFNPKSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD
Seq. ID No: 154-TRX[E31P]-82GS linker-Taq-TBD[S555N] +H914F, pATG7698
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTREYVAGAPYTPVEHVVFNPNSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD
Seq. ID No: 155-TRX[E31P]-82GS linker-Taq-TBD[E537K] +H914F, pATG7714
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTRKYVAGAPYTPVEHVVFNPSSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD
Seq. ID No: 156-TRX[E31P]-82GS linker-Taq-TBD[E537L] +H914F, pATG7699
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTRLYVAGAPYTPVEHVVFNPSSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD
Seq. ID No: 157-TRX[E31P]-82GS linker-Taq-TBD[T489K] +H914F, pATG7712
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGKEMFCHPRTGKPLPKYPRIKIPKVGGIFK
KPKNKAQREGREPCELDTREYVAGAPYTPVEHVVFNPSSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD
Seq. ID No: 158-TRX[E31P]-82GS linker-Taq-TBD[E537V] +H914F, pATG7713
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVERLAGGSWYQPKGGTEMFCHPRTGKPLPKYPRIKIPKVGGIFK
KPKNKAQREGREPCELDTRVYVAGAPYTPVEHVVFNPSSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD
Seq. ID No: 159-TRX[E31P]-82GS linker-Taq-TBD[E548K] +H914F, pATG7717
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTREYVAGAPYTPVKHVVFNPSSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD
Seq. ID No: 160-TRX[E31P]-82GS linker-Taq-TBD[T535K] +H914F, pATG7718
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDKREYVAGAPYTPVEHVVFNPSSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD
Seq. ID No: 161-TRX[E31P]-82GS linker-Taq-TBD[R506K] +H914F, pATG7743
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPKIKIPKVGGIFK
KPKNKAQREGREPCELDTREYVAGAPYTPVEHVVFNPSSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD
Seq. ID No: 162-TRX[E31P]-82GS linker-Taq-TBD[S555R] +H914F, pATG7853
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTREYVAGAPYTPVEHVVFNPRSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD
Seq. ID No: 163-TRX L104I-82GS linker-Taq-TBD, pATG7643
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFIDANLAGSSGSGSGSS
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTREYVAGAPYTPVEHVVFNPSSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAHRLSQELAIPYE
EAQAFIERYFQSFPKVRAWIEKTLEEGRRRGYVETLFGRRRYVPDLEARVKSVREAAER
MAFNMPVQGTAADLMKLAMVKLFPRLEEMGARMLLQVHDELVLEAPKERAEAVARL
AKEVMEGVYPLAVPLEVEVGIGEDWLSAKGD
Seq. ID No: 164-TRX[L104I]-73GS linker-Taq-TBD, pATG7644
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFIDANLAGSSGSGSGSS
GGSGGGSGSSGGSGSSGGSSGGGGSGSGSGSSGGSGSSGSGSSGSSGGGGSGGSSGSSGG
SGSRGMLPLFEPKGRVLLVDGHHLAYRTFHALKGLTTSRGEPVQAVYGFAKSLLKALKE
DGDAVIVVFDAKAPSFRHEAYGGYKAGRAPTPEDFPRQLALIKELVDLLGLARLEVPGY
EADDVLASLAKKAEKEGYEVRILTADKDLYQLLSDRIHVLHPEGYLITPAWLWEKYGLR
PDQWADYRALTGDESDNLPGVKGIGEKTARKLLEEWGSLEALLKNLDRLKPAIREKILA
HMDDLKLSWDLAKVRTDLPLEVDFAKRREPDRERLRAFLERLEFGSLLHEFGLLESPKA
LEEAPWPPPEGAFVGFVLSRKEPMWADLLALAAARGGRVHRAPEPYKALRDLKEARGL
LAKDLSVLALREGLGLPPGDDPMLLAYLLDPSNTTPEGVARRYGGEWTEEAGERAALSE
RLFANLWGRLEGEERLLWLYREVERPLSAVLAHMEATGVRLDVAYLRALSLEVAEEIA
RLEAEVFRLAGGSWYQPKGGTEMFCHPRTGKPLPKYPRIKIPKVGGIFKKPKNKAQREG
REPCELDTREYVAGAPYTPVEHVVFNPSSRDQLERVLFDELGLPAIGKTEKTGKRSTSAA
VLEALREAHPIVEKILQYRELTKLKSTYIDPLPDLIHPRTGRLHTRFNQTATATGRLSSSDP
NLQNIPVRTPLGQRIRRAFIAEEGWLLVALDYSQIELRVLAHLSGDENLIRVFQEGRDIHT
ETASWMFGVPREAVDPLMRRAAKTINFGVLYGMSAHRLSQELAIPYEEAQAFIERYFQS
FPKVRAWIEKTLEEGRRRGYVETLFGRRRYVPDLEARVKSVREAAERMAFNMPVQGTA
ADLMKLAMVKLFPRLEEMGARMLLQVHDELVLEAPKERAEAVARLAKEVMEGVYPLA
VPLEVEVGIGEDWLSAKGD
Seq. ID No: 165-TRX[E31P]-73GS linker-Taq-TBD, pATG7645
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSGSGSS
GGSGGGSGSSGGSGSSGGSSGGGGSGSGSGSSGGSGSSGSGSSGSSGGGGSGGSSGSSGG
SGSRGMLPLFEPKGRVLLVDGHHLAYRTFHALKGLTTSRGEPVQAVYGFAKSLLKALKE
DGDAVIVVFDAKAPSFRHEAYGGYKAGRAPTPEDFPRQLALIKELVDLLGLARLEVPGY
EADDVLASLAKKAEKEGYEVRILTADKDLYQLLSDRIHVLHPEGYLITPAWLWEKYGLR
PDQWADYRALTGDESDNLPGVKGIGEKTARKLLEEWGSLEALLKNLDRLKPAIREKILA
HMDDLKLSWDLAKVRTDLPLEVDFAKRREPDRERLRAFLERLEFGSLLHEFGLLESPKA
LEEAPWPPPEGAFVGFVLSRKEPMWADLLALAAARGGRVHRAPEPYKALRDLKEARGL
LAKDLSVLALREGLGLPPGDDPMLLAYLLDPSNTTPEGVARRYGGEWTEEAGERAALSE
RLFANLWGRLEGEERLLWLYREVERPLSAVLAHMEATGVRLDVAYLRALSLEVAEEIA
RLEAEVFRLAGGSWYQPKGGTEMFCHPRTGKPLPKYPRIKIPKVGGIFKKPKNKAQREG
REPCELDTREYVAGAPYTPVEHVVFNPSSRDQLERVLFDELGLPAIGKTEKTGKRSTSAA
VLEALREAHPIVEKILQYRELTKLKSTYIDPLPDLIHPRTGRLHTRFNQTATATGRLSSSDP
NLQNIPVRTPLGQRIRRAFIAEEGWLLVALDYSQIELRVLAHLSGDENLIRVFQEGRDIHT
ETASWMFGVPREAVDPLMRRAAKTINFGVLYGMSAHRLSQELAIPYEEAQAFIERYFQS
FPKVRAWIEKTLEEGRRRGYVETLFGRRRYVPDLEARVKSVREAAERMAFNMPVQGTA
ADLMKLAMVKLFPRLEEMGARMLLQVHDELVLEAPKERAEAVARLAKEVMEGVYPLA
VPLEVEVGIGEDWLSAKGD
Seq. ID No: 166-TRX E31P-82GS linker-Taq-TBD [537V/S555N] +H914F, T7 Clamp,
pATG8130
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLGEMKGEYKDDFK
RMLEEQGEEYVDGLEFGSLLHEFGLLESPKALEEAPWPPPEGAFVGFVLSRKEPMWADL
LALAAARGGRVHRAPEPYKALRDLKEARGLLAKDLSVLALREGLGLPPGDDPMLLAYL
LDPSNTTPEGVARRYGGEWTEEAGERAALSERLFANLWGRLEGEERLLWLYREVERPLS
AVLAHMEATGVRLDVAYLRALSLEVAEEIARLEAEVFRLAGGSWYQPKGGTEMFCHPR
TGKPLPKYPRIKIPKVGGIFKKPKNKAQREGREPCELDTRVYVAGAPYTPVEHVVENPNS
RDQLERVLFDELGLPAIGKTEKTGKRSTSAAVLEALREAHPIVEKILQYRELTKLKSTYID
PLPDLIHPRTGRLHTRFNQTATATGRLSSSDPNLQNIPVRTPLGQRIRRAFIAEEGWLLVA
LDYSQIELRVLAHLSGDENLIRVFQEGRDIHTETASWMFGVPREAVDPLMRRAAKTINFG
VLYGMSAFRLSQELAIPYEEAQAFIERYFQSFPKVRAWIEKTLEEGRRRGYVETLFGRRR
YVPDLEARVKSVREAAERMAFNMPVQGTAADLMKLAMVKLFPRLEEMGARMLLQVH
DELVLEAPKERAEAVARLAKEVMEGVYPLAVPLEVEVGIGEDWLSAKGD
Seq. ID No: 167-TRX-60GS linker-Taq-TBD +H914F, pATG7569
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGGGGSG
GGSGSSGGSGSSGGSSGGGGSGGGSGSSGGSGSSGSGSSGSSGGGGSGGSSRGMLPLFEP
KGRVLLVDGHHLAYRTFHALKGLTTSRGEPVQAVYGFAKSLLKALKEDGDAVIVVEDA
KAPSFRHEAYGGYKAGRAPTPEDFPRQLALIKELVDLLGLARLEVPGYEADDVLASLAK
KAEKEGYEVRILTADKDLYQLLSDRIHVLHPEGYLITPAWLWEKYGLRPDQWADYRAL
TGDESDNLPGVKGIGEKTARKLLEEWGSLEALLKNLDRLKPAIREKILAHMDDLKLSWD
LAKVRTDLPLEVDFAKRREPDRERLRAFLERLEFGSLLHEFGLLESPKALEEAPWPPPEG
AFVGFVLSRKEPMWADLLALAAARGGRVHRAPEPYKALRDLKEARGLLAKDLSVLAL
REGLGLPPGDDPMLLAYLLDPSNTTPEGVARRYGGEWTEEAGERAALSERLFANLWGR
LEGEERLLWLYREVERPLSAVLAHMEATGVRLDVAYLRALSLEVAEEIARLEAEVFRLA
GGSWYQPKGGTEMFCHPRTGKPLPKYPRIKIPKVGGIFKKPKNKAQREGREPCELDTRE
YVAGAPYTPVEHVVFNPSSRDQLERVLFDELGLPAIGKTEKTGKRSTSAAVLEALREAHP
IVEKILQYRELTKLKSTYIDPLPDLIHPRTGRLHTRFNQTATATGRLSSSDPNLQNIPVRTP
LGQRIRRAFIAEEGWLLVALDYSQIELRVLAHLSGDENLIRVFQEGRDIHTETASWMFGV
PREAVDPLMRRAAKTINFGVLYGMSAFRLSQELAIPYEEAQAFIERYFQSFPKVRAWIEK
TLEEGRRRGYVETLFGRRRYVPDLEARVKSVREAAERMAFNMPVQGTAADLMKLAMV
KLFPRLEEMGARMLLQVHDELVLEAPKERAEAVARLAKEVMEGVYPLAVPLEVEVGIG
EDWLSAKGD
Seq. ID No: 168-TRX-77GS linker-Taq-TBD, pATG7640
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGSGGSGG
GSGSSGSSGSSGGGSGGGSGSSGGSGSSGGSSGGGGSGSGSGSSGGSGSSGSGSSGSSGG
GGSGGSSRGMLPLFEPKGRVLLVDGHHLAYRTFHALKGLTTSRGEPVQAVYGFAKSLL
KALKEDGDAVIVVFDAKAPSFRHEAYGGYKAGRAPTPEDFPRQLALIKELVDLLGLARL
EVPGYEADDVLASLAKKAEKEGYEVRILTADKDLYQLLSDRIHVLHPEGYLITPAWLWE
KYGLRPDQWADYRALTGDESDNLPGVKGIGEKTARKLLEEWGSLEALLKNLDRLKPAI
REKILAHMDDLKLSWDLAKVRTDLPLEVDFAKRREPDRERLRAFLERLEFGSLLHEFGL
LESPKALEEAPWPPPEGAFVGFVLSRKEPMWADLLALAAARGGRVHRAPEPYKALRDL
KEARGLLAKDLSVLALREGLGLPPGDDPMLLAYLLDPSNTTPEGVARRYGGEWTEEAG
ERAALSERLFANLWGRLEGEERLLWLYREVERPLSAVLAHMEATGVRLDVAYLRALSL
EVAEEIARLEAEVFRLAGGSWYQPKGGTEMFCHPRTGKPLPKYPRIKIPKVGGIFKKPKN
KAQREGREPCELDTREYVAGAPYTPVEHVVFNPSSRDQLERVLFDELGLPAIGKTEKTG
KRSTSAAVLEALREAHPIVEKILQYRELTKLKSTYIDPLPDLIHPRTGRLHTRFNQTATAT
GRLSSSDPNLQNIPVRTPLGQRIRRAFIAEEGWLLVALDYSQIELRVLAHLSGDENLIRVF
QEGRDIHTETASWMFGVPREAVDPLMRRAAKTINFGVLYGMSAHRLSQELAIPYEEAQA
FIERYFQSFPKVRAWIEKTLEEGRRRGYVETLFGRRRYVPDLEARVKSVREAAERMAFN
MPVQGTAADLMKLAMVKLFPRLEEMGARMLLQVHDELVLEAPKERAEAVARLAKEV
MEGVYPLAVPLEVEVGIGEDWLSAKGD
Seq. ID No: 169-TRX-90GS linker-Taq-TBD H914F, pATG7622
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSGSSGGSGSRGMLPLFEPKGRVLLVDGHHLAYRTFHALKGLTTSRGEP
VQAVYGFAKSLLKALKEDGDAVIVVFDAKAPSFRHEAYGGYKAGRAPTPEDFPRQLALI
KELVDLLGLARLEVPGYEADDVLASLAKKAEKEGYEVRILTADKDLYQLLSDRIHVLHP
EGYLITPAWLWEKYGLRPDQWADYRALTGDESDNLPGVKGIGEKTARKLLEEWGSLEA
LLKNLDRLKPAIREKILAHMDDLKLSWDLAKVRTDLPLEVDFAKRREPDRERLRAFLER
LEFGSLLHEFGLLESPKALEEAPWPPPEGAFVGFVLSRKEPMWADLLALAAARGGRVHR
APEPYKALRDLKEARGLLAKDLSVLALREGLGLPPGDDPMLLAYLLDPSNTTPEGVARR
YGGEWTEEAGERAALSERLFANLWGRLEGEERLLWLYREVERPLSAVLAHMEATGVRL
DVAYLRALSLEVAEEIARLEAEVFRLAGGSWYQPKGGTEMFCHPRTGKPLPKYPRIKIPK
VGGIFKKPKNKAQREGREPCELDTREYVAGAPYTPVEHVVFNPSSRDQLERVLFDELGL
PAIGKTEKTGKRSTSAAVLEALREAHPIVEKILQYRELTKLKSTYIDPLPDLIHPRTGRLHT
RFNQTATATGRLSSSDPNLQNIPVRTPLGQRIRRAFIAEEGWLLVALDYSQIELRVLAHLS
GDENLIRVFQEGRDIHTETASWMFGVPREAVDPLMRRAAKTINFGVLYGMSAFRLSQEL
AIPYEEAQAFIERYFQSFPKVRAWIEKTLEEGRRRGYVETLFGRRRYVPDLEARVKSVRE
AAERMAFNMPVQGTAADLMKLAMVKLFPRLEEMGARMLLQVHDELVLEAPKERAEA
VARLAKEVMEGVYPLAVPLEVEVGIGEDWLSAKGD
Seq. ID No: 170-TRX E31P-60GS linker-Taq-TBD [537V/S555N] +H914F, pATG8169
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGGGGSG
GGSGSSGGSGSSGGSSGGGGSGSGSGSSGGSGSSGSGSSGSSGGGGSGGSSRGMLPLFEP
KGRVLLVDGHHLAYRTFHALKGLTTSRGEPVQAVYGFAKSLLKALKEDGDAVIVVFDA
KAPSFRHEAYGGYKAGRAPTPEDFPRQLALIKELVDLLGLARLEVPGYEADDVLASLAK
KAEKEGYEVRILTADKDLYQLLSDRIHVLHPEGYLITPAWLWEKYGLRPDQWADYRAL
TGDESDNLPGVKGIGEKTARKLLEEWGSLEALLKNLDRLKPAIREKILAHMDDLKLSWD
LAKVRTDLPLEVDFAKRREPDRERLRAFLERLEFGSLLHEFGLLESPKALEEAPWPPPEG
AFVGFVLSRKEPMWADLLALAAARGGRVHRAPEPYKALRDLKEARGLLAKDLSVLAL
REGLGLPPGDDPMLLAYLLDPSNTTPEGVARRYGGEWTEEAGERAALSERLFANLWGR
LEGEERLLWLYREVERPLSAVLAHMEATGVRLDVAYLRALSLEVAEEIARLEAEVFRLA
GGSWYQPKGGTEMFCHPRTGKPLPKYPRIKIPKVGGIFKKPKNKAQREGREPCELDTRV
YVAGAPYTPVEHVVFNPNSRDQLERVLFDELGLPAIGKTEKTGKRSTSAAVLEALREAH
PIVEKILQYRELTKLKSTYIDPLPDLIHPRTGRLHTRFNQTATATGRLSSSDPNLQNIPVRT
PLGQRIRRAFIAEEGWLLVALDYSQIELRVLAHLSGDENLIRVFQEGRDIHTETASWMFG
VPREAVDPLMRRAAKTINFGVLYGMSAFRLSQELAIPYEEAQAFIERYFQSFPKVRAWIE
KTLEEGRRRGYVETLFGRRRYVPDLEARVKSVREAAERMAFNMPVQGTAADLMKLAM
VKLFPRLEEMGARMLLQVHDELVLEAPKERAEAVARLAKEVMEGVYPLAVPLEVEVGI
GEDWLSAKGD
Seq. ID No: 171-TRX E31P-82GS linker-Taq-TBD [T535K, S555N] +H914F, pATG7863
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDKREYVAGAPYTPVEHVVFNPNSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD
Seq. ID No: 172-TRX E31P-82GS linker-Taq-TBD [T535K, E537V, S555N] +H914F,
pATG7841
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDKRVYVAGAPYTPVEHVVFNPNSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAFRLSQELAIPYEE
AQAFIERYFQSFPKVRAWIEKTLEEGRRRGYVETLFGRRRYVPDLEARVKSVREAAERM
AFNMPVQGTAADLMKLAMVKLFPRLEEMGARMLLQVHDELVLEAPKERAEAVARLAK
EVMEGVYPLAVPLEVEVGIGEDWLSAKGD
SEQ ID No: 173-TRX-60GS linker-TaqS Alternate TBD #1, pATG7567
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGGGGSG
GGSGSSGGSGSSGGSSGGGGSGSGSGSSGGSGSSGSGSSGSSGGGGSGGSSRGMLPLFEP
KGRVLLVDGHHLAYRTFHALKGLTTSRGEPVQAVYGFAKSLLKALKEDGDAVIVVFDA
KAPSFRHEAYGGYKAGRAPTPEDFPRQLALIKELVDLLGLARLEVPGYEADDVLASLAK
KAEKEGYEVRILTADKDLYQLLSDRIHVLHPEGYLITPAWLWEKYGLRPDQWADYRAL
TGDESDNLPGVKGIGEKTARKLLEEWGSLEALLKNLDRLKPAIREKILAHMDDLKLSWD
LAKVRTDLPLEVDFAKRREPDRERLRAFLERLEFGSLLHEFGLLESPKALEEAPWPPPEG
AFVGFVLSRKEPMWADLLALAAARGGRVHRAPEPYKALRDLKEARGLLAKDLSVLAL
REGLGLPPGDDPMLLAYLLDPSNTTPEGVARRYGGEWTEEAGERAALSERLFANLWGR
LEGEERLLWLYREVERPLSAVLAHMEATGVRLDVAYLRALSLEVAEEIARLEAEVFRLA
GSWYQPKGGTEMFCHPRTGKPLPKYPRIKIPKVGGIFKKPKNKAQREGREPCELDTREY
VAGAPYTPVEHVVFNLNSRDQLERVLFDELGLPAIGKTEKTGKRSTSAAVLEALREAHPI
VEKILQYRELTKLKSTYIDPLPDLIHPRTGRLHTRFNQTATATGRLSSSDPNLQNIPVRTPL
GQRIRRAFIAEEGWLLVALDYSQIELRVLAHLSGDENLIRVFQEGRDIHTETASWMFGVP
REAVDPLMRRAAKTINFGVLYGMSAHRLSQELAIPYEEAQAFIERYFQSFPKVRAWIEKT
LEEGRRRGYVETLFGRRRYVPDLEARVKSVREAAERMAFNMPVQGTAADLMKLAMVK
LFPRLEEMGARMLLQVHDELVLEAPKERAEAVARLAKEVMEGVYPLAVPLEVEVGIGE
DWLSAKGD
SEQ ID No: 174-TRX-60GS linker-Taq-Alt_TBD_5 (GGSWY_TBD_FNLN),pATG7682
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGGGGSG
GGSGSSGGSGSSGGSSGGGGSGSGSGSSGGSGSSGSGSSGSSGGGGSGGSSRGMLPLFEP
KGRVLLVDGHHLAYRTFHALKGLTTSRGEPVQAVYGFAKSLLKALKEDGDAVIVVEDA
KAPSFRHEAYGGYKAGRAPTPEDFPRQLALIKELVDLLGLARLEVPGYEADDVLASLAK
KAEKEGYEVRILTADKDLYQLLSDRIHVLHPEGYLITPAWLWEKYGLRPDQWADYRAL
TGDESDNLPGVKGIGEKTARKLLEEWGSLEALLKNLDRLKPAIREKILAHMDDLKLSWD
LAKVRTDLPLEVDFAKRREPDRERLRAFLERLEFGSLLHEFGLLESPKALEEAPWPPPEG
AFVGFVLSRKEPMWADLLALAAARGGRVHRAPEPYKALRDLKEARGLLAKDLSVLAL
REGLGLPPGDDPMLLAYLLDPSNTTPEGVARRYGGEWTEEAGERAALSERLFANLWGR
LEGEERLLWLYREVERPLSAVLAHMEATGVRLDVAYLRALSLEVAEEIARLEAEVFRLA
GGSWYQPKGGTEMFCHPRTGKPLPKYPRIKIPKVGGIFKKPKNKAQREGREPCELDTRE
YVAGAPYTPVEHVVFNLNSRDQLERVLFDELGLPAIGKTEKTGKRSTSAAVLEALREAH
PIVEKILQYRELTKLKSTYIDPLPDLIHPRTGRLHTRFNQTATATGRLSSSDPNLQNIPVRT
PLGQRIRRAFIAEEGWLLVALDYSQIELRVLAHLSGDENLIRVFQEGRDIHTETASWMFG
VPREAVDPLMRRAAKTINFGVLYGMSAHRLSQELAIPYEEAQAFIERYFQSFPKVRAWIE
KTLEEGRRRGYVETLFGRRRYVPDLEARVKSVREAAERMAFNMPVQGTAADLMKLAM
VKLFPRLEEMGARMLLQVHDELVLEAPKERAEAVARLAKEVMEGVYPLAVPLEVEVGI
GEDWLSAKGD
SEQ ID No: 175-TRX-60GS linker-Taq- Alt_TBD_6(GSWY_TBD_FNPS), pATG7683
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGGGGSG
GGSGSSGGSGSSGGSSGGGGSGSGSGSSGGSGSSGSGSSGSSGGGGSGGSSRGMLPLFEP
KGRVLLVDGHHLAYRTFHALKGLTTSRGEPVQAVYGFAKSLLKALKEDGDAVIVVFDA
KAPSFRHEAYGGYKAGRAPTPEDFPRQLALIKELVDLLGLARLEVPGYEADDVLASLAK
KAEKEGYEVRILTADKDLYQLLSDRIHVLHPEGYLITPAWLWEKYGLRPDQWADYRAL
TGDESDNLPGVKGIGEKTARKLLEEWGSLEALLKNLDRLKPAIREKILAHMDDLKLSWD
LAKVRTDLPLEVDFAKRREPDRERLRAFLERLEFGSLLHEFGLLESPKALEEAPWPPPEG
AFVGFVLSRKEPMWADLLALAAARGGRVHRAPEPYKALRDLKEARGLLAKDLSVLAL
REGLGLPPGDDPMLLAYLLDPSNTTPEGVARRYGGEWTEEAGERAALSERLFANLWGR
LEGEERLLWLYREVERPLSAVLAHMEATGVRLDVAYLRALSLEVAEEIARLEAEVFRLA
GSWYQPKGGTEMFCHPRTGKPLPKYPRIKIPKVGGIFKKPKNKAQREGREPCELDTREY
VAGAPYTPVEHVVFNPSSRDQLERVLFDELGLPAIGKTEKTGKRSTSAAVLEALREAHPI
VEKILQYRELTKLKSTYIDPLPDLIHPRTGRLHTRFNQTATATGRLSSSDPNLQNIPVRTPL
GQRIRRAFIAEEGWLLVALDYSQIELRVLAHLSGDENLIRVFQEGRDIHTETASWMFGVP
REAVDPLMRRAAKTINFGVLYGMSAHRLSQELAIPYEEAQAFIERYFQSFPKVRAWIEKT
LEEGRRRGYVETLFGRRRYVPDLEARVKSVREAAERMAFNMPVQGTAADLMKLAMVK
LFPRLEEMGARMLLQVHDELVLEAPKERAEAVARLAKEVMEGVYPLAVPLEVEVGIGE
DWLSAKGD
Seq ID No: 176-TRX-82GS linker-Taq-TBD, pATG7359
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFCHPRTGKPLPKYPRIKIPKVGGIFK
KPKNKAQREGREPCELDTREYVAGAPYTPVEHVVFNPSSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAHRLSQELAIPYE
EAQAFIERYFQSFPKVRAWIEKTLEEGRRRGYVETLFGRRRYVPDLEARVKSVREAAER
MAFNMPVQGTAADLMKLAMVKLFPRLEEMGARMLLQVHDELVLEAPKERAEAVARL
AKEVMEGVYPLAVPLEVEVGIGEDWLSAKGD.
Seq ID No: 177-TRX-60GS linker-Taq-TBD + D816N E980A, pATG8332
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGGGGSG
GGSGSSGGSGSSGGSSGGGGSGSGSGSSGGSGSSGSGSSGSSGGGGSGGSSRGMLPLFEP
KGRVLLVDGHHLAYRTFHALKGLTTSRGEPVQAVYGFAKSLLKALKEDGDAVIVVEDA
KAPSFRHEAYGGYKAGRAPTPEDFPRQLALIKELVDLLGLARLEVPGYEADDVLASLAK
KAEKEGYEVRILTADKDLYQLLSDRIHVLHPEGYLITPAWLWEKYGLRPDQWADYRAL
TGDESDNLPGVKGIGEKTARKLLEEWGSLEALLKNLDRLKPAIREKILAHMDDLKLSWD
LAKVRTDLPLEVDFAKRREPDRERLRAFLERLEFGSLLHEFGLLESPKALEEAPWPPPEG
AFVGFVLSRKEPMWADLLALAAARGGRVHRAPEPYKALRDLKEARGLLAKDLSVLAL
REGLGLPPGDDPMLLAYLLDPSNTTPEGVARRYGGEWTEEAGERAALSERLFANLWGR
LEGEERLLWLYREVERPLSAVLAHMEATGVRLDVAYLRALSLEVAEEIARLEAEVFRLA
GGSWYQPKGGTEMFCHPRTGKPLPKYPRIKIPKVGGIFKKPKNKAQREGREPCELDTRE
YVAGAPYTPVEHVVFNPSSRDQLERVLFDELGLPAIGKTEKTGKRSTSAAVLEALREAHP
IVEKILQYRELTKLKSTYIDPLPDLIHPRTGRLHTRFNQTATATGRLSSSNPNLQNIPVRTP
LGQRIRRAFIAEEGWLLVALDYSQIELRVLAHLSGDENLIRVFQEGRDIHTETASWMFGV
PREAVDPLMRRAAKTINFGVLYGMSAHRLSQELAIPYEEAQAFIERYFQSFPKVRAWIEK
TLEEGRRRGYVETLFGRRRYVPDLEARVKSVRAAAERMAFNMPVQGTAADLMKLAMV
KLFPRLEEMGARMLLQVHDELVLEAPKERAEAVARLAKEVMEGVYPLAVPLEVEVGIG
EDWLSAKGD.
Seq ID No: 178-TRX-60GS linker-Taq-TBD + D816N V869A, pATG8333
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGGGGSG
GGSGSSGGSGSSGGSSGGGGSGSGSGSSGGSGSSGSGSSGSSGGGGSGGSSRGMLPLFEP
KGRVLLVDGHHLAYRTFHALKGLTTSRGEPVQAVYGFAKSLLKALKEDGDAVIVVFDA
KAPSFRHEAYGGYKAGRAPTPEDFPRQLALIKELVDLLGLARLEVPGYEADDVLASLAK
KAEKEGYEVRILTADKDLYQLLSDRIHVLHPEGYLITPAWLWEKYGLRPDQWADYRAL
TGDESDNLPGVKGIGEKTARKLLEEWGSLEALLKNLDRLKPAIREKILAHMDDLKLSWD
LAKVRTDLPLEVDFAKRREPDRERLRAFLERLEFGSLLHEFGLLESPKALEEAPWPPPEG
AFVGFVLSRKEPMWADLLALAAARGGRVHRAPEPYKALRDLKEARGLLAKDLSVLAL
REGLGLPPGDDPMLLAYLLDPSNTTPEGVARRYGGEWTEEAGERAALSERLFANLWGR
LEGEERLLWLYREVERPLSAVLAHMEATGVRLDVAYLRALSLEVAEEIARLEAEVFRLA
GGSWYQPKGGTEMFCHPRTGKPLPKYPRIKIPKVGGIFKKPKNKAQREGREPCELDTRE
YVAGAPYTPVEHVVFNPSSRDQLERVLFDELGLPAIGKTEKTGKRSTSAAVLEALREAHP
IVEKILQYRELTKLKSTYIDPLPDLIHPRTGRLHTRFNQTATATGRLSSSNPNLQNIPVRTP
LGQRIRRAFIAEEGWLLVALDYSQIELRVLAHLSGDENLIRAFQEGRDIHTETASWMFGV
PREAVDPLMRRAAKTINFGVLYGMSAHRLSQELAIPYEEAQAFIERYFQSFPKVRAWIEK
TLEEGRRRGYVETLFGRRRYVPDLEARVKSVREAAERMAFNMPVQGTAADLMKLAMV
KLFPRLEEMGARMLLQVHDELVLEAPKERAEAVARLAKEVMEGVYPLAVPLEVEVGIG
EDWLSAKGD.
Seq ID No: 179-TRX-60GS linker-Taq-TBD + E980A V869A, pATG8334
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGGGGSG
GGSGSSGGSGSSGGSSGGGGSGSGSGSSGGSGSSGSGSSGSSGGGGSGGSSRGMLPLFEP
KGRVLLVDGHHLAYRTFHALKGLTTSRGEPVQAVYGFAKSLLKALKEDGDAVIVVEDA
KAPSFRHEAYGGYKAGRAPTPEDFPRQLALIKELVDLLGLARLEVPGYEADDVLASLAK
KAEKEGYEVRILTADKDLYQLLSDRIHVLHPEGYLITPAWLWEKYGLRPDQWADYRAL
TGDESDNLPGVKGIGEKTARKLLEEWGSLEALLKNLDRLKPAIREKILAHMDDLKLSWD
LAKVRTDLPLEVDFAKRREPDRERLRAFLERLEFGSLLHEFGLLESPKALEEAPWPPPEG
AFVGFVLSRKEPMWADLLALAAARGGRVHRAPEPYKALRDLKEARGLLAKDLSVLAL
REGLGLPPGDDPMLLAYLLDPSNTTPEGVARRYGGEWTEEAGERAALSERLFANLWGR
LEGEERLLWLYREVERPLSAVLAHMEATGVRLDVAYLRALSLEVAEEIARLEAEVFRLA
GGSWYQPKGGTEMFCHPRTGKPLPKYPRIKIPKVGGIFKKPKNKAQREGREPCELDTRE
YVAGAPYTPVEHVVFNPSSRDQLERVLFDELGLPAIGKTEKTGKRSTSAAVLEALREAHP
IVEKILQYRELTKLKSTYIDPLPDLIHPRTGRLHTRFNQTATATGRLSSSDPNLQNIPVRTP
LGQRIRRAFIAEEGWLLVALDYSQIELRVLAHLSGDENLIRAFQEGRDIHTETASWMFGV
PREAVDPLMRRAAKTINFGVLYGMSAHRLSQELAIPYEEAQAFIERYFQSFPKVRAWIEK
TLEEGRRRGYVETLFGRRRYVPDLEARVKSVRAAAERMAFNMPVQGTAADLMKLAMV
KLFPRLEEMGARMLLQVHDELVLEAPKERAEAVARLAKEVMEGVYPLAVPLEVEVGIG
EDWLSAKGD.
Seq ID No: 180-TRX-60GS linker-Taq-TBD + D816N H914A, pATG8357
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGGGGSG
GGSGSSGGSGSSGGSSGGGGSGSGSGSSGGSGSSGSGSSGSSGGGGSGGSSRGMLPLFEP
KGRVLLVDGHHLAYRTFHALKGLTTSRGEPVQAVYGFAKSLLKALKEDGDAVIVVEDA
KAPSFRHEAYGGYKAGRAPTPEDFPRQLALIKELVDLLGLARLEVPGYEADDVLASLAK
KAEKEGYEVRILTADKDLYQLLSDRIHVLHPEGYLITPAWLWEKYGLRPDQWADYRAL
TGDESDNLPGVKGIGEKTARKLLEEWGSLEALLKNLDRLKPAIREKILAHMDDLKLSWD
LAKVRTDLPLEVDFAKRREPDRERLRAFLERLEFGSLLHEFGLLESPKALEEAPWPPPEG
AFVGFVLSRKEPMWADLLALAAARGGRVHRAPEPYKALRDLKEARGLLAKDLSVLAL
REGLGLPPGDDPMLLAYLLDPSNTTPEGVARRYGGEWTEEAGERAALSERLFANLWGR
LEGEERLLWLYREVERPLSAVLAHMEATGVRLDVAYLRALSLEVAEEIARLEAEVFRLA
GGSWYQPKGGTEMFCHPRTGKPLPKYPRIKIPKVGGIFKKPKNKAQREGREPCELDTRE
YVAGAPYTPVEHVVFNPSSRDQLERVLFDELGLPAIGKTEKTGKRSTSAAVLEALREAHP
IVEKILQYRELTKLKSTYIDPLPDLIHPRTGRLHTRFNQTATATGRLSSSNPNLQNIPVRTP
LGQRIRRAFIAEEGWLLVALDYSQIELRVLAHLSGDENLIRVFQEGRDIHTETASWMFGV
PREAVDPLMRRAAKTINFGVLYGMSAARLSQELAIPYEEAQAFIERYFQSFPKVRAWIEK
TLEEGRRRGYVETLFGRRRYVPDLEARVKSVREAAERMAFNMPVQGTAADLMKLAMV
KLFPRLEEMGARMLLQVHDELVLEAPKERAEAVARLAKEVMEGVYPLAVPLEVEVGIG
EDWLSAKGD.
Seq ID No: 181-TRX-60GS linker-Taq-TBD + E980A H914A, pATG8358
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGGGGSG
GGSGSSGGSGSSGGSSGGGGSGSGSGSSGGSGSSGSGSSGSSGGGGSGGSSRGMLPLFEP
KGRVLLVDGHHLAYRTFHALKGLTTSRGEPVQAVYGFAKSLLKALKEDGDAVIVVFDA
KAPSFRHEAYGGYKAGRAPTPEDFPRQLALIKELVDLLGLARLEVPGYEADDVLASLAK
KAEKEGYEVRILTADKDLYQLLSDRIHVLHPEGYLITPAWLWEKYGLRPDQWADYRAL
TGDESDNLPGVKGIGEKTARKLLEEWGSLEALLKNLDRLKPAIREKILAHMDDLKLSWD
LAKVRTDLPLEVDFAKRREPDRERLRAFLERLEFGSLLHEFGLLESPKALEEAPWPPPEG
AFVGFVLSRKEPMWADLLALAAARGGRVHRAPEPYKALRDLKEARGLLAKDLSVLAL
REGLGLPPGDDPMLLAYLLDPSNTTPEGVARRYGGEWTEEAGERAALSERLFANLWGR
LEGEERLLWLYREVERPLSAVLAHMEATGVRLDVAYLRALSLEVAEEIARLEAEVFRLA
GGSWYQPKGGTEMFCHPRTGKPLPKYPRIKIPKVGGIFKKPKNKAQREGREPCELDTRE
YVAGAPYTPVEHVVFNPSSRDQLERVLFDELGLPAIGKTEKTGKRSTSAAVLEALREAHP
IVEKILQYRELTKLKSTYIDPLPDLIHPRTGRLHTRFNQTATATGRLSSSDPNLQNIPVRTP
LGQRIRRAFIAEEGWLLVALDYSQIELRVLAHLSGDENLIRVFQEGRDIHTETASWMFGV
PREAVDPLMRRAAKTINFGVLYGMSAARLSQELAIPYEEAQAFIERYFQSFPKVRAWIEK
TLEEGRRRGYVETLFGRRRYVPDLEARVKSVRAAAERMAFNMPVQGTAADLMKLAMV
KLFPRLEEMGARMLLQVHDELVLEAPKERAEAVARLAKEVMEGVYPLAVPLEVEVGIG
EDWLSAKGD.
Seq ID No: 182-TRX-60GS linker-Taq-TBD + V869A H914A, pATG8359
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGGGGSG
GGSGSSGGSGSSGGSSGGGGSGSGSGSSGGSGSSGSGSSGSSGGGGSGGSSRGMLPLFEP
KGRVLLVDGHHLAYRTFHALKGLTTSRGEPVQAVYGFAKSLLKALKEDGDAVIVVFDA
KAPSFRHEAYGGYKAGRAPTPEDFPRQLALIKELVDLLGLARLEVPGYEADDVLASLAK
KAEKEGYEVRILTADKDLYQLLSDRIHVLHPEGYLITPAWLWEKYGLRPDQWADYRAL
TGDESDNLPGVKGIGEKTARKLLEEWGSLEALLKNLDRLKPAIREKILAHMDDLKLSWD
LAKVRTDLPLEVDFAKRREPDRERLRAFLERLEFGSLLHEFGLLESPKALEEAPWPPPEG
AFVGFVLSRKEPMWADLLALAAARGGRVHRAPEPYKALRDLKEARGLLAKDLSVLAL
REGLGLPPGDDPMLLAYLLDPSNTTPEGVARRYGGEWTEEAGERAALSERLFANLWGR
LEGEERLLWLYREVERPLSAVLAHMEATGVRLDVAYLRALSLEVAEEIARLEAEVFRLA
GGSWYQPKGGTEMFCHPRTGKPLPKYPRIKIPKVGGIFKKPKNKAQREGREPCELDTRE
YVAGAPYTPVEHVVFNPSSRDQLERVLFDELGLPAIGKTEKTGKRSTSAAVLEALREAHP
IVEKILQYRELTKLKSTYIDPLPDLIHPRTGRLHTRFNQTATATGRLSSSDPNLQNIPVRTP
LGQRIRRAFIAEEGWLLVALDYSQIELRVLAHLSGDENLIRAFQEGRDIHTETASWMFGV
PREAVDPLMRRAAKTINFGVLYGMSAARLSQELAIPYEEAQAFIERYFQSFPKVRAWIEK
TLEEGRRRGYVETLFGRRRYVPDLEARVKSVREAAERMAFNMPVQGTAADLMKLAMV
KLFPRLEEMGARMLLQVHDELVLEAPKERAEAVARLAKEVMEGVYPLAVPLEVEVGIG
EDWLSAKGD.
Seq ID No: 183-TRX-60GS linker-Taq-TBD + D816N V869A H914A, pATG8360
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGGGGSG
GGSGSSGGSGSSGGSSGGGGSGSGSGSSGGSGSSGSGSSGSSGGGGSGGSSRGMLPLFEP
KGRVLLVDGHHLAYRTFHALKGLTTSRGEPVQAVYGFAKSLLKALKEDGDAVIVVEDA
KAPSFRHEAYGGYKAGRAPTPEDFPRQLALIKELVDLLGLARLEVPGYEADDVLASLAK
KAEKEGYEVRILTADKDLYQLLSDRIHVLHPEGYLITPAWLWEKYGLRPDQWADYRAL
TGDESDNLPGVKGIGEKTARKLLEEWGSLEALLKNLDRLKPAIREKILAHMDDLKLSWD
LAKVRTDLPLEVDFAKRREPDRERLRAFLERLEFGSLLHEFGLLESPKALEEAPWPPPEG
AFVGFVLSRKEPMWADLLALAAARGGRVHRAPEPYKALRDLKEARGLLAKDLSVLAL
REGLGLPPGDDPMLLAYLLDPSNTTPEGVARRYGGEWTEEAGERAALSERLFANLWGR
LEGEERLLWLYREVERPLSAVLAHMEATGVRLDVAYLRALSLEVAEEIARLEAEVFRLA
GGSWYQPKGGTEMFCHPRTGKPLPKYPRIKIPKVGGIFKKPKNKAQREGREPCELDTRE
YVAGAPYTPVEHVVFNPSSRDQLERVLFDELGLPAIGKTEKTGKRSTSAAVLEALREAHP
IVEKILQYRELTKLKSTYIDPLPDLIHPRTGRLHTRFNQTATATGRLSSSNPNLQNIPVRTP
LGQRIRRAFIAEEGWLLVALDYSQIELRVLAHLSGDENLIRAFQEGRDIHTETASWMFGV
PREAVDPLMRRAAKTINFGVLYGMSAARLSQELAIPYEEAQAFIERYFQSFPKVRAWIEK
TLEEGRRRGYVETLFGRRRYVPDLEARVKSVREAAERMAFNMPVQGTAADLMKLAMV
KLFPRLEEMGARMLLQVHDELVLEAPKERAEAVARLAKEVMEGVYPLAVPLEVEVGIG
EDWLSAKGD.
Seq ID No: 184-TRX-60GS linker-Taq-TBD + D816N E980A H914A, pATG8361
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGGGGSG
GGSGSSGGSGSSGGSSGGGGSGSGSGSSGGSGSSGSGSSGSSGGGGSGGSSRGMLPLFEP
KGRVLLVDGHHLAYRTFHALKGLTTSRGEPVQAVYGFAKSLLKALKEDGDAVIVVEDA
KAPSFRHEAYGGYKAGRAPTPEDFPRQLALIKELVDLLGLARLEVPGYEADDVLASLAK
KAEKEGYEVRILTADKDLYQLLSDRIHVLHPEGYLITPAWLWEKYGLRPDQWADYRAL
TGDESDNLPGVKGIGEKTARKLLEEWGSLEALLKNLDRLKPAIREKILAHMDDLKLSWD
LAKVRTDLPLEVDFAKRREPDRERLRAFLERLEFGSLLHEFGLLESPKALEEAPWPPPEG
AFVGFVLSRKEPMWADLLALAAARGGRVHRAPEPYKALRDLKEARGLLAKDLSVLAL
REGLGLPPGDDPMLLAYLLDPSNTTPEGVARRYGGEWTEEAGERAALSERLFANLWGR
LEGEERLLWLYREVERPLSAVLAHMEATGVRLDVAYLRALSLEVAEEIARLEAEVFRLA
GGSWYQPKGGTEMFCHPRTGKPLPKYPRIKIPKVGGIFKKPKNKAQREGREPCELDTRE
YVAGAPYTPVEHVVFNPSSRDQLERVLFDELGLPAIGKTEKTGKRSTSAAVLEALREAHP
IVEKILQYRELTKLKSTYIDPLPDLIHPRTGRLHTRFNQTATATGRLSSSNPNLQNIPVRTP
LGQRIRRAFIAEEGWLLVALDYSQIELRVLAHLSGDENLIRVFQEGRDIHTETASWMFGV
PREAVDPLMRRAAKTINFGVLYGMSAARLSQELAIPYEEAQAFIERYFQSFPKVRAWIEK
TLEEGRRRGYVETLFGRRRYVPDLEARVKSVRAAAERMAFNMPVQGTAADLMKLAMV
KLFPRLEEMGARMLLQVHDELVLEAPKERAEAVARLAKEVMEGVYPLAVPLEVEVGIG
EDWLSAKGD.
Seq ID No: 185-TRX-60GS linker-Taq-TBD + D816N E980A V869A H914A, pATG8362
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGGGGSG
GGSGSSGGSGSSGGSSGGGGSGSGSGSSGGSGSSGSGSSGSSGGGGSGGSSRGMLPLFEP
KGRVLLVDGHHLAYRTFHALKGLTTSRGEPVQAVYGFAKSLLKALKEDGDAVIVVEDA
KAPSFRHEAYGGYKAGRAPTPEDFPRQLALIKELVDLLGLARLEVPGYEADDVLASLAK
KAEKEGYEVRILTADKDLYQLLSDRIHVLHPEGYLITPAWLWEKYGLRPDQWADYRAL
TGDESDNLPGVKGIGEKTARKLLEEWGSLEALLKNLDRLKPAIREKILAHMDDLKLSWD
LAKVRTDLPLEVDFAKRREPDRERLRAFLERLEFGSLLHEFGLLESPKALEEAPWPPPEG
AFVGFVLSRKEPMWADLLALAAARGGRVHRAPEPYKALRDLKEARGLLAKDLSVLAL
REGLGLPPGDDPMLLAYLLDPSNTTPEGVARRYGGEWTEEAGERAALSERLFANLWGR
LEGEERLLWLYREVERPLSAVLAHMEATGVRLDVAYLRALSLEVAEEIARLEAEVFRLA
GGSWYQPKGGTEMFCHPRTGKPLPKYPRIKIPKVGGIFKKPKNKAQREGREPCELDTRE
YVAGAPYTPVEHVVFNPSSRDQLERVLFDELGLPAIGKTEKTGKRSTSAAVLEALREAHP
IVEKILQYRELTKLKSTYIDPLPDLIHPRTGRLHTRFNQTATATGRLSSSNPNLQNIPVRTP
LGQRIRRAFIAEEGWLLVALDYSQIELRVLAHLSGDENLIRAFQEGRDIHTETASWMFGV
PREAVDPLMRRAAKTINFGVLYGMSAARLSQELAIPYEEAQAFIERYFQSFPKVRAWIEK
TLEEGRRRGYVETLFGRRRYVPDLEARVKSVRAAAERMAFNMPVQGTAADLMKLAMV
KLFPRLEEMGARMLLQVHDELVLEAPKERAEAVARLAKEVMEGVYPLAVPLEVEVGIG
EDWLSAKGD.
Seq ID No: 186-TRX-60GS linker-Taq-TBD + E980A V869A H914A, pATG8363
MSDKIIHLTDDSFDTDVLKADGAILVDFWAEWCGPCKMIAPILDEIADEYQGKLTVAKL
NIDQNPGTAPKYGIRGIPTLLLFKNGEVAATKVGALSKGQLKEFLDANLAGSSGGGGSG
GGSGSSGGSGSSGGSSGGGGSGSGSGSSGGSGSSGSGSSGSSGGGGSGGSSRGMLPLFEP
KGRVLLVDGHHLAYRTFHALKGLTTSRGEPVQAVYGFAKSLLKALKEDGDAVIVVFDA
KAPSFRHEAYGGYKAGRAPTPEDFPRQLALIKELVDLLGLARLEVPGYEADDVLASLAK
KAEKEGYEVRILTADKDLYQLLSDRIHVLHPEGYLITPAWLWEKYGLRPDQWADYRAL
TGDESDNLPGVKGIGEKTARKLLEEWGSLEALLKNLDRLKPAIREKILAHMDDLKLSWD
LAKVRTDLPLEVDFAKRREPDRERLRAFLERLEFGSLLHEFGLLESPKALEEAPWPPPEG
AFVGFVLSRKEPMWADLLALAAARGGRVHRAPEPYKALRDLKEARGLLAKDLSVLAL
REGLGLPPGDDPMLLAYLLDPSNTTPEGVARRYGGEWTEEAGERAALSERLFANLWGR
LEGEERLLWLYREVERPLSAVLAHMEATGVRLDVAYLRALSLEVAEEIARLEAEVFRLA
GGSWYQPKGGTEMFCHPRTGKPLPKYPRIKIPKVGGIFKKPKNKAQREGREPCELDTRE
YVAGAPYTPVEHVVFNPSSRDQLERVLFDELGLPAIGKTEKTGKRSTSAAVLEALREAHP
IVEKILQYRELTKLKSTYIDPLPDLIHPRTGRLHTRFNQTATATGRLSSSDPNLQNIPVRTP
LGQRIRRAFIAEEGWLLVALDYSQIELRVLAHLSGDENLIRAFQEGRDIHTETASWMFGV
PREAVDPLMRRAAKTINFGVLYGMSAARLSQELAIPYEEAQAFIERYFQSFPKVRAWIEK
TLEEGRRRGYVETLFGRRRYVPDLEARVKSVRAAAERMAFNMPVQGTAADLMKLAMV
KLFPRLEEMGARMLLQVHDELVLEAPKERAEAVARLAKEVMEGVYPLAVPLEVEVGIG
EDWLSAKGD.
Seq ID No: 187-TRX E31P- 18GS(EAAAK repeat)18GS linker-Taq TBD [537V 555N]
+H936A V891A, pATG8320
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGAEAAAKEAAAKEAAAKEAAAKALEAEAAAKEAAAKEAAAKEAAAKASG
SGSSGSSGGGGSGGSSRGMLPLFEPKGRVLLVDGHHLAYRTFHALKGLTTSRGEPVQAV
YGFAKSLLKALKEDGDAVIVVFDAKAPSFRHEAYGGYKAGRAPTPEDFPRQLALIKELV
DLLGLARLEVPGYEADDVLASLAKKAEKEGYEVRILTADKDLYQLLSDRIHVLHPEGYL
ITPAWLWEKYGLRPDQWADYRALTGDESDNLPGVKGIGEKTARKLLEEWGSLEALLKN
LDRLKPAIREKILAHMDDLKLSWDLAKVRTDLPLEVDFAKRREPDRERLRAFLERLEFGS
LLHEFGLLESPKALEEAPWPPPEGAFVGFVLSRKEPMWADLLALAAARGGRVHRAPEPY
KALRDLKEARGLLAKDLSVLALREGLGLPPGDDPMLLAYLLDPSNTTPEGVARRYGGE
WTEEAGERAALSERLFANLWGRLEGEERLLWLYREVERPLSAVLAHMEATGVRLDVA
YLRALSLEVAEEIARLEAEVFRLAGGSWYQPKGGTEMFCHPRTGKPLPKYPRIKIPKVGG
IFKKPKNKAQREGREPCELDTRVYVAGAPYTPVEHVVFNPNSRDQLERVLFDELGLPAIG
KTEKTGKRSTSAAVLEALREAHPIVEKILQYRELTKLKSTYIDPLPDLIHPRTGRLHTRFN
QTATATGRLSSSDPNLQNIPVRTPLGQRIRRAFIAEEGWLLVALDYSQIELRVLAHLSGDE
NLIRAFQEGRDIHTETASWMFGVPREAVDPLMRRAAKTINFGVLYGMSAARLSQELAIP
YEEAQAFIERYFQSFPKVRAWIEKTLEEGRRRGYVETLFGRRRYVPDLEARVKSVREAA
ERMAFNMPVQGTAADLMKLAMVKLFPRLEEMGARMLLQVHDELVLEAPKERAEAVA
RLAKEVMEGVYPLAVPLEVEVGIGEDWLSAKGD
Seq ID No: 188-TRX E31P-18GS(EAAAK repeat)18GS linker-Taq TBD [537V 555N]
+H936A D838N, pATG8321
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGAEAAAKEAAAKEAAAKEAAAKALEAEAAAKEAAAKEAAAKEAAAKASG
SGSSGSSGGGGSGGSSRGMLPLFEPKGRVLLVDGHHLAYRTFHALKGLTTSRGEPVQAV
YGFAKSLLKALKEDGDAVIVVFDAKAPSFRHEAYGGYKAGRAPTPEDFPRQLALIKELV
DLLGLARLEVPGYEADDVLASLAKKAEKEGYEVRILTADKDLYQLLSDRIHVLHPEGYL
ITPAWLWEKYGLRPDQWADYRALTGDESDNLPGVKGIGEKTARKLLEEWGSLEALLKN
LDRLKPAIREKILAHMDDLKLSWDLAKVRTDLPLEVDFAKRREPDRERLRAFLERLEFGS
LLHEFGLLESPKALEEAPWPPPEGAFVGFVLSRKEPMWADLLALAAARGGRVHRAPEPY
KALRDLKEARGLLAKDLSVLALREGLGLPPGDDPMLLAYLLDPSNTTPEGVARRYGGE
WTEEAGERAALSERLFANLWGRLEGEERLLWLYREVERPLSAVLAHMEATGVRLDVA
YLRALSLEVAEEIARLEAEVFRLAGGSWYQPKGGTEMFCHPRTGKPLPKYPRIKIPKVGG
IFKKPKNKAQREGREPCELDTRVYVAGAPYTPVEHVVFNPNSRDQLERVLFDELGLPAIG
KTEKTGKRSTSAAVLEALREAHPIVEKILQYRELTKLKSTYIDPLPDLIHPRTGRLHTRFN
QTATATGRLSSSNPNLQNIPVRTPLGQRIRRAFIAEEGWLLVALDYSQIELRVLAHLSGDE
NLIRVFQEGRDIHTETASWMFGVPREAVDPLMRRAAKTINFGVLYGMSAARLSQELAIP
YEEAQAFIERYFQSFPKVRAWIEKTLEEGRRRGYVETLFGRRRYVPDLEARVKSVREAA
ERMAFNMPVQGTAADLMKLAMVKLFPRLEEMGARMLLQVHDELVLEAPKERAEAVA
RLAKEVMEGVYPLAVPLEVEVGIGEDWLSAKGD
Seq ID No: 189-TRX E31P-18GS(EAAAK repeat)18GS linker-Taq TBD [537V 555N]
+H914A E1002A, pATG8322
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGAEAAAKEAAAKEAAAKEAAAKALEAEAAAKEAAAKEAAAKEAAAKASG
SGSSGSSGGGGSGGSSRGMLPLFEPKGRVLLVDGHHLAYRTFHALKGLTTSRGEPVQAV
YGFAKSLLKALKEDGDAVIVVFDAKAPSFRHEAYGGYKAGRAPTPEDFPRQLALIKELV
DLLGLARLEVPGYEADDVLASLAKKAEKEGYEVRILTADKDLYQLLSDRIHVLHPEGYL
ITPAWLWEKYGLRPDQWADYRALTGDESDNLPGVKGIGEKTARKLLEEWGSLEALLKN
LDRLKPAIREKILAHMDDLKLSWDLAKVRTDLPLEVDFAKRREPDRERLRAFLERLEFGS
LLHEFGLLESPKALEEAPWPPPEGAFVGFVLSRKEPMWADLLALAAARGGRVHRAPEPY
KALRDLKEARGLLAKDLSVLALREGLGLPPGDDPMLLAYLLDPSNTTPEGVARRYGGE
WTEEAGERAALSERLFANLWGRLEGEERLLWLYREVERPLSAVLAHMEATGVRLDVA
YLRALSLEVAEEIARLEAEVFRLAGGSWYQPKGGTEMFCHPRTGKPLPKYPRIKIPKVGG
IFKKPKNKAQREGREPCELDTRVYVAGAPYTPVEHVVFNPNSRDQLERVLFDELGLPAIG
KTEKTGKRSTSAAVLEALREAHPIVEKILQYRELTKLKSTYIDPLPDLIHPRTGRLHTRFN
QTATATGRLSSSDPNLQNIPVRTPLGQRIRRAFIAEEGWLLVALDYSQIELRVLAHLSGDE
NLIRVFQEGRDIHTETASWMFGVPREAVDPLMRRAAKTINFGVLYGMSAARLSQELAIP
YEEAQAFIERYFQSFPKVRAWIEKTLEEGRRRGYVETLFGRRRYVPDLEARVKSVRAAA
ERMAFNMPVQGTAADLMKLAMVKLFPRLEEMGARMLLQVHDELVLEAPKERAEAVA
RLAKEVMEGVYPLAVPLEVEVGIGEDWLSAKGD
Seq ID No: 190-TRX E31P-18GS(EAAAK repeat)18GS linker-Taq TBD [537V 555N]
+H936A D838N E1002A, pATG8329
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGAEAAAKEAAAKEAAAKEAAAKALEAEAAAKEAAAKEAAAKEAAAKASG
SGSSGSSGGGGSGGSSRGMLPLFEPKGRVLLVDGHHLAYRTFHALKGLTTSRGEPVQAV
YGFAKSLLKALKEDGDAVIVVFDAKAPSFRHEAYGGYKAGRAPTPEDFPRQLALIKELV
DLLGLARLEVPGYEADDVLASLAKKAEKEGYEVRILTADKDLYQLLSDRIHVLHPEGYL
ITPAWLWEKYGLRPDQWADYRALTGDESDNLPGVKGIGEKTARKLLEEWGSLEALLKN
LDRLKPAIREKILAHMDDLKLSWDLAKVRTDLPLEVDFAKRREPDRERLRAFLERLEFGS
LLHEFGLLESPKALEEAPWPPPEGAFVGFVLSRKEPMWADLLALAAARGGRVHRAPEPY
KALRDLKEARGLLAKDLSVLALREGLGLPPGDDPMLLAYLLDPSNTTPEGVARRYGGE
WTEEAGERAALSERLFANLWGRLEGEERLLWLYREVERPLSAVLAHMEATGVRLDVA
YLRALSLEVAEEIARLEAEVFRLAGGSWYQPKGGTEMFCHPRTGKPLPKYPRIKIPKVGG
IFKKPKNKAQREGREPCELDTRVYVAGAPYTPVEHVVFNPNSRDQLERVLFDELGLPAIG
KTEKTGKRSTSAAVLEALREAHPIVEKILQYRELTKLKSTYIDPLPDLIHPRTGRLHTRFN
QTATATGRLSSSNPNLQNIPVRTPLGQRIRRAFIAEEGWLLVALDYSQIELRVLAHLSGDE
NLIRVFQEGRDIHTETASWMFGVPREAVDPLMRRAAKTINFGVLYGMSAARLSQELAIP
YEEAQAFIERYFQSFPKVRAWIEKTLEEGRRRGYVETLFGRRRYVPDLEARVKSVRAAA
ERMAFNMPVQGTAADLMKLAMVKLFPRLEEMGARMLLQVHDELVLEAPKERAEAVA
RLAKEVMEGVYPLAVPLEVEVGIGEDWLSAKGD
Seq ID No: 191-TRX E31P-18GS(EAAAK repeat)18GS linker-Taq TBD [537V 555N]
+H936A D838N V891A, pATG8330
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGAEAAAKEAAAKEAAAKEAAAKALEAEAAAKEAAAKEAAAKEAAAKASG
SGSSGSSGGGGSGGSSRGMLPLFEPKGRVLLVDGHHLAYRTFHALKGLTTSRGEPVQAV
YGFAKSLLKALKEDGDAVIVVFDAKAPSFRHEAYGGYKAGRAPTPEDFPRQLALIKELV
DLLGLARLEVPGYEADDVLASLAKKAEKEGYEVRILTADKDLYQLLSDRIHVLHPEGYL
ITPAWLWEKYGLRPDQWADYRALTGDESDNLPGVKGIGEKTARKLLEEWGSLEALLKN
LDRLKPAIREKILAHMDDLKLSWDLAKVRTDLPLEVDFAKRREPDRERLRAFLERLEFGS
LLHEFGLLESPKALEEAPWPPPEGAFVGFVLSRKEPMWADLLALAAARGGRVHRAPEPY
KALRDLKEARGLLAKDLSVLALREGLGLPPGDDPMLLAYLLDPSNTTPEGVARRYGGE
WTEEAGERAALSERLFANLWGRLEGEERLLWLYREVERPLSAVLAHMEATGVRLDVA
YLRALSLEVAEEIARLEAEVFRLAGGSWYQPKGGTEMFCHPRTGKPLPKYPRIKIPKVGG
IFKKPKNKAQREGREPCELDTRVYVAGAPYTPVEHVVFNPNSRDQLERVLFDELGLPAIG
KTEKTGKRSTSAAVLEALREAHPIVEKILQYRELTKLKSTYIDPLPDLIHPRTGRLHTRFN
QTATATGRLSSSNPNLQNIPVRTPLGQRIRRAFIAEEGWLLVALDYSQIELRVLAHLSGDE
NLIRAFQEGRDIHTETASWMFGVPREAVDPLMRRAAKTINFGVLYGMSAARLSQELAIP
YEEAQAFIERYFQSFPKVRAWIEKTLEEGRRRGYVETLFGRRRYVPDLEARVKSVREAA
ERMAFNMPVQGTAADLMKLAMVKLFPRLEEMGARMLLQVHDELVLEAPKERAEAVA
RLAKEVMEGVYPLAVPLEVEVGIGEDWLSAKGD
Seq ID No: 192-TRX E31P-18GS(EAAAK repeat)18GS linker-Taq TBD [537V 555N]
+H936A E1002A V891A, pATG8331
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGAEAAAKEAAAKEAAAKEAAAKALEAEAAAKEAAAKEAAAKEAAAKASG
SGSSGSSGGGGSGGSSRGMLPLFEPKGRVLLVDGHHLAYRTFHALKGLTTSRGEPVQAV
YGFAKSLLKALKEDGDAVIVVFDAKAPSFRHEAYGGYKAGRAPTPEDFPRQLALIKELV
DLLGLARLEVPGYEADDVLASLAKKAEKEGYEVRILTADKDLYQLLSDRIHVLHPEGYL
ITPAWLWEKYGLRPDQWADYRALTGDESDNLPGVKGIGEKTARKLLEEWGSLEALLKN
LDRLKPAIREKILAHMDDLKLSWDLAKVRTDLPLEVDFAKRREPDRERLRAFLERLEFGS
LLHEFGLLESPKALEEAPWPPPEGAFVGFVLSRKEPMWADLLALAAARGGRVHRAPEPY
KALRDLKEARGLLAKDLSVLALREGLGLPPGDDPMLLAYLLDPSNTTPEGVARRYGGE
WTEEAGERAALSERLFANLWGRLEGEERLLWLYREVERPLSAVLAHMEATGVRLDVA
YLRALSLEVAEEIARLEAEVFRLAGGSWYQPKGGTEMFCHPRTGKPLPKYPRIKIPKVGG
IFKKPKNKAQREGREPCELDTRVYVAGAPYTPVEHVVFNPNSRDQLERVLFDELGLPAIG
KTEKTGKRSTSAAVLEALREAHPIVEKILQYRELTKLKSTYIDPLPDLIHPRTGRLHTRFN
QTATATGRLSSSDPNLQNIPVRTPLGQRIRRAFIAEEGWLLVALDYSQIELRVLAHLSGDE
NLIRAFQEGRDIHTETASWMFGVPREAVDPLMRRAAKTINFGVLYGMSAARLSQELAIP
YEEAQAFIERYFQSFPKVRAWIEKTLEEGRRRGYVETLFGRRRYVPDLEARVKSVRAAA
ERMAFNMPVQGTAADLMKLAMVKLFPRLEEMGARMLLQVHDELVLEAPKERAEAVA
RLAKEVMEGVYPLAVPLEVEVGIGEDWLSAKGD
Seq ID No: 193-TRX E31P-18GS(EAAAK repeat)18GS linker-Taq TBD [537V 555N]
+H936A D838N E1002A V891A, pATG8339
MSDKIIHLTDDSFDTDVLKADGAILVDFWAPWCGPCKMIAPILDEIADEYQGKLTVAKL
NIDQNPGTAPKYGIRGIPTLLLFKNGEVAATKVGALSKGQLKEFLDANLAGSSGSSGSSG
GGSGGGSGAEAAAKEAAAKEAAAKEAAAKALEAEAAAKEAAAKEAAAKEAAAKASG
SGSSGSSGGGGSGGSSRGMLPLFEPKGRVLLVDGHHLAYRTFHALKGLTTSRGEPVQAV
YGFAKSLLKALKEDGDAVIVVFDAKAPSFRHEAYGGYKAGRAPTPEDFPRQLALIKELV
DLLGLARLEVPGYEADDVLASLAKKAEKEGYEVRILTADKDLYQLLSDRIHVLHPEGYL
ITPAWLWEKYGLRPDQWADYRALTGDESDNLPGVKGIGEKTARKLLEEWGSLEALLKN
LDRLKPAIREKILAHMDDLKLSWDLAKVRTDLPLEVDFAKRREPDRERLRAFLERLEFGS
LLHEFGLLESPKALEEAPWPPPEGAFVGFVLSRKEPMWADLLALAAARGGRVHRAPEPY
KALRDLKEARGLLAKDLSVLALREGLGLPPGDDPMLLAYLLDPSNTTPEGVARRYGGE
WTEEAGERAALSERLFANLWGRLEGEERLLWLYREVERPLSAVLAHMEATGVRLDVA
YLRALSLEVAEEIARLEAEVFRLAGGSWYQPKGGTEMFCHPRTGKPLPKYPRIKIPKVGG
IFKKPKNKAQREGREPCELDTRVYVAGAPYTPVEHVVFNPNSRDQLERVLFDELGLPAIG
KTEKTGKRSTSAAVLEALREAHPIVEKILQYRELTKLKSTYIDPLPDLIHPRTGRLHTRFN
QTATATGRLSSSNPNLQNIPVRTPLGQRIRRAFIAEEGWLLVALDYSQIELRVLAHLSGDE
NLIRAFQEGRDIHTETASWMFGVPREAVDPLMRRAAKTINFGVLYGMSAARLSQELAIP
YEEAQAFIERYFQSFPKVRAWIEKTLEEGRRRGYVETLFGRRRYVPDLEARVKSVRAAA
ERMAFNMPVQGTAADLMKLAMVKLFPRLEEMGARMLLQVHDELVLEAPKERAEAVA
RLAKEVMEGVYPLAVPLEVEVGIGEDWLSAKGD
Seq ID No: 194-Tne polymerase (v1)-Aeromonas TBD, pATG8302
MARLFLFDGTALAYRAYYALDRSLSTSTGIPTNAVYGVARMLVKFIKEHIIPEKDYAAV
AFDKKAATFRHKLLEAYKAQRPKTPDLLVQQLPYIKRLIEALGFKVLELEGYEADDIIAT
LAVKGCTFFDEIFIITGDKDMLQLVNEKIKVWRIVKGISDLELYDSKKVKERYGVEPHQIP
DLLALTGDEIDNIPGVTGIGEKTAVQLLGKYRNLEDILEHARELPQRVRKALLRDREVAI
LSKKLATLVTNAPVEVDWEEMKYRGYDKRKLLPILKELEFASIMKELQLYEEAEPTGYEI
VKDHKTFEDLIEKLKEVPSFALDLETSSLDPFNCEIVGISVSFKPKTAYYIPLHHRNAQNL
DETLVLSKLKEILEDPSSKIVGQNLKYDYKVLMVKGISPVYPHFDTMIAAYLLEPNEKKF
NLEDLSLKFLGYKMTSYQELMSFSSPLFGFSFADVPVDKAANYSCEDADITYRLYKILSM
KLHEAELENVFYRIEMPLVNVLARMELNGVYVDTEFLKKLSEEYGKKLEELAEKIYQIA
GSSWYRPKGGKAFFRHPVTGKDLTNYPRVIYPKAGEIYTKGGKLAKTLYCKDRPFTPIE
YTVFNPGSPKQVSKILFEKLGIKPRGKTTKTGEYSTRIEVLEEIANEHEIVPLILEYRKIQKL
KSTYIDTLPKLVNPKTGRIHASFHQTGTATGRLSSSDPNLQNLPTKSEEGKEIRKAIVPQD
PDWWIVSADYSQIELRILAHLSGDENLVKAFEEGIDVHTLTASRIYNVKPEEVNEEMRRV
GKMVNFSIIYGVTPYGLSVRLGIPVKEAEKMIISYFTLYPKVRSYIQQVVAEAKEKGYVR
TLFGRKRDIPQLMARDKNTQSEGERIAINTPIQGTAADIIKLAMIDIDEELRKRNMKSRMII
QVHDELVFEVPDEEKEELVDLVKNKMTNVVKLSVPLEVDISIGKSWS
Seq ID No: 195-Tne polymerase (v1)-Klebsiella TBD, pATG8353
MARLFLFDGTALAYRAYYALDRSLSTSTGIPTNAVYGVARMLVKFIKEHIIPEKDYAAV
AFDKKAATFRHKLLEAYKAQRPKTPDLLVQQLPYIKRLIEALGFKVLELEGYEADDIIAT
LAVKGCTFFDEIFIITGDKDMLQLVNEKIKVWRIVKGISDLELYDSKKVKERYGVEPHQIP
DLLALTGDEIDNIPGVTGIGEKTAVQLLGKYRNLEDILEHARELPQRVRKALLRDREVAI
LSKKLATLVTNAPVEVDWEEMKYRGYDKRKLLPILKELEFASIMKELQLYEEAEPTGYEI
VKDHKTFEDLIEKLKEVPSFALDLETSSLDPFNCEIVGISVSFKPKTAYYIPLHHRNAQNL
DETLVLSKLKEILEDPSSKIVGQNLKYDYKVLMVKGISPVYPHFDTMIAAYLLEPNEKKF
NLEDLSLKFLGYKMTSYQELMSFSSPLFGFSFADVPVDKAANYSCEDADITYRLYKILSM
KLHEAELENVFYRIEMPLVNVLARMELNGVYVDTEFLKKLSEEYGKKLEELAEKIYQIA
GGTWYQPKGGTELFLHPRTGKPLGKYPRVKYPKQGGIYKKPKNKAQREGREPCDLDTR
DYVEGAPYTPVEHVVFNPSSPKQVSKILFEKLGIKPRGKTTKTGEYSTRIEVLEEIANEHEI
VPLILEYRKIQKLKSTYIDTLPKLVNPKTGRIHASFHQTGTATGRLSSSDPNLQNLPTKSEE
GKEIRKAIVPQDPDWWIVSADYSQIELRILAHLSGDENLVKAFEEGIDVHTLTASRIYNVK
PEEVNEEMRRVGKMVNFSIIYGVTPYGLSVRLGIPVKEAEKMIISYFTLYPKVRSYIQQVV
AEAKEKGYVRTLFGRKRDIPQLMARDKNTQSEGERIAINTPIQGTAADIIKLAMIDIDEEL
RKRNMKSRMIIQVHDELVFEVPDEEKEELVDLVKNKMTNVVKLSVPLEVDISIGKSWS.
Seq ID No: 196-Tne polymerase (v1)-Salmonella TBD, pATG8355
MARLFLFDGTALAYRAYYALDRSLSTSTGIPTNAVYGVARMLVKFIKEHIIPEKDYAAV
AFDKKAATFRHKLLEAYKAQRPKTPDLLVQQLPYIKRLIEALGFKVLELEGYEADDIIAT
LAVKGCTFFDEIFIITGDKDMLQLVNEKIKVWRIVKGISDLELYDSKKVKERYGVEPHQIP
DLLALTGDEIDNIPGVTGIGEKTAVQLLGKYRNLEDILEHARELPQRVRKALLRDREVAI
LSKKLATLVTNAPVEVDWEEMKYRGYDKRKLLPILKELEFASIMKELQLYEEAEPTGYEI
VKDHKTFEDLIEKLKEVPSFALDLETSSLDPFNCEIVGISVSFKPKTAYYIPLHHRNAQNL
DETLVLSKLKEILEDPSSKIVGQNLKYDYKVLMVKGISPVYPHFDTMIAAYLLEPNEKKF
NLEDLSLKFLGYKMTSYQELMSFSSPLFGFSFADVPVDKAANYSCEDADITYRLYKILSM
KLHEAELENVFYRIEMPLVNVLARMELNGVYVDTEFLKKLSEEYGKKLEELAEKIYQIA
GGSWYAPKGGKEFFRHPRTGKDLPKYPRVVYPKVGGIFKKPKNKAQRLGLEPCERDTR
DTMEGAPFTPITYVEFNPGSPKQVSKILFEKLGIKPRGKTTKTGEYSTRIEVLEEIANEHEI
VPLILEYRKIQKLKSTYIDTLPKLVNPKTGRIHASFHQTGTATGRLSSSDPNLQNLPTKSEE
GKEIRKAIVPQDPDWWIVSADYSQIELRILAHLSGDENLVKAFEEGIDVHTLTASRIYNVK
PEEVNEEMRRVGKMVNFSIIYGVTPYGLSVRLGIPVKEAEKMIISYFTLYPKVRSYIQQVV
AEAKEKGYVRTLFGRKRDIPQLMARDKNTQSEGERIAINTPIQGTAADIIKLAMIDIDEEL
RKRNMKSRMIIQVHDELVFEVPDEEKEELVDLVKNKMTNVVKLSVPLEVDISIGKSWS.
Seq ID No: 197-Tne polymerase (v2)-Klebsiella TBD, pATG8352
MKELQLYEEAEPTGYEIVKDHKTFEDLIEKLKEVPSFALALETSSLDPFNCEIVGISVSFKP
KTAYYIPLHHRNAQNLDETLVLSKLKEILEDPSSKIVGQNLKYAYKVLMVKGISPVYPHF
DTMIAAYLLEPNEKKFNLEDLSLKFLGYKMTSYQELMSFSSPLFGFSFADVPVDKAANY
SCEDADITYRLYKILSMKLHEAELENVFYRIEMPLVNVLARMELNGVYVDTEFLKKLSE
EYGKKLEELAEKIYQIAGGTWYQPKGGTELFLHPRTGKPLGKYPRVKYPKQGGIYKKPK
NKAQREGREPCDLDTRDYVEGAPYTPVEHVVFNPSSPKQVSKILFEKLGIKPRGKTTKTG
AYSTRIEVLEEIANEHEIVPLILEYRKIQKLKSTYIDTLPKLVNPKTGRIHASFHQTGTATG
RLSSSDPNLQNLPTKSEEGKEIRKAIVPQDPDWWIVSADYSQIELRILAHLSGDENLVKAF
EEGIDVHTLTASRIYNVKPEEVNEEMRRVGKMVNYSIIYGVTPYGLSVRLGIPVKEAEKM
IISYFTLYPKVRSYIQQVVAEAKEKGYVRTLFGRKRDIPQLMARDKNTQSEGERIAINTPI
QGTAADIIKLAMIDIDEELRKRNMKSRMIIQVHDELVFEVPDEEKEELVDLVKNKMTNV
VKLSVPLEVDISIGKSWS.
Seq ID No: 198-Tne polymerase (v2)-Salmonella TBD, pATG8354
MKELQLYEEAEPTGYEIVKDHKTFEDLIEKLKEVPSFALALETSSLDPFNCEIVGISVSFKP
KTAYYIPLHHRNAQNLDETLVLSKLKEILEDPSSKIVGQNLKYAYKVLMVKGISPVYPHF
DTMIAAYLLEPNEKKFNLEDLSLKFLGYKMTSYQELMSFSSPLFGFSFADVPVDKAANY
SCEDADITYRLYKILSMKLHEAELENVFYRIEMPLVNVLARMELNGVYVDTEFLKKLSE
EYGKKLEELAEKIYQIAGGSWYAPKGGKEFFRHPRTGKDLPKYPRVVYPKVGGIFKKPK
NKAQRLGLEPCERDTRDTMEGAPFTPITYVEFNPGSPKQVSKILFEKLGIKPRGKTTKTG
AYSTRIEVLEEIANEHEIVPLILEYRKIQKLKSTYIDTLPKLVNPKTGRIHASFHQTGTATG
RLSSSDPNLQNLPTKSEEGKEIRKAIVPQDPDWWIVSADYSQIELRILAHLSGDENLVKAF
EEGIDVHTLTASRIYNVKPEEVNEEMRRVGKMVNYSIIYGVTPYGLSVRLGIPVKEAEKM
IISYFTLYPKVRSYIQQVVAEAKEKGYVRTLFGRKRDIPQLMARDKNTQSEGERIAINTPI
QGTAADIIKLAMIDIDEELRKRNMKSRMIIQVHDELVFEVPDEEKEELVDLVKNKMTNV
VKLSVPLEVDISIGKSWS.
SEQ ID NO: 199-Reducing-agent-free thioredoxin binding domain (RAF-TBD)
GSWYQPKGGTEMFX₁HPRTGKPLPKYPRIKIPKVGGIFKKPKNKAQREGREPX₂ELDTRE
YVAGAPYX₃PVEHVVENPS;
wherein X₁ is leucine (14);
wherein X₂ is valine (52); and
wherein X₃ is threonine, valine, or cysteine (66)
SEQ ID NO: 200-Reducing-agent-free thioredoxin (RAF-TRX)
SDKIIHLTDDSFDTDVLKADGAILVDFWAEWX₁GPX₂KMIAPILDEIADEYQGKLTVAKL
NIDQNPGTAPKYGIRGIPTLLLFKNGEVAATKVGALSKGQLKEFLDANLA;
wherein X₁ is threonine, valine, or cysteine; (32) and
wherein X₂ is not cysteine (35)
Seq. ID No: 201-TRX-linker-Taq-TBD [C36S, C683L, C721V relative to pATG8100]
pATG8416
MSDKIIHLTDDSFDTDVLKADGAILVDFWAPWCGPSKMIAPILDEIADEYQGKLTVAKLN
IDQNPGTAPKYGIRGIPTLLLFKNGEVAATKVGALSKGQLKEFLDANLAGSSGSSGSSGG
GSGGGSGSSGSSGSSGGGSGGGSGSSGGSGSSGGSSGGGGSGSGSGSSGGSGSSGSGSSG
SSGGGGSGGSSRGMLPLFEPKGRVLLVDGHHLAYRTFHALKGLTTSRGEPVQAVYGFA
KSLLKALKEDGDAVIVVFDAKAPSFRHEAYGGYKAGRAPTPEDFPRQLALIKELVDLLG
LARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFLHPRTGKPLPKYPRIKIPKVGGIFK
KPKNKAQREGREPVELDTRVYVAGAPYTPVEHVVFNPNSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAARLSQELAIPYE
EAQAFIERYFQSFPKVRAWIEKTLEEGRRRGYVETLFGRRRYVPDLEARVKSVREAAER
MAFNMPVQGTAADLMKLAMVKLFPRLEEMGARMLLQVHDELVLEAPKERAEAVARL
AKEVMEGVYPLAVPLEVEVGIGEDWLSAKGD
Seq. ID No: 202-TRX-linker-Taq-TBD [C33A, relative to pATG8416]
MSDKIIHLTDDSFDTDVLKADGAILVDFWAPWAGPSKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITPA
WLWEKYGLRPDQWADYRALTGDESDNLPGVKGIGEKTARKLLEEWGSLEALLKNLDR
LKPAIREKILAHMDDLKLSWDLAKVRTDLPLEVDFAKRREPDRERLRAFLERLEFGSLLH
EFGLLESPKALEEAPWPPPEGAFVGFVLSRKEPMWADLLALAAARGGRVHRAPEPYKA
LRDLKEARGLLAKDLSVLALREGLGLPPGDDPMLLAYLLDPSNTTPEGVARRYGGEWT
EEAGERAALSERLFANLWGRLEGEERLLWLYREVERPLSAVLAHMEATGVRLDVAYLR
ALSLEVAEEIARLEAEVFRLAGGSWYQPKGGTEMFLHPRTGKPLPKYPRIKIPKVGGIFK
KPKNKAQREGREPVELDTRVYVAGAPYTPVEHVVFNPNSRDQLERVLFDELGLPAIGKT
EKTGKRSTSAAVLEALREAHPIVEKILQYRELTKLKSTYIDPLPDLIHPRTGRLHTRFNQT
ATATGRLSSSDPNLQNIPVRTPLGQRIRRAFIAEEGWLLVALDYSQIELRVLAHLSGDENL
IRVFQEGRDIHTETASWMFGVPREAVDPLMRRAAKTINFGVLYGMSAARLSQELAIPYE
EAQAFIERYFQSFPKVRAWIEKTLEEGRRRGYVETLFGRRRYVPDLEARVKSVREAAER
MAFNMPVQGTAADLMKLAMVKLFPRLEEMGARMLLQVHDELVLEAPKERAEAVARL
AKEVMEGVYPLAVPLEVEVGIGEDWLSAKGD
Seq. ID No: 203-TRX-linker-Taq-TBD [C33A, T735V, relative to pATG8416]
MSDKIIHLTDDSFDTDVLKADGAILVDFWAPWAGPSKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITP
AWLWEKYGLRPDQWADYRALTGDESDNLPGVKGIGEKTARKLLEEWGSLEALLKNLD
RLKPAIREKILAHMDDLKLSWDLAKVRTDLPLEVDFAKRREPDRERLRAFLERLEFGSL
LHEFGLLESPKALEEAPWPPPEGAFVGFVLSRKEPMWADLLALAAARGGRVHRAPEPY
KALRDLKEARGLLAKDLSVLALREGLGLPPGDDPMLLAYLLDPSNTTPEGVARRYGGE
WTEEAGERAALSERLFANLWGRLEGEERLLWLYREVERPLSAVLAHMEATGVRLDVA
YLRALSLEVAEEIARLEAEVFRLAGGSWYQPKGGTEMFLHPRTGKPLPKYPRIKIPKVGG
IFKKPKNKAQREGREPVELDTRVYVAGAPYVPVEHVVFNPNSRDQLERVLFDELGLPAI
GKTEKTGKRSTSAAVLEALREAHPIVEKILQYRELTKLKSTYIDPLPDLIHPRTGRLHTRF
NQTATATGRLSSSDPNLQNIPVRTPLGQRIRRAFIAEEGWLLVALDYSQIELRVLAHLSG
DENLIRVFQEGRDIHTETASWMFGVPREAVDPLMRRAAKTINFGVLYGMSAARLSQEL
AIPYEEAQAFIERYFQSFPKVRAWIEKTLEEGRRRGYVETLFGRRRYVPDLEARVKSVRE
AAERMAFNMPVQGTAADLMKLAMVKLFPRLEEMGARMLLQVHDELVLEAPKERAEA
VARLAKEVMEGVYPLAVPLEVEVGIGEDWLSAKGD
Seq. ID No: 204-TRX-linker-Taq-TBD [T735C, relative to pATG8416]
MSDKIIHLTDDSFDTDVLKADGAILVDFWAPWCGPSKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITP
AWLWEKYGLRPDQWADYRALTGDESDNLPGVKGIGEKTARKLLEEWGSLEALLKNLD
RLKPAIREKILAHMDDLKLSWDLAKVRTDLPLEVDFAKRREPDRERLRAFLERLEFGSL
LHEFGLLESPKALEEAPWPPPEGAFVGFVLSRKEPMWADLLALAAARGGRVHRAPEPY
KALRDLKEARGLLAKDLSVLALREGLGLPPGDDPMLLAYLLDPSNTTPEGVARRYGGE
WTEEAGERAALSERLFANLWGRLEGEERLLWLYREVERPLSAVLAHMEATGVRLDVA
YLRALSLEVAEEIARLEAEVFRLAGGSWYQPKGGTEMFLHPRTGKPLPKYPRIKIPKVGG
IFKKPKNKAQREGREPVELDTRVYVAGAPYCPVEHVVFNPNSRDQLERVLFDELGLPAI
GKTEKTGKRSTSAAVLEALREAHPIVEKILQYRELTKLKSTYIDPLPDLIHPRTGRLHTRF
NOTATATGRLSSSDPNLQNIPVRTPLGQRIRRAFIAEEGWLLVALDYSQIELRVLAHLSG
DENLIRVFQEGRDIHTETASWMFGVPREAVDPLMRRAAKTINFGVLYGMSAARLSQEL
AIPYEEAQAFIERYFQSFPKVRAWIEKTLEEGRRRGYVETLFGRRRYVPDLEARVKSVRE
AAERMAFNMPVQGTAADLMKLAMVKLFPRLEEMGARMLLQVHDELVLEAPKERAEA
VARLAKEVMEGVYPLAVPLEVEVGIGEDWLSAKGD
Seq. ID No: 205-TRX-linker-Taq-TBD [C33D, relative to pATG8416]
MSDKIIHLTDDSFDTDVLKADGAILVDFWAPWDGPSKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITP
AWLWEKYGLRPDQWADYRALTGDESDNLPGVKGIGEKTARKLLEEWGSLEALLKNLD
RLKPAIREKILAHMDDLKLSWDLAKVRTDLPLEVDFAKRREPDRERLRAFLERLEFGSL
LHEFGLLESPKALEEAPWPPPEGAFVGFVLSRKEPMWADLLALAAARGGRVHRAPEPY
KALRDLKEARGLLAKDLSVLALREGLGLPPGDDPMLLAYLLDPSNTTPEGVARRYGGE
WTEEAGERAALSERLFANLWGRLEGEERLLWLYREVERPLSAVLAHMEATGVRLDVA
YLRALSLEVAEEIARLEAEVFRLAGGSWYQPKGGTEMFLHPRTGKPLPKYPRIKIPKVGG
IFKKPKNKAQREGREPVELDTRVYVAGAPYTPVEHVVFNPNSRDQLERVLFDELGLPAI
GKTEKTGKRSTSAAVLEALREAHPIVEKILQYRELTKLKSTYIDPLPDLIHPRTGRLHTRF
NQTATATGRLSSSDPNLQNIPVRTPLGQRIRRAFIAEEGWLLVALDYSQIELRVLAHLSG
DENLIRVFQEGRDIHTETASWMFGVPREAVDPLMRRAAKTINFGVLYGMSAARLSQEL
AIPYEEAQAFIERYFQSFPKVRAWIEKTLEEGRRRGYVETLFGRRRYVPDLEARVKSVRE
AAERMAFNMPVQGTAADLMKLAMVKLFPRLEEMGARMLLQVHDELVLEAPKERAEA
VARLAKEVMEGVYPLAVPLEVEVGIGEDWLSAKGD
Seq. ID No: 206-TRX-linker-Taq-TBD [C33G, relative to pATG8416]
MSDKIIHLTDDSFDTDVLKADGAILVDFWAPWGGPSKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITP
AWLWEKYGLRPDQWADYRALTGDESDNLPGVKGIGEKTARKLLEEWGSLEALLKNLD
RLKPAIREKILAHMDDLKLSWDLAKVRTDLPLEVDFAKRREPDRERLRAFLERLEFGSL
LHEFGLLESPKALEEAPWPPPEGAFVGFVLSRKEPMWADLLALAAARGGRVHRAPEPY
KALRDLKEARGLLAKDLSVLALREGLGLPPGDDPMLLAYLLDPSNTTPEGVARRYGGE
WTEEAGERAALSERLFANLWGRLEGEERLLWLYREVERPLSAVLAHMEATGVRLDVA
YLRALSLEVAEEIARLEAEVFRLAGGSWYQPKGGTEMFLHPRTGKPLPKYPRIKIPKVGG
IFKKPKNKAQREGREPVELDTRVYVAGAPYTPVEHVVFNPNSRDQLERVLFDELGLPAI
GKTEKTGKRSTSAAVLEALREAHPIVEKILQYRELTKLKSTYIDPLPDLIHPRTGRLHTRF
NOTATATGRLSSSDPNLQNIPVRTPLGQRIRRAFIAEEGWLLVALDYSQIELRVLAHLSG
DENLIRVFQEGRDIHTETASWMFGVPREAVDPLMRRAAKTINFGVLYGMSAARLSQEL
AIPYEEAQAFIERYFQSFPKVRAWIEKTLEEGRRRGYVETLFGRRRYVPDLEARVKSVRE
AAERMAFNMPVQGTAADLMKLAMVKLFPRLEEMGARMLLQVHDELVLEAPKERAEA
VARLAKEVMEGVYPLAVPLEVEVGIGEDWLSAKGD
Seq. ID No: 207-TRX-linker-Taq-TBD [C33P, relative to pATG8416]
MSDKIIHLTDDSFDTDVLKADGAILVDFWAPWPGPSKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITP
AWLWEKYGLRPDQWADYRALTGDESDNLPGVKGIGEKTARKLLEEWGSLEALLKNLD
RLKPAIREKILAHMDDLKLSWDLAKVRTDLPLEVDFAKRREPDRERLRAFLERLEFGSL
LHEFGLLESPKALEEAPWPPPEGAFVGFVLSRKEPMWADLLALAAARGGRVHRAPEPY
KALRDLKEARGLLAKDLSVLALREGLGLPPGDDPMLLAYLLDPSNTTPEGVARRYGGE
WTEEAGERAALSERLFANLWGRLEGEERLLWLYREVERPLSAVLAHMEATGVRLDVA
YLRALSLEVAEEIARLEAEVFRLAGGSWYQPKGGTEMFLHPRTGKPLPKYPRIKIPKVGG
IFKKPKNKAQREGREPVELDTRVYVAGAPYTPVEHVVFNPNSRDQLERVLFDELGLPAI
GKTEKTGKRSTSAAVLEALREAHPIVEKILQYRELTKLKSTYIDPLPDLIHPRTGRLHTRF
NOTATATGRLSSSDPNLQNIPVRTPLGQRIRRAFIAEEGWLLVALDYSQIELRVLAHLSG
DENLIRVFQEGRDIHTETASWMFGVPREAVDPLMRRAAKTINFGVLYGMSAARLSQEL
AIPYEEAQAFIERYFQSFPKVRAWIEKTLEEGRRRGYVETLFGRRRYVPDLEARVKSVRE
AAERMAFNMPVQGTAADLMKLAMVKLFPRLEEMGARMLLQVHDELVLEAPKERAEA
VARLAKEVMEGVYPLAVPLEVEVGIGEDWLSAKGD
Seq. ID No: 208-TRX-linker-Taq-TBD [C33S, T735N, relative to pATG8416]
MSDKIIHLTDDSFDTDVLKADGAILVDFWAPWSGPSKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITP
AWLWEKYGLRPDQWADYRALTGDESDNLPGVKGIGEKTARKLLEEWGSLEALLKNLD
RLKPAIREKILAHMDDLKLSWDLAKVRTDLPLEVDFAKRREPDRERLRAFLERLEFGSL
LHEFGLLESPKALEEAPWPPPEGAFVGFVLSRKEPMWADLLALAAARGGRVHRAPEPY
KALRDLKEARGLLAKDLSVLALREGLGLPPGDDPMLLAYLLDPSNTTPEGVARRYGGE
WTEEAGERAALSERLFANLWGRLEGEERLLWLYREVERPLSAVLAHMEATGVRLDVA
YLRALSLEVAEEIARLEAEVFRLAGGSWYQPKGGTEMFLHPRTGKPLPKYPRIKIPKVGG
IFKKPKNKAQREGREPVELDTRVYVAGAPYNPVEHVVFNPNSRDQLERVLFDELGLPAI
GKTEKTGKRSTSAAVLEALREAHPIVEKILQYRELTKLKSTYIDPLPDLIHPRTGRLHTRF
NQTATATGRLSSSDPNLQNIPVRTPLGQRIRRAFIAEEGWLLVALDYSQIELRVLAHLSG
DENLIRVFQEGRDIHTETASWMFGVPREAVDPLMRRAAKTINFGVLYGMSAARLSQEL
AIPYEEAQAFIERYFQSFPKVRAWIEKTLEEGRRRGYVETLFGRRRYVPDLEARVKSVRE
AAERMAFNMPVQGTAADLMKLAMVKLFPRLEEMGARMLLQVHDELVLEAPKERAEA
VARLAKEVMEGVYPLAVPLEVEVGIGEDWLSAKGD
Seq. ID No: 209-TRX-linker-Taq-TBD [C33S, relative to pATG8416]
MSDKIIHLTDDSFDTDVLKADGAILVDFWAPWSGPSKMIAPILDEIADEYQGKLTVAKL
NIDQNPGTAPKYGIRGIPTLLLFKNGEVAATKVGALSKGQLKEFLDANLAGSSGSSGSSG
GGSGGGSGSSGSSGSSGGGSGGGSGSSGGSGSSGGSSGGGGSGSGSGSSGGSGSSGSGSS
GSSGGGGSGGSSRGMLPLFEPKGRVLLVDGHHLAYRTFHALKGLTTSRGEPVQAVYGF
AKSLLKALKEDGDAVIVVFDAKAPSFRHEAYGGYKAGRAPTPEDFPRQLALIKELVDLL
GLARLEVPGYEADDVLASLAKKAEKEGYEVRILTADKDLYQLLSDRIHVLHPEGYLITP
AWLWEKYGLRPDQWADYRALTGDESDNLPGVKGIGEKTARKLLEEWGSLEALLKNLD
RLKPAIREKILAHMDDLKLSWDLAKVRTDLPLEVDFAKRREPDRERLRAFLERLEFGSL
LHEFGLLESPKALEEAPWPPPEGAFVGFVLSRKEPMWADLLALAAARGGRVHRAPEPY
KALRDLKEARGLLAKDLSVLALREGLGLPPGDDPMLLAYLLDPSNTTPEGVARRYGGE
WTEEAGERAALSERLFANLWGRLEGEERLLWLYREVERPLSAVLAHMEATGVRLDVA
YLRALSLEVAEEIARLEAEVFRLAGGSWYQPKGGTEMFLHPRTGKPLPKYPRIKIPKVGG
IFKKPKNKAQREGREPVELDTRVYVAGAPYTPVEHVVFNPNSRDQLERVLFDELGLPAI
GKTEKTGKRSTSAAVLEALREAHPIVEKILQYRELTKLKSTYIDPLPDLIHPRTGRLHTRF
NOTATATGRLSSSDPNLQNIPVRTPLGQRIRRAFIAEEGWLLVALDYSQIELRVLAHLSG
DENLIRVFQEGRDIHTETASWMFGVPREAVDPLMRRAAKTINFGVLYGMSAARLSQEL
AIPYEEAQAFIERYFQSFPKVRAWIEKTLEEGRRRGYVETLFGRRRYVPDLEARVKSVRE
AAERMAFNMPVQGTAADLMKLAMVKLFPRLEEMGARMLLQVHDELVLEAPKERAEA
VARLAKEVMEGVYPLAVPLEVEVGIGEDWLSAKGD

1. A DNA polymerase system comprising:
(a) a DNA polymerase domain;
(b) a thioredoxin binding domain (TBD), wherein the TBD has non-cysteine amino acids at positions corresponding to positions 14 and 52 of SEQ ID NO: 15; and
(c) a thioredoxin (TRX) domain, wherein the TRX domain has a non-cysteine amino acid at a position corresponding to position 35 of SEQ ID NO: 16.

2. The DNA polymerase system of claim 1, wherein the amino acid at the position corresponding to position 14 of SEQ ID NO: 15 is a leucine.

3. The DNA polymerase system of claim 1, wherein the amino acid at the position corresponding to position 52 of SEQ ID NO: 15 is a valine.

4. The DNA polymerase system of claim 1, wherein the amino acid at the position corresponding to position 35 of SEQ ID NO: 16 is a serine.

5. The DNA polymerase system of claim 1, wherein the TBD has a substitution at a position corresponding to position 66 of SEQ ID NO: 15.

6. The DNA polymerase system of claim 5, wherein the amino acid at a position corresponding to position 66 of SEQ ID NO: 15 is threonine, valine, or cysteine.

7. The DNA polymerase system of claim 1, wherein the TRX has a substitution at a position corresponding to position 32 of SEQ ID NO: 16.

8. The DNA polymerase system of claim 7, wherein the amino acid at a position corresponding to position 32 of SEQ ID NO: 16 is alanine, cysteine, aspartic acid, glycine, proline, or serine.

9. The composition of claim 1, wherein the DNA polymerase system is capable of synthesizing a DNA product from deoxynucleoside triphosphates in the presence of a DNA template and under appropriate reaction conditions.

10. The composition of claim 1, wherein the DNA polymerase system exhibits reduced stutter proclivity compared to a DNA polymerase comprising the DNA polymerase domain in the absence of the TBD and/or TRX.

11. The composition of claim 10, wherein the DNA polymerase system exhibits at least 2-fold reduced stutter proclivity compared to a DNA polymerase comprising the DNA polymerase domain in the absence of the TBD and/or TRX.

12. The composition of claim 1, wherein the DNA polymerase system comprises the DNA polymerase domain conjugated to the TBD and/or TRX.

13. The composition of claim 1, wherein the DNA polymerase system comprises the DNA polymerase domain genetically fused to the TBD and/or TRX.

14. The composition of claim 13, wherein the DNA polymerase system comprises a genetic fusion of the DNA polymerase domain, TBD, and TRX.

15. The composition of claim 1, wherein one or more of the DNA polymerase domain, TBD, and TRX are not conjugated to the other components of the system.

16. The composition of claim 15, comprising a free TRX and a DNA polymerase domain conjugated or genetically fused to a TBD.

17. The composition of claim 15, comprising a free TBD and a DNA polymerase domain conjugated or genetically fused to a TRX.

18. The composition of claim 15, comprising a free DNA polymerase domain and a TRX conjugated or genetically fused to a TBD.

19. The composition of claim 15, comprising a DNA polymerase domain, TRX, and TBD conjugated or genetically fused together.

20-130. (canceled)

131. A method of amplifying a DNA target sequence comprising exposing the composition of claim 1 to amplification reagents sufficient to amplify a DNA target sequence and polymerase chain reaction thermal cycling conditions.

132-159. (canceled)
